(19) United States
(12) Patent Application Publication
(10) Pub. No.: US 2026/0167723 A1
(43) Pub. Date: Jun. 18, 2026

(54) TCR TARGETING MOLECULES AND USES THEREOF

(71) Applicant: Marengo Therapeutics, Inc., Cambridge, MA (US)

(72) Inventors: Madan Katragadda, Acton, MA (US); Andrew Bayliffe, London (GB)

(21) Appl. No.: 19/333,088

(22) Filed: Sep. 18, 2025

Related U.S. Application Data
(63) Continuation of application No. PCT/US24/20796, filed on Mar. 20, 2024.
(60) Provisional application No. 63/491,399, filed on Mar. 21, 2023.

(51) Int. Cl. C07K 16/28; A61K 39/00; A61K 45/06; A61P 37/06

(52) U.S. Cl. CPC C07K 16/2809; A61K 45/06; A61P 37/06; C07K 16/2803; A61K 2039/505; C07K 2317/31; C07K 2317/52; C07K 2317/565; C07K 2317/72; C07K 2317/732; C07K 2317/734

(57) ABSTRACT

Disclosed herein, in some aspects, are methods of treating autoimmune diseases or conditions using TCR targeting molecules or cells expressing TCR targeting molecules. TCR targeting molecules can comprise a moiety that binds to a TCR variable beta chain (TCRBV) or a TCR variable alpha chain (TCRAV). TCR targeting molecules can further comprise one, two or all of: (ii) an immune cell engager; (iii) a cytokine inhibitor molecule; (iv) a cytotoxic agent; and/or (v) a death receptor signal enhancer. Additionally, disclosed herein, in some aspects, are nucleic acids encoding the same, and methods of producing the aforesaid molecules.

### CROSS-REFERENCE TO RELATED APPLICATIONS

This application is a continuation of International Application No. PCT/US2024/020796 filed on Mar. 20, 2024, which claims priority to U.S. Provisional Patent Application No. 63/491,399, filed on Mar. 21, 2023, the entire content of which is incorporated herein by reference.

### REFERENCE TO A SEQUENCE LISTING

The instant application contains a Sequence Listing which has been submitted electronically in XML format and is hereby incorporated by reference in its entirety. Said XML copy, created on Feb. 19, 2026, is named 53676-763_301_SL.xml and is 2,466,417 bytes in size.

### BACKGROUND

T cell mediated antigen recognition depends on the interaction of the T cell receptor (TCR) with the antigen-major histocompatibility complex (MHC). The heterodimeric TCRs consist of a combination of α and β chains (αβ TCR) expressed by the majority of T cells, or γδ chains (γδ TCR) present only in about 1-5% of the T cells. A highly diverse TCR repertoire is a fundamental property of an effective immune system. However, the immune repertoire can change greatly with the onset and progression of diseases, such as cancer, autoimmune, inflammatory, and infectious diseases.

Autoimmunity may result from abnormal regulation of the immune system. This may be manifested by autoreactive TCR clones that attack a patient's own cells. There is a need for improved therapies for autoimmune diseases.

### SUMMARY

Disclosed herein, in some aspects, is a method of treating a disease or condition or reducing a risk of developing the disease or condition in a subject in need thereof, comprising administering to the subject a therapeutically effective amount of an agent comprising a moiety that binds to a TCR variable region,
wherein the TCR variable region is a T cell receptor alpha variable region (TCRαV) or a T cell receptor variable beta region (TCRβV), and
wherein:
   (a) the disease or condition is multiple sclerosis, and the moiety that binds to the TCR variable region binds to a TCRα V1 subfamily;
   (b) the disease or condition is Crohn's disease, and the moiety that binds to the TCR variable region binds to a TCRα V2 subfamily;
   (c) the disease or condition is celiac disease, and the moiety that binds to the TCR variable region binds to a TCRα V4 subfamily;
   (d) the disease or condition is Type I diabetes, and the moiety that binds to the TCR variable region binds to a TCRα V12 subfamily;
   (e) the disease or condition is Sjogren's syndrome, and the moiety that binds to the TCR variable region binds to a TCRα V13 subfamily;
   (f) the disease or condition is celiac disease, and the moiety that binds to the TCR variable region binds to a TCRα V20 subfamily;
   (g) the disease or condition is ankylosing spondylitis, and the moiety that binds to the TCR variable region binds to a TCRα V21 subfamily;
   (h) the disease or condition is Crohn's disease, and the moiety that binds to the TCR variable region binds to a TCRα V22 subfamily;
   (i) the disease or condition is celiac disease, and the moiety that binds to the TCR variable region binds to a TCRα V26 subfamily;
   (j) the disease or condition is Crohn's disease, and the moiety that binds to the TCR variable region binds to a TCRα V40 subfamily;
   (k) the disease or condition is joint implant failure, and the moiety that binds to the TCR variable region binds to a TCRα V41 subfamily;
   (l) the disease or condition is celiac disease, and the moiety that binds to the TCR variable region binds to a TCRβ V4 subfamily;
   (m) the disease or condition is primary Sjogren's syndrome, and the moiety that binds to the TCR variable region binds to a TCRβ V6 subfamily;
   (n) the disease or condition is amyotrophic lateral sclerosis, and the moiety that binds to the TCR variable region binds to a TCRβ V7 subfamily;
   (o) the disease or condition is celiac disease, and the moiety that binds to the TCR variable region binds to a TCRβ V7 subfamily;
   (p) the disease or condition is multiple sclerosis, and the moiety that binds to the TCR variable region binds to a TCRβ V7 subfamily;
   (q) the disease or condition is ankylosing spondylitis, and the moiety that binds to the TCR variable region binds to a TCRβ V9 subfamily;
   (r) the disease or condition is multiple sclerosis, and the moiety that binds to the TCR variable region binds to a TCRβ V10 subfamily;
   (s) the disease or condition is COVID-induced multisystem inflammatory syndrome in children, and the moiety that binds to the TCR variable region binds to a TCRβ VII subfamily;
   (t) the disease or condition is Type I diabetes, and the moiety that binds to the TCR variable region binds to a TCRβ V12 subfamily;
   (u) the disease or condition is multiple sclerosis, and the moiety that binds to the TCR variable region binds to a TCRβ V10 subfamily;
   (v) the disease or condition is Amyotrophic lateral sclerosis, and the moiety that binds to the TCR variable region binds to a TCRβ V23 subfamily; or
   (w) the disease or condition is multiple sclerosis, and the moiety that binds to the TCR variable region binds to a TCRβ V29 subfamily.

In some embodiments, the administration of the therapeutically effective amount of the agent inhibits or prevents activation or expansion of autoreactive T cells in the subject.

In some embodiments, the autoreactive T cells target an autologous cell in the subject.

In some embodiments, the autoreactive T cells express TCRαV and/or TCRβV.

In some embodiments, the agent comprises an antibody molecule or antigen binding domain thereof.

In some embodiments, the antibody molecule comprises an Fc region.

In some embodiments, the Fc region comprises a binding mutation.

In some embodiments, the agent is functionally linked to a cytotoxic agent.

In some embodiments, the cytotoxic agent comprises calicheamicin, monomethyl auristatin E, maytansine derivative, or exatecan derivative.

In some embodiments, the Fc region comprises no binding mutation.

In some embodiments, the cytotoxic agent mediates killing of the autoreactive T cells in the subject.

In some embodiments, the Fc region has enhanced effector function.

In some embodiments, the enhanced effector function comprises antibody dependent cell-mediated cytotoxicity (ADCC), antibody-dependent cellular phagocytosis (ADCP) or complement dependent cytotoxicity (CDC).

In some embodiments, the enhanced effector function mediates killing of the autoreactive T cells in the subject.

In some embodiments, the agent comprises an another moiety.

In some embodiments, the another moiety comprises a NK cell engager, a T cell engager, a B cell engager, a dendritic cell engager, or a macrophage cell engager.

In some embodiments, the another moiety comprises the NK cell engager, and wherein said NK cell engager binds to NKp30.

In some embodiments, binding of the another moiety to NKp30 mediates killing of the autoreactive T cells in the subject.

In some embodiments, the another moiety comprises the T cell engager, and wherein said T cell engager binds to an antigen expressed on a CD8+ and/or CD4+ T cell.

In some embodiments, the another moiety comprises the T cell engager, and wherein said T cell engager binds to a TCRαV or a TCRβV.

In some embodiments, the TCRαV or the TCRβV comprises a different TCRαV or a TCRβV from the TCR variable region that the moiety of the agent binds to.

In some embodiments, the another moiety comprises the T cell engager, and wherein said T cell engager binds to CD3.

In some embodiments, binding of the T cell engager to a CD8+ and/or CD4+ T cell mediates killing of the autoreactive T cells in the subject.

In some embodiments, the disease or condition is multiple sclerosis, and wherein the moiety that binds to the TCR variable region binds to TCRαV1-1 or TCRαV1-2.

In some embodiments, the disease or condition is type I diabetes, and wherein the moiety that binds to the TCR variable region binds to TCRαV12-3.

In some embodiments, the disease or condition is Sjogren's syndrome, and wherein the moiety that binds to the TCR variable region binds to TCRαV13-1 or TCRαV13-2.

In some embodiments, the disease or condition is celiac disease, and wherein the moiety that binds to the TCR variable region binds to TCRαV26-1 or TCRαV26-2.

In some embodiments, the disease or condition is primary Sjogren's syndrome, and wherein the moiety that binds to the TCR variable region binds to TCRβV6-1, TCRβV6-2/3, or TCRβV6-5.

In some embodiments, the disease or condition is amyotrophic lateral sclerosis, and wherein the moiety that binds to the TCR variable region binds to TCRβV7-1 or TCRβV7-8/9.

In some embodiments, the disease or condition is celiac disease, and wherein the moiety that binds to the TCR variable region binds to TCRβV7-1 or TCRβV7-8/9.

In some embodiments, the disease or condition is multiple sclerosis, and wherein the moiety that binds to the TCR variable region binds to TCRβV7-1 or TCRβV7-8/9.

In some embodiments, the disease or condition is multiple sclerosis, and wherein the moiety that binds to the TCR variable region binds to TCRβV10-3.

In some embodiments, the disease or condition is COVID-induced multisystem inflammatory syndrome, and wherein the moiety that binds to the TCR variable region binds to TCRβV11-2.

In some embodiments, the subject is a child.

In some embodiments, the disease or condition is type I diabetes, and wherein the moiety that binds to the TCR variable region binds to TCRβV12-3/4.

In some embodiments, the disease or condition is multiple sclerosis, and wherein the moiety that binds to the TCR variable region binds to TCRβV20-1.

In some embodiments, the disease or condition is multiple sclerosis, and wherein the moiety that binds to the TCR variable region binds to TCRβV29-1.

Disclosed herein, in some aspects, is a composition comprising a recombinant T cell receptor or a chimeric antigen receptor (CAR) comprising a moiety that binds to a TCR variable region.

Disclosed herein, in some aspects, is a composition comprising a T cell comprising a recombinant T cell receptor or a chimeric antigen receptor (CAR), wherein the recombinant T cell receptor or the CAR comprises a moiety that binds to a TCR variable region.

In some embodiments, the recombinant T cell receptor or the CAR comprises
(a) an extracellular domain comprising the moiety that binds to the TCR variable region,
(b) a transmembrane domain; and
(c) an intracellular domain comprising a intracellular signaling domain.

In some embodiments, the extracellular domain comprises a CD8 or CD28 extracellular domain.

In some embodiments, the transmembrane domain comprises a CD8 or CD28 transmembrane domain.

In some embodiments, the intracellular domain comprises a CD3 zeta intracellular signaling domain.

In some embodiments, the moiety that binds to the TCR variable region binds a TCRαV subfamily selected from the group consisting of: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, and a TCRα V41 subfamily, as well as family members of said subfamilies, and variants thereof.

In some embodiments, the moiety that binds to the TCR variable region binds to a TCRβV subfamily selected from the group consisting of: a TCRβ V2 subfamily, a TCRβ V3 subfamily, a TCRβ V4 subfamily, a TCRβ V5 subfamily, a TCRβ V6 subfamily, a TCRβ V9 subfamily, a TCRβ V10 subfamily, a TCRβ V11 subfamily, a TCRβ V12 subfamily, a TCRβ V13 subfamily, a TCRβ V16 subfamily, a TCRβ V19 subfamily, a TCRβ V21 subfamily, a TCRβ V23 subfamily, a TCRβ V27 subfamily, a TCRβ V28 subfamily, TCRβ V29 subfamily, and TCRβ V30 subfamily, as well as family members of said subfamilies, and variants thereof.

In another aspect, provided herein is a pharmaceutical composition comprising a composition disclosed herein, and a pharmaceutically acceptable diluent, carrier, excipient, or stabilizer.

In another aspect, provided herein is a method of treating a disease or condition, or reducing a risk of developing the disease or condition in a subject in need thereof comprising administering a therapeutically effective amount of a pharmaceutical composition disclosed herein to the subject.

In some embodiments, the disease or condition is an autoimmune disease.

In some embodiments, the autoimmune disease is selected from the group consisting of amyotrophic lateral sclerosis (ALS), coeliac disease (CD), ankylosing spondylitis (AS), Covid-induced multisystem inflammatory syndrome in children (MIS-C), primary Sjogren's syndrome (PSS), Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellardegeneration (PCD), Lambert-Eaton syndrome, psoriasis, scleroderma, CREST (calcinosis,Raynaud phenomenon, esophageal dysmotility, sclerodactyly, and telangiectasia) syndrome,dermatitis herpetiformis, dermatomyositis, bullous pemphigoid, cicatricial pemphigoid/benignmucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis),Takayasu's arteritis, polyarteritis nodosa, microscopic polyangiitis (MPA), leukocytoclasticvasculitis, Cogan's syndrome, uveitis, peripheral uveitis (Pars planitis), scleritis, autoimmuneinner ear disease (AIED), Crohn's, ulcerative colitis (UC), Dressler's syndrome, Rheumaticfever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), ocular cicatricial pemphigoid, or Hashimoto's thyroiditis.

In some embodiments:
(a) the disease or condition is multiple sclerosis, and the moiety that binds to the TCR variable region binds to a TCRα V1 subfamily;
(b) the disease or condition is Crohn's disease, and the moiety that binds to the TCR variable region binds to a TCRα V2 subfamily;
(c) the disease or condition is celiac disease, and the moiety that binds to the TCR variable region binds to a TCRα V4 subfamily;
(d) the disease or condition is Type I diabetes, and the moiety that binds to the TCR variable region binds to a TCRα V12 subfamily;
(e) the disease or condition is Sjogren's syndrome, and the moiety that binds to the TCR variable region binds to a TCRα V13 subfamily;
(f) the disease or condition is celiac disease, and the moiety that binds to the TCR variable region binds to a TCRα V20 subfamily;
(g) the disease or condition is ankylosing spondylitis, and the moiety that binds to the TCR variable region binds to a TCRα V21 subfamily;
(h) the disease or condition is Crohn's disease, and the moiety that binds to the TCR variable region binds to a TCRα V22 subfamily;
(i) the disease or condition is celiac disease, and the moiety that binds to the TCR variable region binds to a TCRα V26 subfamily;
(j) the disease or condition is Crohn's disease, and the moiety that binds to the TCR variable region binds to a TCRα V40 subfamily;
(k) the disease or condition is joint implant failure, and the moiety that binds to the TCR variable region binds to a TCRα V41 subfamily;
(1) the disease or condition is celiac disease, and the moiety that binds to the TCR variable region binds to a TCRβ V4 subfamily;
(m) the disease or condition is primary Sjogren's syndrome, and the moiety that binds to the TCR variable region binds to a TCRβ V6 subfamily;
(n) the disease or condition is amyotrophic lateral sclerosis, and the moiety that binds to the TCR variable region binds to a TCRβ V7 subfamily;
(o) the disease or condition is celiac disease, and the moiety that binds to the TCR variable region binds to a TCRβ V7 subfamily;
(p) the disease or condition is multiple sclerosis, and the moiety that binds to the TCR variable region binds to a TCRβ V7 subfamily;
(q) the disease or condition is ankylosing spondylitis, and the moiety that binds to the TCR variable region binds to a TCRβ V9 subfamily;
(r) the disease or condition is multiple sclerosis, and the moiety that binds to the TCR variable region binds to a TCRβ V10 subfamily;
(s) the disease or condition is COVID-induced multisystem inflammatory syndrome in children, and the moiety that binds to the TCR variable region binds to a TCRβ VII subfamily;
(t) the disease or condition is Type I diabetes, and the moiety that binds to the TCR variable region binds to a TCRβ V12 subfamily;
(u) the disease or condition is multiple sclerosis, and the moiety that binds to the TCR variable region binds to a TCRβ V10 subfamily;
(v) the disease or condition is Amyotrophic lateral sclerosis, and the moiety that binds to the TCR variable region binds to a TCRβ V23 subfamily; or
(w) the disease or condition is multiple sclerosis, and the moiety that binds to the TCR variable region binds to a TCRβ V29 subfamily.

In some embodiments, the disease or condition is multiple sclerosis, and wherein the moiety that binds to the TCR variable region binds to TCRαV1-1 or TCRαV1-2.

In some embodiments, the disease or condition is type I diabetes, and wherein the moiety that binds to the TCR variable region binds to TCRαV12-3.

In some embodiments, the disease or condition is Sjogren's syndrome, and wherein the moiety that binds to the TCR variable region binds to TCRαV13-1 or TCRαV13-2.

In some embodiments, the disease or condition is celiac disease, and wherein the moiety that binds to the TCR variable region binds to TCRαV26-1 or TCRαV26-2.

In some embodiments, the disease or condition is primary Sjogren's syndrome, and wherein the moiety that binds to the TCR variable region binds to TCRβV6-1, TCRβV6-2/3, or TCRβV6-5.

In some embodiments, the disease or condition is amyotrophic lateral sclerosis, and wherein the moiety that binds to the TCR variable region binds to TCRβV7-1 or TCRβV7-8/9.

In some embodiments, the disease or condition is celiac disease, and wherein the moiety that binds to the TCR variable region binds to TCRβV7-1 or TCRβV7-8/9.

In some embodiments, the disease or condition is multiple sclerosis, and wherein the moiety that binds to the TCR variable region binds to TCRβV7-1 or TCRβV7-8/9.

In some embodiments, the disease or condition is multiple sclerosis, and wherein the moiety that binds to the TCR variable region binds to TCRβV10-3.

In some embodiments, the disease or condition is COVID-induced multisystem inflammatory syndrome, and wherein the moiety that binds to the TCR variable region binds to TCRβV11-2.

In some embodiments, the subject is a child.

In some embodiments, the disease or condition is type I diabetes, and wherein the moiety that binds to the TCR variable region binds to TCRβV12-3/4.

In some embodiments, the disease or condition is multiple sclerosis, and wherein the moiety that binds to the TCR variable region binds to TCRβV20-1.

In some embodiments, the disease or condition is multiple sclerosis, and wherein the moiety that binds to the TCR variable region binds to TCRβV29-1.

In some embodiments, the methods provided herein further comprise administering a second therapeutic agent or therapy to the subject.

In some embodiments, the second therapeutic agent or therapy comprises a chemotherapeutic agent, a biologic agent, an immunosuppressive agent, or radiation.

In some embodiments, the second therapeutic agent or therapy is administered in combination with an agent provided herein, a composition provided herein or a pharmaceutical composition provided herein, sequentially, simultaneously, or concurrently.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject suffers from multiple sclerosis, and the moiety that binds to the TCR variable region binds to a TCR variable region in TCRα V1 subfamily; (b) the subject suffers from Crohn's disease, and the moiety that binds to the TCR variable region binds to a TCR variable region in TCRα V2 subfamily; (c) the subject suffers from celiac disease, and the moiety that binds to the TCR variable region binds to a TCR variable region in TCRα V4 subfamily; (d) the subject suffers from Type I diabetes, and the moiety that binds to the TCR variable region binds to a TCR variable region in TCRα V12 subfamily; (e) the subject suffers from Sjögren's syndrome, and the moiety that binds to the TCR variable region binds to a TCR variable region in TCRα V13 subfamily; (f) the subject suffers from celiac disease, and the moiety that binds to the TCR variable region binds to a TCR variable region in TCRα V20 subfamily; (g) the subject suffers from ankylosing spondylitis, and the moiety that binds to the TCR variable region binds to a TCR variable region in TCRα V21 subfamily; (h) the subject suffers from Crohn's disease, and the moiety that binds to the TCR variable region binds to a TCR variable region in TCRα V22 subfamily; (i) the subject suffers from celiac disease, and the moiety that binds to the TCR variable region binds to a TCR variable region in TCRα V26 subfamily; (j) the subject suffers from Crohn's disease, and the moiety that binds to the TCR variable region binds to a TCR variable region in TCRα V40 subfamily; (k) the subject suffers from joint implant failure, and the moiety that binds to the TCR variable region binds to a TCR variable region in TCRα V41 subfamily; (1) the subject suffers from celiac disease, and the moiety that binds to the TCR variable region binds to a TCR variable region in TCRβ V4 subfamily; (m) the subject suffers from primary Sjogren's syndrome, and the moiety that binds to the TCR variable region binds to a TCR variable region in TCRβ V6 subfamily; (n) the subject suffers from amyotrophic lateral sclerosis, and the moiety that binds to the TCR variable region binds to a TCR variable region in TCRβ V7 subfamily; (o) the subject suffers from celiac disease, and the moiety that binds to the TCR variable region binds to a TCR variable region in TCRβ V7 subfamily; (p) the subject suffers from multiple sclerosis, and the moiety that binds to the TCR variable region binds to a TCR variable region in TCRβ V7 subfamily; (q) the subject suffers from ankylosing spondylitis, and the moiety that binds to the TCR variable region binds to a TCR variable region in TCRβ V9 subfamily; (r) the subject suffers from multiple sclerosis, and the moiety that binds to the TCR variable region binds to a TCR variable region in TCRβ V10 subfamily; (s) the subject suffers from COVID-induced multisystem inflammatory syndrome in children, and the moiety that binds to the TCR variable region binds to a TCR variable region in TCRβ VII subfamily; (t) the subject suffers from Type I diabetes, and the moiety that binds to the TCR variable region binds to a TCR variable region in TCRβ V12 subfamily; (u) the subject suffers from multiple sclerosis, and the moiety that binds to the TCR variable region binds to a TCR variable region in TCRβ V10 subfamily; (v) the subject suffers from Amyotrophic lateral sclerosis, and the moiety that binds to the TCR variable region binds to a TCR variable region in TCRβ V23 subfamily; or (w) the subject suffers from multiple sclerosis, and the moiety that binds to the TCR variable region binds to a TCR variable region in TCRβ V29 subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject suffers from multiple sclerosis, and the moiety that binds to the TCR variable region binds to a TCR variable region in TCRα V1 subfamily; (b) the subject suffers from Crohn's disease, and the moiety that binds to the TCR variable region binds to a TCR variable region in TCRα V2 subfamily; (c) the subject suffers from celiac disease, and the moiety that binds to the TCR variable region binds to a TCR variable region in TCRα V4 subfamily; (d) the subject suffers from Type I diabetes, and the moiety that binds to the TCR variable region binds to a TCR variable region in TCRα V12 subfamily; (e) the subject suffers from Sjögren's syndrome, and the moiety that binds to the TCR variable region binds to a TCR variable region in TCRα V13 subfamily; (f) the subject suffers from celiac disease, and the moiety that binds to the TCR variable region binds to a TCR variable region in TCRα V20 subfamily; (g) the subject suffers from ankylosing spondylitis, and the moiety that binds to the TCR variable region binds to a TCR variable region in TCRα V21 subfamily; (h) the subject suffers from Crohn's disease, and the moiety that binds to the TCR variable region binds to a TCR variable region in TCRα V22 subfamily; (i) the subject suffers from celiac disease, and the moiety that binds to the TCR variable region binds to a TCR variable region in TCRα V26 subfamily; j) the subject suffers from Crohn's disease, and the moiety that binds to the TCR variable region binds to a TCR variable region in TCRα V40 subfamily; (k) the subject suffers from joint implant failure, and the moiety that binds to the TCR variable region binds to a TCR variable region in TCRα V41 subfamily; (1) the subject suffers from celiac disease, and the moiety that binds to the TCR variable region binds to a TCR variable region in TCRβ V4 subfamily; (m) the subject suffers from primary Sjogren's syndrome, and the moiety that binds to the TCR variable region binds to a TCR variable region in TCRβ V6 subfamily; (n) the subject suffers from amyotrophic lateral sclerosis, and the moiety that binds to the TCR variable region binds to a TCR variable region in TCRβ V7 subfamily; (o) the subject suffers from celiac disease, and the moiety that binds to the TCR variable region binds to a TCR variable region in TCRβ V7 subfamily; (p) the subject suffers from multiple sclerosis, and the moiety that binds to the TCR variable region binds to a TCR variable region in TCRβ V7 subfamily; (q) the subject suffers from ankylosing spondylitis, and the moiety that binds to the TCR variable region binds to a TCR variable region in TCRβ V9 subfamily; (r) the subject suffers from multiple sclerosis, and the moiety that binds to the TCR variable region binds to a TCR variable region in TCRβ V10 subfamily; (s) the subject suffers from COVID-induced multisystem inflammatory syndrome in children, and the moiety that binds to the TCR variable region binds to a TCR variable region in TCRβ VII subfamily; (t) the subject suffers from Type I diabetes, and the moiety that binds to the TCR variable region binds to a TCR variable region in TCRβ V12 subfamily; (u) the subject suffers from multiple sclerosis, and the moiety that binds to the TCR variable region binds to a TCR variable region in TCRβ V10 subfamily; (v) the subject suffers from Amyotrophic lateral sclerosis, and the moiety that binds to the TCR variable region binds to a TCR variable region in TCRβ V23 subfamily; or (w) the subject suffers from multiple sclerosis, and the moiety that binds to the TCR variable region binds to a TCR variable region in TCRβ V29 subfamily.

In some embodiments, the method further comprises determining that the subject suffers from multiple sclerosis that is associated with a biased TCR clonotype that comprises the TCR variable region in TCRα V1 subfamily, TCRβ V7 subfamily, TCRβ V10 subfamily, TCRβ V20 subfamily or TCRβ V29 subfamily, and wherein the moiety that binds to the TCR variable region binds to the TCR variable domain in TCRα V1 subfamily, TCRβ V7 subfamily, TCRβ V10 subfamily, TCRβ V20 subfamily or TCRβ V29 subfamily.

In some embodiments, the method further comprises determining that the subject suffers from amyotrophic lateral sclerosis that is associated with a biased TCR clonotype that comprises the TCR variable region in TCRβ V7 subfamily or TCRβ V23 subfamily, and wherein the moiety that binds to the TCR variable region binds to the TCR variable domain in TCRβ V7 subfamily or TCRβ V23 subfamily.

In some embodiments, the method further comprises determining that the subject suffers from celiac disease that is associated with a biased TCR clonotype that comprises the TCR variable region in TCRα V26 subfamily, TCRα V4 subfamily, TCRβ V7 subfamily, or TCRβ V4 subfamily, and wherein the moiety that binds to the TCR variable region binds to the TCR variable domain in TCRα V26 subfamily, TCRα V4 subfamily, TCRβ V7 subfamily, or TCRβ V4 subfamily.

In some embodiments, the method further comprises determining that the subject suffers from ankylosing spondylitis that is associated with a biased TCR clonotype that comprises the TCR variable region in TCRα V21 subfamily or TCRβ V9 subfamily, and wherein the moiety that binds to the TCR variable region binds to the TCR variable domain in TCRα V21 subfamily or TCRβ V9 subfamily.

In some embodiments, the method further comprises determining that the subject suffers from COVID-induced multisystem inflammatory syndrome in children that is associated with a biased TCR clonotype that comprises the TCR variable region in TCRβ VII subfamily, and wherein the moiety that binds to the TCR variable region binds to the TCR variable domain in TCRβ VII subfamily.

In some embodiments, the method further comprises determining that the subject suffers from primary Sjogren's syndrome that is associated with a biased TCR clonotype that comprises the TCR variable region in TCRβ V6 subfamily, and wherein the moiety that binds to the TCR variable region binds to the TCR variable domain in TCRβ V6 subfamily.

In some embodiments, the method further comprises determining that the subject suffers from Type I diabetes that is associated with a biased TCR clonotype that comprises the TCR variable region in TCRα V12 subfamily or TCRβ V12 subfamily, and wherein the moiety that binds to the TCR variable region binds to the TCR variable domain in TCRα V12 subfamily or TCRβ V12 subfamily.

In some embodiments, the agent comprises an antibody molecule or antigen binding fragment thereof. In some embodiments, the antibody molecule comprises an Fc region.

In some embodiments, the Fc region comprises a binding mutation. In some embodiments, the agent is functionally linked to a cytotoxic agent. In some embodiments, the cytotoxic agent comprises calicheamicin, monomethyl auristatin E, maytansine derivative, or exatecan derivative.

In some embodiments, the Fc region comprises no mutation that affects binding of the Fc region to a Fc receptor. In some embodiments, the cytotoxic agent mediates killing of the autoreactive T cells in the subject. In some embodiments, the Fc region comprises an amino acid mutation that enhances an effector function. In some embodiments, the effector function comprises antibody dependent cell-mediated cytotoxicity (ADCC), antibody-dependent cellular phagocytosis (ADCP) or complement dependent cytotoxicity (CDC). In some embodiments, the effector function mediates killing of the autoreactive T cells in the subject.

In some embodiments, the agent comprises an another moiety. In some embodiments, the another moiety comprises a NK cell engager, a T cell engager, a B cell engager, a dendritic cell engager, or a macrophage cell engager. In some embodiments, the another moiety comprises the NK cell engager, and wherein the NK cell engager binds to an antigen selected from the group consisting of NKp30, NKp40, NKp44, NKp46, NKG2D, DNAM1, DAP10, CD16, CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, and CD160. In some embodiments, binding of the another moiety to NKp30 activates an NK cell and mediates killing of the autoreactive T cells in the subject. In some embodiments, the another moiety comprises the NK cell engager, and wherein the NK cell engager binds to a KIR receptor and/or CD94-NKG2A. In some embodiments, binding of the another moiety to the KIR receptor and/or CD94-NKG2A disinhibits an NK cell and mediates killing of the autoreactive T cells in the subject.

In some embodiments, the another moiety comprises the T cell engager, and wherein the T cell engager binds to an antigen expressed on a CD8+ and/or CD4+ T cell. In some embodiments, the another moiety comprises the T cell engager, and wherein the T cell engager binds to a TCRαV or a TCRβV. In some embodiments, the TCRαV or the TCRβV that the T cell engager binds to is different from the TCR variable region that the moiety of the agent binds to. In some embodiments, the another moiety that binds to the TCRαV or a TCRβV binds a TCRαV in a subfamily selected from the group consisting of: TCRα V1 subfamily, TCRα V2 subfamily, TCRα V3 subfamily, TCRα V4, TCRα V5 subfamily, TCRα V6 subfamily, TCRα V7 subfamily, TCRα V8 subfamily, TCRα V9 subfamily, TCRα V10 subfamily, TCRα V12 subfamily, TCRα V13 subfamily, TCRα V14 subfamily, TCRα V16 subfamily, TCRα V17 subfamily, TCRα V18 subfamily, TCRα V19 subfamily, TCRα V20 subfamily, TCRα V21 subfamily, TCRα V22 subfamily, TCRα V23 subfamily, TCRα V24 subfamily, TCRα V25 subfamily, TCRα V26 subfamily, TCRα V27 subfamily, TCRα V29 subfamily, TCRα V30 subfamily, TCRα V34 subfamily, TCRα V35 subfamily, TCRα V36 subfamily, TCRα V38 subfamily, TCRα V39 subfamily, TCRα V40 subfamily, and TCRα V41 subfamily, as well as family members of the subfamilies, and variants thereof.

In some embodiments, the another moiety that binds to the TCRαV or the TCRβV binds to a TCRβV in a subfamily selected from the group consisting of: TCRβ V2 subfamily, TCRβ V3 subfamily, TCRβ V4 subfamily, TCRβ V5 subfamily, TCRβ V6 subfamily, TCRβ V9 subfamily, TCRβ V10 subfamily, TCRβ V11 subfamily, TCRβ V12 subfamily, TCRβ V13 subfamily, TCRβ V16 subfamily, TCRβ V19 subfamily, TCRβ V21 subfamily, TCRβ V23 subfamily, TCRβ V27 subfamily, TCRβ V28 subfamily, TCRβ V29 subfamily, and TCRβ V30 subfamily, as well as family members of the subfamilies, and variants thereof.

In some embodiments, the another moiety comprises the T cell engager, and wherein the T cell engager binds to CD3.

In some embodiments, binding of the T cell engager to the CD8+ and/or CD4+ T cell mediates killing of the autoreactive T cells in the subject.

In some embodiments, the subject suffers from multiple sclerosis, and wherein the moiety that binds to the TCR variable region binds to the TCR variable region in TCRαV1-1 or TCRαV1-2 family. In some embodiments, the subject suffers from type I diabetes, and wherein the moiety that binds to the TCR variable region binds to the TCR variable region in TCRαV12-3 family. In some embodiments, the subject suffers from Sjogren's syndrome, and wherein the moiety that binds to the TCR variable region binds to the TCR variable region in TCRαV13-1 or TCRαV13-2 family. In some embodiments, the subject suffers from celiac disease, and wherein the moiety that binds to the TCR variable region binds to the TCR variable region in TCRαV26-1 or TCRαV26-2 family. In some embodiments, the subject suffers from primary Sjogren's syndrome, and wherein the moiety that binds to the TCR variable region binds to the TCR variable region in TCRβV6-1, TCRβV6-2/3, or TCRβV6-5 family. In some embodiments, the subject suffers from amyotrophic lateral sclerosis, and wherein the moiety that binds to the TCR variable region binds to the TCR variable region in TCRβV7-1 or TCRβV7-8/9 family. In some embodiments, the subject suffers from celiac disease, and wherein the moiety that binds to the TCR variable region binds to the TCR variable region in TCRβV7-1 or TCRβV7-8/9 family. In some embodiments, the subject suffers from multiple sclerosis, and wherein the moiety that binds to the TCR variable region binds to the TCR variable region in TCRβV7-1 or TCRβV7-8/9 family. In some embodiments, the subject suffers from multiple sclerosis, and wherein the moiety that binds to the TCR variable region binds to the TCR variable region in TCRβV10-3 family. In some embodiments, the subject suffers from COVID-induced multisystem inflammatory syndrome, and wherein the moiety that binds to the TCR variable region binds to the TCR variable region in TCRβV11-2 family. In some embodiments, the subject is a child. In some embodiments, the subject suffers from type I diabetes, and wherein the moiety that binds to the TCR variable region binds to the TCR variable region in TCRβV12-3/4 family. In some embodiments, the subject suffers from multiple sclerosis, and wherein the moiety that binds to the TCR variable region binds to the TCR variable region in TCRβV20-1 family. In some embodiments, the subject suffers from multiple sclerosis, and wherein the moiety that binds to the TCR variable region binds to the TCR variable region in TCRβV29-1 family.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having multiple sclerosis or a symptom of multiple sclerosis that is associated with a biased TCR clonotype that comprises the TCR variable region in a TCRα V1 subfamily, and wherein the moiety that binds to the TCR variable region binds to the TCR variable region in the TCRα V1 subfamily, (b) the subject is identified as having multiple sclerosis or a symptom of multiple sclerosis that is associated with a biased TCR clonotype that comprises the TCR variable region in a TCRβ V7 subfamily, and wherein the moiety that binds to the TCR variable region binds to the TCR variable region in the TCRβ V7 subfamily, (c) the subject is identified as having multiple sclerosis or a symptom of multiple sclerosis that is associated with a biased TCR clonotype that comprises the TCR variable region in a TCRβ V10 subfamily, and wherein the moiety that binds to the TCR variable region binds to the TCR variable region in the TCRβ V10 subfamily, (d) the subject is identified as having multiple sclerosis or a symptom of multiple sclerosis that is associated with a biased TCR clonotype that comprises the TCR variable region in a TCRβ V20 subfamily, and wherein the moiety that binds to the TCR variable region binds to the TCR variable region in the TCRβ V20 subfamily, or (e) the subject is identified as having multiple sclerosis or a symptom of multiple sclerosis that is associated with a biased TCR clonotype that comprises the TCR variable region in a TCRβ V29 subfamily, and wherein the moiety that binds to the TCR variable region binds to the TCR variable region in the TCRβ V29 subfamily, and wherein the moiety does not bind to a different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having multiple sclerosis or a symptom of multiple sclerosis that is associated with a biased TCR clonotype that comprises the TCR variable region in a TCRα V1 subfamily, and wherein the moiety that binds to the TCR variable region binds to the TCR variable region in the TCRα V1 subfamily, (b) the subject is identified as having multiple sclerosis or a symptom of multiple sclerosis that is associated with a biased TCR clonotype that comprises the TCR variable region in a TCRβ V7 subfamily, and wherein the moiety that binds to the TCR variable region binds to the TCR variable region in the TCRβ V7 subfamily, (c) the subject is identified as having multiple sclerosis or a symptom of multiple sclerosis that is associated with a biased TCR clonotype that comprises the TCR variable region in a TCRβ V10 subfamily, and wherein the moiety that binds to the TCR variable region binds to the TCR variable region in the TCRβ V10 subfamily, (d) the subject is identified as having multiple sclerosis or a symptom of multiple sclerosis that is associated with a biased TCR clonotype that comprises the TCR variable region in a TCRβ V20 subfamily, and wherein the moiety that binds to the TCR variable region binds to the TCR variable region in the TCRβ V20 subfamily, or (e) the subject is identified as having multiple sclerosis or a symptom of multiple sclerosis that is associated with a biased TCR clonotype that comprises the TCR variable region in a TCRβ V29 subfamily, and wherein the moiety that binds to the TCR variable region binds to the TCR variable region in the TCRβ V29 subfamily, and wherein the moiety does not bind to a different TCRαV and/or TCRβV subfamily.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having amyotrophic lateral sclerosis or a symptom of amyotrophic lateral sclerosis that is associated with a biased TCR clonotype that comprises the TCR variable region in TCRβ V7 subfamily or TCRβ V23 subfamily, and wherein the moiety that binds to the TCR variable region binds to the TCR variable domain in TCRβ V7 subfamily or TCRβ V23 subfamily, or (b) the subject is identified as having amyotrophic lateral sclerosis or a symptom of amyotrophic lateral sclerosis that is associated with a biased TCR clonotype that comprises the TCR variable region in TCRβ V7 subfamily or TCRβ V23 subfamily, and wherein the moiety that binds to the TCR variable region binds to the TCR variable domain in TCRβ V7 subfamily or TCRβ V23 subfamily, and wherein the moiety does not bind to any other different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having amyotrophic lateral sclerosis or a symptom of amyotrophic lateral sclerosis that is associated with a biased TCR clonotype that comprises the TCR variable region in TCRβ V7 subfamily or TCRβ V23 subfamily, and wherein the moiety that binds to the TCR variable region binds to the TCR variable domain in TCRβ V7 subfamily or TCRβ V23 subfamily, or (b) the subject is identified as having amyotrophic lateral sclerosis or a symptom of amyotrophic lateral sclerosis that is associated with a biased TCR clonotype that comprises the TCR variable region in TCRβ V7 subfamily or TCRβ V23 subfamily, and wherein the moiety that binds to the TCR variable region binds to the TCR variable domain in TCRβ V7 subfamily or TCRβ V23 subfamily, and wherein the moiety does not bind to any other different TCRαV and/or TCRβV subfamily.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having celiac disease or a symptom of celiac disease that is associated with a biased TCR clonotype that comprises the TCR variable region in TCRα V26 subfamily, TCRα V4 subfamily, TCRβ V7 subfamily, or TCRβ V4 subfamily, and wherein the moiety that binds to the TCR variable region binds to the TCR variable domain in TCRα V26 subfamily, TCRα V4 subfamily, TCRβ V7 subfamily, or TCRβ V4 subfamily, or (b) the subject is identified as having celiac disease or a symptom of celiac disease that is associated with a biased TCR clonotype that comprises the TCR variable region in TCRα V26 subfamily, TCRα V4 subfamily, TCRβ V7 subfamily, or TCRβ V4 subfamily, and wherein the moiety that binds to the TCR variable region binds to the TCR variable domain in TCRα V26 subfamily, TCRα V4 subfamily, TCRβ V7 subfamily, or TCRβ V4 subfamily, and wherein the moiety does not bind to any other different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having celiac disease or a symptom of celiac disease that is associated with a biased TCR clonotype that comprises the TCR variable region in TCRα V26 subfamily, TCRα V4 subfamily, TCRβ V7 subfamily, or TCRβ V4 subfamily, and wherein the moiety that binds to the TCR variable region binds to the TCR variable domain in TCRα V26 subfamily, TCRα V4 subfamily, TCRβ V7 subfamily, or TCRβ V4 subfamily, or (b) the subject is identified as having celiac disease or a symptom of celiac disease that is associated with a biased TCR clonotype that comprises the TCR variable region in TCRα V26 subfamily, TCRα V4 subfamily, TCRβ V7 subfamily, or TCRβ V4 subfamily, and wherein the moiety that binds to the TCR variable region binds to the TCR variable domain in TCRα V26 subfamily, TCRα V4 subfamily, TCRβ V7 subfamily, or TCRβ V4 subfamily, and wherein the moiety does not bind to any other different TCRαV and/or TCRβV subfamily.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having ankylosing spondylitis or a symptom of ankylosing spondylitis that is associated with a biased TCR clonotype that comprises the TCR variable region in TCRα V21 subfamily or TCRβ V9 subfamily, and wherein the moiety that binds to the TCR variable region binds to the TCR variable domain in TCRα V21 subfamily or TCRβ V9 subfamily, or (b) the subject is identified as having ankylosing spondylitis or a symptom of ankylosing spondylitis that is associated with a biased TCR clonotype that comprises the TCR variable region in TCRα V21 subfamily or TCRβ V9 subfamily, and wherein the moiety that binds to the TCR variable region binds to the TCR variable domain in TCRα V21 subfamily or TCRβ V9 subfamily, and wherein the moiety does not bind to any other different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having ankylosing spondylitis or a symptom of ankylosing spondylitis that is associated with a biased TCR clonotype that comprises the TCR variable region in TCRα V21 subfamily or TCRβ V9 subfamily, and wherein the moiety that binds to the TCR variable region binds to the TCR variable domain in TCRα V21 subfamily or TCRβ V9 subfamily, or (b) the subject is identified as having ankylosing spondylitis or a symptom of ankylosing spondylitis that is associated with a biased TCR clonotype that comprises the TCR variable region in TCRα V21 subfamily or TCRβ V9 subfamily, and wherein the moiety that binds to the TCR variable region binds to the TCR variable domain in TCRα V21 subfamily or TCRβ V9 subfamily, and wherein the moiety does not bind to any other different TCRαV and/or TCRβV subfamily.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having COVID-induced multisystem inflammatory syndrome in children or a symptom of COVID-induced multisystem inflammatory syndrome in children that is associated with a biased TCR clonotype that comprises the TCR variable region in TCRβ VII subfamily, and wherein the moiety that binds to the TCR variable region binds to the TCR variable domain in TCRβ VII subfamily, or (b) the subject is identified as having COVID-induced multisystem inflammatory syndrome in children or a symptom of COVID-induced multisystem inflammatory syndrome in children that is associated with a biased TCR clonotype that comprises the TCR variable region in TCRβ VII subfamily, and wherein the moiety that binds to the TCR variable region binds to the TCR variable domain in TCRβ VII subfamily, and wherein the moiety does not bind to any other different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having COVID-induced multisystem inflammatory syndrome in children or a symptom of COVID-induced multisystem inflammatory syndrome in children that is associated with a biased TCR clonotype that comprises the TCR variable region in TCRβ VII subfamily, and wherein the moiety that binds to the TCR variable region binds to the TCR variable domain in TCRβ VII subfamily, or (b) the subject is identified as having COVID-induced multisystem inflammatory syndrome in children or a symptom of COVID-induced multisystem inflammatory syndrome in children that is associated with a biased TCR clonotype that comprises the TCR variable region in TCRβ VII subfamily, and wherein the moiety that binds to the TCR variable region binds to the TCR variable domain in TCRβ VII subfamily, and wherein the moiety does not bind to any other different TCRαV and/or TCRβV subfamily.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having primary Sjogrens syndrome or a symptom of primary Sjogrens syndrome that is associated with a biased TCR clonotype that comprises the TCR variable region in TCRβ V6 subfamily, and wherein the moiety that binds to the TCR variable region binds to the TCR variable domain in TCRβ V6 subfamily, or (b) the subject is identified as having primary Sjogrens syndrome or a symptom of primary Sjogrens syndrome that is associated with a biased TCR clonotype that comprises the TCR variable region in TCRβ V6 subfamily, and wherein the moiety that binds to the TCR variable region binds to the TCR variable domain in TCRβ V6 subfamily, and wherein the moiety does not bind to any other different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having primary Sjogrens syndrome or a symptom of primary Sjogrens syndrome that is associated with a biased TCR clonotype that comprises the TCR variable region in TCRβ V6 subfamily, and wherein the moiety that binds to the TCR variable region binds to the TCR variable domain in TCRβ V6 subfamily, or (b) the subject is identified as having primary Sjogrens syndrome or a symptom of primary Sjogrens syndrome that is associated with a biased TCR clonotype that comprises the TCR variable region in TCRβ V6 subfamily, and wherein the moiety that binds to the TCR variable region binds to the TCR variable domain in TCRβ V6 subfamily, and wherein the moiety does not bind to any other different TCRαV and/or TCRβV subfamily.

In some aspects, provided herein is a method of inhibiting or preventing activation and/or expansion of autoreactive T cells in a subject in need thereof, the method comprising administering to the subject a therapeutically effective amount of an agent comprising a moiety that binds to a TCR variable region, thereby inhibiting or preventing the activation and/or expansion of the autoreactive T cells in the subject, wherein the TCR variable region is a T cell receptor alpha variable region (TCRαV) or a T cell receptor beta variable region (TCRβV), wherein the autoreactive T cells target an autologous cell in the subject, wherein the autoreactive T cells express TCRαV and/or TCRβV, wherein: (a) the subject is identified as having Type I diabetes or a symptom Type I diabetes that is associated with a biased TCR clonotype that comprises the TCR variable region in TCRβ V12 subfamily or TCRα V12 subfamily, and wherein the moiety that binds to the TCR variable region binds to the TCR variable domain in TCRβ V12 subfamily or TCRα V12 subfamily, or (b) the subject is identified as having Type I diabetes or a symptom Type I diabetes that is associated with a biased TCR clonotype that comprises the TCR variable region in TCRβ V12 subfamily or TCRα V12 subfamily, and wherein the moiety that binds to the TCR variable region binds to the TCR variable domain in TCRβ V12 subfamily or TCRα V12 subfamily, and wherein the moiety does not bind to any other different TCRαV and/or TCRβV subfamily.

In some aspects, provided herein is a method of reducing or depleting autoreactive T cells in a subject in need thereof, the method comprising administering to the subject a therapeutically effective amount of an agent comprising a moiety that binds to a TCR variable region, thereby inhibiting or preventing the activation and/or expansion of autoreactive T cells in the subject, wherein the TCR variable region is a T cell receptor alpha variable region (TCRαV) or a T cell receptor variable beta region (TCRβV), wherein the autoreactive T cells target an autologous cell in the subject, wherein the autoreactive T cells express TCRαV and/or TCRβV, and wherein: (a) the subject is identified as having Type I diabetes or a symptom Type I diabetes that is associated with a biased TCR clonotype that comprises the TCR variable region in TCRβ V12 subfamily or TCRα V12 subfamily, and wherein the moiety that binds to the TCR variable region binds to the TCR variable domain in TCRβ V12 subfamily or TCRα V12 subfamily, or (b) the subject is identified as having Type I diabetes or a symptom Type I diabetes that is associated with a biased TCR clonotype that comprises the TCR variable region in TCRβ V12 subfamily or TCRα V12 subfamily, and wherein the moiety that binds to the TCR variable region binds to the TCR variable domain in TCRβ V12 subfamily or TCRα V12 subfamily, and wherein the moiety does not bind to any other different TCRαV and/or TCRβV subfamily.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B show the structure and sequence of eight TCRβV proteins from seven different subfamilies: TCRβV6 subfamily (TCRβV6-5 and TCRβV6-4 are shown), TCRβV28 subfamily, TCRβV19 subfamily, TCRβV9 subfamily, TCRβV5 subfamily, TCRβV20 subfamily and TCRβV12 subfamily. FIG. 1A shows the structural alignment of the different TCRβV proteins. The circled area represents the outward facing region comprising the proposed binding site for the anti-TCRβV antibodies as described herein. FIG. 1B shows the amino acid sequence alignment of the proteins shown in FIG. 1A (full-length sequences disclosed as SEQ ID NOS 3449-3456, respectively, in order of appearance and “3MFG” fragment disclosed as SEQ ID NO: 7517). The various TCRαV proteins (from 7 different TCRαV subfamilies) have diverse sequences but share a conserved (similar) structure and function.

FIG. 2 shows an exemplary embodiment of an anti-TCRαV molecule binding to an autoreactive T cell, e.g., a T cell expressing a TCRαV associated with an autoimmune disease, in order to inhibit the activation of or to kill the autoreactive T cell.

FIG. 3 shows an exemplary embodiment of an anti-TCRαV molecule functionally linked to a cytotoxic agent. The derivatized molecule can bind to a target autoreactive T cell, e.g. a T cell expressing a TCRαV associated with an autoimmune disease, and initiate killing of the target autoreactive T cell.

FIG. 4 shows an exemplary embodiment of a multifunctional molecule engineered to bind to a TCRαV on a target autoreactive T cell and to further contain a natural killer (NK) cell engager that binds to NKp30 expressed on NK cell.

FIG. 5 shows an exemplary embodiment of a multispecific molecule that binds to a TCRαV or TCRβV on a target autoreactive T cell and and to further contain a T cell engager that binds to a CD3 complex on a healthy effector T cell in the subject.

FIG. 6 shows an exemplary embodiment of an antibody molecule specific for TCRαV and/or TCRβV with Fe mutations to increase effector functions, such as antibody dependent cell-mediated cytotoxicity (ADCC), antibody-dependent cellular phagocytosis (ADCP), and/or complement dependent cytotoxicity (CDC).

FIG. 7 shows a representation of a CAR T cell with an antigen recognition domain for a TCRαV and/or TCRβV of a target autoreactive T cell, where the T cell expresses a TCRαV and/or TCRβV associated with an autoimmune disease.

FIG. 8 shows the frequency of TRBV13-1, 13-2, 13-3 transcript counts relative to all TRBV counts. Two animals per timepoint were dosed.

### DETAILED DESCRIPTION

Disclosed herein, in some aspects, are methods of treating autoimmune diseases or conditions using TCR targeting molecules or cells expressing TCR targeting molecules. TCR targeting molecules can comprise a moiety that binds to a TCR variable beta chain (TCRBV) or a TCR variable alpha chain (TCRAV). TCR targeting molecules can further comprise one, two or all of: (ii) an immune cell engager; (iii) a cytokine inhibitor molecule; (iv) a cytotoxic agent; and/or (v) a death receptor signal enhancer.

Without wishing to be bound by theory, a TCR bias may exist in autoimmune diseases. This bias may be associated with dominant autoreactive TCR clones responsible for disease or associated with symptoms. Re-balancing the TCR repertoire, e.g., by eliminating or depleting T cells comprising an autoreactive clonotype, may treat the associated autoimmune disease and/or reduce symptoms of the autoimmune disease. Accordingly, the multispecific or multifunctional molecules disclosed herein are expected to target (e.g., localize, bridge and/or activate) an immune cell (e.g., an immune effector cell chosen from an NK cell, a T cell, a B cell, a dendritic cell or a macrophage), at a target cell (e.g., a T cell comprising a biased TCRAV or TCRBV clonotype or comprising a TCRAV or TCRBV antigen corresponding to a biased TCRAV or TCRBV clonotype). Increasing the proximity and/or activity of the immune cell using the multispecific molecules described herein is expected to enhance an immune response against the target cell (e.g., the T cell comprising a TCRAV or TCRBV, e.g., a TCRAV or TCRBV antigen corresponding to a biased TCRAV or TCRBV clonotype), thereby providing a more effective therapy (e.g., a more effective autoimmune disease therapy). Without being bound by theory, a targeted, localized immune response against the target cell (e.g., a T cell comprising a biased TCRAV or TCRBV clonotype is believed to reduce the effects of systemic toxicity of the multispecific molecules described herein. A targeted immune response against the autoreactive T cell population that targets non-autoreactive T cells to a lesser degree (e.g., does not target non-autoreactive T cells) is believed to have fewer deleterious effects than systemic ablation of all T cells.

Accordingly, provided herein are, inter alia, TCR targeting molecules (e.g., multispecific or multifunctional antibody molecules) that include the aforesaid moieties, nucleic acids encoding the same, methods of producing the aforesaid molecules, and methods of treating autoimmune disease using the aforesaid molecules. Also provided herein are anti-TCRαV or anti-TCRβV antibody molecules, nucleic acids encoding the same, methods of producing the aforesaid molecules, and methods of treating autoimmune disease using the anti-TCRαV or anti-TCRAV antibody molecules.

Further provided are methods for depletion (e.g., in vivo depletion) of biased TCRBV clonotypes, e.g., in the context of autoimmune disease with a multispecific molecule or an anti-TCRαV or anti-TCRβV antibody molecule. In some embodiments, the method involves identifying in a patient a clonal bias in TCRAV or TCRBV usage, e.g., associated with the autoreactive subpopulation, and responsive to this analysis administering a multifunctional molecule targeted to the TCRAV or TCRBV antigen corresponding to the biased TCRAV or TCRBV clonotype to decrease, e.g., eliminate, the clonal bias and promote, e.g., establish, a normal TCRBV distribution.

Accordingly, in one aspect, the disclosure features a multifunctional molecule, comprising: (i) a first antigen binding domain that binds to, e.g., selectively binds to, T cell receptor variable alpha (TCRAV), e.g., a TCRAV antigen, and (ii) one, two, or all of: (a) an immune cell engager chosen from an NK cell engager, a T cell engager, a B cell engager, a dendritic cell engager, or a macrophage cell engager; (b) a cytokine molecule or cytokine inhibitor molecule; and (c) a death receptor signal engager.

In another aspect, the disclosure features a multifunctional molecule, comprising: (i) a first antigen binding domain that binds to, e.g., selectively binds to, T cell receptor variable beta (TCRBV), e.g., a TCRBV antigen, and (ii) one, two, or all of: (a) an immune cell engager chosen from an NK cell engager, a T cell engager, a B cell engager, a dendritic cell engager, or a macrophage cell engager; (b) a cytokine molecule or cytokine inhibitor molecule; and (c) a death receptor signal engager.

In some embodiments, first antigen binding domain comprises an anti-TCRαV antibody molecule, e.g., as described herein.

In some embodiments, first antigen binding domain comprises an anti-TCRβV antibody molecule, e.g., as described herein.

In another aspect, the disclosure features a nucleic acid molecule encoding a multifunctional molecule disclosed herein.

In another aspect, the disclosure features a vector, e.g., an expression vector, comprising the nucleic acid molecules disclosed herein.

In another aspect, the disclosure features a host cell comprising a nucleic acid molecule or vector disclosed herein.

In another aspect, the disclosure features a method of making, e.g., producing, a multifunctional molecule disclosed herein, comprising culturing a host cell disclosed herein under suitable conditions, e.g., conditions suitable for gene expression and/or homo- or heterodimerization.

In another aspect, the disclosure features a pharmaceutical composition comprising a multifunctional molecule disclosed herein.

In another aspect, the disclosure features a method of treating a TCR bias, comprising administering to a subject in need thereof a multifunctional molecule disclosed herein, wherein the multifunctional molecule is administered in an amount effective to treat the TCR bias.

In another aspect, the disclosure features a method of treating an autoimmune disease (e.g., an autoimmune disease associated with a TCR bias), comprising administering to a subject in need thereof a multifunctional molecule disclosed herein, wherein the multifunctional molecule is administered in an amount effective to treat the autoimmune disease.

In another aspect, the disclosure features a method of identifying a subject in need of treatment for TCR bias or an autoimmune disease (e.g., associated with a TCR bias) using a multifunctional molecule disclosed herein, comprising determining (e.g., directly determining or indirectly determining, e.g., obtaining information regarding) whether a subject has a TCR bias (e.g., a biased TCRAV or TCRBV clonotype) and/or an autoimmune disease associated with said bias, wherein: responsive to determining that the subject has a TCR bias (e.g., a biased TCRAV or TCRBV clonotype) and/or an autoimmune disease associated with said bias, identifying the subject as a candidate for treatment using a multifunctional molecule comprising an antigen binding domain that binds to the TCRAV or TCRBV antigen.

In another aspect, the disclosure features a method of evaluating a subject in need of treatment for a TCR bias (e.g., a biased TCRAV or TCRBV clonotype) and/or an autoimmune disease associated with said bias, comprising determining (e.g., directly determining or indirectly determining, e.g., obtaining information regarding) whether a subject has a TCR bias (e.g., a biased TCRAV or TCRBV clonotype).

In yet another aspect, disclosed herein is a method of treating an autoimmune disease (e.g., an autoimmune disease associated with a TCR bias), in a subject in need thereof, comprising administering to said subject an effective amount, e.g., a therapeutically effective amount, of an antibody molecule which binds (e.g., specifically binds) to a T cell receptor alpha variable region (TCRαV) (“anti-TCRαV antibody molecule”) or a T cell receptor beta variable region (TCRαV) (“anti-TCRβV antibody molecule”), thereby treating the disorder.

In another aspect, the disclosure provides a method of depleting a population of T cells in a subject having an autoimmune disorder (e.g., an autoimmune disease associated with a TCR bias), comprising, contacting the T cell population with an effective amount of an antibody molecule which binds (e.g., specifically binds) to a T cell receptor alpha variable region (TCRαV) (“anti-TCRαV antibody molecule”) or a T cell receptor beta variable region (TCRβV) (“anti-TCRβV antibody molecule”).

In another aspect, the disclosure features a nucleic acid molecule encoding an anti-TCRαV or anti-TCRβV antibody molecule disclosed herein.

In another aspect, the disclosure features a vector, e.g., an expression vector, comprising the nucleic acid molecules disclosed herein.

In another aspect, the disclosure features a host cell comprising a nucleic acid molecule or vector disclosed herein.

In another aspect, the disclosure features a method of making, e.g., producing, an anti-TCRαV or anti-TCRβV antibody molecule disclosed herein, comprising culturing a host cell disclosed herein under suitable conditions, e.g., conditions suitable for gene expression and/or homo- or heterodimerization.

In another aspect, the disclosure features a pharmaceutical composition comprising an anti-TCRβV antibody molecule disclosed herein.

Disclosed herein, in some aspects, are TCR targeting molecules that comprise an antigen binding domain that binds to, e.g., selectively binds to, T cell receptor variable beta (TCRBV), e.g., a TCRBV antigen. In some cases, the TCR targeting molecules comprise a Fc region that comprises one or more binding mutations that enhance effector function of the Fc region, such as antibody dependent cell-mediated cytotoxicity (ADCC), antibody-dependent cellular phagocytosis (ADCP) or complement dependent cytotoxicity (CDC). In some cases, the TCR targeting molecules comprise a Fc region that does not comprise a binding mutation.

Disclosed herein, in some aspects, are TCR targeting molecules that include a plurality of (e.g., two or more) functionalities (or binding specificities) (also referred to herein as multifunctional molecules or multispecific molecules), comprising (i) an antigen binding domain that binds to, e.g., selectively binds to, T cell receptor variable beta (TCRBV), e.g., a TCRBV antigen, and (ii) one, two, or all of: (a) an immune cell engager chosen from a T cell engager, an NK cell engager (e.g., a molecule that binds to NKp30, NKp46, NKG2D, or CD16), a B cell engager, a dendritic cell engager, or a macrophage cell engager; (b) a cytokine inhibitor molecule; (c) a cytotoxic agent; (d) a death receptor signal engager.

In an embodiment, the TCR targeting molecule is a bispecific (or bifunctional) molecule, a trispecific (or trifunctional) molecule, or a tetraspecific (or tetrafunctional) molecule.

In some embodiments, the TCR targeting molecule comprises an antigen binding domain that binds a TCRAV or TCRBV antigen on the surface of a lymphocyte, e.g., T cell. In some embodiments, the TCRAV or TCRBV antigen corresponds to a biased TCRAV or TCRBV clonotype, e.g., TCRs comprising the TCRAV or TCRBV antigen may be over-represented in the TCR repertoire or lymphocyte (e.g., T cell) pool of a subject (e.g., subjects with autoimmune disease associated with TCR bias), or expressed at a level that is higher than the level in other subjects (e.g., non-autoimmune disease subjects).

Without being bound by theory, the TCR targeting molecules disclosed herein are expected to localize (e.g., bridge) and/or activate an immune cell (e.g., an immune effector cell chosen from a T cell, an NK cell, a B cell, a dendritic cell or a macrophage), in the presence of a cell (e.g., a lymphocyte, e.g., T cell) expressing the TCRAV or TCRBV antigen (e.g., a TCRAV or TCRBV antigen corresponding to a biased TCRAV or TCRBV clonotype), e.g., on the cell surface. Increasing the proximity and/or activity of the immune cell, in the presence of the cell (e.g., a lymphocyte, e.g., T cell) expressing the TCRAV or TCRBV antigen (e.g., a TCRAV or TCRBV antigen corresponding to a biased TCRAV or TCRBV clonotype) using the TCR targeting molecules described herein is expected to enhance an immune response against the target cell, thereby providing a more effective therapy (e.g., by decreasing the level of the biased TCR and/or T cell expressing the biased TCR). In another embodiment, targeting a cell (e.g., a lymphocyte, e.g., T cell) expressing the TCRAV or TCRBV antigen (e.g., a TCRAV or TCRBV antigen corresponding to a biased TCRAV or TCRBV clonotype) with a TCR targeting molecule also comprising a cell death inducing moiety (e.g., a death receptor signal engager) is thought to promote the death of the target cell (e.g., by decreasing the level of the biased TCR and/or T cell expressing the biased TCR).

Without being bound by theory, by utilizing, in some embodiments, a TCR targeting molecule specific for a particular TCRAV or TCRBV antigen (e.g., a TCRAV or TCRBV antigen corresponding to a biased TCRAV or TCRBV clonotype), but not with specificity for other or all types of T cell receptors, it is expected that the deleterious effects of increasing the proximity or activity of immune cells toward T cells generally or promoting cell death in T cells generally may be mitigated. In this way, it is thought that use of the TCR targeting molecules disclosed herein may increase the proximity or activity of immune cells toward cells comprising TCRAV or TCRBV antigen corresponding to a biased TCRAV or TCRBV clonotype without necessarily increasing proximity or activity of immune cells toward T cells generally, or promote cell death in cells comprising TCRAV or TCRBV antigen corresponding to a biased TCRBV clonotype without necessarily increasing cell death in T cells generally.

Accordingly, provided herein are, inter alia, TCR targeting molecules (e.g., multifunctional antibody molecules) that include the aforesaid moieties, nucleic acids encoding the same, methods of producing the aforesaid molecules, and methods of treating a disease or disorder, e.g., an autoimmune disease or a TCR bias, using the aforesaid molecules.

### Certain Terminology

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of 20% or in some instances ±10%, or in some instances ±5%, or in some instances ±1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2, FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FRI, CDR1, FR2, CDR2, FR3, CDR3, and FR4.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRβV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.geg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.geg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

In some embodiments, the agent is the TCR targeting molecule as provided herein.

### Human T Cell Receptor (TCR) Complex

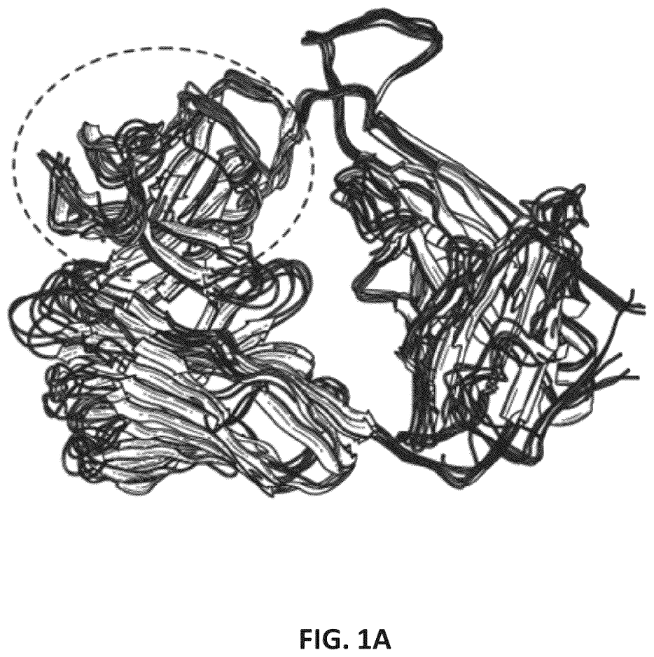

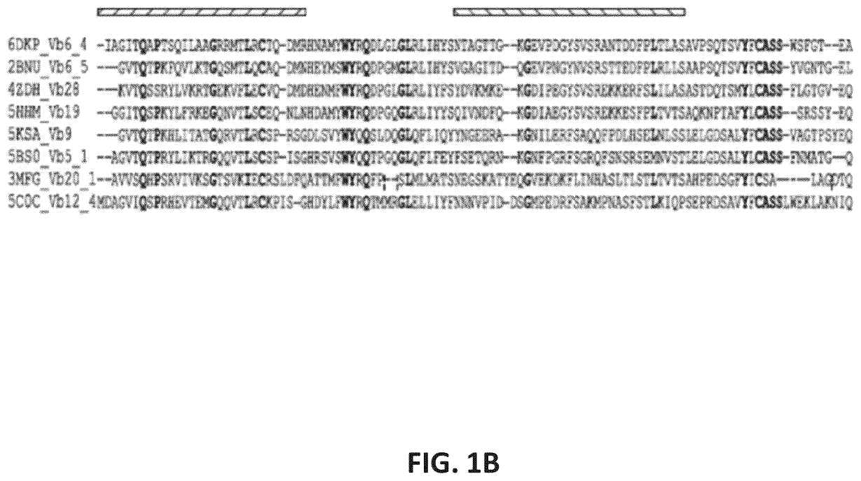

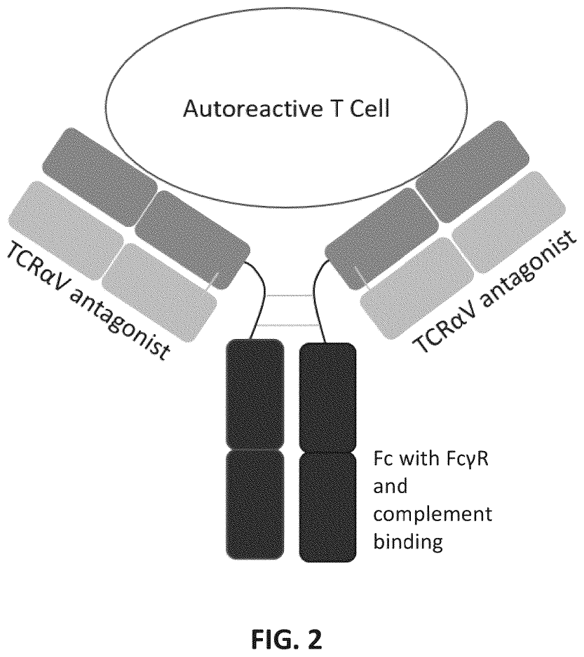

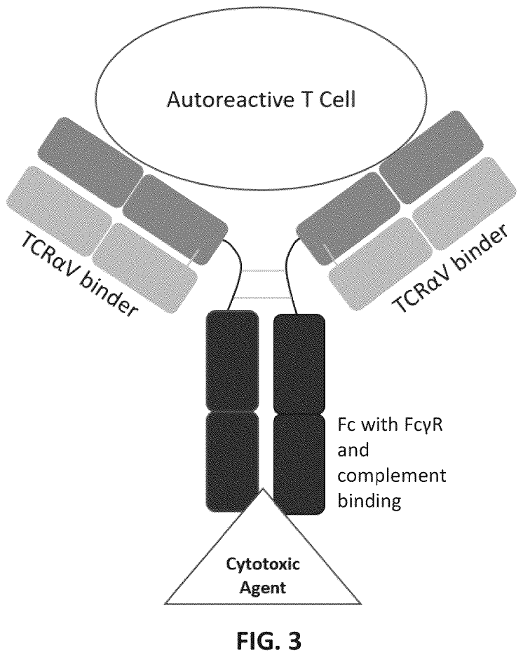

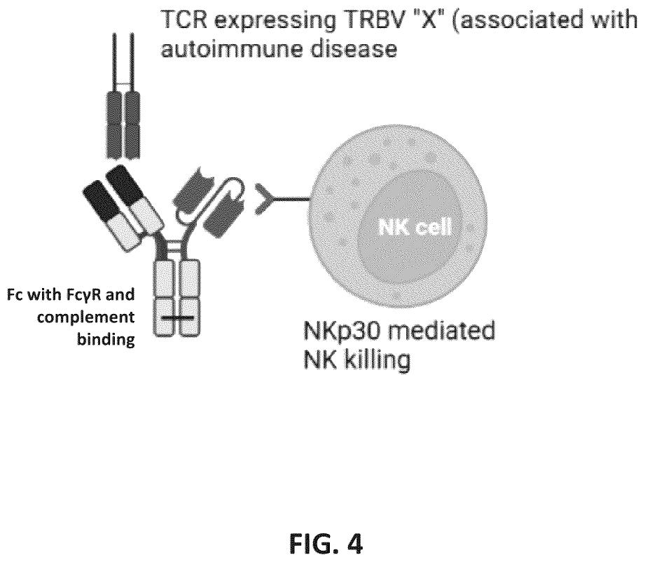

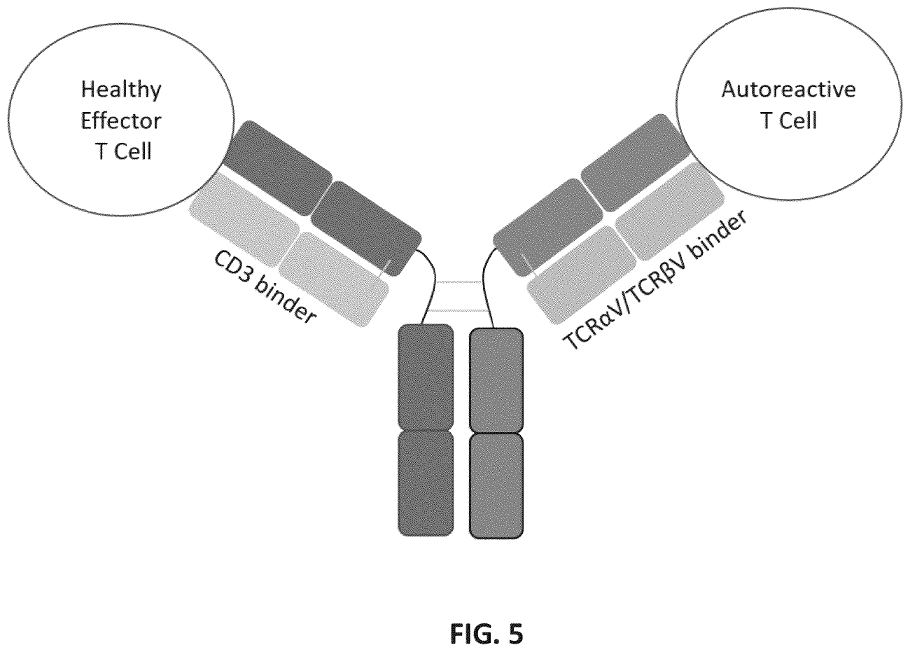

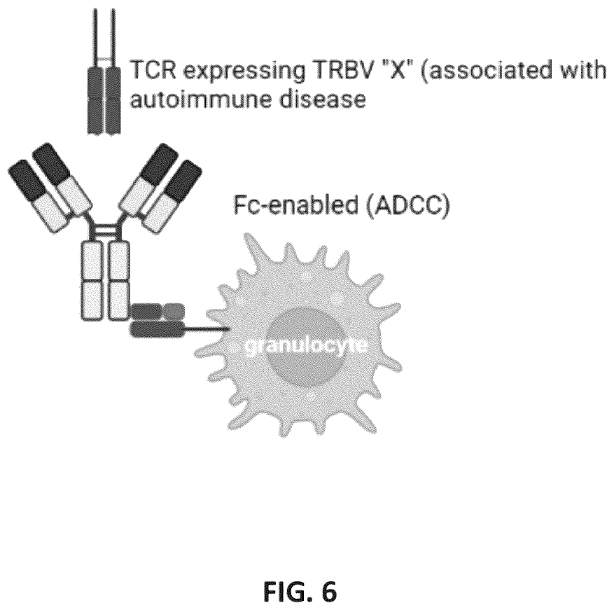

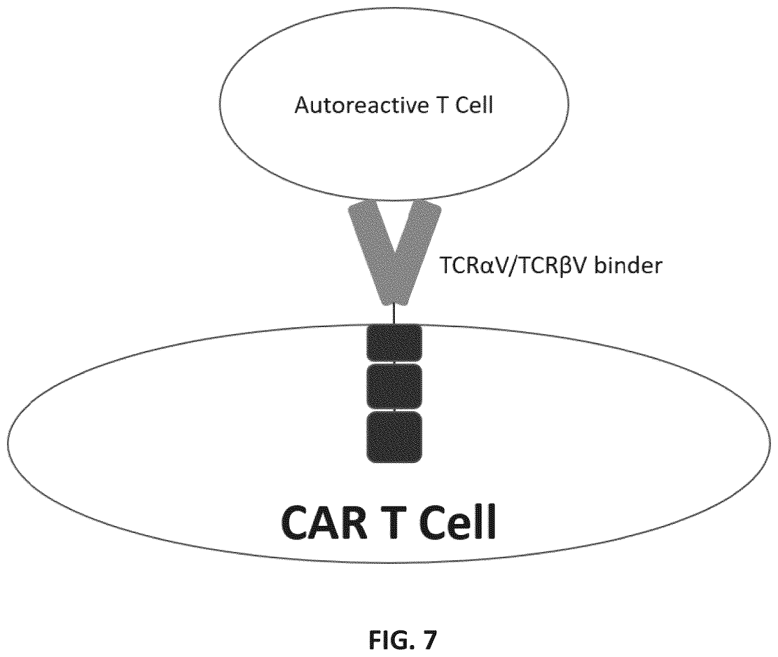

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRβV chains can be further classified into 30 subfamilies (TRBV1-30). Despite their high structural and functional homology, the amino acid sequence homology in the TRBV genes is very low. Only 4 amino acids out of approximately 95 are identical while 10 additional amino acids are conserved among all subfamilies (see, an alignment of TCRBV amino acid sequences in Table 8). Nevertheless, TCRs formed between alpha and beta chains of highly diverse sequences show a remarkable structural homology (FIGS. 1A and 1B) and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor alpha variable chain” or “TCRαV,” or “TRAV,” refers to an extracellular region of the T cell receptor alpha chain which can comprise a portion of the antigen recognition domain of the T cell receptor. The term TCRαV includes isoforms, mammalian, e.g., human TCRαV, species homologs of human and analogs comprising at least one common epitope with TCRαV. Human TCRαV comprises a gene family comprising subfamilies including, but not limited to: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof (e.g., a structural or functional variant thereof).

In some embodiments, the TCRα V1 subfamily comprises: TCRαV1-1 or TCRαV1-2, or a variant thereof.

In some embodiments, the TCRα V8 subfamily comprises: TCRαV8-1, TCRαV8-2, TCRαV8-3, TCRαV8-4, or TCRαV8-6, or a variant thereof.

In some embodiments, the TCRα V9 subfamily comprises: TCRαV9-1 or TCRαV9-2, or a variant thereof.

In some embodiments, the TCRα V12 subfamily comprises: TCRαV12-1, TCRαV12-2, or TCRαV12-3, or a variant thereof.

In some embodiments, the TCRα V13 subfamily comprises: TCRαV13-1 or TCRαV13-2, or a variant thereof.

In some embodiments, the TCRα V14 subfamily comprises: TCRαV14/DV4, or a variant thereof.

In some embodiments, the TCRα V23 subfamily comprises: TCRαV23/DV6, or a variant thereof.

In some embodiments, the TCRα V26 subfamily comprises: TCRαV26-1 or TCRαV26-2, or a variant thereof.

In some embodiments, the TCRα V29 subfamily comprises: TCRαV29/DV5, or a variant thereof.

In some embodiments, the TCRα V36 subfamily comprises: TCRαV236/DV7, or a variant thereof.

In some embodiments, the TCRα V38 subfamily comprises: TCRαV38-1 or TCRαV38-2/DV8, or a variant thereof.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, as well as family members of said subfamilies, and variants thereof (e.g., a structural or functional variant thereof). In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01, or a variant thereof, e.g., a variant having 85%, 90%, 95%, 99% or more identity the naturally-occurring sequence. TCRβ V6-5*01 is also known as TRBV65; TCRBV6S5; TCRBV13S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or a sequence having 85%, 90%, 95%, 99% or more identity thereof.

SEQ ID NO: 43
ATGAGCATCGGCCTCCTGTGCTGTGCAGCCTTGTCTCTCCTGTGG
GCAGGTCCAGTGAATGCTGGTGTCACTCAGACCCCAAAATTCCAG
GTCCTGAAGACAGGACAGAGCATGACACTGCAGTGTGCCCAGGAT
ATGAACCATGAATACATGTCCTGGTATCGACAAGACCCAGGCATG
GGGCTGAGGCTGATTCATTACTCAGTTGGTGCTGGTATCACTGAC
CAAGGAGAAGTCCCCAATGGCTACAATGTCTCCAGATCAACCACA
GAGGATTTCCCGCTCAGGCTGCTGTCGGCTGCTCCCTCCCAGACA
TCTGTGTACTTCTGTGCCAGCAGTTACTC
SEQ ID NO: 44
MSIGLLCCAALSLLWAGPVNAGVTQTPKFQVLKTGQSMTLQCAQD
MNHEYMSWYRQDPGMGLRLIHYSVGAGITDQGEVPNGYNVSRSTT
EDFPLRLLSAAPSQTSVYFCASSY

### TCR Alpha V (TCRαV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR alpha gene undergoes gene arrangement to generate diversity.

The TCR V alpha repertoire varies between individuals and populations because of, e.g., 7 frequently occurring inactivating polymorphisms in functional gene segments and a large insertion/deletion-related polymorphism encompassing 2 V alpha gene segments.

Provided herein are, inter alia, antibody molecules and fragments thereof, that bind, e.g., specifically bind, to a human TCR alpha V chain (TCRαV), e.g., a TCRαV gene family (also referred to as a group), e.g., a TCRαV subfamily (also referred to as a subgroup), e.g., as described herein. TCR alpha V families and subfamilies are known in the art, e.g., as described in Yassai et al., (2009) Immunogenetics 61(7)pp:493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRAV, TCRVA, TRAV, TCRαV, TCRVα or TRαV are used interchangeably herein and refer to a TCR alpha V chain, e.g., as described herein.

In some embodiments, provided herein is an anti-TCRαV antibody molecule that binds to human TCRαV, e.g., a TCRαV family, e.g., gene family or a variant thereof.

Exemplary amino acid sequences for TCRαV subfamily members can be found on the ImMunoGeneTics Information System website: www.imgt.org, or in a similar resource.

### TCR Beta V (TCRβV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

Provided herein are, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7)pp:493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRBV, TCRVB, TRBV, TCRβV, TCRVβ or TRβV are used interchangeably herein and refer to a TCR beta V chain, e.g., as described herein.

In some embodiments, provided herein is an anti-TCRβV antibody molecule that binds to human TCRβV, e.g., a TCRβV family, e.g., gene family or a variant thereof. In some embodiments a TCRBV gene family comprises one or more subfamilies, e.g., as described herein, e.g., in Table 6 or Table 7. In some embodiments, the TCRβV gene family comprises: a TCRβ V6 subfamily, a TCRβ V10 subfamily, a TCRβ V12 subfamily, a TCRβ V5 subfamily, a TCRβ V7 subfamily, a TCRβ VII subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30:

In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01, or a variant thereof.

In some embodiments, the TCRβ VII subfamily comprises: TCRβ V11-1*01, TCRβ V11-2*01 or TCRβ V11-3*01, or a variant thereof. In some embodiments, the TCRβ V14 subfamily comprises TCRβ V14*01, or a variant thereof. In some embodiments, the TCRβ V16 subfamily comprises TCRβ V16*01, or a variant thereof. In some embodiments, the TCRβ V18 subfamily comprises TCRβ V18*01, or a variant thereof. In some embodiments, the TCRβ V9 subfamily comprises TCRβ V9*01 or TCRβ V9*02, or a variant thereof. In some embodiments, the TCRβ V13 subfamily comprises TCRβ V13*01, or a variant thereof. In some embodiments, the TCRβ V4 subfamily comprises TCRβ V4-2*01, TCRβ V4-3*01, or TCRβ V4-1*01, or a variant thereof. In some embodiments, the TCRβ V3 subfamily comprises TCRβ V3-1*01, or a variant thereof. In some embodiments, the TCRβ V2 subfamily comprises TCRβ V2*01, or a variant thereof. In some embodiments, the TCRβ V15 subfamily comprises TCRβ V15*01, or a variant thereof. In some embodiments, the TCRβ V30 subfamily comprises TCRβ V30*01, or TCRβ V30*02, or a variant thereof. In some embodiments, the TCRβ V19 subfamily comprises TCRβ V19*01, or TCRβ V19*02, or a variant thereof. In some embodiments, the TCRβ V27 subfamily comprises TCRβ V27*01, or a variant thereof. In some embodiments, the TCRβ V28 subfamily comprises TCRβ V28*01, or a variant thereof. In some embodiments, the TCRβ V24 subfamily comprises TCRβ V24-1*01, or a variant thereof. In some embodiments, the TCRβ V20 subfamily comprises TCRβ V20-1*01, or TCRβ V20-1*02, or a variant thereof. In some embodiments, the TCRβ V25 subfamily comprises TCRβ V25-1*01, or a variant thereof. In some embodiments, the TCRβ V29 subfamily comprises TCRβ V29-1*01, or a variant thereof.

Exemplary amino acid sequences for TCRβV subfamily members can be found on the ImMunoGeneTics Information System website: www.imgt.org, or in a similar resource.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the molecules or the compositions as described herein may vary depending on cell type to which delivery of the molecules or the compositions is desired. The molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the molecules or the compositions as described herein are administered with one or more agents capable of promoting penetration of the subject the molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the molecules or the compositions as described herein are linked or conjugated with agents that provide desirable pharmaceutical or pharmacodynamic properties. In some embodiments, the molecules or the compositions as described herein are coupled to a substance, known in the art to promote penetration or transport across the blood-brain barrier, e.g., an antibody to the transferrin receptor. In some embodiments, the molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Methods of Treatment

Described herein, in certain embodiments, is a method of treating a condition or disease in a subject in need therefor comprising administering to the subject a therapeutically effective amount of the antibody molecule, multifunctional molecule, or multispecific molecule as described herein, the nucleic acid molecules as described herein, the vector as described herein, the cell as described herein, the pharmaceutical composition as described herein, or a combination thereof, wherein the administering is effective to treat the condition or disease in the subject. Any condition or disease that is related to TCRαV and/or TCRαV may be subject of the methods of treatment disclosed herein. For example, a condition or disease wherein autoreactive T cells of a subject that expresses a TCRαV and/or a TCRαV can be treated with a CAR-T cell containing a CAR with an anti-TCRαV and/or anti-TCRβV binding domain that binds to the TCRαV and/or TCRβV expressed by autoreactive T cells. Examples of TCRαV- and TCRβV-related diseases include, but are not limited to, those listed in Table 5.

In some embodiments, the condition or disease is an autoimmune disease. In some embodiments, the autoimmune disease is selected from the group consisting of the autoimmune disease is selected from amyotrophic lateral sclerosis (ALS), coeliac disease (CD), ankylosing spondylitis (AS), Covid-induced multisystem inflammatory syndrome in children (MIS-C), primary Sjogren's syndrome (PSS), Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellardegeneration (PCD), Lambert-Eaton syndrome, psoriasis, scleroderma, CREST (calcinosis,Raynaud phenomenon, esophageal dysmotility, sclerodactyly, and telangiectasia) syndrome,dermatitis herpetiformis, dermatomyositis, bullous pemphigoid, cicatricial pemphigoid/benignmucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis),Takayasu's arteritis, polyarteritis nodosa, microscopic polyangiitis (MPA), leukocytoclasticvasculitis, Cogan's syndrome, uveitis, peripheral uveitis (Pars planitis), scleritis, autoimmuneinner ear disease (AIED), Crohn's, ulcerative colitis (UC), Dressler's syndrome, Rheumaticfever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), ocular cicatricial pemphigoid, or Hashimoto's thyroiditis.

In some embodiments, the molecules or the composition disclosed herein (e.g., antibody molecules or multispecific molecules (or pharmaceutical composition) provided herein, the nucleic acid molecules as described herein, the vector as described herein, the cell as described herein, the pharmaceutical composition as described herein, or a combination thereof), are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the molecules or the composition disclosed herein to be administered can be determined by a physician with consideration of individual differences in severity/character of immune disorder, extent of infection or metastasis, age, weight, and condition of the subject.

In some embodiments, binding of the molecules or the composition disclosed herein results in cell killing, e.g., target cell killing, e.g. autoreactive T cell killing. In some embodiments, the autoreactive T cell is a CD4+ T cell. In some embodiments, the autoreactive T cell is a CD8+ T cell. In some embodiments, binding of the molecules or the composition disclosed herein results in cell killing in vitro or in vivo.

In embodiments, the molecules or the composition disclosed herein is administered to the subject parenterally. In embodiments, the cells disclosed herein are administered to the subject intravenously, subcutaneously, intranodally, intramuscularly, intradermally, or intraperitoneally. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

Without wishing to be bound by any particular theory, the immune response elicited by the anti-TCRVα and/or anti-TCRβV TCR targeting molecules may be an active or a passive immune response. In one embodiment, the anti-TCRVα and/or anti-TCRβV TCR targeting molecules of the present disclosure may be a type of vaccine for ex vivo immunization and/or in vivo therapy in a mammal. With respect to ex vivo immunization, at least one of the following occurs in vitro prior to administering the cell into a mammal: i) expansion of the cells, ii) introducing a nucleic acid encoding an anti-TCRVα and/or anti-TCRβV TCR targeting molecules to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing an anti-TCRVα and/or anti-TCRβV TCR targeting molecule disclosed herein.

Methods described herein include treating an autoimmune disease in a subject by using the molecules or the composition disclosed herein, e.g., using a pharmaceutical composition described herein. Also provided are methods for reducing or ameliorating a symptom of an autoimmune disease in a subject. In some embodiments, the methods described herein re-balance a T cell repertoire in a subject (e.g. reduce or deplete autoreactive T cells and preserve healthy T cells to establish a T cell repertoire more similar to a person without an autoimmune disease).

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject, thereby inhibiting or preventing the activation or expansion of autoreactive T cells in the subject. In some embodiments, the agent comprises an anti-TCRαV antibody comprising a variant Fc region. In some embodiments, the agent comprises an anti-TCRβV antibody comprising a variant Fc region. In some embodiments, the variant Fc region has increased binding to C1q complement. In some embodiments, the variant Fc region has increased effector function (e.g., increased antibody dependent cell-mediated cytotoxicity (ADCC), antibody-dependent cellular phagocytosis (ADCP) and/or complement dependent cytotoxicity (CDC).

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject, thereby inhibiting or preventing the activation or expansion of autoreactive T cells in the subject. In some embodiments, the agent comprises an anti-TCRαV antibody comprising a variant Fc region. In some embodiments, the agent comprises an anti-TCRβV antibody comprising a variant Fc region. In some embodiments, the variant Fc region has increased binding to C1q complement. In some embodiments, the variant Fc region has increased effector function (e.g., increased antibody dependent cell-mediated cytotoxicity (ADCC), antibody-dependent cellular phagocytosis (ADCP) and/or complement dependent cytotoxicity (CDC). In some embodiments, the disease or condition is an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the agent comprises an antibody-drug conjugate. In some embodiments, the antibody-drug conjugate comprises a TCRαV antigen binding domain. In some embodiments, the antibody-drug conjugate comprises a TCRαV antigen binding domain. In some embodiments, the antibody-drug conjugate has no mutation to an Fc region. In some embodiments, the antibody-drug conjugate is functionally linked to a cytotoxic agent. In some embodiments, the cytotoxic agent is an inhibitor of tubulin polymerization. In some embodiments, the cytotoxic agent is selected from the group consisting of maytansine, dolastatin, auristatin drug analogs, duocarmycin derivatives (e.g. CC-1065 analogs and duocarmycin), esperamicin, calicheamicin, and pyrrolobenzodiazepine (PBD).

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject. In some embodiments, the agent comprises an antibody-drug conjugate. In some embodiments, the antibody-drug conjugate comprises a TCRαV antigen binding domain. In some embodiments, the antibody-drug conjugate comprises a TCRβV antigen binding domain. In some embodiments, the antibody-drug conjugate has no mutation to an Fc region. In some embodiments, the antibody-drug conjugate is functionally linked to a cytotoxic agent. In some embodiments, the cytotoxic agent is an inhibitor of tubulin polymerization. In some embodiments, the cytotoxic agent is selected from the group consisting of maytansine, dolastatin, auristatin drug analogs, duocarmycin derivatives (e.g. CC-1065 analogs and duocarmycin), esperamicin, calicheamicin, and pyrrolobenzodiazepine (PBD). In some embodiments, the disease or condition is an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the agent is a CAR T cell comprising a CAR with an anti-TCRαV binding domain that binds to the TCRαV expressed by the autoreactive T cells. In some embodiments, the agent is a CAR T cell comprising a CAR with an anti-TCRβV binding domain that binds to the TCRβV expressed by the autoreactive T cells. In some embodiments, the CAR comprises a transmembrane domain as provided herein. In some embodiments, the CAR comprises an intracellular domain as provided herein.

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject. In some embodiments, the agent is a CAR T cell comprising a CAR with an anti-TCRαV binding domain that binds to the TCRαV expressed by the autoreactive T cells. In some embodiments, the agent is a CAR T cell comprising a CAR with an anti-TCRβV binding domain that binds to the TCRβV expressed by the autoreactive T cells. In some embodiments, the CAR comprises a transmembrane domain as provided herein. In some embodiments, the CAR comprises an intracellular domain as provided herein. In some embodiments, the disease or condition is an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the subject is administered a multispecific molecule comprising a TCRVα binding region and a NK cell engager. In some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is a ligand of CD16, which is a CD16a/b ligand, e.g., a CD16a/b ligand further comprising an antibody Fc region. In some embodiments, the NK cell engager is an agonist for a NK activating receptor. In some embodiments, the NK cell engager is an antagonist for a NK inhibitory receptor. In some embodiments, the multispecific molecule has no Fc region mutation.

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject. In some embodiments, the subject is administered a multispecific molecule comprising a TCRVα binding region and a NK cell engager. In some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is a ligand of CD16, which is a CD16a/b ligand, e.g., a CD16a/b ligand further comprising an antibody Fc region. In some embodiments, the NK cell engager is an agonist for a NK activating receptor. In some embodiments, the NK cell engager is an antagonist for a NK inhibitory receptor. In some embodiments, the multispecific molecule has no Fc region mutation. In some embodiments, the subject has an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the subject is administered an agent comprising a multispecific molecule comprising an antigen binding domain that binds to TCRVα and a T cell engager. In some embodiments, the subject is administered an agent comprising a multispecific molecule comprising an antigen binding domain that binds to TCRVβ and a T cell engager. In some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager binds to (e.g. activates) a CD4+ and/or a CD8+ T cell. In some embodiments, the T cell engager binds to (e.g. activates) a CD4+ T cell. In some embodiments, the T cell engager binds to (e.g. activates) a CD8+ T cell. In some embodiments, the disease or condition is an autoimmune disease.

Also described herein are methods of treating a subject having an autoimmune disease, comprising: acquiring a status of one or more TCRαV and/or TCRβV molecules in a sample from the subject, and determining the one or more TCRαV and/or TCRβV molecules is expressed in the autoreactive T cell population, in a sample from the subject, wherein responsive to said determination, administering to the subject an effective amount of an anti-TCRαV antibody molecule and/or anti-TCRβV antibody molecule, e.g., an agonistic anti-TCRαV antibody and/or an agonistic anti-TCRβV antibody molecule, e.g., as described herein.

In some embodiments, acquiring a value for the status, e.g., presence, level and/or activity, of one or more TCRαV and/or TCRβV molecules comprises acquiring a measure of the T cell receptor (TCR) repertoire of a sample. In some embodiments, the value comprises a measure of the clonotype of a population of T cells in the sample.

In some embodiments, a value for the status of one or more TCRαV and/or TCRβV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRαV and/or TCRβV molecules is obtained, e.g., measured, using flow cytometry.

In some embodiments, the subject is administered the multifunctional polypeptide molecule as described herein comprising an anti-TCRαV and/or anti-TCRβV binding moiety.

In some embodiments, provided herein is a method of treating a subject that suffers from amyotrophic lateral sclerosis (ALS) by administering to the subject a TCR targeting molecule disclosed herein that specifically binds to a TCRβV7 subfamily molecule, a nucleic acid molecule encoding the TCR targeting molecule, or a cell that expresses the TCR targeting molecule (e.g., a CAR-T cell disclosed herein). In some embodiments, the TCRβV7 subfamily molecule comprises a TCRβV7-1 molecule. In some embodiments, provided herein is a method of treating a subject that suffers from amyotrophic lateral sclerosis (ALS) by administering to the subject a TCR targeting molecule disclosed herein that specifically binds to a TCRβV23 subfamily molecule, a nucleic acid molecule encoding the TCR targeting molecule, or a cell that expresses the TCR targeting molecule (e.g., a CAR-T cell disclosed herein).

In some embodiments, provided herein is a method of treating a subject that suffers from Celiac disease by administering to the subject a TCR targeting molecule disclosed herein that specifically binds to a TCRβV4 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Celiac disease by administering to the subject a TCR targeting molecule disclosed herein that specifically binds to a TCRβV7 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Celiac disease by administering to the subject a TCR targeting molecule disclosed herein that specifically binds to a TCRαV4 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Celiac disease by administering to the subject a TCR targeting molecule disclosed herein that specifically binds to a TCRαV26 subfamily molecule, a nucleic acid molecule encoding the TCR targeting molecule, or a cell that expresses the TCR targeting molecule (e.g., a CAR-T cell disclosed herein).

In some embodiments, provided herein is a method of treating a subject that suffers from ankylosing spondylitis by administering to the subject a TCR targeting molecule disclosed herein that specifically binds to a TCRβV9 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from ankylosing spondylitis by administering to the subject a TCR targeting molecule disclosed herein that specifically binds to a TCRαV21 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Covid-induced multisystem inflammatory syndrome in children (MIS-C) by administering to the subject a TCR targeting molecule disclosed herein that specifically binds to a TCRβV11 subfamily molecule, a nucleic acid molecule encoding the TCR targeting molecule, or a cell that expresses the TCR targeting molecule (e.g., a CAR-T cell disclosed herein). In some embodiments, the TCRβV11 subfamily molecule comprises a TCRβV11-2 molecule. In some embodiments, provided herein is a method of treating a subject that suffers from primary Sjogren's syndrome by administering to the subject a TCR targeting molecule disclosed herein that specifically binds to a TCRβV6 subfamily molecule, a nucleic acid molecule encoding the TCR targeting molecule, or a cell that expresses the TCR targeting molecule (e.g., a CAR-T cell disclosed herein). In some embodiments, the TCRβV6 subfamily molecule comprises a TCRβV6-1 molecule, a TCRβV6-2/3 molecule, or a TCRβV6-5 molecule.

In some embodiments, provided herein is a method of treating a subject that suffers from multiple sclerosis by administering to the subject a TCR targeting molecule disclosed herein that specifically binds to a TCRβV20 subfamily molecule, a nucleic acid molecule encoding the TCR targeting molecule, or a cell that expresses the TCR targeting molecule (e.g., a CAR-T cell disclosed herein). In some embodiments, the TCRβV20 subfamily molecule is a TCRβV20-1 molecule. In some embodiments, provided herein is a method of treating a subject that suffers from multiple sclerosis by administering to the subject a TCR targeting molecule disclosed herein that specifically binds to a TCRβV29 subfamily molecule, a nucleic acid molecule encoding the TCR targeting molecule, or a cell that expresses the TCR targeting molecule (e.g., a CAR-T cell disclosed herein). In some embodiments, the TCRβV20 subfamily molecule is a TCRβV29-1 molecule. In some embodiments, provided herein is a method of treating a subject that suffers from multiple sclerosis by administering to the subject a TCR targeting molecule disclosed herein that specifically binds to a TCRβV7 subfamily molecule, a nucleic acid molecule encoding the TCR targeting molecule, or a cell that expresses the TCR targeting molecule (e.g., a CAR-T cell disclosed herein). In some embodiments, the TCRβV7 subfamily molecule is a TCRβV7-8/9 molecule. In some embodiments, provided herein is a method of treating a subject that suffers from multiple sclerosis by administering to the subject a TCR targeting molecule disclosed herein that specifically binds to a TCRβV10 subfamily molecule, a nucleic acid molecule encoding the TCR targeting molecule, or a cell that expresses the TCR targeting molecule (e.g., a CAR-T cell disclosed herein). In some embodiments, the TCRβV10 subfamily molecule is a TCRβV10-3 molecule.

In some embodiments, provided herein is a method of treating a subject that suffers from type I diabetes by administering to the subject a TCR targeting molecule disclosed herein that specifically binds to a TCRβV12 subfamily molecule, a nucleic acid molecule encoding the TCR targeting molecule, or a cell that expresses the TCR targeting molecule (e.g., a CAR-T cell disclosed herein). In some embodiments, the TCRβV12 subfamily molecule comprises a TCRβV12-3/4 molecule. In some embodiments, provided herein is a method of treating a subject that suffers from type I diabetes by administering to the subject a TCR targeting molecule disclosed herein that specifically binds to a TCRαV12 subfamily molecule, a nucleic acid molecule encoding the TCR targeting molecule, or a cell that expresses the TCR targeting molecule (e.g., a CAR-T cell disclosed herein). In some embodiments, the TCRαV12 subfamily molecule comprises a TCRαV12-3 molecule.

In some embodiments, a subject has amyotrophic lateral sclerosis. In some embodiments, the subject having amyotrophic lateral sclerosis has a population of autoreactive T cells that express a TCRβV7 subfamily comprising TCRβV7-1. In some embodiments, the subject is administered the agent, antibody molecule, multispecific molecule, and/or multifunctional molecule as described herein comprising an anti-TCRβV molecule that binds to one or more members of the TCRβV7 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV7 subfamily. In some embodiments, administration of the agent, antibody molecule, multispecific molecule, and/or multifunctional molecule as described herein results in reducing or depleting the population of autoreactive T cells expressing one or more members of the TCRβV7 subfamily. In some embodiments, the subject having amyotrophic lateral sclerosis has a population of autoreactive T cells that express a TCRβV23 subfamily. In some embodiments, the subject is administered the agent, antibody molecule, multispecific molecule, and/or multifunctional molecule as described herein comprising an anti-TCRβV molecule that binds to one or more members of the TCRβV23 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV23 subfamily. In some embodiments, administration of the agent, antibody molecule, multispecific molecule, and/or multifunctional molecule as described herein results in reducing or depleting the population of autoreactive T cells expressing one or more members of the TCRβV23 subfamily.

In some embodiments, a subject has Celiac disease. In some embodiments, the subject having Celiac disease has a population of autoreactive T cells that express a TCRβV4 subfamily. In some embodiments, the subject is administered the agent, antibody molecule, multispecific molecule, and/or multifunctional molecule as described herein comprising an anti-TCRβV molecule that binds to one or more members of the TCRβV4 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV4 subfamily. In some embodiments, administration of the agent, antibody molecule, multispecific molecule, and/or multifunctional molecule as described herein results in reducing or depleting the population of autoreactive T cells expressing one or more members of the TCRβV4 subfamily. In some embodiments, the subject having Celiac disease has a population of autoreactive T cells that express a TCRβV7 subfamily. In some embodiments, the subject is administered the agent, antibody molecule, multispecific molecule, and/or multifunctional molecule as described herein comprising an anti-TCRβV molecule that binds to one or more members of the TCRβV7 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV7 subfamily. In some embodiments, administration of the agent, antibody molecule, multispecific molecule, and/or multifunctional molecule as described herein results in reducing or depleting the population of autoreactive T cells expressing one or more members of the TCRβV7 subfamily. In some embodiments, the subject having Celiac disease has a population of autoreactive T cells that express a TCRαV4 subfamily. In some embodiments, the subject is administered the agent, antibody molecule, multispecific molecule, and/or multifunctional molecule as described herein comprising an anti-TCRαV molecule that binds to one or more members of the TCRαV4 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV4 subfamily. In some embodiments, administration of the agent, antibody molecule, multispecific molecule, and/or multifunctional molecule as described herein results in reducing or depleting the population of autoreactive T cells expressing one or more members of the TCRαV4 subfamily. In some embodiments, the subject having Celiac disease has a population of autoreactive T cells that express a TCRαV26 subfamily. In some embodiments, the subject is administered the agent, antibody molecule, multispecific molecule, and/or multifunctional molecule as described herein comprising an anti-TCRαV molecule that binds to one or more members of the TCRαV26 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV26 subfamily. In some embodiments, administration of the agent, antibody molecule, multispecific molecule, and/or multifunctional molecule as described herein results in reducing or depleting the population of autoreactive T cells expressing one or more members of the TCRαV26 subfamily.

In some embodiments, a subject has ankylosing spondylitis. In some embodiments, the subject having ankylosing spondylitis has a population of autoreactive T cells that express a TCRβV9 subfamily. In some embodiments, the subject is administered the agent, antibody molecule, multispecific molecule, and/or multifunctional molecule as described herein comprising an anti-TCRβV molecule that binds to one or more members of the TCRβV9 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV9 subfamily. In some embodiments, administration of the agent, antibody molecule, multispecific molecule, and/or multifunctional molecule as described herein results in reducing or depleting the population of autoreactive T cells expressing one or more members of the TCRβV9 subfamily. In some embodiments, the subject having ankylosing spondylitis has a population of autoreactive T cells that express a TCRαV21 subfamily. In some embodiments, the subject is administered the agent, antibody molecule, multispecific molecule, and/or multifunctional molecule as described herein comprising an anti-TCRαV molecule that binds to one or more members of the TCRαV21 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV21 subfamily. In some embodiments, administration of the agent, antibody molecule, multispecific molecule, and/or multifunctional molecule as described herein results in reducing or depleting the population of autoreactive T cells expressing one or more members of the TCRαV21 subfamily.

In some embodiments, a subject has Covid-induced multisystem inflammatory syndrome in children (MIS-C). In some embodiments, the subject having MIS-C has a population of autoreactive T cells that express a TCRβV11 subfamily comprising TCRβV11-2. In some embodiments, the subject is administered the agent, antibody molecule, multispecific molecule, and/or multifunctional molecule as described herein comprising an anti-TCRβV molecule that binds to one or more members of the TCRβV11 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV11 subfamily. In some embodiments, administration of the agent, antibody molecule, multispecific molecule, and/or multifunctional molecule as described herein results in reducing or depleting the population of autoreactive T cells expressing one or more members of the TCRβV11 subfamily.

In some embodiments, a subject has primary Sjogren's syndrome. In some embodiments, the subject having primary Sjogren's syndrome has a population of autoreactive T cells that express a TCRβV6 subfamily comprising TCRβV6-1, TCRβV6-2/3, or TCRβV6-5. In some embodiments, the subject is administered the agent, antibody molecule, multispecific molecule, and/or multifunctional molecule as described herein comprising an anti-TCRβV molecule that binds to one or more members of the TCRβV6 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV6 subfamily. In some embodiments, administration of the agent, antibody molecule, multispecific molecule, and/or multifunctional molecule as described herein results in reducing or depleting the population of autoreactive T cells expressing one or more members of the TCRβV6 subfamily.

In some embodiments, a subject has multiple sclerosis. In some embodiments, the subject having multiple sclerosis has a population of autoreactive T cells that express a TCRβV20 subfamily comprising TCRβV20-1. In some embodiments, the subject is administered the agent, antibody molecule, multispecific molecule, and/or multifunctional molecule as described herein comprising an anti-TCRβV molecule that binds to one or more members of the TCRβV20 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV20 subfamily. In some embodiments, administration of the agent, antibody molecule, multispecific molecule, and/or multifunctional molecule as described herein results in reducing or depleting the population of autoreactive T cells expressing one or more members of the TCRβV20 subfamily. In some embodiments, the subject having multiple sclerosis has a population of autoreactive T cells that express a TCRβV29 subfamily comprising TCRβV29-1. In some embodiments, the subject is administered the agent, antibody molecule, multispecific molecule, and/or multifunctional molecule as described herein comprising an anti-TCRβV molecule that binds to one or more members of the TCRβV29 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV29 subfamily. In some embodiments, administration of the agent, antibody molecule, multispecific molecule, and/or multifunctional molecule as described herein results in reducing or depleting the population of autoreactive T cells expressing one or more members of the TCRβV29 subfamily. In some embodiments, the subject having multiple sclerosis has a population of autoreactive T cells that express a TCRβV7 subfamily comprising TCRβV7-8/9. In some embodiments, the subject is administered the agent, antibody molecule, multispecific molecule, and/or multifunctional molecule as described herein comprising an anti-TCRβV molecule that binds to one or more members of the TCRβV7 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV7 subfamily. In some embodiments, administration of the agent, antibody molecule, multispecific molecule, and/or multifunctional molecule as described herein results in reducing or depleting the population of autoreactive T cells expressing one or more members of the TCRβV7 subfamily. In some embodiments, the subject having multiple sclerosis has a population of autoreactive T cells that express a TCRβV10 subfamily comprising TCRβV10-3. In some embodiments, the subject is administered the agent, antibody molecule, multispecific molecule, and/or multifunctional molecule as described herein comprising an anti-TCRαV molecule that binds to one or more members of the TCRβV10 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV10 subfamily. In some embodiments, administration of the agent, antibody molecule, multispecific molecule, and/or multifunctional molecule as described herein results in reducing or depleting the population of autoreactive T cells expressing one or more members of the TCRβV10 subfamily.

In some embodiments, a subject has type I diabetes. In some embodiments, the subject having type I diabetes has a population of autoreactive T cells that express a TCRβV12 subfamily comprising TCRβV12-3/4. In some embodiments, the subject is administered the agent, antibody molecule, multispecific molecule, and/or multifunctional molecule as described herein comprising an anti-TCRβV molecule that binds to one or more members of the TCRβV12 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV12 subfamily. In some embodiments, administration of the agent, antibody molecule, multispecific molecule, and/or multifunctional molecule as described herein results in reducing or depleting the population of autoreactive T cells expressing one or more members of the TCRβV12 subfamily. In some embodiments, the subject having type I diabetes has a population of autoreactive T cells that express a TCRαV12 subfamily comprising TCRαV12-3. In some embodiments, the subject is administered the agent, antibody molecule, multispecific molecule, and/or multifunctional molecule as described herein comprising an anti-TCRαV molecule that binds to one or more members of the TCRαV12 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV12 subfamily. In some embodiments, administration of the agent, antibody molecule, multispecific molecule, and/or multifunctional molecule as described herein results in reducing or depleting the population of autoreactive T cells expressing one or more members of the TCRαV12 subfamily.

In some aspects, the present disclosure provides methods for depleting or reducing T cells in a subject in need thereof. In some embodiments, the T cells are autoreactive T cells. In some embodiments, the autoreactive T cells target autologous T cells of the subject. In some embodiments, the autoreactive T cells express TCRαV, TCRβV, or any combination thereof. In some embodiments, the subject suffers from an autoimmune disease. In some embodiments, the methods comprise administering to the subject an agent comprising a moiety. In some embodiments, the moiety binds to a TCR variable region.

In some embodiments, administering the agent depletes a T cell population in a subject by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater than about 99% compared to a baseline measure of the T cell population (e.g., a measure of T cell population prior to administering of the agent). In some embodiments, administering the agent depletes a T cell population in a subject by at most about 99%, at most about 98%, at most about 97%, at most about 96%, at most about 95%, at most about 94%, at most about 93%, at most about 92%, at most about 91%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, or less than about 20% compared to a baseline measure of the T cell population (e.g., a measure of T cell population prior to administering of the agent).

In some embodiments, administering the agent depletes a T cell population in a subject from about 20% to about 99% compared to a baseline measure of the T cell population (e.g., a measure of T cell population prior to administering of the agent). In some embodiments, administering the agent depletes a T cell population in a subject from about 20% to about 30%, about 20% to about 40%, about 20% to about 50%, about 20% to about 60%, about 20% to about 70%, about 20% to about 75%, about 20% to about 80%, about 20% to about 85%, about 20% to about 90%, about 20% to about 95%, about 20% to about 99%, about 30% to about 40%, about 30% to about 50%, about 30% to about 60%, about 30% to about 70%, about 30% to about 75%, about 30% to about 80%, about 30% to about 85%, about 30% to about 90%, about 30% to about 95%, about 30% to about 99%, about 40% to about 50%, about 40% to about 60%, about 40% to about 70%, about 40% to about 75%, about 40% to about 80%, about 40% to about 85%, about 40% to about 90%, about 40% to about 95%, about 40% to about 99%, about 50% to about 60%, about 50% to about 70%, about 50% to about 75%, about 50% to about 80%, about 50% to about 85%, about 50% to about 90%, about 50% to about 95%, about 50% to about 99%, about 60% to about 70%, about 60% to about 75%, about 60% to about 80%, about 60% to about 85%, about 60% to about 90%, about 60% to about 95%, about 60% to about 99%, about 70% to about 75%, about 70% to about 80%, about 70% to about 85%, about 70% to about 90%, about 70% to about 95%, about 70% to about 99%, about 75% to about 80%, about 75% to about 85%, about 75% to about 90%, about 75% to about 95%, about 75% to about 99%, about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99% compared to a baseline measure of the T cell population (e.g., a measure of T cell population prior to administering of the agent).

In some embodiments, administering the agent to a subject in need thereof provides sustained reducing and/or depletion of a targeted T cell or T cell population. In some embodiments, administering the agent to a subject in need thereof can deplete T cells for at least about 30 minutes, at least about 1 hour, at least about 2 hours, at least about 3 hours, at least about 4 hours, at least about 5 hours, at least about 6 hours, at least about 12 hours, at least about 1 day, at least about 2 days, at least about 3 days, at least about 4 days, at least about 5 days, at least about 6 days, at least about 7 days, at least about 2 weeks, at least about 3 weeks, at least about 4 weeks, at least about 2 months, at least about 3 months, at least about 4 months, at least about 5 months, at least about 6 months, or greater than about 6 months.

The methods herein may also provide identifying a subject based on a TCR clonotype. Identifying a subject may comprise genotypying a subject who suffers from a disease or condition. The disease may be an autoimmune disease. The autoimmune disease may be selected from the group consisting of amyotrophic lateral sclerosis (ALS), coeliac disease (CD), ankylosing spondylitis (AS), Covid-induced multisystem inflammatory syndrome in children (MIS-C), primary Sjogren's syndrome (PSS), Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellardegeneration (PCD), Lambert-Eaton syndrome, psoriasis, scleroderma, CREST (calcinosis,Raynaud phenomenon, esophageal dysmotility, sclerodactyly, and telangiectasia) syndrome,dermatitis herpetiformis, dermatomyositis, bullous pemphigoid, cicatricial pemphigoid/benignmucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis),Takayasu's arteritis, polyarteritis nodosa, microscopic polyangiitis (MPA), leukocytoclasticvasculitis, Cogan's syndrome, uveitis, peripheral uveitis (Pars planitis), scleritis, autoimmuneinner ear disease (AIED), Crohn's, ulcerative colitis (UC), Dressler's syndrome, Rheumaticfever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), ocular cicatricial pemphigoid, or Hashimoto's thyroiditis.

A sample may be obtained from a subject and sequenced to determining a TCR clonotype associated with the autoimmune disease. The TCR clonotype can comprise a T cell receptor alpha variable region (TCRαV) or a T cell receptor variable beta region (TCRβV). In some embodiments, the genotype may be determined. In some embodiments, commercial methodologies available for genotyping, for example, SNP genotyping, can be used, but are not limited to, TaqMan genotyping assays (Applied Biosystems), SNPlex platforms (Applied Biosystems), gel electrophoresis, capillary electrophoresis, size exclusion chromatography, mass spectrometry, for example, MassARRAY system (Sequenom), minisequencing methods, real-time Polymerase Chain Reaction (PCR), Bio-Plex system (BioRad), CEQ and SNPstream systems (Beckman), array hybridization technology, for example, Affymetrix GeneChip (Perlegen), BeadArray Technologies, for example, Illumina GoldenGate and Infinium assays, array tag technology, Multiplex Ligation-dependent Probe Amplification (MLPA), and endonuclease-based fluorescence hybridization technology (Invader assay, either using unamplified or amplified genomic DNA, or unamplified total RNA, or unamplified or amplified cDNA; Third Wave/Hologic).

### Combination Therapies

In some embodiments, the method as described herein further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the present invention may be used in combination with immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludarabine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In some embodiments, the compositions and pharmaceutical compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In some embodiments, the second therapeutic agent or therapy is administered in combination with the agent as described herein, the nucleic acid molecules as described herein, the vector as described herein, the cell as described herein, the pharmaceutical composition as described herein, sequentially, simultaneously, or concurrently.

The molecules or the composition as described herein can be used in combination with a second therapeutic agent or procedure.

In some embodiments, the molecules or the composition as described herein and the second therapeutic agent or procedure are administered/performed after a subject has been diagnosed with an autoimmune disease, e.g., before the autoimmune disease has been eliminated from the subject. In some embodiments, the molecules or the composition as described herein and the second therapeutic agent or procedure are administered/performed simultaneously or concurrently. For example, the delivery of one treatment is still occurring when the delivery of the second commences, e.g., there is an overlap in administration of the treatments. In other embodiments, the molecules or the composition as described herein and the second therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first and second treatment is more effective (e.g., leads to a greater reduction in symptoms and/or autoreactive T cells) than the first or second treatment alone. In some embodiments, the combination therapy permits use of a lower dose of the first or the second treatment compared to the dose of the first or second treatment normally required to achieve similar effects when administered as a monotherapy. In some embodiments, the combination therapy has a partially additive effect, wholly additive effect, or greater than additive effect.

In some embodiments, the anti-TCRαV antibody and/or the anti-TCRβV antibody, multispecific or multifunctional molecule is administered in combination with a therapy, e.g., a autoimmune disease therapy (e.g., one or more of immunotherapy, photodynamic therapy (PDT), surgery and/or radiation). The terms “chemotherapeutic,” “chemotherapeutic agent,” and “anti-cancer agent” are used interchangeably herein. The administration of the multispecific or multifunctional molecule and the therapy, e.g., the autoimmune disease therapy, can be sequential (with or without overlap) or simultaneous. Administration of the anti-TCRαV antibody and/or the anti-TCRβV antibody, multispecific or multifunctional molecule can be continuous or intermittent during the course of therapy (e.g., autoimmune therapy).

Methods described herein include treating an autoimmune disease in a subject by using the molecules or the composition as described herein, e.g., using a pharmaceutical composition as described herein. Also provided are methods for reducing or ameliorating a symptom of an autoimmune disease in a subject, as well as methods for inhibiting or preventing the activation and/or expansion of autoreactive T cell and/or reducing, depleting, or killing one or more autoreactive T cells. In some embodiments, the methods described herein decrease the number of autoreactive T cells in a subject administered with a described herein or a pharmaceutical composition described herein.

In some embodiments, the molecules or the composition as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in extent of disease symptoms, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

### Antibodies Against TCR Variable Region

### Anti-TCRαV Antibodies

Current bispecific constructs designed utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRαV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRαV antibody molecules as described herein, which despite having low sequence similarity (e.g., low sequence identity among the different antibody molecules that recognize different TCRαV subfamilies), recognize a structurally conserved, yet sequence-wise variable, region, e.g., domain, on the TCRαV protein and have a similar function (e.g., activation of T cells and a similar cytokine profile as described herein). Thus, the anti-TCRαV antibody molecules as described herein share a structure-function relationship.

In some embodiments, the anti-TCRαV antibody molecules as described herein do not recognize, e.g., bind to, an interface of a TCRβV:TCRalpha complex. In some embodiments, the anti-TCRαV antibody molecules as described herein do not recognize, e.g., bind to, a constant region of a TCRβV protein. In some embodiments, the anti-TCRαV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the alpha subunit of TCR (TCRαV) which bind and, e.g., activate a subset of T cells. The anti-TCRαV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRαV antibodies as described herein have a cytokine profile, e.g., as described herein, which differs from a cytokine profile of a T cell engager that binds to a receptor or molecule other than a TCRαV region (“a non-TCRαV-binding T cell engager”). In some embodiments, the non-TCRαV-binding T cell engager comprises an antibody that binds to a CD3 molecule (e.g., CD3 epsilon (CD3e) molecule); or a TCR alpha (TCRα) molecule. In some embodiments, the non-TCRαV-binding T cell engager is an OKT3 antibody or an SP34-2 antibody.

In some embodiments, the anti-TCRαV antibodies as described herein result in expansion of TCRαV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRαV antibody molecules and uses thereof. Also described herein are multispecific molecules, e.g., bispecific molecules comprising said anti-TCRαV antibody molecules. In some embodiments, compositions comprising anti-TCRαV antibody molecules of the present disclosure, can be used, e.g., to: (1) inhibit or prevent the activation and/or expansion of autoreactive T cells; and/or (2) reduce or deplete autoreactive T cells. In some embodiments, compositions comprising anti-TCRαV antibody molecules as described herein limit the harmful side-effects of CRS and/or NT, e.g., CRS and/or NT associated with anti-CD3e targeting.

In some embodiments, the anti-TCRαV antibody molecule is a full antibody or fragment thereof (e.g., a Fab, F(ab′)₂, Fv, single domain antibody, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgAg1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

The various TCRαV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference.

In some embodiments, the anti-TCRαV antibody molecule is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule is isolated or recombinant.

In some embodiments, the anti-TCRαV antibody molecule comprises a heavy chain constant region for an IgG4, e.g., a human IgG4. In still another embodiment, the anti-TCRαV antibody molecule includes a heavy chain constant region for an IgG1, e.g., a human IgG1. In some embodiments, the heavy chain constant region comprises an amino sequence set forth in Table 1, or a sequence substantially identical (e.g., at least 80%, 85%, 90%, 92%, 95%, 97%, 98%, 99% or higher identical) thereto.

In some embodiments, the anti-TCRαV antibody molecule includes a kappa light chain constant region, e.g., a human kappa light chain constant region. In some embodiments, the light chain constant region comprises an amino sequence set forth in Table 1, or a sequence substantially identical (e.g., at least 80%, 85%, 90%, 92%, 95%, 97%, 98%, 99% or higher identical) thereto.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule, is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule, is isolated or recombinant.

In some embodiments, the anti-TCRαV antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule comprises a light chain variable domain comprising: (a) a framework region 1 (FRI) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRαV antibody molecule is a full antibody or fragment thereof (e.g., a Fab, F(ab′)₂, Fv, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgAg1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

### Anti-TCRα V12 Antibodies

In one aspect, provided herein is an anti-TCRαV antibody molecule that binds to a human TCRα V12 subfamily member. In some embodiments, TCRα V12 subfamily is also known as TCRα V12. In some embodiments, the TCRα V12 subfamily comprises: TCRα V12-1, TCRα V12-2 or TCRα V12-3, or a variant thereof.

### Anti-TCRα V4 Antibodies

In one aspect, provided herein is an anti-TCRαV antibody molecule that binds to a human TCRα V4 subfamily member. In some embodiments, TCRα V4 subfamily is also known as TCRα V4.

### Anti-TCRα V21 Antibodies

In one aspect, provided herein is an anti-TCRαV antibody molecule that binds to a human TCRα V21 subfamily member. In some embodiments, TCRα V21 subfamily is also known as TCRα V21.

### Anti-TCRα V26 Antibodies

In one aspect, provided herein is an anti-TCRαV antibody molecule that binds to a human TCRα V26 subfamily member. In some embodiments, TCRα V26 subfamily is also known as TCRα V26. In some embodiments, the TCRα V26 subfamily comprises: TCRα V26-1, TCRα V26-2, or a variant thereof.

### Antibodies Against TCRβV

Described herein are molecules targeting the TCRBV chain of TCR and methods thereof. Without wishing to be bound by theory, such molecules are capable of binding, suppressing, and/or eliminating only a subset of T cells, and reducing immunoresponse mediated either directly or indirectly by the T cells that are bound by such molecules.

Described herein is a class of antibodies, i.e., anti-TCRβV antibody molecules as described herein, which despite having low sequence similarity (e.g., low sequence identity among the different antibody molecules that recognize different TCRBV subfamilies), recognize a structurally conserved, yet sequence-wise variable, region, e.g., domain, on the TCRBV protein (as denoted by the circled area in FIG. 1A) and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRβV antibody molecules as described herein bind to an outward facing epitope of a TCRBV protein when it is in a complex with a TCRalpha protein, e.g., as denoted by the circled area in FIG. 1A. In some embodiments, the anti-TCRBV antibody molecules as described herein recognize (e.g., bind to), a domain (e.g., an epitope) on the TCRβV protein that is: (1) structurally conserved among different TCRBV subfamilies; and (2) has minimal sequence identity among the different TCRβV subfamilies. As shown in Table 8, TCRBV proteins from the different TCRBV subfamilies share minimal sequence similarity. However, as shown in FIGS. 1A-1B, TCRβV proteins which have minimal sequence similarity, share a similar 3D conformation and structure.

The alignment of TCRBV amino acid sequences in Table 8 underscores the diversity of TCR sequences. In particular, the TRBV sequences from different subfamilies are considerably different from each other.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRBV region is JOVI.1 as de-scribed in Viney et al., (Hybridoma. 1992 December; 11(6):701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., suppress or eliminate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in inhibition or depletion of a biased TCRβV+ clonotype or comprising a TCRBV antigen corresponding to a biased TCRBV clonotype. Without wishing to be bound by theory, a TCR bias may exist in autoimmune diseases. This bias may be associated with dominant autoreactive TCR clones responsible for disease or associated with symptoms. Re-balancing the TCR repertoire, e.g., by eliminating or depleting T cells comprising an autoreactive clonotype, may treat the associated autoimmune disease and/or reduce symptoms of the autoimmune disease. Accordingly, provided herein are methods of making said anti-TCRβV antibody molecules and uses thereof.

In some embodiments, the anti-TCRβV antibody molecule binds to one or more of TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1 and TRBV30. In some embodiments, the anti-TCRβV antibody molecule binds to one or more of TRBV6-1, TRBV6-2, TRBV6-3, TRBV6-4, TRBV6-5, TRBV6-6, TRBV6-8 and TRBV6-9. In some embodiments, the anti-TCRβV antibody molecule is an anti-TRBV2, anti-TRBV3-1, anti-TRBV4-1, anti-TRBV4-2, anti-TRBV4-3, anti-TRBV5-1, anti-TRBV5-4, anti-TRBV5-5, anti-TRBV5-6, anti-TRBV5-8, anti-TRBV6-1, anti-TRBV6-2, anti-TRBV6-3, anti-TRBV6-4, anti-TRBV6-5, anti-TRBV6-6, anti-TRBV6-8, anti-TRBV6-9, anti-TRBV7-2, anti-TRBV7-3, anti-TRBV7-4, anti-TRBV7-6, anti-TRBV7-7, anti-TRBV7-8, anti-TRBV7-9, anti-TRBV9, anti-TRBV10-1, anti-TRBV10-2, anti-TRBV10-3, anti-TRBV11-1, anti-TRBV11-2, anti-TRBV11-3, anti-TRBV12-3, anti-TRBV12-4, anti-TRBV12-5, anti-TRBV13, anti-TRBV14, anti-TRBV15, anti-TRBV16, anti-TRBV18, anti-TRBV19, anti-TRBV20-1, anti-TRBV24-1, anti-TRBV25-1, anti-TRBV27, anti-TRBV28, anti-TRBV29-1, or anti-TRBV30. Exemplary anti-TCRβV antibody molecules and the corresponding TCRβV subfamilies recognized by said anti-TCRβV antibody molecules are disclosed in Table 9.

In some embodiments, the anti-TCRβV antibody molecule binds specifically to TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1 or TRBV30. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9.

In some embodiments, the anti-TCRβV antibody molecule binds specifically to a TRBV that is associated with an autoimmune disease, such as TRBV4, TRBV6-1, TRBV6-2, TRBV6-3, TRBV6-5, TRBV6-2/3, TRBV7.1, TRBV7, TRBV7-8, TRBV7-9, TRBV7-8/9, TRBV9, TRBV10-3, TRBV11-2, TRBV12-3, TRBV12-4, TRBV12-3/4, TRBV20-1, TRBV23, or TRBV29-1. In some embodiments, the anti-TCRβV antibody molecule binds specifically to TRBV4. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-5. In some embodiments, the anti-TCRβV antibody molecule binds specifically to TRBV6-2/3. In some embodiments, the anti-TCRβV antibody molecule binds specifically to TRBV7.1. In some embodiments, the anti-TCRβV antibody molecule binds specifically to TRBV7. In some embodiments, the anti-TCRβV antibody molecule binds specifically to TRBV7-8. In some embodiments, the anti-TCRβV antibody molecule binds specifically to TRBV7-9. In some embodiments, the anti-TCRβV antibody molecule binds specifically to TRBV7-8/9. In some embodiments, the anti-TCRβV antibody molecule binds specifically to TRBV9. In some embodiments, the anti-TCRβV antibody molecule binds specifically to TRBV10-3. In some embodiments, the anti-TCRβV antibody molecule binds specifically to TRBV11-2. In some embodiments, the anti-TCRβV antibody molecule binds specifically to TRBV12-3. In some embodiments, the anti-TCRβV antibody molecule binds specifically to TRBV12-4. In some embodiments, the anti-TCRβV antibody molecule binds specifically to TRBV12-3/4. In some embodiments, the anti-TCRβV antibody molecule binds specifically to TRBV20-1. In some embodiments, the anti-TCRβV antibody molecule binds specifically to TRBV23. In some embodiments, the anti-TCRβV antibody molecule binds specifically to TRBV29-1.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FRI), comprising a change, e.g., a substitution (e.g., a conservative substitution) at position 10 according to Kabat numbering. In some embodiments, the FRI comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position as described herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FRI) comprising a Phenylalanine at position 10, e.g., a substitution at position 10 according to Kabat numbering, e.g., a Serine to Phenyalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FRI) comprising a change, e.g., a substitution (e.g., a conservative substitution) at one or more (e.g., all) positions as described herein according to Kabat numbering, (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position as described herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11. In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 1, or encoded by the nucleotide sequence in Table 1; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 1. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)₂, Fv, single domain antibody, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a TCR targeting molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, 99%, 99.5%, 99.9%, or 100% sequence identity thereto. Antibody A-H.69 comprises the amino acid sequence of SEQ ID NO: 1500, or a sequence having at least 85%, 90%, 95%, 96%, 97%, 98%, 99%, 99.5%, 99.9%, or 100% sequence identity thereto.

Additional exemplary humanized anti-TCRB V6 antibodies are provided in Table 1. In some embodiments, the anti-TCRβ V6 is antibody A, e.g., humanized antibody A (antibody A-H), as provided in Table 1. In some embodiments, the anti-TCRβV antibody comprises one or more (e.g., all three) of a LC CDR1, LC CDR2, and LC CDR3 provided in Table 1; and/or one or more (e.g., all three) of a HC CDR1, HC CDR2, and HC CDR3 provided in Table 1, or a sequence with at least 85%, 90%, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 1, or a sequence with at least 85%, 90%, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCRβV subfamilies recognized by said anti-TCRβV antibody molecules are disclosed in Table 9.

The various TCRβV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference. For example, TCRβ V6-5 is represented in approximately 3-6% healthy donors.

### Anti-TCRβ V6 Antibodies

In one aspect, provided herein is an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or an amino acid sequence having 85%, 90%, 95%, 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

-Consensus VH
SEQ ID NO: 231
QVQLVQSGAEVKKPGSSVKVSCKASGH/T/G/YD/T/SFH/R/D/K/
TL/D/K/T/NW/F/T/I/Y/GYIHWVRQAPGQGLEWMGR/WV/I/FF/
S/YA/PGSGN/ST/V/Y/IK/RYNEKFKGRVTITADTSTSTAYMELSSL
RSEDTAVYYCAG/VSY/IYSY/AD/GVLDYWGQGTTVTVSS

### SEQ ID NO: 3290—Consensus VH

QVQLVQSGAEVKKPGSSVKVSCKASGX₁X₂FX₃X₄X₅YIHWVRQAPGQGLEWMGXX₇X₈X₉GSGX₁₀X₁₁X₁₂YNEKFKGRVTITADTSTSTAYMELSSLRSEDTAVYYCAX₁₃SX₁₄YSX₁₅X₁₆VLDYWGQGTT VTVSS, where-in: X1 is H or T or G or Y; X2 is D or T or S; X3 is H or R or D or K or T; X4 is L or D or K or T or N; X5 is W or F or T or I or Y or G; X6 is R or W; X7 is V or I or F; X8 is F or S or Y; X9 is A or P; X10 is N or S; X11 is T or V or Y or I; X12 is K or R; X13 is G or V; X14 is Y or I; X15 is Y or A; and X16 is D or G.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 230 or 3289.

-Consensus VL
SEQ ID NO: 230
DIQMTQSPSFLSASVGDRVTITCKASQNVG/E/A/DN/DR/KVAWY/
HQQKPGKAPKALIYSSSHRYK/SGVPSRFSGSGSGTEFTLTISSLQPED
FATYFCQQFKSYPLTFGQGTKLEIK

### SEQ ID NO: 3289—Consensus VL

DIQMTQSPSFLSASVGDRVTITCKASQNVX₁X₂X₃VAWX₄QQKPGKAPKALIYSSSHRYXSGVPSRF SGSGSGTEFTLTISSLQPEDFATYFCQQFKSYPLTFGQGTKLEIK, wherein X1 is G, E, A or D; X2 is N or D; X3 is R or K; X4 is Y or H; and X5 is K or S

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a heavy chain variable region of an antibody described herein, e.g., an antibody chosen from any one of A-H.1 to A-H.85, e.g., A-H.1, A-H.2 or A-H.68, or as described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 1.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 1.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 1 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 1. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 1.

In some embodiments, e.g., an embodiment comprising a variable region, a CDR (e.g., a combined CDR, Chothia CDR or Kabat CDR), or other sequence referred to herein, e.g., in Table 1, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a TCR targeting molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises a VH and/or a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1, or a sequence with at least 80%, 85%, 90%, 95%, 96%, 97%, 98%, 99% or more identity thereto.

In some embodiments, an anti-TCRVb antibody as described herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody as described herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Anti-TCRβ V12 Antibodies

In one aspect, provided herein is an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two heavy chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two light chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, molecule includes all six CDRs from an antibody described herein, e.g., an antibody as described in Table 2, or encoded by the nucleotide sequence in Table 2, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V12 antibody molecule includes at least one, two, three, four, five, or six CDRs according to Kabat et al. (e.g., at least one, two, three, four, five, or six CDRs according to the Kabat definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 2.

In some embodiments, the anti-TCRβV antibody molecule, e.g., anti-TCRβ V12 antibody molecule includes all six CDRs according to Kabat et al. (e.g., all six CDRs according to the Kabat definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three hypervariable loops that have the same canonical structures as the corresponding hypervariable loop of an antibody described herein, e.g., an antibody described in Table 2, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V12 antibody molecule includes at least one, two, three, four, five, or six CDRs according to Chothia et al. (e.g., at least one, two, three, four, five, or six CDRs according to the Chothia definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 2.

In some embodiments, the anti-TCRβV antibody molecule, e.g., anti-TCRβ V12 antibody molecule includes all six CDRs according to Chothia et al. (e.g., all six CDRs according to the Chothia definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 2.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 2.

In some embodiments, the anti-TCRβV antibody molecule, e.g., anti-TCRβ V12 antibody molecule includes at least one, two, three, four, five, or six CDRs according to a combined CDR. (e.g., at least one, two, three, four, five, or six CDRs according to the combined CDR definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 2.

In some embodiments, the anti-TCRβV antibody molecule, e.g., anti-TCRβ V12 antibody molecule includes all six CDRs according to a combined CDR (e.g., all six CDRs according to the combined CDR definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 2. In some embodiments, the anti-TCRβV antibody molecule, e.g., anti-TCRβ V12 antibody molecule may include any CDR described herein.

In some embodiments, a combined CDR as set out in Table 1 is a CDR that comprises a Kabat CDR and a Chothia CDR.

In some embodiments, the anti-TCRβV antibody molecule, e e.g., anti-TCRβ V12 antibody molecule, molecule includes a combination of CDRs or hypervariable loops identified as combined CDRs in Table 1. In some embodiments, the anti-TCRβV antibody molecule, e.g., anti-TCRβ V12 antibody molecule, can contain any combination of CDRs or hypervariable loops according the “combined” CDRs are described in Table 1.

In some embodiments, the anti-TCRβV antibody molecule, e.g., anti-TCRβ V12 antibody molecule includes a combination of CDRs or hypervariable loops defined according to the Kabat et al. and Chothia et al., or as described in Table 1.

In some embodiments, the anti-TCRβV antibody molecule, e.g., anti-TCRβ V12 antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, e.g., in Table 2, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a TCR targeting molecule, e.g., a bispecific molecule, e.g., as described herein.

In some embodiments, the anti-TCRβV antibody molecule, e.g., anti-TCRβ V12 antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24 or SEQ ID NO: 25.

In some embodiments, the anti-TCRβV antibody molecule, e.g., anti-TCRβ V12 antibody molecule comprises: (i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or (ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or (ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβ V12 antibody molecule comprises: (i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or (ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the light or the heavy chain variable framework (e.g., the region encompassing at least FRI, FR2, FR3, and optionally FR4) of the anti-TCRβV antibody molecule, e.g., anti-TCRβ V12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FRI, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V12 antibody molecule, comprises a heavy chain variable domain having at least one, two, three, four, five, six, seven, ten, fifteen, twenty or more changes, e.g., amino acid substitutions or deletions, from an amino acid sequence described in Table 2 e.g., the amino acid sequence of the FR region in the entire variable region, e.g., SEQ ID NOs: 23-25.

Alternatively, or in combination with the heavy chain substitutions described herein the anti-TCRβV antibody molecule, e.g., anti-TCRβ V12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein .e.g., the amino acid sequence of the FR region in the entire variable region, e.g., SEQ ID NOs: 26-30.

In some embodiments, the anti-TCRβV antibody molecule, e.g., anti-TCRβ V12 antibody molecule includes one, two, three, or four heavy chain framework regions, or a sequence substantially identical thereto.

In some embodiments, the anti-TCRβV antibody molecule, e.g., anti-TCRβ V12 antibody molecule includes one, two, three, or four light chain framework regions, or a sequence substantially identical thereto. In some embodiments, the anti-TCRβV antibody molecule, e.g., anti-TCRβ V12 antibody molecule comprises the light chain framework region 1. In some embodiments, the anti-TCRβV antibody molecule, e.g., anti-TCRβ V12 antibody molecule comprises the light chain framework region 2. In some embodiments, the anti-TCRβV antibody molecule, e.g., anti-TCRβ V12 antibody molecule comprises the light chain framework region 3. In some embodiments, the anti-TCRβV antibody molecule, e.g., anti-TCRβ V12 antibody molecule comprises the light chain framework region 4.

In some embodiments, the anti-TCRβV antibody molecule, e.g., anti-TCRβ V12 antibody molecule comprises a light chain comprising a framework region, e.g., framework region 1 (FRI), comprising a change, e.g., a substitution (e.g., a conservative substitution) at one or more, e.g., all, position as described herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FRI comprises an Asparagine at position 2, e.g., a substitution at position 2 according to Kabat numbering, e.g., an Isoleucine to Asparagine substitution, Serine to Asparagine substitution or Tyrosine to Asparagine substitution. In some embodiments, FRI comprises a Leucine at position 4, e.g., a substitution at position 4 according to Kabat numbering, e.g., a Methionine to Leucine substitution.

In some embodiments, the anti-TCRβV antibody molecule, e.g., anti-TCRβ V12 antibody molecule comprises a light chain comprising a framework region, e.g., framework region 1 (FRI), comprising a substitution at position 1 according to Kabat numbering, e.g., an Alanine to Aspartic Acid substitution, a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FRI), comprising a substitution at position 1 according to Kabat numbering, e.g., an Alanine to Aspartic Acid substitution, and a substitution at position 2 according to Kabat numbering, e.g., an Isoleucine to Asparagine substitution, Serine to Asparagine substitution or Tyrosine to Asparagine substitution. In some embodiments, the anti-TCRβV antibody molecule, e.g., anti-TCRβ V12 antibody molecule comprises a light chain comprising a framework region, e.g., framework region 1 (FRI), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FRI), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβ V12 antibody molecule comprises a light chain comprising a framework region, e.g., framework region 3 (FR3), comprising a change, e.g., a substitution (e.g., a conservative substitution) at one or more, e.g., all, position as described herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments, FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβ V12 antibody molecule comprises a light chain comprising: a framework region 1 (FRI) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FRI) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FRI) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FRI) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FRI) comprising a substitution at position 2 according to Kabat numbering, e.g., a Tyrosine to Asparagine substitution; and (b) a framework region 3 (FR3) comprising a substitution at position 66 according to Kabat numbering, e.g., a Serine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain variable domain comprising: (a) a framework region 1 (FRI) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V12 antibody molecule comprises the heavy chain framework region 1. In some embodiments, the anti-TCRβV antibody molecule, e.g., anti-TCRβ V12 antibody molecule comprises the heavy chain framework region 2. In some embodiments, the anti-TCRβV antibody molecule, e.g., anti-TCRβ V12 antibody molecule comprises the heavy chain framework region 3. In some embodiments, the anti-TCRβV antibody molecule, e.g., anti-TCRβ V12 antibody molecule comprises the heavy chain framework region 4. In some embodiments, the anti-TCRβV antibody molecule, e.g., anti-TCRβ V12 antibody molecule comprises the heavy chain framework regions 1-4, e.g., SEQ ID NOS: 20-23. In some embodiments, the anti-TCRβV antibody molecule, e.g., anti-TCRβ V12 antibody molecule comprises the light chain framework regions 1-4, e.g., SEQ ID NOs: 26-30.

In some embodiments, the anti-TCRβV antibody molecule, e.g., anti-TCRβ V12 antibody molecule comprises the heavy chain framework regions 1-4, e.g., SEQ ID NOs: 23-25; and the light chain framework regions 1-4, e.g., SEQ ID NOs: 26-30.

In some embodiments, the heavy or light chain variable domain, or both, of, the anti-TCRβV antibody molecule, e.g., anti-TCRβ V12 antibody molecule includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody as described in Table 2, or encoded by the nucleotide sequence in Table 2; or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule comprises at least one, two, three, or four antigen-binding regions, e.g., variable regions, having an amino acid sequence as set forth in Table 2, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 2. In another embodiment, the anti-TCRβV antibody molecule, e.g., anti-TCRβ V12 antibody molecule includes a VH and/or VL domain encoded by a nucleic acid having a nucleotide sequence as set forth in Table 2, or a sequence substantially identical thereto (e.g., a sequence at least about 85%, 90%, 95%, 99% or more identical thereto, or which differs by no more than 3, 6, 15, 30, or 45 nucleotides from the sequences shown in Table 2.

In some embodiments, the anti-TCRβV antibody molecule, e.g., anti-TCRβ V12 antibody molecule comprises: a VH domain comprising an amino acid sequence chosen from the amino acid sequence of SEQ ID NO: 23, SEQ ID NO:24 or SEQ ID NO:25, an amino acid sequence at least about 85%, 90%, 95%, 99% or more identical to the amino acid sequence SEQ ID NO: 23, SEQ ID NO:24 or SEQ ID NO:25, or an amino acid sequence which differs by no more than 1, 2, 5, 10, or 15 amino acid residues from the amino acid sequence of SEQ ID NO: 23, SEQ ID NO:24 or SEQ ID NO:25; and/or a VL domain comprising an amino acid sequence chosen from the amino acid sequence of SEQ ID NO: 26, SEQ ID NO: 27, SEQ ID NO: 28, SEQ ID NO: 29 or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29 or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29 or SEQ ID NO: 30. In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule is a full antibody or fragment thereof (e.g., a Fab, F(ab′)₂, Fv, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule is a humanized antibody molecule. The heavy and light chains of the anti-TCRβV antibody molecule, e.g., anti-TCRβ V12 antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule is in the form of a TCR targeting molecule, e.g., a bispecific molecule, e.g., as described herein.

In some embodiments, the anti-TCRβV antibody molecule, e.g., anti-TCRβ V12 antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβ V12 antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V12 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβ V12 antibodies are provided in Table 2. In some embodiments, the anti-TCRβ V12 is antibody B, e.g., humanized antibody B (antibody B-H), as provided in Table 2. In some embodiments, the anti-TCRβV antibody comprises one or more (e.g., all three) of a LC CDR1, LC CDR2, and LC CDR3 provided in Table 2; and/or one or more (e.g., all three) of a HC CDR1, HC CDR2, and HC CDR3 provided in Table 2, or a sequence with at least 95% sequence identity thereto. In some embodiments, antibody B comprises a variable heavy chain (VH) and/or a variable light chain (VL) provided in Table 2, or a sequence with at least 95% sequence identity thereto.

In some embodiments, the anti-TCRVB 12 antibody molecule (e.g., anti-TCRVB 12-3 or anti-TCRVB 12-4 antibody molecule) comprises a VH of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRVB 12 antibody molecule (e.g., anti-TCRVB 12-3 or anti-TCRVB 12-4 antibody molecule) comprises a VL of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRVB 12 antibody molecule (e.g., anti-TCRVB 12-3 or anti-TCRVB 12-4 antibody molecule) comprises a VH of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto; and a VL of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

### Anti-TCRβ V5 Antibodies

In one aspect, provided herein is an anti-TCRβV antibody molecule that binds to human TCRβ V5. In some embodiments, the TCRβ V5 subfamily comprises TCRβ V5-5*01, TCRβ V5-6*01, TCRβ V5-4*01, TCRβ V5-8*01, TCRβ V5-1*01, or a variant thereof.

Exemplary anti-TCRβ V5 antibodies are provided in Table 10. In some embodiments, the anti-TCRβ V5 is antibody C, e.g., humanized antibody C (antibody C-H), as provided in Table 10. In some embodiments, the anti-TCRβV antibody comprises one or more (e.g., all three) of a LC CDR1, LC CDR2, and LC CDR3 provided in Table 10; and/or one or more (e.g., all three) of a HC CDR1, HC CDR2, and HC CDR3 provided in Table 10, or a sequence with at least 95% sequence identity thereto. In some embodiments, antibody C comprises a variable heavy chain (VH) and/or a variable light chain (VL) provided in Table 10, or a sequence with at least 95% sequence identity thereto.

Exemplary anti-TCRβ V5 antibodies are provided in Table 11. In some embodiments, the anti-TCRβ V5 is antibody E, e.g., humanized antibody E (antibody E-H), as provided in Table 11. In some embodiments, the anti-TCRβV antibody comprises one or more (e.g., all three) of a LC CDR1, LC CDR2, and LC CDR3 provided in Table 11; and/or one or more (e.g., all three) of a HC CDR1, HC CDR2, and HC CDR3 provided in Table 11, or a sequence with at least 95% sequence identity thereto. In some embodiments, antibody E comprises a variable heavy chain (VH) and/or a variable light chain (VL) provided in Table 11, or a sequence with at least 95% sequence identity thereto.

In some embodiments, antibody E comprises a heavy chain comprising the amino acid sequence of SEQ ID NO: 3284 and/or a light chain comprising the amino acid sequence of SEQ ID NO: 3285, or a sequence with at least 95% sequence identity thereto.

In some embodiments, the anti-TCRβ V5 antibody molecule comprises a VH and/or a VL of an antibody described in Table 10, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 10, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 11, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 11, or a sequence with at least 80%, 85%, 90%, 95%, 96%, 97%, 98%, 99% or more identity thereto.

### Anti-TCRβ V10 Antibodies

In one aspect, provided herein is an anti-TCRβV antibody molecule that binds to a human TCRβ V10 subfamily member. In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

Exemplary anti-TCRβ V10 antibodies are provided in Table 12. In some embodiments, the anti-TCRβ V10 is antibody D, e.g., humanized antibody D (antibody D-H), as provided in Table 12. In some embodiments, antibody D comprises one or more (e.g., three) light chain CDRs and/or one or more (e.g., three) heavy chain CDRs provided in Table 12, or a sequence with at least 95% sequence identity thereto. In some embodiments, antibody D comprises a variable heavy chain (VH) and/or a variable light chain (VL) provided in Table 12, or a sequence with at least 95% sequence identity thereto.

In some embodiments, the anti-TCRβ V10 antibody molecule comprises a VH or a VL of an antibody described in Table 12, or a sequence with at least 80%, 85%, 90%, 95%, 96%, 97%, 98%, 99% or more identity thereto.

In some embodiments, the anti-TCRβ V10 antibody molecule comprises a VH and a VL of an antibody described in Table 12, or a sequence with at least 80%, 85%, 90%, 95%, 96%, 97%, 98%, 99% or more identity thereto.

### Anti-TCRβ V13 Antibodies

In one aspect, provided herein is an anti-TCRβV antibody molecule that binds to a mouse TCRβ V13 subfamily member. In some embodiments, the mouse TCRβ V13 subfamily comprises: TCRβ V13-1, TCRβ V13-2, TCRβ V13-3, or a variant thereof.

Exemplary anti-TCRβ V13 antibodies are provided in Table 13. In some embodiments, the anti-TCRβ V13 is antibody MR5-2, as provided in Table 13. In some embodiments, antibody MR5-2 comprises one or more (e.g., three) light chain CDRs and/or one or more (e.g., three) heavy chain CDRs provided in Table 13, or a sequence with at least 95% sequence identity thereto. In some embodiments, antibody MR5-2 comprises a variable heavy chain (VH) and/or a variable light chain (VL) provided in Table 13, or a sequence with at least 95% sequence identity thereto.

In some embodiments, the anti-TCRβ V13 antibody molecule comprises a VH or a VL of an antibody described in Table 13, or a sequence with at least 80%, 85%, 90%, 95%, 96%, 97%, 98%, 99% or more identity thereto. In some embodiments, the anti-TCRβ V13 antibody molecule comprises a VH and a VL of an antibody described in Table 12, or a sequence with at least 80%, 85%, 90%, 95%, 96%, 97%, 98%, 99% or more identity thereto.

In some embodiments, an anti-TCRβ V13 antibody as described herein may comprise a humanized antibody. In some embodiments, an anti-TCRβ V13 antibody as described herein comprises one or more (e.g., three) light chain CDRs and/or one or more (e.g., three) heavy chain CDRs provided in Table 13, or a sequence with at least 95% sequence identity thereto. In some embodiments, a sequence of a HC CDR1 of the anti-TCRβ V13 antibody can comprise at least about 95%, 96%, 97%, 98%, 99%, 99.5%, 99.9%, or 100% sequence identity to an amino acid sequence as set forth in any one of SEQ ID NOs: 7500, 7503, or 7505. In some embodiments, a sequence of a HC CDR2 of the anti-TCRβ V13 antibody can comprise at least about 95%, 96%, 97%, 98%, 99%, 99.5%, 99.9%, or 100% sequence identity to an amino acid sequence as set forth in any one of SEQ ID NOs: 7501 or 7504. In some embodiments, a sequence of a HC CDR3 of the anti-TCRβ V13 antibody can comprise at least about 95%, 96%, 97%, 98%, 99%, 99.5%, 99.9%, or 100% sequence identity to an amino acid sequence as set forth in SEQ ID NO: 7502. In some embodiments, a sequence of a LC CDR1 of the anti-TCRβ V13 antibody can comprise at least about 95%, 96%, 97%, 98%, 99%, 99.5%, 99.9%, or 100% sequence identity to an amino acid sequence as set forth in SEQ ID NO: 7506. In some embodiments, a sequence of a LC CDR2 of the anti-TCRβ V13 antibody can comprise at least about 95%, 96%, 97%, 98%, 99%, 99.5%, 99.9%, or 100% sequence identity to an amino acid sequence as set forth in SEQ ID NO: 7507. In some embodiments, a sequence of a LC CDR3 of the anti-TCRβ V13 antibody can comprise at least about 95%, 96%, 97%, 98%, 99%, 99.5%, 99.9%, or 100% sequence identity to an amino acid sequence as set forth in SEQ ID NO: 7508.

In some embodiments, an anti-TCRβ V13 antibody described herein can comprise a VH comprising at least about 80%, 85%, 90%, 91%, 92%, 93%, 94%, 95%, 96%, 97%, 98%, 98.5%, 99%, 99.5%, 99.9%, or 100% sequence identity to an amino acid sequence as set forth in SEQ ID NO: 7509. In some embodiments, an anti-TCRβ V13 antibody described herein can comprise a VL comprising at least about 80%, 85%, 90%, 91%, 92%, 93%, 94%, 95%, 96%, 97%, 98%, 98.5%, 99%, 99.5%, 99.9%, or 100% sequence identity to an amino acid sequence as set forth in SEQ ID NO: 3527. In some embodiments, an anti-TCRβ V13 antibody described herein can comprise a heavy chain comprising at least about 70%, 75%, 80%, 85%, 90%, 91%, 92%, 93%, 94%, 95%, 96%, 97%, 98%, 98.5%, 99%, 99.5%, 99.9%, or 100% sequence identity to an amino acid sequence as set forth in SEQ ID NO: 7511. In some embodiments, an anti-TCRβ V13 antibody described herein can comprise a light chain comprising at least about 70%, 75%, 80%, 85%, 90%, 91%, 92%, 93%, 94%, 95%, 96%, 97%, 98%, 98.5%, 99%, 99.5%, 99.9%, or 100% sequence identity to an amino acid sequence as set forth in SEQ ID NO: 7512.

### Additional Anti-TCRVβ Antibodies

Additional exemplary anti-TCRβV antibodies are provided in Table 13. In some embodiments, the anti-TCRβV antibody is a humanized antibody, e.g., as provided in Table 13. In some embodiments, the anti-TCRβV antibody comprises one or more (e.g., all three) of a LC CDR1, LC CDR2, and LC CDR3 provided in Table 13; and/or one or more (e.g., all three) of a HC CDR1, HC CDR2, and HC CDR3 provided in Table 13, or a sequence with at least 95% sequence identity thereto. In some embodiments, the anti-TCRβV antibody comprises a variable heavy chain (VH) and/or a variable light chain (VL) provided in Table 13, or a sequence with at least 95% sequence identity thereto.

### Antibody-Like Frameworks or Scaffolds

A wide variety of antibody/immunoglobulin frameworks or scaffolds can be employed in the anti-TCRVA or anti-TCRVB antibody molecules as described herein or multifunctional formats thereof so long as the resulting polypeptide includes at least one binding region which specifically binds to the target antigen, e.g., a TCRVA or TCRVB. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRVA or anti-TCRVB antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRVA or TCRVB). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel c-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRVA or anti-TCRVB antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Antibody Effector Function and Fc Region

In some embodiments, an anti-TCRVA or anti-TCRVB antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fe Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVA or anti-TCRVB antibody comprising a wild-type Fc region or a variant Fc region that has enhanced affinity for an Fc receptor, e.g., an Fc receptor described herein. In some embodiments, the enhanced affinity is compared to an otherwise similar antibody with a wildtype Fc region.

In some embodiments, the anti-TCRVA or anti-TCRVB antibody molecule disclosed herein has a light chain constant region chosen from, e.g., the light chain constant regions of kappa or lambda, preferably kappa (e.g., human kappa). In some embodiments, the constant region is altered, e.g., mutated, to modify the properties of the anti-TCRαV or anti-TCRβV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

Disclosed herein are methods of inhibiting or preventing the activation and/or expansion of autoreactive T cells in a subject comprising administering an anti-TCRVα antibody comprising a variant Fc region. In some embodiments, the administering occurs in vivo or in vitro. In some embodiments, the anti-TCRVα antibody comprising a variant Fc region has increased binding to C1q complement. In some embodiments, the anti-TCRVα antibody comprising a variant Fc region has increased effector function (e.g., increased antibody dependent cell-mediated cytotoxicity (ADCC), antibody-dependent cellular phagocytosis (ADCP) and/or complement dependent cytotoxicity (CDC). In some embodiments, the subject has an autoimmune disease.

Disclosed herein are methods of reducing or depleting autoreactive T cells in a subject comprising administering an anti-TCRVα antibody comprising a variant Fc region. In some embodiments, the administering occurs in vivo or in vitro. In some embodiments, the anti-TCRVα antibody comprising a variant Fc region has increased binding to C1q complement. In some embodiments, the anti-TCRVα antibody comprising a variant Fc region has increased effector function (e.g., increased antibody dependent cell-mediated cytotoxicity (ADCC), antibody-dependent cellular phagocytosis (ADCP) and/or complement dependent cytotoxicity (CDC). In some embodiments, the subject has an autoimmune disease.

In some embodiments, the first TCRβV-binding moiety, the second TCRβV-binding moiety, or a combination thereof binds to one or more of a TCRβV subfamily selected from the group consisting of: (i) TCRβ V2 subfamily comprising TCRβ V2*01; (ii) TCRβ V3 subfamily comprising TCRβ V3-1*01; (iii) TCRβ V4 subfamily comprising one or more selected from TCRβ V4-1, TCRβ V4-2, and TCRβ V4-3; (iv) TCRβ V5 subfamily comprising one or more selected from TCRβ V5-6*01, TCRβ V5-4*01, TCRβ V5-1*01, and TCRβ V5-8*01; (v) the TCRβ V6 subfamily comprising one or more selected from TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, and TCRβ V6-1*01; (vi) TCRβ V9 subfamily; (vii) TCRβ V10 subfamily comprising one or more selected from TCRβ V10-1*01, TCRβ V10-1*02, TCRβ V10-3*01, and TCRβ V10-2*01; (viii) TCRβ V11 subfamily comprising TCRβ VII-2; (ix) TCRβ V12 subfamily comprising one or more selected from TCRβ V12-4*01, TCRβ V12-3*01, and TCRβ V12-5*01; (x) TCRβ V13 subfamily comprising TCRβ V13*01; (xi) TCRβ V16 subfamily comprising TCRβ V16*01; (xii) TCRβ V19 subfamily comprising one or more selected from TCRβ V19*01 and TCRβ V19*02; (xiii) TCRβ V21 subfamily. (xiv) TCRβ V23 subfamily. (xv) TCRβ V27 subfamily; (xvi) TCRβ V28 subfamily, (xvii) TCRβ V29 subfamily, and (vxviii) TCRβ V30 subfamily.

### CARs and Cells Comprising the CAR

Chimeric antigen receptors (CARs) and engineered T cell receptors (TCRs), which comprise binding domains capable of interacting with a particular antigen, can allow T cells to suppress and/or kill target cells that express the particular antigen. Besides the ability of genetically modified T cells expressing a CAR or an engineered TCR to recognize and destroy respective target cells in vitro/ex vivo, CAR T cells show favorable kinetics compared to conventional drugs because they expand and persist in the patient's body. Without wishing to be bound by any particular theory, the molecules disclosed herein comprise a recombinant T cell receptor or chimeric antigen receptor (CAR) that binds to a TCRαV and/or a TCRβV and may be used to suppress and/or kill autoreactive T cells expressing the TCRαV and/or the TCRβV in a subject in need thereof. T cells isolated from the subject can be modified to express the appropriate anti-TCRαV and/or anti-TCRβV CAR, expanded ex vivo, and then reinfused into the subject. The modified CAR T cells can recognize the target cells expressing the target TCRV region, become activated, and kill the target cells.

Disclosed herein, in some embodiments, is a recombinant T cell receptor or a CAR that may comprise an extracellular domain (e.g., an antigen binding domain) that binds TCRαV and/or TCRβV, a transmembrane domain, and an intracellular signaling domain, wherein the intracellular signaling domain may comprise a costimulatory signaling region.

In some embodiments, a genetically modified cell, such as a helper T cell, a cytotoxic T cell, a memory T cell, gamma delta T cell, a natural killer cell, cytokine induced killer cell, a cell line thereof, a T memory stem cell, and other effector cell, comprises a chimeric antibody (anti-TCRαV and/or anti-TCRβV CAR), wherein the anti-TCRαV and/or the anti-TCRβV CAR comprises an extracellular domain comprising an autoantigen or fragment thereof, a transmembrane domain, and an intracellular signaling domain. In some embodiments, the genetically modified cell comprises the anti-TCRαV and/or anti-TCRβV CAR described herein. In another embodiment, the cell expresses the anti-TCRαV and/or anti-TCRβV CAR. In some embodiments, the cell has a high affinity for a TCRαV and/or a TCRβV. As a result, the cell can induce killing of autoreactive T cells expressing the TCRαV and/or the TCRβV.

It is also useful for the CAR T cell to have limited toxicity toward healthy cells, e.g., not autoreactive cell, and specificity to cells expressing other TCR (e.g. gamma delta T cells). Such specificity prevents or reduces off-target toxicity.

In some embodiments, the CAR T cell comprises a T cell, such as a primary cell, an expanded T cell derived from primary T cells, a T cell derived from stem cells differentiated in vitro, a T cell line such as Jurkat cells, other sources of T cells, combinations thereof, and other effector cells. Further assessment of efficacy and safety of the anti-GD TCR CAR can be performed, for example, as follows: Constructs can be transiently transfected into human cells, such as 293T/17. The surface expression can be detected with monoclonal antibodies (either IgG or ScFv) specific for extracellular domains, a linker between the domains, or other structure included in the anti-TCRαV and/or the anti-TCRβV CAR. Binding can be verified with specific secondary antibodies and quantified by flow cytometry. Additional target cell lines can be produced as needed by expression of human monoclonal antibodies on the surface of K562 cells.

### The Extracellular Domain

The extracellular domain that binds TCRαV and/or TCRβV may be any anti-TCRαV antibody, any anti-TCRβV antibody, or any fragments thereof disclosed herein. In certain embodiments, the extracellular domain/antigen binding domain may comprise a heavy chain variable region that may comprise three heavy chain complementarity determining regions (HCDRs), and a light chain variable region that may comprise three light chain complementarity determining regions (LCDRs). In some embodiment, the extracellular domain that binds TCRαV and/or TCRβV may be a Fab or a scFv.

In some instances, it is beneficial that the antigen binding domain is derived from the same species in which the CAR will ultimately be used. For example, for use in humans, it may be beneficial that the antigen binding domain of the CAR may comprise a human antigen receptor that binds a human antigen or a fragment thereof. In one exemplary embodiment, the CAR may bind a TCRαV and/or TCRβV in a mammal (e.g., a human).

In some embodiments, the extracellular domain that binds a TCRαV binds to one or more of a TCRαV subfamily selected from the group consisting of: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof.

In some embodiments, the extracellular domain that binds a TCRβV binds to one or more of a TCRβV subfamily selected from the group consisting of: a TCRβ V1 subfamily, a TCRβ V2 subfamily, a TCRβ V3 subfamily, a TCRβ V4, a TCRβ V5 subfamily, a TCRβ V6 subfamily, a TCRβ V7 subfamily, a TCRβ V8 subfamily, a TCRβ V9 subfamily, a TCRβ V10 subfamily, a TCRβ VII subfamily, a TCRβ V12 subfamily, a TCRβ V13 subfamily, a TCRβ V14 subfamily, a TCRβ V15 subfamily, a TCRβ V16 subfamily, a TCRβ V17 subfamily, a TCRβ V18 subfamily, a TCRβ V20 subfamily, a TCRβ V21 subfamily, a TCRβ V22 subfamily, a TCRβ V23 subfamily, a TCRβ V24 subfamily, TCRβ V25 subfamily, a TCRβ V26 subfamily, a TCRβ V27 subfamily, a TCRβ V29 subfamily, a TCRβ V30 subfamily, as well as family members of said subfamilies, and variants thereof.

Transmembrane Domain and/or Hinge Domain

With respect to the transmembrane domain, in various embodiments, the CAR may comprise a transmembrane domain that is fused to the extracellular domain of the CAR. In one embodiment, the CAR may comprise a transmembrane domain that naturally is associated with one of the domains in the CAR. In some embodiments, the transmembrane domain is selected or modified by amino acid substitution to avoid binding to the transmembrane domains of the same or different surface membrane proteins in order to minimize interactions with other members of the receptor complex.

The transmembrane domain may be derived either from a natural or from a synthetic source. When the source is natural, the domain may be derived from any membrane-bound or transmembrane protein. In one embodiment, the transmembrane domain may be synthetic, in which case it may comprise predominantly hydrophobic residues such as leucine and valine. In one aspect, a triplet of phenylalanine, tryptophan and valine may be found at each end of a synthetic transmembrane domain. Optionally, a short oligo- or polypeptide linker, between 2 and 10 amino acids in length may form the linkage between the transmembrane domain and the cytoplasmic signaling domain of the CAR. A glycine-serine (GS) doublet provides a particularly suitable linker.

In some embodiments, a variety of human hinges can be employed as well, including, but not limited to, the human Ig (immunoglobulin) hinge domain and the CD8 alpha hinge domain. Examples of the hinge and/or transmembrane domain include, but are not limited to, a hinge and/or transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD 134, CD137, CD154, KIR, 0X40, CD2, CD27, LFA-1 (CD 11 a, CD18), ICOS (CD278), 4-1BB (CD 137), GITR, CD40, BAFFR, HVEM (LIGHTR), SLAMF7, NKp80 (KLRFl), CD160, CD 19, IL2R beta, IL2R gamma, IL7R a, ITGA1, VLA1, CD49a, ITGA4, IA4, CD49D, ITGA6, VLA-6, CD49f, ITGAD, CDlld, ITGAE, CD103, ITGAL, CDlla, LFA-1, IT GAM, CDllb, ITGAX, CDllc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD 160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

### Intracellular Signaling Domain

The CAR of the present disclosure may comprise an intracellular signaling domain, wherein the intracellular signaling domain may comprise a costimulatory signaling region. The intracellular signaling domain of the CAR is responsible for activation of at least one of the effector functions of the cell in which the CAR is expressed. The intracellular domain transduces the effector function signal and directs the cell to perform its specialized function. Examples of an intracellular signaling domain include, but are not limited to, the cytoplasmic portion of a surface receptor, a co-stimulatory molecule, and any molecule that acts in concert to initiate signal transduction in the T cell, as well as any derivative or variant of these elements and any synthetic sequence that has the same functional capability.

As used herein, a “costimulatory molecule,” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. Exemplary costimulatory molecules including but are not limited to CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, or a ligand that specifically binds with CD83.

Examples of the intracellular signaling domain include, without limitation, the ζ chain of the T cell receptor complex or any of its homologs, e.g., η chain, FcsRFγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In certain embodiments, the intracellular signaling domain of the CAR includes any portion of one or more co-stimulatory molecules, such as at least one signaling domain from CD2, CD3, CD8, CD27, CD28, ICOS (CD278), 4-1BB, PD-1, any derivative or variant thereof, any synthetic sequence thereof that has the same functional capability, and any combination thereof. In certain embodiments, the intracellular domain may comprise a costimulatory domain of a protein selected from the group consisting of proteins in the TNFR superfamily, CD28, 4-IBB (CD 137), 0X40 (CD 134), PD-1, CD7, LIGHT, CD83L, DAPIO, DAP 12, CD27, CD2, CD5, ICAM-1, LFA-1, Lek, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In one embodiment, the CAR of the disclosure may comprise a CD137 (4-1BB) signaling domain. For example, inclusion of the CD137 (4-1BB) signaling domain significantly increased CAR mediated activity and in vivo persistence of CAR T cells compared to an otherwise identical CAR T cell not engineered to express CD137 (4-1BB). However, the disclosure is not limited to a specific CAR. Rather, any CAR that targets TCRαV and/or TCRβV, can be used in the present disclosure. Compositions and methods of making and using CARs have been described in PCT/US11/64191, which is incorporated by reference in its entirety herein. In certain embodiments, the intracellular signaling domain may comprise CD3zeta. In certain embodiments, the intracellular signaling domain may comprise CD28 and CD3zeta. In certain embodiments, the intracellular signaling domain may comprise 4-1BB and CD3zeta.

In certain embodiments, the intracellular signaling domain may comprise CD3zeta. In certain embodiments, the intracellular signaling domain may comprise CD28 and CD3zeta. In certain embodiments, the intracellular signaling domain may comprise 4-1BB and CD3zeta.

In another aspect, provided herein is a T cell genetically modified to express a recombinant T cell receptor, wherein the recombinant T cell receptor comprises a domain that binds a TCRαV and/or TCRβV. In some embodiments, the domain that binds a Va region of a T cell receptor may be an α/β heterodimer of the recombinant T cell receptor.

In some embodiments, provide herein are T cells genetically modified to express any of the TCRαV-specific CAR disclosed herein. In some embodiments, the cell may have high affinity for cells expressing TCRαV.

In some embodiments, provide herein are T cells genetically modified to express any of the TCRβV-specific CAR disclosed herein. In some embodiments, the cell may have high affinity for cells expressing TCRβV.

In some embodiments, the genetically modified cell may be a T cell, such as a helper T cell, a cytotoxic T cell, a memory T ceil, regulatory T cell, gamma delta T cell, a natural killer cell, cytokine induced killer cell, a cell line thereof, a T memory stem cell, or other T effector cell. It may be also useful for the T cell to have limited toxicity toward healthy cells and to have specificity to cells expressing the TCRαV and/or TCRβV. In some embodiments, the genetically modified T cell may be specific for the TCRαV and/or TCRβV from a specific T cell clone. Such specificity may prevent or reduce off-target toxicity that is prevalent in current therapies that are not specific. In one embodiment, the T cell may have limited toxicity toward healthy cells. In one embodiment the T cell may be an autologous cell. In another embodiment, the T cell may be an allogeneic cell.

In some embodiments, the disclosure includes genetically modified immune cells derived from pluripotent stem cells that were differentiated in vitro. In other embodiments, the disclosure includes T cells, such as primary cells, expanded T cells derived from primary T cells, T cells derived from stem cells differentiated in vitro, T cell lines such as Jurkat cells, other sources of T cells, combinations thereof, and other effector cells.

Disclosed herein are methods of inhibiting or preventing the activation and/or expansion of autoreactive T cells in a subject comprising administering a CAR T cell comprising a CAR with an anti-TCRαV binding domain and/or an anti-TCRαV binding domain that binds to the TCRαV and/or TCRβV expressed by the autoreactive T cells. In some embodiments, the CAR T cell comprises a CAR comprising an anti-TCRαV binding domain that binds to the TCRαV expressed by the autoreactive T cells. In some embodiments, the CAR T cell comprises a CAR with an anti-TCRβV binding domain that binds to the TCRαV expressed by the autoreactive T cells In some embodiments, the subject has an autoimmune disease. In some embodiments, the CAR comprises a transmembrane domain as provided herein. In some embodiments, the CAR comprises an intracellular domain as provided herein.

Disclosed herein are methods of reducing or depleting autoreactive T cells in a subject comprising administering a CAR T cell comprising a CAR with an anti-TCRαV binding domain and/or an anti-TCRβV binding domain that binds to the TCRαV and/or TCRβV expressed by the autoreactive T cells. In some embodiments, the CAR T cell comprises a CAR comprising an anti-TCRαV binding domain that binds to the TCRαV expressed by the autoreactive T cells. In some embodiments, the CAR T cell comprises a CAR with an anti-TCRβV binding domain that binds to the TCRαV expressed by the autoreactive T cells In some embodiments, the subject has an autoimmune disease. In some embodiments, the CAR comprises a transmembrane domain as provided herein. In some embodiments, the CAR comprises an intracellular domain as provided herein.

### Cytokine Inhibitor Molecules

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

### TGF-Beta Inhibitors

The present disclosure further provides, inter alia, TCR targeting molecules, that include, e.g., are engineered to contain, one or more cytokine inhibitor molecules, e.g., inhibitors of immunomodulatory (e.g., proinflammatory) cytokines and variants, e.g., functional variants, thereof. Accordingly, in some embodiments, the cytokine inhibitor molecule is a TGF-beta inhibitor. In some embodiments, the TGF-beta inhibitor binds to and inhibits TGF-beta, e.g., reduces the activity of TGF-beta. In some embodiments, the TGF-beta inhibitor inhibits (e.g., reduces the activity of) TGF-beta 1. In some embodiments, the TGF-beta inhibitor inhibits (e.g., reduces the activity of) TGF-beta 2. In some embodiments, the TGF-beta inhibitor inhibits (e.g., reduces the activity of) TGF-beta 3. In some embodiments, the TGF-beta inhibitor inhibits (e.g., reduces the activity of) TGF-beta 1 and TGF-beta 3. In some embodiments, the TGF-beta inhibitor inhibits (e.g., reduces the activity of) TGF-beta 1, TGF-beta 2, and TGF-beta 3.

In some embodiments, the TGF-beta inhibitor comprises a portion of a TGF-beta receptor (e.g., an extracellular domain of a TGF-beta receptor) that is capable of inhibiting (e.g., reducing the activity of) TGF-beta, or functional fragment or variant thereof. In some embodiments, the TGF-beta inhibitor comprises a TGFBR1 polypeptide (e.g., an extracellular domain of TGFBR1 or functional variant thereof). In some embodiments, the TGF-beta inhibitor comprises a TGFBR2 polypeptide (e.g., an extracellular domain of TGFBR2 or functional variant thereof). In some embodiments, the TGF-beta inhibitor comprises a TGFBR3 polypeptide (e.g., an extracellular domain of TGFBR3 or functional variant thereof). In some embodiments, the TGF-beta inhibitor comprises a TGFBR1 polypeptide (e.g., an extracellular domain of TGFBR1 or functional variant thereof) and a TGFBR2 polypeptide (e.g., an extracellular domain of TGFBR2 or functional variant thereof). In some embodiments, the TGF-beta inhibitor comprises a TGFBR1 polypeptide (e.g., an extracellular domain of TGFBR1 or functional variant thereof) and a TGFBR3 polypeptide (e.g., an extracellular domain of TGFBR3 or functional variant thereof). In some embodiments, the TGF-beta inhibitor comprises a TGFBR2 polypeptide (e.g., an extracellular domain of TGFBR2 or functional variant thereof) and a TGFBR3 polypeptide (e.g., an extracellular domain of TGFBR3 or functional variant thereof).

Exemplary TGF-beta receptor polypeptides that can be used as TGF-beta inhibitors have been disclosed in U.S. Pat. Nos. 8,993,524, 9,676,863, 8,658,135, US20150056199, US20070184052, and WO2017037634, all of which are herein incorporated by reference in their entirety.

In some embodiments, the TGF-beta inhibitor comprises an extracellular domain of TGFBR1 or a sequence substantially identical thereto (e.g., a sequence that is at least 80%, 85%, 90%, or 95% identical thereto). In some embodiments, the TGF-beta inhibitor comprises an extracellular domain of SEQ ID NO: 7257, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7258, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7259, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7266, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7267, or a sequence substantially identical thereto (e.g., a sequence that is at least 80%, 85%, 90%, or 95% identical thereto).

In some embodiments, the TGF-beta inhibitor comprises an extracellular domain of TGFBR2 or a sequence substantially identical thereto (e.g., a sequence that is at least 80%, 85%, 90%, or 95% identical thereto). In some embodiments, the TGF-beta inhibitor comprises an extracellular domain of SEQ ID NO: 7260, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7261, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7262, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7263, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7264, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7265, or a sequence substantially identical thereto (e.g., a sequence that is at least 80%, 85%, 90%, or 95% identical thereto).

In some embodiments, the TGF-beta inhibitor comprises an extracellular domain of TGFBR3 or a sequence substantially identical thereto (e.g., a sequence that is at least 80%, 85%, 90%, or 95% identical thereto). In some embodiments, the TGF-beta inhibitor comprises an extracellular domain of SEQ ID NO: 7268, or a sequence substantially identical thereto (e.g., a sequence that is at least 80%, 85%, 90%, or 95% identical thereto). In some embodiments, the TGF-beta inhibitor comprises an extracellular domain of SEQ ID NO: 7269, or a sequence substantially identical thereto (e.g., a sequence that is at least 80%, 85%, 90%, or 95% identical thereto). In some embodiments, the TGF-beta inhibitor comprises the amino acid sequence of SEQ ID NO: 7270, or a sequence substantially identical thereto (e.g., a sequence that is at least 80%, 85%, 90%, or 95% identical thereto).

In some embodiments, the TGF-beta inhibitor comprises no more than one TGF-beta receptor extracellular domain. In some embodiments, the TGF-beta inhibitor comprises two or more (e.g., two, three, four, five, or more) TGF-beta receptor extracellular domains, linked together, e.g., via a linker.

### Immune Cell Engagers

The immune cell engagers of the TCR targeting molecules disclosed herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. For example, the NCRs can initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fe region of IgG, which binds complexed or aggregated IgG and also monomeric IgG and thereby mediates antibody-dependent cellular cytotoxicity (ADCC) and other antibody-dependent responses, such as phagocytosis. Without wishing to be bound by theory, it is thought that NK cell engagers that bind, recruit, and/or activate receptors like those disclosed above (e.g., NKp30, NKp36, NKG2D, or CD16) may target immune system activity to a target cell, e.g., a cell comprising a TCRBV antigen (e.g., a TCRBV antigen corresponding to a biased TCRBV clonotype), e.g., promote cell death or lysis of a target cell.

The present disclosure provides, inter alia, TCR targeting molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in Tables 7-10. In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in U.S. Pat. Nos. 6,979,546, 9,447,185, PCT Application No. WO2015121383A1, PCT Application No. WO2016110468A1, PCT Application No. WO2004056392A1, or U.S. Application Publication No. US20070231322A1, the sequences of which are hereby incorporated by reference. In some embodiments, binding of the NK cell engager, e.g., antigen binding domain that binds to NKp30, to the NK cell activates the NK cell. An antigen binding domain that binds to NKp30 (e.g., NKp30 present, e.g., expressed or displayed, on the surface of an NK cell) may be said to target NKp30, the NK cell, or both.

In some embodiments, the antigen binding domain that binds to NKp30 comprises one or more CDRs (e.g., VHCDR1, VHCDR2, VHCDR3, VLCDR1, VLCDR2, and/or VLCDR3) disclosed in Table 7A, Table 7B, or Table 8A, or a sequence having at least 85%, 90%, 95%, or 99% identity thereto. In some embodiments, the antigen binding domain that binds to NKp30 comprises one or more framework regions (e.g., VHFWR1, VHFWR2, VHFWR3, VHFWR4, VLFWR1, VLFWR2, VLFWR3, and/or VLFWR4) disclosed in Table 7A, Table 7B, or Table 8A, or a sequence having at least 85%, 90%, 95%, or 99% identity thereto. In some embodiments, the antigen binding domain that binds to NKp30 comprises a VH and/or a VL disclosed in Table 9A, or a sequence having at least 85%, 90%, 95%, or 99% identity thereto. In some embodiments, any of the VH domains disclosed in Table 9A may be paired with any of the VL domains disclosed in Table 9A to form the antigen binding domain that binds to NKp30. In some embodiments, the antigen binding domain that binds to NKp30 comprises an amino acid sequence disclosed in Table 10A, or a sequence having at least 85%, 90%, 95%, or 99% identity thereto.

In some embodiments, the antigen binding domain that binds to NKp30 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 7313, 6001, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6001, and 6002, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 6008, and 6009, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7385, and 7315, respectively (or a sequence having at least 85%, 90%, 95%, or 99% identity thereto). In some embodiments, the VHCDR1, VHCDR2, and VHCDR3 comprise the amino acid sequences of SEQ ID NOs: 7313, 7318, and 6009, respectively (or a sequence having at least 85%, 90%, 95%, or 99% identity thereto).

In some embodiments, the VLCDR1, VLCDR2, and VLCDR3 comprise the amino acid sequences of SEQ ID NOs: 7326, 7327, and 7329, respectively (or a sequence having at least 85%, 90%, 95%, or 99% identity thereto). In some embodiments, the VLCDR1, VLCDR2, and VLCDR3 comprise the amino acid sequences of SEQ ID NOs: 6063, 6064, and 7293, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6071, and 6072, respectively (or a sequence having at least 85%, 90%, 95%, or 99% identity thereto). In some embodiments, the VLCDR1, VLCDR2, and VLCDR3 comprise the amino acid sequences of SEQ ID NOs: 6070, 6064, and 7321, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 7313, 6001, 7315, 7326, 7327, and 7329, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6001, 6002, 6063, 6064, and 7293, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6008, 6009, 6070, 6071, and 6072, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7385, 7315, 6070, 6064, and 7321, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7318, 6009, 6070, 6064, and 7321, respectively (or a sequence having at least 85%, 90%, 95%, or 99% identity thereto).

In some embodiments, the VH comprises an amino acid sequence selected from the group consisting of SEQ ID NOs: 7298 or 7300-7304 (or a sequence having at least 85%, 90%, 95%, or 99% identity thereto) and/or the VL comprises an amino acid sequence selected from the group consisting of SEQ ID NOs: 7299 or 7305-7309 (or a sequence having at least 85%, 90%, 95%, or 99% identity thereto). In some embodiments, the VH and VL comprise the amino acid sequences of SEQ ID NOs: 7302 and 7305, respectively (or a sequence having at least 85%, 90%, 95%, or 99% identity thereto). In some embodiments, the VH and VL comprise the amino acid sequences of SEQ ID NOs: 7302 and 7309, respectively (or a sequence having at least 85%, 90%, 95%, or 99% identity thereto).

In some embodiments, the VH comprises an amino acid sequence selected from the group consisting of SEQ ID NOs: 6121 or 6123-6128 (or a sequence having at least 85%, 90%, 95%, or 99% identity thereto) and/or the VL comprises an amino acid sequence selected from the group consisting of SEQ ID NOs: 7294 or 6137-6141 (or a sequence having at least 85%, 90%, 95%, or 99% identity thereto). In some embodiments, the VH comprises an amino acid sequence selected from the group consisting of SEQ ID NOs: 6122 or 6129-6134 (or a sequence having at least 85%, 90%, 95%, or 99% identity thereto) and/or the VL comprises an amino acid sequence selected from the group consisting of SEQ ID NOs: 6136 or 6142-6147 (or a sequence having at least 85%, 90%, 95%, or 99% identity thereto). In some embodiments, the VH and VL comprise the amino acid sequences of SEQ ID NOs: 7295 and 7296, respectively (or a sequence having at least 85%, 90%, 95%, or 99% identity thereto). In some embodiments, the VH and VL comprise the amino acid sequences of SEQ ID NOs: 7297 and 7296, respectively (or a sequence having at least 85%, 90%, 95%, or 99% identity thereto). In some embodiments, the VH and VL comprise the amino acid sequences of SEQ ID NOs: 6122 and 6136, respectively (or a sequence having at least 85%, 90%, 95%, or 99% identity thereto).

In some embodiments, the antigen binding domain that binds to NKp30 comprises the amino acid sequence of SEQ ID NO: 7310 (or a sequence having at least 85%, 90%, 95%, or 99% identity thereto). In some embodiments, the antigen binding domain that binds to NKp30 comprises the amino acid sequence of SEQ ID NO: 7311 (or a sequence having at least 85%, 90%, 95%, or 99% identity thereto). In some embodiments, the antigen binding domain that binds to NKp30 comprises the amino acid sequence of SEQ ID NO: 6187, 6188, 6189 or 6190 (or a sequence having at least 85%, 90%, 95%, or 99% identity thereto). In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002.

In some embodiments, the antigen binding domain that targets NKp30 comprises a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002, and a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009.

In some embodiments, the antigen binding domain that targets NKp30 comprises a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009, and a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006.

In some embodiments, the antigen binding domain that targets NKp30 comprises a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 6006, and a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006.

In some embodiments, the antigen binding domain that targets NKp30 comprises a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6006, and a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013.

In some embodiments, the antigen binding domain that targets NKp30 comprises a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013, and a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013.

In some embodiments, the antigen binding domain that targets NKp30 comprises a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013, and a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, and/or a VHFWR4 amino acid sequence of SEQ ID NO: 6017.

In some embodiments, the antigen binding domain that targets NKp30 comprises a VH comprising a VHFWR1 amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6017.

In some embodiments, the antigen binding domain that targets NKp30 comprises a VL comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6077 (or a sequence with no more than 1, 2, or 3 mutations, e.g., substitutions, additions, or deletions), a VLFWR2 amino acid sequence of SEQ ID NO: 6078 (or a sequence with no more than 1 mutation, e.g., substitution, addition, or deletion), a VLFWR3 amino acid sequence of SEQ ID NO: 6079 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, and/or a VHFWR4 amino acid sequence of SEQ ID NO: 6021.

In some embodiments, the antigen binding domain that targets NKp30 comprises a VH comprising a VHFWR1 amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6021.

In some embodiments, the antigen binding domain that targets NKp30 comprises a VL comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, and/or a VLFWR4 amino acid sequence of SEQ ID NO: 6084.

In some embodiments, the antigen binding domain that targets NKp30 comprises a VL comprising a VLFWR1 amino acid sequence of SEQ ID NO: 6081 (or a sequence with no more than 1, 2, or 3 mutations, e.g., substitutions, additions, or deletions), a VLFWR2 amino acid sequence of SEQ ID NO: 6082 (or a sequence with no more than 1 mutation, e.g., substitution, addition, or deletion), a VLFWR3 amino acid sequence of SEQ ID NO: 6083 (or a sequence with no more than 1 mutation, e.g., substitution, addition, or deletion), and/or a VLFWR4 amino acid sequence of SEQ ID NO: 6084.

In some embodiments, the antigen binding domain that targets NKp30 comprises a VH comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 6024, and/or a VHFWR4 amino acid sequence of SEQ ID NO: 6025.

In some embodiments, the antigen binding domain that targets NKp30 comprises a VH comprising a VHFWR1 amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 6024 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6025.

In some embodiments, the antigen binding domain that targets NKp30 comprises a VL comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, and/or a VLFWR4 amino acid sequence of SEQ ID NO: 6088.

In some embodiments, the antigen binding domain that targets NKp30 comprises a VL comprising a VLFWR1 amino acid sequence of SEQ ID NO: 6085 (or a sequence with no more than 1, 2, or 3 mutations, e.g., substitutions, additions, or deletions), a VLFWR2 amino acid sequence of SEQ ID NO: 6086 (or a sequence with no more than 1 mutation, e.g., substitution, addition, or deletion), a VLFWR3 amino acid sequence of SEQ ID NO: 6087 (or a sequence with no more than 1 mutation, e.g., substitution, addition, or deletion), and/or a VLFWR4 amino acid sequence of SEQ ID NO: 6088.

In some embodiments, the antigen binding domain that targets NKp30 comprises a VH comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, and/or a VHFWR4 amino acid sequence of SEQ ID NO: 6029.

In some embodiments, the antigen binding domain that targets NKp30 comprises a VH comprising a VHFWR1 amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6029.

In some embodiments, the antigen binding domain that targets NKp30 comprises a VL comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, and/or a VLFWR4 amino acid sequence of SEQ ID NO: 6092.

In some embodiments, the antigen binding domain that targets NKp30 comprises a VL comprising a VLFWR1 amino acid sequence of SEQ ID NO: 6089 (or a sequence with no more than 1, 2, or 3 mutations, e.g., substitutions, additions, or deletions), a VLFWR2 amino acid sequence of SEQ ID NO: 6090 (or a sequence with no more than 1 mutation, e.g., substitution, addition, or deletion), a VLFWR3 amino acid sequence of SEQ ID NO: 6091 (or a sequence with no more than 1 mutation, e.g., substitution, addition, or deletion), and/or a VLFWR4 amino acid sequence of SEQ ID NO: 6092.

In some embodiments, the antigen binding domain that targets NKp30 comprises a VH comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, and/or a VHFWR4 amino acid sequence of SEQ ID NO: 6034.

In some embodiments, the antigen binding domain that targets NKp30 comprises a VH comprising a VHFWR1 amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6034.

In some embodiments, the antigen binding domain that targets NKp30 comprises a VL comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, and/or a VLFWR4 amino acid sequence of SEQ ID NO: 6096.

In some embodiments, the antigen binding domain that targets NKp30 comprises a VL comprising a VLFWR1 amino acid sequence of SEQ ID NO: 6093 (or a sequence with no more than 1, 2, or 3 mutations, e.g., substitutions, additions, or deletions), a VLFWR2 amino acid sequence of SEQ ID NO: 6094 (or a sequence with no more than 1 mutation, e.g., substitution, addition, or deletion), a VLFWR3 amino acid sequence of SEQ ID NO: 6095 (or a sequence with no more than 1 mutation, e.g., substitution, addition, or deletion), and/or a VLFWR4 amino acid sequence of SEQ ID NO: 6096.

In some embodiments, the antigen binding domain that targets NKp30 comprises a VH comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, and/or a VHFWR4 amino acid sequence of SEQ ID NO: 6038.

In some embodiments, the antigen binding domain that targets NKp30 comprises a VH comprising a VHFWR1 amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6038.

In some embodiments, the antigen binding domain that targets NKp30 comprises a VH comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, and/or a VHFWR4 amino acid sequence of SEQ ID NO: 6042.

In some embodiments, the antigen binding domain that targets NKp30 comprises a VH comprising a VHFWR1 amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6042.

In some embodiments, the antigen binding domain that targets NKp30 comprises a VL comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, and/or a VLFWR4 amino acid sequence of SEQ ID NO: 6100.

In some embodiments, the antigen binding domain that targets NKp30 comprises a VL comprising a VLFWR1 amino acid sequence of SEQ ID NO: 6097 (or a sequence with no more than 1, 2, or 3 mutations, e.g., substitutions, additions, or deletions), a VLFWR2 amino acid sequence of SEQ ID NO: 6098 (or a sequence with no more than 1 mutation, e.g., substitution, addition, or deletion), a VLFWR3 amino acid sequence of SEQ ID NO: 6099 (or a sequence with no more than 1 mutation, e.g., substitution, addition, or deletion), and/or a VLFWR4 amino acid sequence of SEQ ID NO: 6100.

In some embodiments, the antigen binding domain that targets NKp30 comprises a VH comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, and/or a VHFWR4 amino acid sequence of SEQ ID NO: 6046.

In some embodiments, the antigen binding domain that targets NKp30 comprises a VH comprising a VHFWR1 amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6046.

In some embodiments, the antigen binding domain that targets NKp30 comprises a VL comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, and/or a VLFWR4 amino acid sequence of SEQ ID NO: 6104.

In some embodiments, the antigen binding domain that targets NKp30 comprises a VL comprising a VLFWR1 amino acid sequence of SEQ ID NO: 6101 (or a sequence with no more than 1, 2, or 3 mutations, e.g., substitutions, additions, or deletions), a VLFWR2 amino acid sequence of SEQ ID NO: 6102 (or a sequence with no more than 1 mutation, e.g., substitution, addition, or deletion), a VLFWR3 amino acid sequence of SEQ ID NO: 6103 (or a sequence with no more than 1 mutation, e.g., substitution, addition, or deletion), and/or a VLFWR4 amino acid sequence of SEQ ID NO: 6104.

In some embodiments, the antigen binding domain that targets NKp30 comprises a VH comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 6049, and/or a VHFWR4 amino acid sequence of SEQ ID NO: 6050.

In some embodiments, the antigen binding domain that targets NKp30 comprises a VH comprising a VHFWR1 amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 6049 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6050.

In some embodiments, the antigen binding domain that targets NKp30 comprises a VL comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, and/or a VLFWR4 amino acid sequence of SEQ ID NO: 6108.

In some embodiments, the antigen binding domain that targets NKp30 comprises a VL comprising a VLFWR1 amino acid sequence of SEQ ID NO: 6105 (or a sequence with no more than 1, 2, or 3 mutations, e.g., substitutions, additions, or deletions), a VLFWR2 amino acid sequence of SEQ ID NO: 6106 (or a sequence with no more than 1 mutation, e.g., substitution, addition, or deletion), a VLFWR3 amino acid sequence of SEQ ID NO: 6107 (or a sequence with no more than 1 mutation, e.g., substitution, addition, or deletion), and/or a VLFWR4 amino acid sequence of SEQ ID NO: 6108.

In some embodiments, the antigen binding domain that targets NKp30 comprises a VH comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, and/or a VHFWR4 amino acid sequence of SEQ ID NO: 6054.

In some embodiments, the antigen binding domain that targets NKp30 comprises a VH comprising a VHFWR1 amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6054.

In some embodiments, the antigen binding domain that targets NKp30 comprises a VL comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, and/or a VLFWR4 amino acid sequence of SEQ ID NO: 6112.

In some embodiments, the antigen binding domain that targets NKp30 comprises a VL comprising a VLFWR1 amino acid sequence of SEQ ID NO: 6109 (or a sequence with no more than 1, 2, or 3 mutations, e.g., substitutions, additions, or deletions), a VLFWR2 amino acid sequence of SEQ ID NO: 6110 (or a sequence with no more than 1 mutation, e.g., substitution, addition, or deletion), a VLFWR3 amino acid sequence of SEQ ID NO: 6111 (or a sequence with no more than 1 mutation, e.g., substitution, addition, or deletion), and/or a VLFWR4 amino acid sequence of SEQ ID NO: 6112.

In some embodiments, the antigen binding domain that targets NKp30 comprises a VH comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, and/or a VHFWR4 amino acid sequence of SEQ ID NO: 6058.

In some embodiments, the antigen binding domain that targets NKp30 comprises a VH comprising a VHFWR1 amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6058.

In some embodiments, the antigen binding domain that targets NKp30 comprises a VL comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, and/or a VLFWR4 amino acid sequence of SEQ ID NO: 6116.

In some embodiments, the antigen binding domain that targets NKp30 comprises a VL comprising a VLFWR1 amino acid sequence of SEQ ID NO: 6113 (or a sequence with no more than 1, 2, or 3 mutations, e.g., substitutions, additions, or deletions), a VLFWR2 amino acid sequence of SEQ ID NO: 6114 (or a sequence with no more than 1 mutation, e.g., substitution, addition, or deletion), a VLFWR3 amino acid sequence of SEQ ID NO: 6115 (or a sequence with no more than 1 mutation, e.g., substitution, addition, or deletion), and/or a VLFWR4 amino acid sequence of SEQ ID NO: 6116.

In some embodiments, the antigen binding domain that targets NKp30 comprises a VH comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, and/or a VHFWR4 amino acid sequence of SEQ ID NO: 6062.

In some embodiments, the antigen binding domain that targets NKp30 comprises a VH comprising a VHFWR1 amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6062.

In some embodiments, the antigen binding domain that targets NKp30 comprises a VL comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, and/or a VLFWR4 amino acid sequence of SEQ ID NO: 6120.

In some embodiments, the antigen binding domain that targets NKp30 comprises a VL comprising a VLFWR1 amino acid sequence of SEQ ID NO: 6117 (or a sequence with no more than 1, 2, or 3 mutations, e.g., substitutions, additions, or deletions), a VLFWR2 amino acid sequence of SEQ ID NO: 6118 (or a sequence with no more than 1 mutation, e.g., substitution, addition, or deletion), a VLFWR3 amino acid sequence of SEQ ID NO: 6119 (or a sequence with no more than 1 mutation, e.g., substitution, addition, or deletion), and/or a VLFWR4 amino acid sequence of SEQ ID NO: 6120.

In some embodiments, the antigen binding domain that targets NKp30 comprises a VH comprising the amino acid sequence of SEQ ID NO: 6148 (or an amino acid sequence having at least about 77%, 80%, 85%, 90%, 95%, or 99% sequence identity to SEQ ID NO: 6148). In some embodiments, the antigen binding domain that targets NKp30 comprises a VH comprising the amino acid sequence of SEQ ID NO: 6149 (or an amino acid sequence having at least about 77%, 80%, 85%, 90%, 95%, or 99% sequence identity to SEQ ID NO: 6149). In some embodiments, the antigen binding domain that targets NKp30 comprises a VL comprising the amino acid sequence of SEQ ID NO: 6150 (or an amino acid sequence having at least about 93%, 95%, or 99% sequence identity to SEQ ID NO: 6150). In some embodiments, antigen binding domain that targets NKp30 comprises a VH comprising the amino acid sequence of SEQ ID NO: 6148. In some embodiments, antigen binding domain that targets NKp30 comprises a VH comprising the amino acid sequence of SEQ ID NO: 6149. In some embodiments, the antigen binding domain that targets NKp30 comprises a VL comprising the amino acid sequence of SEQ ID NO: 6150.

In some embodiments, the antigen binding domain that targets NKp30 comprises a VH comprising the amino acid sequence of SEQ ID NO: 6148, and a VL comprising the amino acid sequence of SEQ ID NO: 6150. In some embodiments, the antigen binding domain that targets NKp30 comprises a VH comprising the amino acid sequence of SEQ ID NO: 6149, and a VL comprising the amino acid sequence of SEQ ID NO: 6150.

In some embodiments, the antigen binding domain that targets NKp30 comprises a VH comprising the amino acid sequence of SEQ ID NO: 6151 (or an amino acid sequence having at least about 77%, 80%, 85%, 90%, 95%, or 99% sequence identity to SEQ ID NO: 6151). In some embodiments, the antigen binding domain that targets NKp30 comprises a VH comprising the amino acid sequence of SEQ ID NO: 6152 (or an amino acid sequence having at least about 77%, 80%, 85%, 90%, 95%, or 99% sequence identity to SEQ ID NO: 6152). In some embodiments, the antigen binding domain that targets NKp30 comprises a VL comprising the amino acid sequence of SEQ ID NO: 6153 (or an amino acid sequence having at least about 93%, 95%, or 99% sequence identity to SEQ ID NO: 6153). In some embodiments, antigen binding domain that targets NKp30 comprises a VH comprising the amino acid sequence of SEQ ID NO: 6151. In some embodiments, antigen binding domain that targets NKp30 comprises a VH comprising the amino acid sequence of SEQ ID NO: 6152. In some embodiments, the antigen binding domain that targets NKp30 comprises a VL comprising the amino acid sequence of SEQ ID NO: 6153.

In some embodiments, the antigen binding domain that targets NKp30 comprises a VH comprising the amino acid sequence of SEQ ID NO: 6151, and a VL comprising the amino acid sequence of SEQ ID NO: 6153. In some embodiments, the antigen binding domain that targets NKp30 comprises a VH comprising the amino acid sequence of SEQ ID NO: 6152, and a VL comprising the amino acid sequence of SEQ ID NO: 6153.

In some embodiments, the antigen binding domain that targets NKp30 comprises an scFv. In some embodiments, the scFv comprises an amino acid sequence selected from SEQ ID NOs: 6187-6190, or an amino acid sequence having at least about 93%, 95%, or 99% sequence identity thereto.

TABLE 7A
Exemplary heavy chain CDRs and FWRs of NKp30-targeting antigen binding domains

| Ab ID | VHFWR1 | VHCDR1 | VHFWR2 | VHCDR2 | VHFWR3 | VHCDR3 | VHFWR4 |
|---|---|---|---|---|---|---|---|
| 9G1-HC | QIQLQES | TGGYHW | WIRQFP | YIYSSGS | RISITRDT | GNWHYF | WGQGT |
| | GPGLVK | N (SEQ ID | GKKLEW | TSYNPSL | SKNQFFL | DF (SEQ | MVTVSS |
| | PSQSLSL | NO: 6000) | MG (SEQ | KS (SEQ | QLNSVT | ID NO: | (SEQ ID |
| | TCSVTGF | | ID NO: | ID NO: | TEDTAT | 6002) | NO: 6006) |
| | SIN (SEQ | | 6004) | 6001) | YYCAR | | |
| | ID NO: | | | | (SEQ ID | | |
| | 6003) | | | | NO: 6005) | | |
| 15H6- | QIQLQES | TGGYHW | WIRQFP | YIYSSGT | RISITRDT | GNWHYF | WGQGTL |
| HC | GPGLVK | N (SEQ ID | GKKLEW | TRYNPSL | SKNQFFL | DY (SEQ | VAVSS |
| | PSQSLSL | NO: 6007) | MG (SEQ | KS (SEQ | QLNSVT | ID NO: | (SEQ ID |
| | TCSVTGF | | ID NO: | ID NO: | PEDTAT | 6009) | NO: 6013) |
| | SIN (SEQ | | 6011) | 6008) | YYCTR | | |
| | ID NO: | | | | (SEQ ID | | |
| | 6010) | | | | NO: 6012) | | |
| 9G1- | QIQLQES | TGGYHW | WIRQPA | YIYSSGS | RVTMSR | GNWHYF | WGQGT |
| HC_1 | GPGLVK | N (SEQ ID | GKGLEW | TSYNPSL | DTSKNQ | DF (SEQ | MVTVSS |
| | PSETLSL | NO: 6000) | IG (SEQ | KS (SEQ | FSLKLSS | ID NO: | (SEQ ID |
| | TCTVSGF | | ID NO: | ID NO: | VTAADT | 6002) | NO: 6017) |
| | SIN (SEQ | | 6015) | 6001) | AVYYCA | | |
| | ID NO: | | | | R (SEQ ID | | |
| | 6014) | | | | NO: 6016) | | |
| 9G1- | QIQLQES | TGGYHW | WIRQHP | YIYSSGS | LVTISRD | GNWHYF | WGQGT |
| | GPGLVK | N (SEQ ID | GKGLEW | TSYNPSL | TSKNQFS | DF (SEQ | MVTVSS |
| HC_2 | PSQTLSL | NO: 6000) | IG (SEQ | KS (SEQ | LKLSSVT | ID NO: | (SEQ ID |
| | TCTVSGF | | ID NO: | ID NO: | AADTAV | 6002) | NO: 6021) |
| | SIN (SEQ | | 6019) | 6001) | YYCAR | | |
| | ID NO: | | | | (SEQ ID | | |
| | 6018) | | | | NO: 6020) | | |
| 9G1- | EIQLLES | TGGYHW | WVRQAP | YIYSSGS | RFTISRD | GNWHYF | WGQGT |
| HC_3 | GGGLVQ | N (SEQ ID | GKGLEW | TSYNPSL | TSKNTF | DF (SEQ | MVTVSS |
| | PGGSLRL | NO: 6000) | VG (SEQ | KS (SEQ | YLQMNS | ID NO: | (SEQ ID |
| | SCAVSG | | ID NO: | ID NO: | LRAEDT | 6002) | NO: 6025) |
| | FSIN | | 6023) | 6001) | AVYYCA | | |
| | (SEQ ID | | | | R (SEQ ID | | |
| | NO: 6022) | | | | NO: 6024) | | |
| 9G1- | QIQLVQS | TGGYHW | WVRQAP | YIYSSGS | RVTITRD | GNWHYF | WGQGT |
| HC_4 | GAEVKK | N (SEQ ID | GQGLEW | TSYNPSL | TSTNTFY | DF (SEQ | MVTVSS |
| | PGSSVK | NO: 6000) | MG (SEQ | KS (SEQ | MELSSL | ID NO: | (SEQ ID |
| | VSCKVS | | ID NO: | ID NO: | RSEDTA | 6002) | NO: 6029) |
| | GFSIN | | 6027) | 6001) | VYYCAR | | |
| | (SEQ ID | | | | (SEQ ID | | |
| | NO: 6026) | | | | NO: 6028) | | |
| 9G1- | EIQLVES | TGGYHW | WVRQAP | YIYSSGS | RFTISRD | GNWHYF | WGQGT |
| HC_5 | GGGLVQ | N (SEQ ID | GKGLEW | TSYNPSL | TAKNSF | DF (SEQ | MVTVSS |
| | PGGSLRL | NO: 6000) | VG (SEQ | KS (SEQ | YLQMNS | ID NO: | (SEQ ID |
| | SCAVSG | | ID NO: | ID NO: | LRAEDT | 6002) | NO: 6034) |
| | FSIN | | 6032) | 6001) | AVYYCA | | |
| | (SEQ ID | | | | R (SEQ ID | | |
| | NO: 6030) | | | | NO: 6033) | | |
| 9G1- | QIQLVQS | TGGYHW | WVRQAP | YIYSSGS | RVTMTR | GNWHYF | WGQGT |
| HC_6 | GAEVKK | N (SEQ ID | GQGLEW | TSYNPSL | DTSTNTF | DF (SEQ | MVTVSS |
| | PGASVK | NO: 6000) | MG (SEQ | KS (SEQ | YMELSS | ID NO: | (SEQ ID |
| | VSCKVS | | ID NO: | ID NO: | LRSEDTA | 6002) | NO: 6038) |
| | GFSIN | | 6036) | 6001) | VYYCAR | | |
| | (SEQ ID | | | | (SEQ ID | | |
| | NO: 6035) | | | | NO: 6037) | | |
| 15H6- | QIQLQES | TGGYHW | WIRQHP | YIYSSGT | LVTISRD | GNWHYF | WGQGTL |
| HC_1 | GPGLVK | N (SEQ ID | GKGLEW | TRYNPSL | TSKNQFS | DY (SEQ | VTVSS |
| | PSQTLSL | NO: 6007) | IG (SEQ | KS (SEQ | LKLSSVT | ID NO: | (SEQ ID |
| | TCTVSGF | | ID NO: | ID NO: | AADTAV | 6009) | NO: 6042) |
| | SIN (SEQ | | 6040) | 6008) | YYCAR | | |
| | ID NO: | | | | (SEQ ID | | |
| | 6039) | | | | NO: 6041) | | |
| 15H6- | QIQLQES | TGGYHW | WIRQPA | YIYSSGT | RVTMSR | GNWHYF | WGQGTL |
| HC_2 | GPGLVK | N (SEQ ID | GKGLEW | TRYNPSL | DTSKNQ | DY (SEQ | VTVSS |
| | PSETLSL | NO: 6007) | IG (SEQ | KS (SEQ | FSLKLSS | ID NO: | (SEQ ID |
| | TCTVSGF | | ID NO: | ID NO: | VTAADT | 6009) | NO: 6046) |
| | SIN (SEQ | | 6044) | 6008) | AVYYCA | | |
| | ID NO: | | | | R (SEQ ID | | |
| | 6043) | | | | NO: 6045) | | |
| 15H6- | EIQLLES | TGGYHW | WVRQAP | YIYSSGT | RFTISRD | GNWHYF | WGQGTL |
| HC 3 | GGGLVQ | N (SEQ ID | GKGLEW | TRYNPSL | TSKNTF | DY (SEQ | VTVSS |
| | PGGSLRL | NO: 6007) | VG (SEQ | KS (SEQ | YLQMNS | ID NO: | (SEQ ID |
| | SCAVSG | | ID NO: | ID NO: | LRAEDT | 6009) | NO: 6050) |
| | FSIN | | 6048) | 6008) | AVYYCA | | |
| | (SEQ ID | | | | R (SEQ ID | | |
| | NO: 6047) | | | | NO: 6049) | | |
| 15H6- | QIQLVES | TGGYHW | WIRQAP | YIYSSGT | RFTISRD | GNWHYF | WGQGTL |
| HC_4 | GGGLVK | N (SEQ ID | GKGLEW | TRYNPSL | TAKNSF | DY (SEQ | VTVSS |
| | PGGSLRL | NO: 6007) | VG (SEQ | KS (SEQ | YLQMNS | ID NO: | (SEQ ID |
| | SCAVSG | | ID NO: | ID NO: | LRAEDT | 6009) | NO: 6054) |
| | FSIN | | 6052) | 6008) | AVYYCA | | |
| | (SEQ ID | | | | R (SEQ ID | | |
| | NO: 6051) | | | | NO: 6053) | | |
| 15H6- | QIQLVQS | TGGYHW | WVRQAP | YIYSSGT | RVTMTR | GNWHYF | WGQGTL |
| HC_5 | GAEVKK | N (SEQ ID | GQGLEW | TRYNPSL | DTSTNTF | DY (SEQ | VTVSS |
| | PGASVK | NO: 6007) | MG (SEQ | KS (SEQ | YMELSS | ID NO: | (SEQ ID |
| | VSCKVS | | ID NO: | ID NO: | LRSEDTA | 6009) | NO: 6058) |
| | GFSIN | | 6056) | 6008) | VYYCAR | | |
| | (SEQ ID | | | | (SEQ ID | | |
| | NO: 6055) | | | | NO: 6057) | | |
| 15H6- | EIQLVQS | TGGYHW | WVQQAP | YIYSSGT | RVTITRD | GNWHYF | WGQGTL |
| HC_6 | GAEVKK | N (SEQ ID | GKGLEW | TRYNPSL | TSTNTFY | DY (SEQ | VTVSS |
| | PGATVKI | NO: 6007) | MG (SEQ | KS (SEQ | MELSSL | ID NO: | (SEQ ID |
| | SCKVSG | | ID NO: | ID NO: | RSEDTA | 6009) | NO: 6062) |
| | FSIN | | 6060) | 6008) | VYYCAR | | |
| | (SEQ ID | | | | (SEQ ID | | |
| | NO: 6059) | | | | NO: 6061) | | |

TABLE 7B
Exemplary heavy chain CDRs and FWRs of NKp30-targeting antigen binding domains
(according to the Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| 9G1-HC | QIQLQES | GYHWN | WIRQFP | YIYSSGS | RISITRDT | GNWHYF | WGQGT |
| | GPGLVK | (SEQ ID | GKKLEW | TSYNPSL | SKNQFFL | DF (SEQ | MVTVSS |
| | PSQSLSL | NO: 7313) | MG (SEQ | KS (SEQ | QLNSVT | ID NO: | (SEQ ID |
| | TCSVTGF | | ID NO: | ID NO: | TEDTAT | 6002) | NO: 6006) |
| | SINTG | | 6004) | 6001) | YYCAR | | |
| | (SEQ ID | | | | (SEQ ID | | |
| | NO: 7317) | | | | NO: 6005) | | |
| 15H6- | QIQLQES | GYHWN | WIRQFP | YIYSSGT | RISITRDT | GNWHYF | WGQGTL |
| HC | GPGLVK | (SEQ ID | GKKLEW | TRYNPSL | SKNQFFL | DY (SEQ | VAVSS |
| | PSQSLSL | NO: 7313) | MG (SEQ | KS (SEQ | QLNSVT | ID NO: | (SEQ ID |
| | TCSVTGF | | ID NO: | ID NO: | PEDTAT | 6009) | NO: 6013) |
| | SINTG | | 6011) | 6008) | YYCTR | | |
| | (SEQ ID | | | | (SEQ ID | | |
| | NO: 7317) | | | | NO: 6012) | | |
| 9G1- | QIQLQES | GYHWN | WIRQPA | YIYSSGS | RVTMSR | GNWHYF | WGQGT |
| HC_1 | GPGLVK | (SEQ ID | GKGLEW | TSYNPSL | DTSKNQ | DF (SEQ | MVTVSS |
| | PSETLSL | NO: 7313) | IG (SEQ | KS (SEQ | FSLKLSS | ID NO: | (SEQ ID |
| | TCTVSGF | | ID NO: | ID NO: | VTAADT | 6002) | NO: 6017) |
| | SINTG | | 6015) | 6001) | AVYYCA | | |
| | (SEQ ID | | | | R (SEQ ID | | |
| | NO: 7371) | | | | NO: 6016) | | |
| 9G1- | QIQLQES | GYHWN | WIRQHP | YIYSSGS | LVTISRD | GNWHYF | WGQGT |
| HC_2 | GPGLVK | (SEQ ID | GKGLEW | TSYNPSL | TSKNQFS | DF (SEQ | MVTVSS |
| | PSQTLSL | NO: 7313) | IG (SEQ | KS (SEQ | LKLSSVT | ID NO: | (SEQ ID |
| | TCTVSGF | | ID NO: | ID NO: | AADTAV | 6002) | NO: 6021) |
| | SINTG | | 6019) | 6001) | YYCAR | | |
| | (SEQ ID | | | | (SEQ ID | | |
| | NO: 7372) | | | | NO: 6020) | | |
| 9G1- | EIQLLES | GYHWN | WVRQAP | YIYSSGS | RFTISRD | GNWHYF | WGQGT |
| HC_3 | GGGLVQ | (SEQ ID | GKGLEW | TSYNPSL | TSKNTF | DF (SEQ | MVTVSS |
| | PGGSLRL | NO: 7313) | VG (SEQ | KS (SEQ | YLQMNS | ID NO: | (SEQ ID |
| | SCAVSG | | ID NO: | ID NO: | LRAEDT | 6002) | NO: 6025) |
| | FSINTG | | 6023) | 6001) | AVYYCA | | |
| | (SEQ ID | | | | R (SEQ ID | | |
| | NO: 7373) | | | | NO: 6024) | | |
| 9G1- | QIQLVQS | GYHWN | WVRQAP | YIYSSGS | RVTITRD | GNWHYF | WGQGT |
| HC_4 | GAEVKK | (SEQ ID | GQGLEW | TSYNPSL | TSTNTFY | DF (SEQ | MVTVSS |
| | PGSSVK | NO: 7313) | MG (SEQ | KS (SEQ | MELSSL | ID NO: | (SEQ ID |
| | VSCKVS | | ID NO: | ID NO: | RSEDTA | 6002) | NO: 6029) |
| | GFSINTG | | 6027) | 6001) | VYYCAR | | |
| | (SEQ ID | | | | (SEQ ID | | |
| | NO: 7374) | | | | NO: 6028) | | |
| 9G1- | EIQLVES | GYHWN | WVRQAP | YIYSSGS | RFTISRD | GNWHYF | WGQGT |
| HC_5 | GGGLVQ | (SEQ ID | GKGLEW | TSYNPSL | TAKNSF | DF (SEQ | MVTVSS |
| | PGGSLRL | NO: 7313) | VG (SEQ | KS (SEQ | YLQMNS | ID NO: | (SEQ ID |
| | SCAVSG | | ID NOL | ID NO: | LRAEDT | 6002) | NO: 6034) |
| | FSINTG | | 6032) | 6001) | AVYYCA | | |
| | (SEQ ID | | | | R (SEQ ID | | |
| | NO: 7375) | | | | NO: 6033) | | |
| 9G1- | QIQLVQS | GYHWN | WVRQAP | YIYSSGS | RVTMTR | GNWHYF | WGQGT |
| HC_6 | GAEVKK | (SEQ ID | GQGLEW | TSYNPSL | DTSTNTF | DF (SEQ | MVTVSS |
| | PGASVK | NO: 7313) | MG (SEQ | KS (SEQ | YMELSS | ID NO: | (SEQ ID |
| | VSCKVS | | ID NO: | ID NO: | LRSEDTA | 6002) | NO: 6038) |
| | GFSINTG | | 6036) | 6001) | VYYCAR | | |
| | (SEQ ID | | | | (SEQ ID | | |
| | NO: 7376) | | | | NO: 6037) | | |
| 15H6- | QIQLQES | GYHWN | WIRQHP | YIYSSGT | LVTISRD | GNWHYF | WGQGTL |
| HC_1 | GPGLVK | (SEQ ID | GKGLEW | TRYNPSL | TSKNQFS | DY (SEQ | VTVSS |
| | PSQTLSL | NO: 7313) | IG (SEQ | KS (SEQ | LKLSSVT | ID NO: | (SEQ ID |
| | TCTVSGF | | ID NO: | ID NO: | AADTAV | 6009) | NO: 6042) |
| | SINTG | | 6040) | 6008) | YYCAR | | |
| | (SEQ ID | | | | (SEQ ID | | |
| | NO: 7372) | | | | NO: 6041) | | |
| 15H6- | QIQLQES | GYHWN | WIRQPA | YIYSSGT | RVTMSR | GNWHYF | WGQGTL |
| HC_2 | GPGLVK | (SEQ ID | GKGLEW | TRYNPSL | DTSKNQ | DY (SEQ | VTVSS |
| | PSETLSL | NO: 7313) | IG (SEQ | KS (SEQ | FSLKLSS | ID NO: | (SEQ ID |
| | TCTVSGF | | ID NO: | ID NO: | VTAADT | 6009) | NO: 6046) |
| | SINTG | | 6044) | 6008) | AVYYCA | | |
| | (SEQ ID | | | | R (SEQ ID | | |
| | NO: 7371) | | | | NO: 6045) | | |
| 15H6- | EIQLLES | GYHWN | WVRQAP | YIYSSGT | RFTISRD | GNWHYF | WGQGTL |
| HC_3 | GGGLVQ | (SEQ ID | GKGLEW | TRYNPSL | TSKNTF | DY (SEQ | VTVSS |
| | PGGSLRL | NO: 7313) | VG (SEQ | KS (SEQ | YLQMNS | ID NO: | (SEQ ID |
| | SCAVSG | | ID NO: | ID NO: | LRAEDT | 6009) | NO: 6050) |
| | FSINTG | | 6048) | 6008) | AVYYCA | | |
| | (SEQ ID | | | | R (SEQ ID | | |
| | NO: 7373) | | | | NO: 6049) | | |
| 15H6- | QIQLVES | GYHWN | WIRQAP | YIYSSGT | RFTISRD | GNWHYF | WGQGTL |
| HC_4 | GGGLVK | (SEQ ID | GKGLEW | TRYNPSL | TAKNSF | DY (SEQ | VTVSS |
| | PGGSLRL | NO: 7313) | VG (SEQ | KS (SEQ | YLQMNS | ID NO: | (SEQ ID |
| | SCAVSG | | ID NO: | ID NO: | LRAEDT | 6009) | NO: 6054) |
| | FSINTG | | 6052) | 6008) | AVYYCA | | |
| | (SEQ ID | | | | R (SEQ ID | | |
| | NO: 7377) | | | | NO: 6053) | | |
| 15H6- | QIQLVQS | GYHWN | WVRQAP | YIYSSGT | RVTMTR | GNWHYF | WGQGTL |
| HC_5 | GAEVKK | (SEQ ID | GQGLEW | TRYNPSL | DTSTNTF | DY (SEQ | VTVSS |
| | PGASVK | NO: 7313) | MG (SEQ | KS (SEQ | YMELSS | ID NO: | (SEQ ID |
| | VSCKVS | | ID NO: | ID NO: | LRSEDTA | 6009) | NO: 6058) |
| | GFSINTG | | 6056) | 6008) | VYYCAR | | |
| | (SEQ ID | | | | (SEQ ID | | |
| | NO: 7376) | | | | NO: 6057) | | |
| 15H6- | EIQLVQS | GYHWN | WVQQAP | YIYSSGT | RVTITRD | GNWHYF | WGQGTL |
| HC_6 | GAEVKK | (SEQ ID | GKGLEW | TRYNPSL | TSTNTFY | DY (SEQ | VTVSS |
| | PGATVKI | NO: 7313) | MG (SEQ | KS (SEQ | MELSSL | ID NO: | (SEQ ID |
| | SCKVSG | | ID NO: | ID NO: | RSEDTA | 6009) | NO: 6062) |
| | FSINTG | | 6060) | 6008) | VYYCAR | | |
| | (SEQ ID | | | | (SEQ ID | | |
| | NO: 7378) | | | | NO: 6061) | | |
| 9D9-HC | QIQLQES | GYHWN | WIRQFP | YIYSSGT | RISITRDT | GDWHYF | WGQGT |
| | GPGLVK | (SEQ ID | GKKVEW | TKYNPS | SKNQFFL | DY (SEQ | MVAVSS |
| | PSQSLSL | NO: 7313) | MG (SEQ | LKS (SEQ | QLNSVT | ID NO: | (SEQ ID |
| | SCSVTGF | | ID NO: | ID NO: | TEDTAT | 7315) | NO: 7316) |
| | SINTG | | 7314) | 7385) | YYCAR | | |
| | (SEQ ID | | | | (SEQ ID | | |
| | NO: 7312) | | | | NO: 6005) | | |
| 3A12- | QIQLQES | GYHWN | WIRQFP | YIYSSGS | RFSITRD | GNWHYF | WGQGTL |
| HC | GPGLVK | (SEQ ID | GKKLEW | TRYNPSL | TSKNQFF | DY (SEQ | VAVSS |
| | PSQSLSL | NO: 7313) | MG (SEQ | KS (SEQ | LQLNSV | ID NO: | (SEQ ID |
| | TCSVTGF | | ID NO: | ID NO: | TTEDTAT | 6009) | NO: 6013) |
| | SINTG | | 6004) | 7318) | YYCTR | | |
| | (SEQ ID | | | | (SEQ ID | | |
| | NO: 7317) | | | | NO: 7319) | | |
| 12D10- | QIQLQES | GYHWN | WIRQFP | YIYSSGT | RISITRDT | GNWHYF | WGQGTL |
| HC | GPGLVK | (SEQ ID | GKKLEW | TRYNPSL | SKNQFFL | DY (SEQ | VAVSS |
| | PSQSLSL | NO: 7313) | MG (SEQ | KS (SEQ | QLNSVT | ID NO: | (SEQ ID |
| | TCSVTGF | | ID NO: | ID NO: | PEDTAT | 6009) | NO: 6013) |
| | SINTG | | 6004) | 6008) | YYCTR | | |
| | (SEQ ID | | | | (SEQ ID | | |
| | NO: 7317) | | | | NO: 6012) | | |
| 15E1- | QIQLQES | GYHWN | WIRQFP | YIYSSGS | RFSITRD | GDWHYF | WGPGTM |
| HC | GPGLVK | (SEQ ID | GKKLEW | TSYNPSL | TSKNQFF | DY (SEQ | VTVSS |
| | PSQSLSL | NO: 7313) | MG (SEQ | KS (SEQ | LQLNSV | ID NO: | (SEQ ID |
| | SCSVTGF | | ID NO: | ID NO: | TTEDTAT | 7315) | NO: 7324) |
| | SITTT | | 6004) | 6001) | YYCAR | | |
| | (SEQ ID | | | | (SEQ ID | | |
| | NO: 7322) | | | | NO: 7323) | | |
| 15E1_ | QIQLQES | GYHWN | WIRQHP | YIYSSGS | LVTISRD | GDWHYF | WGQGT |
| Humanized | GPGLVK | (SEQ ID | GKGLEW | TSYNPSL | TSKNQFS | DY (SEQ | MVTVSS |
| variant_ | PSQTLSL | NO: 7313) | IG (SEQ | KS (SEQ | LKLSSVT | ID NO: | (SEQ ID |
| VH1 | TCTVSGF | | ID NO: | ID NO: | AADTAV | 7315) | NO: 6006) |
| | SITTT | | 6019) | 6001) | YYCAR | | |
| | (SEQ ID | | | | (SEQ ID | | |
| | NO: 7330) | | | | NO: 6020) | | |
| 15E1_ | QIQLVES | GYHWN | WIRQAP | YIYSSGS | RFTISRD | GDWHYF | WGQGT |
| Humanized | GGGLVK | (SEQ ID | GKGLEW | TSYNPSL | TAKNSF | DY (SEQ | MVTVSS |
| variant_ | PGGSLRL | NO: 7313) | VG (SEQ | KS (SEQ | YLQMNS | ID NO: | (SEQ ID |
| VH2 | SCAVSG | | ID NO: | ID NO: | LRAEDT | 7315) | NO: 6006) |
| | FSITTT | | 6052) | 6001) | AVYYCA | | |
| | (SEQ ID | | | | R (SEQ ID | | |
| | NO: 7331) | | | | NO: 6033) | | |
| 15E1_ | EIQLLES | GYHWN | WVRQAP | YIYSSGS | RFTISRD | GDWHYF | WGQGT |
| Humanized | GGGLVQ | (SEQ ID | GKGLEW | TSYNPSL | TSKNTF | DY (SEQ | MVTVSS |
| variant_ | PGGSLRL | NO: 7313) | VG (SEQ | KS (SEQ | YLQMNS | ID NO: | (SEQ ID |
| VH3 | SCAVSG | | ID NO: | ID NO: | LRAEDT | 7315) | NO: 6006) |
| | FSITTT | | 6023) | 6001) | AVYYCA | | |
| | (SEQ ID | | | | R (SEQ ID | | |
| | NO: 7332) | | | | NO: 6024) | | |
| 15E1_ | EIQLVES | GYHWN | WVRQAP | YIYSSGS | RFTISRD | GDWHYF | WGQGT |
| Humanized | GGGLVQ | (SEQ ID | GKGLEW | TSYNPSL | TAKNSF | DY (SEQ | MVTVSS |
| variant_ | PGGSLRL | NO: 7313) | VG (SEQ | KS (SEQ | YLQMNS | ID NO: | (SEQ ID |
| VH4 | SCAVSG | | ID NO: | ID NO: | LRAEDT | 7315) | NO: 6006) |
| | FSITTT | | 6023) | 6001) | AVYYCA | | |
| | (SEQ ID | | | | R (SEQ ID | | |
| | NO: 7333) | | | | NO: 6033) | | |
| 15E1_ | QIQLVQS | GYHWN | WVRQAP | YIYSSGS | RVTMTR | GDWHYF | WGQGT |
| Humanized | GAEVKK | (SEQ ID | GQGLEW | TSYNPSL | DTSTNTF | DY (SEQ | MVTVSS |
| variant_ | PGASVK | NO: 7313) | MG (SEQ | KS (SEQ | YMELSS | ID NO: | (SEQ ID |
| VH5 | VSCKVS | | ID NO: | ID NO: | LRSEDTA | 7315) | NO: 6006) |
| | GFSITTT | | 6027) | 6001) | VYYCAR | | |
| | (SEQ ID | | | | (SEQ ID | | |
| | NO: 7334) | | | | NO: 6037) | | |

TABLE 8A
Exemplary light chain CDRs and FWRs of NKp30-targeting antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| 9G1-LC | SYTLTQP | SGERLSD | WYQQKP | ENDKRP | GIPDQFS | QSWDST | FGSGTQ |
| | PLLSVAL | KYVH | GRAPVM | S (SEQ ID | GSNSGNI | NSAV | LTVL |
| | GHKATIT | (SEQ ID | VIY (SEQ | NO: 6064) | ATLTISK | (SEQ ID | (SEQ ID |
| | C (SEQ ID | NO: 6063) | ID NO: | | AQAGYE | NO: 7293) | NO: 6069) |
| | NO: 6066) | | 6067) | | ADYYC | | |
| | | | | | (SEQ ID | | |
| | | | | | NO: 7292) | | |
| 15H6- | SYTLTQP | SGENLS | WYQQKP | ENEKRPS | GIPDQFS | HYWESI | FGSGTH |
| LC | PSLSVAP | DKYVH | GRAPVM | (SEQ ID | GSNSGNI | NSVV | LTVL |
| | GQKATII | (SEQ ID | VIY (SEQ | NO: 6071) | ATLTISK | (SEQ ID | (SEQ ID |
| | C (SEQ ID | NO: 6070) | ID NO: | | AQPGSE | NO: 6072) | NO: 6076) |
| | NO: 6073) | | 6074) | | ADYYC | | |
| | | | | | (SEQ ID | | |
| | | | | | NO: 6075) | | |
| 9G1- | QSVTTQ | SGERLSD | WYQQLP | ENDKRP | GVPDRF | QSWDST | FGGGTQ |
| LC_1 | PPSVSGA | KYVH | GTAPKM | S (SEQ ID | SGSNSG | NSAV | LTVL |
| | PGQRVTI | (SEQ ID | LIY (SEQ | NO: 6064) | NSASLAI | (SEQ ID | (SEQ ID |
| | SC (SEQ | NO: 6063) | ID NO: | | TGLQAE | NO: 7293) | NO: 6080) |
| | ID NO: | | 6078) | | DEADYY | | |
| | 6077) | | | | C (SEQ ID | | |
| | | | | | NO: 6079) | | |
| 9G1- | QSVTTQ | SGERLSD | WYQQLP | ENDKRP | GVPDRF | QSWDST | FGGGTQ |
| LC_2 | PPSASGT | KYVH | GTAPKM | S (SEQ ID | SGSNSG | NSAV | LTVL |
| | PGQRVTI | (SEQ ID | LIY (SEQ | NO: 6064) | NSASLAI | (SEQ ID | (SEQ ID |
| | SC (SEQ | NO: 6063) | ID NO: | | SGLQSE | NO: 7293) | NO: 6084) |
| | ID NO: | | 6082) | | DEADYY | | |
| | 6081) | | | | C (SEQ ID | | |
| | | | | | NO: 6083) | | |
| 9G1- | QSVTTQ | SGERLSD | WYQQLP | ENDKRP | GVPDRF | QSWDST | FGGGTQ |
| LC_3 | PPSASGT | KYVH | GTAPKM | S (SEQ ID | SGSNSG | NSAV | LTVL |
| | PGQRVTI | (SEQ ID | LIY (SEQ | NO: 6064) | NSASLAI | (SEQ ID | (SEQ ID |
| | SC (SEQ | NO: 6063) | ID NO: | | SGLRSED | NO: 7293) | NO: 6088) |
| | ID NO: | | 6086) | | EADYYC | | |
| | 6085) | | | | (SEQ ID | | |
| | | | | | NO: 6087) | | |
| 9G1- | SSETTQP | SGERLSD | WYQQKP | ENDKRP | GIPERFS | QSWDST | FGGGTQ |
| LC_4 | HSVSVA | KYVH | GQDPVM | S (SEQ ID | GSNPGN | NSAV | LTVL |
| | TAQMAR | (SEQ ID | VIY (SEQ | NO: 6064) | TATLTIS | (SEQ ID | (SEQ ID |
| | ITC (SEQ | NO: 6063) | ID NO: | | RIEAGDE | NO: 7293) | NO: 6092) |
| | ID NO: | | 6090) | | ADYYC | | |
| | 6089) | | | | (SEQ ID | | |
| | | | | | NO: 6091) | | |
| 9G1- | DIQMTQ | SGERLSD | WYQQKP | ENDKRP | GVPSRFS | QSWDST | FGQGTK |
| LC_5 | SPSTLSA | KYVH | GKAPKM | S (SEQ ID | GSNSGN | NSAV | VEIK |
| | SVGDRV | (SEQ ID | LIY (SEQ | NO: 6064) | EATLTIS | (SEQ ID | (SEQ ID |
| | TITC | NO: 6063) | ID NO: | | SLQPDDF | NO: 7293) | NO: 6096) |
| | (SEQ ID | | 6094) | | ATYYC | | |
| | NO: 6093) | | | | (SEQ ID | | |
| | | | | | NO: 6095) | | |
| 15H6- | QYVLTQ | SGENLS | WYQQLP | ENEKRPS | GVPDRF | HYWESI | FGEGTEL |
| LC_1 | PPSASGT | DKYVH | GTAPKM | (SEQ ID | SGSNSG | NSVV | TVL (SEQ |
| | PGQRVTI | (SEQ ID | LIY (SEQ | NO: 6071) | NSASLAI | (SEQ ID | ID NO: |
| | SC (SEQ | NO: 6070) | ID NO: | | SGLQSE | NO: 6072) | 6100) |
| | ID NO: | | 6098) | | DEADYY | | |
| | 6097) | | | | C (SEQ ID | | |
| | | | | | NO: 6099) | | |
| 15H6- | QYVLTQ | SGENLS | WYQQLP | ENEKRPS | GVPDRF | HYWESI | FGEGTEL |
| LC_2 | PPSASGT | DKYVH | GTAPKM | (SEQ ID | SGSNSG | NSVV | TVL (SEQ |
| | PGQRVTI | (SEQ ID | LIY (SEQ | NO: 6071) | NSASLAI | (SEQ ID | ID NO: |
| | SC (SEQ | NO: 6070) | ID NO: | | SGLRSED | NO: 6072) | 6104) |
| | ID NO: | | 6102) | | EADYYC | | |
| | 6101) | | | | (SEQ ID | | |
| | | | | | NO: 6103) | | |
| 15H6- | SYELTQP | SGENLS | WYQQKP | ENEKRPS | GIPERFS | HYWESI | FGEGTEL |
| LC_3 | PSVSVSP | DKYVH | GQSPVM | (SEQ ID | GSNSGN | NSVV | TVL (SEQ |
| | GQTASIT | (SEQ ID | VIY (SEQ | NO: 6071) | TATLTIS | (SEQ ID | ID NO: |
| | C (SEQ ID | NO: 6070) | ID NO: | | GTQAMD | NO: 6072) | 6108) |
| | NO: 6105) | | 6106) | | EADYYC | | |
| | | | | | (SEQ ID | | |
| | | | | | NO: 6107) | | |
| 15H6- | DYVLTQ | SGENLS | WYLQKP | ENEKRPS | GVPDRF | HYWESI | FGQGTK |
| LC_4 | SPLSLPV | DKYVH | GQSPQM | (SEQ ID | SGSNSG | NSVV | VEIK |
| | TPGEPAS | (SEQ ID | LIY (SEQ | NO: 6071) | NDATLKI | (SEQ ID | (SEQ ID |
| | ISC (SEQ | NO: 6070) | ID NO: | | SRVEAE | NO: 6072) | NO: 6112) |
| | ID NO: | | 6110) | | DVGVYY | | |
| | 6109) | | | | C (SEQ ID | | |
| | | | | | NO: 6111) | | |
| 15H6- | AYQLTQ | SGENLS | WYQQKP | ENEKRPS | GVPSRFS | HYWESI | FGQGTK |
| LC_5 | SPSSLSA | DKYVH | GKAPKM | (SEQ ID | GSNSGN | NSVV | VEIK |
| | SVGDRV | (SEQ ID | LIY (SEQ | NO: 6071) | DATLTIS | (SEQ ID | (SEQ ID |
| | TITC | NO: 6070) | ID NO: | | SLQPEDF | NO: 6072) | NO: 6116) |
| | (SEQ ID | | 6114) | | ATYYC | | |
| | NO: 6113) | | | | (SEQ ID | | |
| | | | | | NO: 6115) | | |
| 15H6- | EYVLTQ | SGENLS | WYQQKP | ENEKRPS | GIPARFS | HYWESI | FGQGTK |
| LC_6 | SPATLSV | DKYVH | GQAPRM | (SEQ ID | GSNSGN | NSVV | VEIK |
| | SPGERAT | (SEQ ID | LIY (SEQ | NO: 6071) | EATLTIS | (SEQ ID | (SEQ ID |
| | LSC (SEQ | NO: 6070) | ID NO: | | SLQSEDF | NO: 6072) | NO: 6120) |
| | ID NO: | | 6118) | | AVYYC | | |
| | 6117) | | | | (SEQ ID | | |
| | | | | | NO: 6119) | | |
| 9D9-LC | SYTLTQP | SGENLS | WYQQKP | ENDKRP | GIPDQFS | HCWDST | FGSGTH |
| | PLVSVA | DKYVH | GRAPVM | S (SEQ ID | GSNSGNI | NSAV | LTVL |
| | LGQKATI | (SEQ ID | VIY (SEQ | NO: 6064) | ATLTISK | (SEQ ID | (SEQ ID |
| | IC (SEQ | NO: 6070) | ID NO: | | AQAGYE | NO: 7321) | NO: 6076) |
| | ID NO: | | 6067) | | ADYYC | | |
| | 7320) | | | | (SEQ ID | | |
| | | | | | NO: 7292) | | |
| 3A12- | SYTLTQP | SGENLS | WYQQKP | ENDKRP | GIPDQFS | HCWDST | FGSGTH |
| LC | PLVSVA | DKYVH | GRAPVM | S (SEQ ID | GSNSGNI | NSAV | LTVL |
| | LGQKATI | (SEQ ID | VIY (SEQ | NO: 6064) | ATLTISK | (SEQ ID | (SEQ ID |
| | IC (SEQ | NO: 6070) | ID NO: | | AQAGYE | NO: 7321) | NO: 6076) |
| | ID NO: | | 6067) | | ADYYC | | |
| | 7320) | | | | (SEQ ID | | |
| | | | | | NO: 7292) | | |
| 12D10- | SYTLTQP | SGENLS | WYQQKP | ENEKRPS | GIPDQFS | HYWESI | FGSGTH |
| LC | PSLSVAP | DKYVH | GRAPVM | (SEQ ID | GSNSGNI | NSVV | LTVL |
| | GQKATII | (SEQ ID | VIY (SEQ | NO: 6071) | ATLTISK | (SEQ ID | (SEQ ID |
| | C (SEQ ID | NO: 6070) | ID NO: | | AQPGSE | NO: 6072) | NO: 6076) |
| | NO: 6073) | | 6074) | | ADYYC | | |
| | | | | | (SEQ ID | | |
| | | | | | NO: 6075) | | |
| 15E1- | SFTLTQP | SGEKLS | WYQQKP | ENDRRP | GIPDQFS | QFWDST | FGGGTQ |
| LC | PLVSVA | DKYVH | GRAPVM | S (SEQ ID | GSNSGNI | NSAV | LTVL |
| | VGQVAT | (SEQ ID | VIY (SEQ | NO: 7327) | ASLTISK | (SEQ ID | (SEQ ID |
| | ITC (SEQ | NO: 7326) | ID NO: | | AQAGDE | NO: 7329) | NO: 6080) |
| | ID NO: | | 6067) | | ADYFC | | |
| | 7325) | | | | (SEQ ID | | |
| | | | | | NO: 7328) | | |
| 15E1_ | SSETTQP | SGEKLS | WYQQKP | ENDRRP | GIPERFS | QFWDST | FGGGTQ |
| Humanized | PSVSVSP | DKYVH | GQSPVM | S (SEQ ID | GSNSGN | NSAV | LTVL |
| variant_ | GQTASIT | (SEQ ID | VIY (SEQ | NO: 7327) | TATLTIS | (SEQ ID | (SEQ ID |
| VL1 | C (SEQ ID | NO: 7326) | ID NO: | | GTQAMD | NO: 7329) | NO: 6080) |
| | NO: 7335) | | 6106) | | EADYFC | | |
| | | | | | (SEQ ID | | |
| | | | | | NO: 7336) | | |
| 15E1_ | SSETTQP | SGEKLS | WYQQKP | ENDRRP | GIPERFS | QFWDST | FGGGTQ |
| Humanized | HSVSVA | DKYVH | GQDPVM | S (SEQ ID | GSNPGN | NSAV | LTVL |
| variant_ | TAQMAR | (SEQ ID | VIY (SEQ | NO: 7327) | TATLTIS | (SEQ ID | (SEQ ID |
| VL2 | ITC (SEQ | NO: 7326) | ID NO: | | RIEAGDE | NO: 7329) | NO: 6080) |
| | ID NO: | | 6090) | | ADYFC | | |
| | 6089) | | | | (SEQ ID | | |
| | | | | | NO: 7337) | | |
| 15E1_ | QSVTTQ | SGEKLS | WYQQLP | ENDRRP | GVPDRF | QFWDST | FGGGTQ |
| Humanized | PPSASGT | DKYVH | GTAPKM | S (SEQ ID | SGSNSG | NSAV | LTVL |
| variant_ | PGQRVTI | (SEQ ID | LIY (SEQ | NO: 7327) | NSASLAI | (SEQ ID | (SEQ ID |
| VL3 | SC (SEQ | NO: 7326) | ID NO: | | SGLRSED | NO: 7329) | NO: 6080) |
| | ID NO: | | 6078) | | EADYFC | | |
| | 6081) | | | | (SEQ ID | | |
| | | | | | NO: 7338) | | |
| 15E1_ | QSVTTQ | SGEKLS | WYQQLP | ENDRRP | GVPDRF | QFWDST | FGGGTQ |
| Humanized | PPSVSGA | DKYVH | GTAPKM | S (SEQ ID | SGSNSG | NSAV | LTVL |
| variant_ | PGQRVTI | (SEQ ID | LIY (SEQ | NO: 7327) | NSASLAI | (SEQ ID | (SEQ ID |
| VL4 | SC (SEQ | NO: 7326) | ID NO: | | TGLQAE | NO: 7329) | NO: 6080) |
| | ID NO: | | 6078) | | DEADYF | | |
| | 6077) | | | | C (SEQ ID | | |
| | | | | | NO: 7339) | | |
| 15E1_ | DSVTTQ | SGEKLS | WYQQRP | ENDRRP | GVPDRF | QFWDST | FGGGTK |
| Humanized | SPLSLPV | DKYVH | GQSPRM | S (SEQ ID | SGSNSG | NSAV | VEIK |
| variant_ | TLGQPA | (SEQ ID | LIY (SEQ | NO: 7327) | NDATLKI | (SEQ ID | (SEQ ID |
| VL5 | SISC | NO: 7326) | ID NO: | | SRVEAE | NO: 7329) | NO: 7514) |
| | (SEQ ID | | 7341) | | DVGVYF | | |
| | NO: 7340) | | | | C (SEQ ID | | |
| | | | | | NO: 7342) | | |

TABLE 9A
Exemplary variable regions of NKp30-targeting antigen binding domains

| SEQ ID | | | |
|---|---|---|---|
| NO | Ab ID | Description | Sequence |
| SEQ ID | 9G1-HC | 9G1 heavy chain | QIQLQESGPGLVKPSQSLSLTCSVTGFSINTGGYHWN |
| NO: 6121 | | variable region | WIRQFPGKKLEWMGYIYSSGSTSYNPSLKSRISITRDT |
| | | | SKNQFFLQLNSVTTEDTATYYCARGNWHYFDFWGQ |
| | | | GTMVTVSS |
| SEQ ID | 15H6-HC | 15H6 heavy | QIQLQESGPGLVKPSQSLSLTCSVTGFSINTGGYHWN |
| NO: 6122 | | chain variable | WIRQFPGKKLEWMGYIYSSGTTRYNPSLKSRISITRDT |
| | | region | SKNQFFLQLNSVTPEDTATYYCTRGNWHYFDYWGQ |
| | | | GTLVAVSS |
| SEQ ID | 9G1- | 9G1 heavy chain | QIQLQESGPGLVKPSETLSLTCTVSGFSINTGGYHWN |
| NO: 6123 | HC_1 | variable region | WIRQPAGKGLEWIGYIYSSGSTSYNPSLKSRVTMSRD |
| | | humanized | TSKNQFSLKLSSVTAADTAVYYCARGNWHYFDFWG |
| | | variant 1 | QGTMVTVSS |
| SEQ ID | 9G1- | 9G1 heavy chain | QIQLQESGPGLVKPSQTLSLTCTVSGFSINTGGYHWN |
| NO: 6124 | HC_2 | variable region | WIRQHPGKGLEWIGYIYSSGSTSYNPSLKSLVTISRDT |
| | | humanized | SKNQFSLKLSSVTAADTAVYYCARGNWHYFDFWGQ |
| | | variant 2 | GTMVTVSS |
| SEQ ID | 9G1- | 9G1 heavy chain | EIQLLESGGGLVQPGGSLRLSCAVSGFSINTGGYHWN |
| NO: 6125 | HC_3 | variable region | WVRQAPGKGLEWVGYIYSSGSTSYNPSLKSRFTISRD |
| | | humanized | TSKNTFYLQMNSLRAEDTAVYYCARGNWHYFDFWG |
| | | variant 3 | QGTMVTVSS |
| SEQ ID | 9G1- | 9G1 heavy chain | QIQLVQSGAEVKKPGSSVKVSCKVSGFSINTGGYHW |
| NO: 6126 | HC_4 | variable region | NWVRQAPGQGLEWMGYIYSSGSTSYNPSLKSRVTIT |
| | | humanized | RDTSTNTFYMELSSLRSEDTAVYYCARGNWHYFDFW |
| | | variant 4 | GQGTMVTVSS |
| SEQ ID | 9G1- | 9G1 heavy chain | EIQLVESGGGLVQPGGSLRLSCAVSGFSINTGGYHWN |
| NO: 6127 | HC_5 | variable region | WVRQAPGKGLEWVGYIYSSGSTSYNPSLKSRFTISRD |
| | | humanized | TAKNSFYLQMNSLRAEDTAVYYCARGNWHYFDFWG |
| | | variant 5 | QGTMVTVSS |
| SEQ ID | 9G1- | 9G1 heavy chain | QIQLVQSGAEVKKPGASVKVSCKVSGFSINTGGYHW |
| NO: 6128 | HC_6 | variable region | NWVRQAPGQGLEWMGYIYSSGSTSYNPSLKSRVTMT |
| | | humanized | RDTSTNTFYMELSSLRSEDTAVYYCARGNWHYFDFW |
| | | variant 6 | GQGTMVTVSS |
| SEQ ID | 15H6- | 15H6 heavy | QIQLQESGPGLVKPSQTLSLTCTVSGFSINTGGYHWN |
| NO: 6129 | HC_1 | chain variable | WIRQHPGKGLEWIGYIYSSGTTRYNPSLKSLVTISRDT |
| | | region | SKNQFSLKLSSVTAADTAVYYCARGNWHYFDYWGQ |
| | | humanized | GTLVTVSS |
| | | variant 1 | |
| SEQ ID | 15H6- | 15H6 heavy | QIQLQESGPGLVKPSETLSLTCTVSGFSINTGGYHWN |
| NO: 6130 | HC_2 | chain variable | WIRQPAGKGLEWIGYIYSSGTTRYNPSLKSRVTMSRD |
| | | region | TSKNQFSLKLSSVTAADTAVYYCARGNWHYFDYWG |
| | | humanized | QGTLVTVSS |
| | | variant 2 | |
| SEQ ID | 15H6- | 15H6 heavy | EIQLLESGGGLVQPGGSLRLSCAVSGFSINTGGYHWN |
| NO: 6131 | HC_3 | chain variable | WVRQAPGKGLEWVGYIYSSGTTRYNPSLKSRFTISRD |
| | | region | TSKNTFYLQMNSLRAEDTAVYYCARGNWHYFDYW |
| | | humanized | GQGTLVTVSS |
| | | variant 3 | |
| SEQ ID | 15H6- | 15H6 heavy | QIQLVESGGGLVKPGGSLRLSCAVSGFSINTGGYHWN |
| NO: 6132 | HC_4 | chain variable | WIRQAPGKGLEWVGYIYSSGTTRYNPSLKSRFTISRD |
| | | region | TAKNSFYLQMNSLRAEDTAVYYCARGNWHYFDYW |
| | | humanized | GQGTLVTVSS |
| | | variant 4 | |
| SEQ ID | 15H6- | 15H6 heavy | QIQLVQSGAEVKKPGASVKVSCKVSGFSINTGGYHW |
| NO: 6133 | HC_5 | chain variable | NWVRQAPGQGLEWMGYIYSSGTTRYNPSLKSRVTM |
| | | region | TRDTSTNTFYMELSSLRSEDTAVYYCARGNWHYFDY |
| | | humanized | WGQGTLVTVSS |
| | | variant 5 | |
| SEQ ID | 15H6- | 15H6 heavy | EIQLVQSGAEVKKPGATVKISCKVSGFSINTGGYHWN |
| NO: 6134 | HC_6 | chain variable | WVQQAPGKGLEWMGYIYSSGTTRYNPSLKSRVTITR |
| | | region | DTSTNTFYMELSSLRSEDTAVYYCARGNWHYFDYW |
| | | humanized | GQGTLVTVSS |
| | | variant 6 | |
| SEQ ID | 9G1-LC | 9G1 light chain | SYTLTQPPLLSVALGHKATITCSGERLSDKYVHWYQQ |
| NO: 7294 | | variable region | KPGRAPVMVIYENDKRPSGIPDQFSGSNSGNIATLTIS |
| | | | KAQAGYEADYYCQSWDSTNSAVFGSGTQLTVL |
| SEQ ID | 15H6-LC | 15H6 light chain | SYTLTQPPSLSVAPGQKATIICSGENLSDKYVHWYQQ |
| NO: 6136 | | variable region | KPGRAPVMVIYENEKRPSGIPDQFSGSNSGNIATLTIS |
| | | | KAQPGSEADYYCHYWESINSVVFGSGTHLTVL |
| SEQ ID | 9G1- | 9G1 light chain | QSVTTQPPSVSGAPGQRVTISCSGERLSDKYVHWYQQ |
| NO: 6137 | LC_1 | variable region | LPGTAPKMLIYENDKRPSGVPDRFSGSNSGNSASLAIT |
| | | humanized | GLQAEDEADYYCQSWDSTNSAVFGGGTQLTVL |
| | | variant 1 | |
| SEQ ID | 9G1- | 9G1 light chain | QSVTTQPPSASGTPGQRVTISCSGERLSDKYVHWYQQ |
| NO: 6138 | LC_2 | variable region | LPGTAPKMLIYENDKRPSGVPDRFSGSNSGNSASLAIS |
| | | humanized | GLQSEDEADYYCQSWDSTNSAVFGGGTQLTVL |
| | | variant 2 | |
| SEQ ID | 9G1- | 9G1 light chain | QSVTTQPPSASGTPGQRVTISCSGERLSDKYVHWYQQ |
| NO: 6139 | LC_3 | variable region | LPGTAPKMLIYENDKRPSGVPDRFSGSNSGNSASLAIS |
| | | humanized | GLRSEDEADYYCQSWDSTNSAVFGGGTQLTVL |
| | | variant 3 | |
| SEQ ID | 9G1- | 9G1 light chain | SSETTQPHSVSVATAQMARITCSGERLSDKYVHWYQ |
| NO: 6140 | LC_4 | variable region | QKPGQDPVMVIYENDKRPSGIPERFSGSNPGNTATLTI |
| | | humanized | SRIEAGDEADYYCQSWDSTNSAVFGGGTQLTVL |
| | | variant 4 | |
| SEQ ID | 9G1- | 9G1 light chain | DIQMTQSPSTLSASVGDRVTITCSGERLSDKYVHWYQ |
| NO: 6141 | LC_5 | variable region | QKPGKAPKMLIYENDKRPSGVPSRFSGSNSGNEATLT |
| | | humanized | ISSLQPDDFATYYCQSWDSTNSAVFGQGTKVEIK |
| | | variant 5 | |
| SEQ ID | 15H6- | 15H6 light chain | QYVLTQPPSASGTPGQRVTISCSGENLSDKYVHWYQ |
| NO: 6142 | LC_1 | variable region | QLPGTAPKMLIYENEKRPSGVPDRFSGSNSGNSASLAI |
| | | humanized | SGLQSEDEADYYCHYWESINSVVFGEGTELTVL |
| | | variant 1 | |
| SEQ ID | 15H6- | 15H6 light chain | QYVLTQPPSASGTPGQRVTISCSGENLSDKYVHWYQ |
| NO: 6143 | LC_2 | variable region | QLPGTAPKMLIYENEKRPSGVPDRFSGSNSGNSASLAI |
| | | humanized | SGLRSEDEADYYCHYWESINSVVFGEGTELTVL |
| | | variant 2 | |
| SEQ ID | 15H6- | 15H6 light chain | SYELTQPPSVSVSPGQTASITCSGENLSDKYVHWYQQ |
| NO: 6144 | LC_3 | variable region | KPGQSPVMVIYENEKRPSGIPERFSGSNSGNTATLTIS |
| | | humanized | GTQAMDEADYYCHYWESINSVVFGEGTELTVL |
| | | variant 3 | |
| SEQ ID | 15H6- | 15H6 light chain | DYVLTQSPLSLPVTPGEPASISCSGENLSDKYVHWYL |
| NO: 6145 | LC_4 | variable region | QKPGQSPQMLIYENEKRPSGVPDRFSGSNSGNDATLK |
| | | humanized | ISRVEAEDVGVYYCHYWESINSVVFGQGTKVEIK |
| | | variant 4 | |
| SEQ ID | 15H6- | 15H6 light chain | AYQLTQSPSSLSASVGDRVTITCSGENLSDKYVHWYQ |
| NO: 6146 | LC_5 | variable region | QKPGKAPKMLIYENEKRPSGVPSRFSGSNSGNDATLT |
| | | humanized | ISSLQPEDFATYYCHYWESINSVVFGQGTKVEIK |
| | | variant 5 | |
| SEQ ID | 15H6- | 15H6 light chain | EYVLTQSPATLSVSPGERATLSCSGENLSDKYVHWY |
| NO: 6147 | LC_6 | variable region | QQKPGQAPRMLIYENEKRPSGIPARFSGSNSGNEATL |
| | | humanized | TISSLQSEDFAVYYCHYWESINSVVFGQGTKVEIK |
| | | variant 6 | |
| SEQ ID | 9D9-HC | 9D9 heavy chain | QIQLQESGPGLVKPSQSLSLSCSVTGFSINTGGYHWN |
| NO: 7295 | | variable region | WIRQFPGKKVEWMGYIYSSGTTKYNPSLKSRISITRDT |
| | | | SKNQFFLQLNSVTTEDTATYYCARGDWHYFDYWGQ |
| | | | GTMVAVSS |
| SEQ ID | 9D9-LC | 9D9 light chain | SYTLTQPPLVSVALGQKATIICSGENLSDKYVHWYQQ |
| NO: 7296 | | variable region | KPGRAPVMVIYENDKRPSGIPDQFSGSNSGNIATLTIS |
| | | | KAQAGYEADYYCHCWDSTNSAVFGSGTHLTVL |
| SEQ ID | 3A12-HC | 3A12 heavy | QIQLQESGPGLVKPSQSLSLTCSVTGFSINTGGYHWN |
| NO: 7297 | | chain variable | WIRQFPGKKLEWMGYIYSSGSTRYNPSLKSRFSITRDT |
| | | region | SKNQFFLQLNSVTTEDTATYYCTRGNWHYFDYWGQ |
| | | | GTLVAVSS |
| SEQ ID | 3A12-LC | 3A12 light chain | SYTLTQPPLVSVALGQKATIICSGENLSDKYVHWYQQ |
| NO: 7296 | | variable region | KPGRAPVMVIYENDKRPSGIPDQFSGSNSGNIATLTIS |
| | | | KAQAGYEADYYCHCWDSTNSAVFGSGTHLTVL |
| SEQ ID | 12D10- | 12D10 heavy | QIQLQESGPGLVKPSQSLSLTCSVTGFSINTGGYHWN |
| NO: 6122 | HC | chain variable | WIRQFPGKKLEWMGYIYSSGTTRYNPSLKSRISITRDT |
| | | region | SKNQFFLQLNSVTPEDTATYYCTRGNWHYFDYWGQ |
| | | | GTLVAVSS |
| SEQ ID | 12D10- | 12D10 light | SYTLTQPPSLSVAPGQKATIICSGENLSDKYVHWYQQ |
| NO: 6136 | LC | chain variable | KPGRAPVMVIYENEKRPSGIPDQFSGSNSGNIATLTIS |
| | | region | KAQPGSEADYYCHYWESINSVVFGSGTHLTVL |
| SEQ ID | 15E1-HC | 15E1 heavy | QIQLQESGPGLVKPSQSLSLSCSVTGFSITTTGYHWN |
| NO: 7298 | | chain variable | WIRQFPGKKLEWMGYIYSSGSTSYNPSLKSRFSITRDT |
| | | region | SKNQFFLQLNSVTTEDTATYYCARGDWHYFDYWGP |
| | | | GTMVTVSS |
| SEQ ID | 15E1-LC | 15E1 light chain | SFTLTQPPLVSVAVGQVATITCSGEKLSDKYVHWYQ |
| NO: 7299 | | variable region | QKPGRAPVMVIYENDRRPSGIPDQFSGSNSGNIASLTI |
| | | | SKAQAGDEADYFCQFWDSTNSAVFGGGTQLTVL |
| SEQ ID | 15E1_ | 15E1 heavy | QIQLQESGPGLVKPSQTLSLTCTVSGFSITTTGYHWN |
| NO: 7300 | Humanized | chain variable | WIRQHPGKGLEWIGYIYSSGSTSYNPSLKSLVTISRDT |
| | variant_ | region | SKNQFSLKLSSVTAADTAVYYCARGDWHYFDYWGQ |
| | VH1 | humanized | GTMVTVSS |
| | | variant 1 | |
| SEQ ID | 15E1_ | 15E1 heavy | QIQLVESGGGLVKPGGSLRLSCAVSGFSITTTGYHWN |
| NO: 7301 | Humanized | chain variable | WIRQAPGKGLEWVGYIYSSGSTSYNPSLKSRFTISRDT |
| | variant_ | region | AKNSFYLQMNSLRAEDTAVYYCARGDWHYFDYWG |
| | VH2 | humanized | QGTMVTVSS |
| | | variant 2 | |
| SEQ ID | 15E1_ | 15E1 heavy | EIQLLESGGGLVQPGGSLRLSCAVSGFSITTTGYHWN |
| NO: 7302 | Humanized | chain variable | WVRQAPGKGLEWVGYIYSSGSTSYNPSLKSRFTISRD |
| | variant_ | region | TSKNTFYLQMNSLRAEDTAVYYCARGDWHYFDYW |
| | VH3 | humanized | GQGTMVTVSS |
| | (BJM0407 | variant 3 | |
| | VH and | | |
| | BJM0411 | | |
| | VH) | | |
| SEQ ID | 15E1_ | 15E1 heavy | EIQLVESGGGLVQPGGSLRLSCAVSGFSITTTGYHWN |
| NO: 7303 | Humanized | chain variable | WVRQAPGKGLEWVGYIYSSGSTSYNPSLKSRFTISRD |
| | variant_ | region | TAKNSFYLQMNSLRAEDTAVYYCARGDWHYFDYW |
| | VH4 | humanized | GQGTMVTVSS |
| | | variant 4 | |
| SEQ ID | 15E1_ | 15E1 heavy | QIQLVQSGAEVKKPGASVKVSCKVSGFSITTTGYHW |
| NO: 7304 | Humanized | chain variable | NWVRQAPGQGLEWMGYIYSSGSTSYNPSLKSRVTMT |
| | variant_ | region | RDTSTNTFYMELSSLRSEDTAVYYCARGDWHYFDY |
| | VH5 | humanized | WGQGTMVTVSS |
| | | variant 5 | |
| SEQ ID | 15E1_ | 15E1 light chain | SSETTQPPSVSVSPGQTASITCSGEKLSDKYVHWYQQ |
| NO: 7305 | Humanized | variable region | KPGQSPVMVIYENDRRPSGIPERFSGSNSGNTATLTIS |
| | variant_ | humanized | GTQAMDEADYFCQFWDSTNSAVFGGGTQLTVL |
| | VL1 | variant 1 | |
| | (BJM0407 | | |
| | VL) | | |
| SEQ ID | 15E1_ | 15E1 light chain | SSETTQPHSVSVATAQMARITCSGEKLSDKYVHWYQ |
| NO: 7306 | mHuanized | variable region | QKPGQDPVMVIYENDRRPSGIPERFSGSNPGNTATLTI |
| | variant_ | humanized | SRIEAGDEADYFCQFWDSTNSAVFGGGTQLTVL |
| | VL2 | variant 2 | |
| SEQ ID | 15E1_ | 15E1 light chain | QSVTTQPPSASGTPGQRVTISCSGEKLSDKYVHWYQQ |
| NO: 7307 | Humanized | variable region | LPGTAPKMLIYENDRRPSGVPDRFSGSNSGNSASLAIS |
| | variant_ | humanized | GLRSEDEADYFCQFWDSTNSAVFGGGTQLTVL |
| | VL3 | variant 3 | |
| SEQ ID | 15E1_ | 15E1 light chain | QSVTTQPPSVSGAPGQRVTISCSGEKLSDKYVHWYQ |
| NO: 7308 | Humanized | variable region | QLPGTAPKMLIYENDRRPSGVPDRFSGSNSGNSASLAI |
| | variant_ | humanized | TGLQAEDEADYFCQFWDSTNSAVFGGGTQLTVL |
| | VL4 | variant 4 | |
| SEQ ID | 15E1_ | 15E1 light chain | DSVTTQSPLSLPVTLGQPASISCSGEKLSDKYVHWYQ |
| NO: 7309 | Humanized | variable region | QRPGQSPRMLIYENDRRPSGVPDRFSGSNSGNDATLK |
| | variant_ | humanized | ISRVEAEDVGVYFCQFWDSTNSAVFGGGTKVEIK |
| | VL5 | variant 5 | |
| | (BJM0411 | | |
| | VL) | | |

TABLE 10A
Exemplary NKp30-targeting antigen binding domains/antibody molecules

| SEQ ID | | | |
|---|---|---|---|
| NO | Ab ID | Description | Sequence |
| SEQ ID | Ch(anti- | 9G1 heavy chain | QIQLQESGPGLVKPSQSLSLTCSVTGFSINTGGYHWN |
| NO: 6148 | NKp30 | | WIRQFPGKKLEWMGYIYSSGSTSYNPSLKSRISITRDT |
| | 9G1)HC | | SKNQFFLQLNSVTTEDTATYYCARGNWHYFDFWGQ |
| | N297A | | GTMVTVSSASTKGPSVFPLAPSSKSTSGGTAALGCLV |
| | | | KDYFPEPVTVSWNSGALTSGVHTFPAVLQSSGLYSLS |
| | | | SVVTVPSSSLGTQTYICNVNHKPSNTKVDKRVEPKSC |
| | | | DKTHTCPPCPAPELLGGPSVFLFPPKPKDTLMISRTPE |
| | | | VTCVVVDVSHEDPEVKFNWYVDGVEVHNAKTKPRE |
| | | | EQYASTYRVVSVLTVLHQDWLNGKEYKCKVSNKAL |
| | | | PAPIEKTISKAKGQPREPQVCTLPPSREEMTKNQVSLS |
| | | | CAVKGFYPSDIAVEWESNGQPENNYKTTPPVLDSDGS |
| | | | FFLVSKLTVDKSRWQQGNVFSCSVMHEALHNHYTQ |
| | | | KSLSLSPGK |
| SEQ ID | Ch(anti- | 9G1 heavy chain | QIQLQESGPGLVKPSQSLSLTCSVTGFSINTGGYHWN |
| NO: 6149 | NKp30 | | WIRQFPGKKLEWMGYIYSSGSTSYNPSLKSRISITRDT |
| | 9G1)HC | | SKNQFFLQLNSVTTEDTATYYCARGNWHYFDFWGQ |
| | | | GTMVTVSSASTKGPSVFPLAPSSKSTSGGTAALGCLV |
| | | | KDYFPEPVTVSWNSGALTSGVHTFPAVLQSSGLYSLS |
| | | | SVVTVPSSSLGTQTYICNVNHKPSNTKVDKRVEPKSC |
| | | | DKTHTCPPCPAPELLGGPSVFLFPPKPKDTLMISRTPE |
| | | | VTCVVVDVSHEDPEVKFNWYVDGVEVHNAKTKPRE |
| | | | EQYNSTYRVVSVLTVLHQDWLNGKEYKCKVSNKAL |
| | | | PAPIEKTISKAKGQPREPQVCTLPPSREEMTKNQVSLS |
| | | | CAVKGFYPSDIAVEWESNGQPENNYKTTPPVLDSDGS |
| | | | FFLVSKLTVDKSRWQQGNVFSCSVMHEALHNHYTQ |
| | | | KSLSLSPGK |
| SEQ ID | Ch(anti- | 9G1 light chain | SYTLTQPPLLSVALGHKATITCSGERLSDKYVHWYQQ |
| NO: 6150 | NKp30 | | KPGRAPVMVIYENDKRPSGIPDQFSGSNSGNIATLTIS |
| | 9G1)LC | | KAQAGYEADYYCQSWDSTNSAVFGSGTQLTVLGQP |
| | | | KANPTVTLFPPSSEELQANKATLVCLISDFYPGAVTV |
| | | | AWKADGSPVKAGVETTKPSKQSNNKYAASSYLSLTP |
| | | | EQWKSHRSYSCQVTHEGSTVEKTVAPTECS |
| SEQ ID | Ch(anti- | 15H6 heavy | QIQLQESGPGLVKPSQSLSLTCSVTGFSINTGGYHWN |
| NO: 6151 | NKp30 | chain | WIRQFPGKKLEWMGYIYSSGTTRYNPSLKSRISITRDT |
| | 15H6)HC | | SKNQFFLQLNSVTPEDTATYYCTRGNWHYFDYWGQ |
| | N297A | | GTLVAVSSASTKGPSVFPLAPSSKSTSGGTAALGCLV |
| | | | KDYFPEPVTVSWNSGALTSGVHTFPAVLQSSGLYSLS |
| | | | SVVTVPSSSLGTQTYICNVNHKPSNTKVDKRVEPKSC |
| | | | DKTHTCPPCPAPELLGGPSVFLFPPKPKDTLMISRTPE |
| | | | VTCVVVDVSHEDPEVKFNWYVDGVEVHNAKTKPRE |
| | | | EQYASTYRVVSVLTVLHQDWLNGKEYKCKVSNKAL |
| | | | PAPIEKTISKAKGQPREPQVCTLPPSREEMTKNQVSLS |
| | | | CAVKGFYPSDIAVEWESNGQPENNYKTTPPVLDSDGS |
| | | | FFLVSKLTVDKSRWQQGNVFSCSVMHEALHNHYTQ |
| | | | KSLSLSPGK |
| SEQ ID | Ch(anti- | 15H6 heavy | QIQLQESGPGLVKPSQSLSLTCSVTGFSINTGGYHWN |
| NO: 6152 | NKp30 | chain | WIRQFPGKKLEWMGYIYSSGTTRYNPSLKSRISITRDT |
| | 15H6)HC | | SKNQFFLQLNSVTPEDTATYYCTRGNWHYFDYWGQ |
| | (hole) | | GTLVAVSSASTKGPSVFPLAPSSKSTSGGTAALGCLV |
| | | | KDYFPEPVTVSWNSGALTSGVHTFPAVLQSSGLYSLS |
| | | | SVVTVPSSSLGTQTYICNVNHKPSNTKVDKRVEPKSC |
| | | | DKTHTCPPCPAPELLGGPSVFLFPPKPKDTLMISRTPE |
| | | | VTCVVVDVSHEDPEVKFNWYVDGVEVHNAKTKPRE |
| | | | EQYNSTYRVVSVLTVLHQDWLNGKEYKCKVSNKAL |
| | | | PAPIEKTISKAKGQPREPQVCTLPPSREEMTKNQVSLS |
| | | | CAVKGFYPSDIAVEWESNGQPENNYKTTPPVLDSDGS |
| | | | FFLVSKLTVDKSRWQQGNVFSCSVMHEALHNHYTQ |
| | | | KSLSLSPGK |
| SEQ ID | Ch(anti- | 15H6 light chain | SYTLTQPPSLSVAPGQKATIICSGENLSDKYVHWYQQ |
| NO: 6153 | NKp30 | | KPGRAPVMVIYENEKRPSGIPDQFSGSNSGNIATLTIS |
| | 15H6)LC | | KAQPGSEADYYCHYWESINSVVFGSGTHLTVLGQPK |
| | | | ANPTVTLFPPSSEELQANKATLVCLISDFYPGAVTVA |
| | | | WKADGSPVKAGVETTKPSKQSNNKYAASSYLSLTPE |
| | | | QWKSHRSYSCQVTHEGSTVEKTVAPTECS |
| SEQ ID | BJM0859 | | EIQLLESGGGLVQPGGSLRLSCAVSGFSITTTGYHWN |
| NO: 7310 | lambda | | WVRQAPGKGLEWVGYIYSSGSTSYNPSLKSRFTISRD |
| | scFv | | TSKNTFYLQMNSLRAEDTAVYYCARGDWHYFDYW |
| | | | GQGTMVTVSSGGGGSGGGGSGGGGSGGGGSSSETTQ |
| | | | PPSVSVSPGQTASITCSGEKLSDKYVHWYQQKPGQSP |
| | | | VMVIYENDRRPSGIPERFSGSNSGNTATLTISGTQAMD |
| | | | EADYFCQFWDSTNSAVFGGGTQLTVL |
| SEQ ID | BJM0860 | | EIQLLESGGGLVQPGGSLRLSCAVSGFSITTTGYHWN |
| NO: 7311 | kappa | | WVRQAPGKGLEWVGYIYSSGSTSYNPSLKSRFTISRD |
| | scFv | | TSKNTFYLQMNSLRAEDTAVYYCARGDWHYFDYW |
| | | | GQGTMVTVSSGGGGSGGGGSGGGGSGGGGSDSVTT |
| | | | QSPLSLPVTLGQPASISCSGEKLSDKYVHWYQQRPGQ |
| | | | SPRMLIYENDRRPSGVPDRFSGSNSGNDATLKISRVEA |
| | | | EDVGVYFCQFWDSTNSAVFGGGTKVEIK |

In some embodiments, the NK cell engager is an antigen binding domain that binds to NKp46 (e.g., NKp46 present, e.g., expressed or displayed, on the surface of an NK cell) and comprises any CDR amino acid sequence, framework region (FWR) amino acid sequence, or variable region amino acid sequence disclosed in Table 10B. In some embodiments, binding of the NK cell engager, e.g., antigen binding domain that binds to NKp46, to the NK cell activates the NK cell. An antigen binding domain that binds to NKp46 (e.g., NKp46 present, e.g., expressed or displayed, on the surface of an NK cell) may be said to target NKp46, the NK cell, or both.

In some embodiments, the NK cell engager is an antigen binding domain that binds to NKG2D (e.g., NKG2D present, e.g., expressed or displayed, on the surface of an NK cell) and comprises any CDR amino acid sequence, framework region (FWR) amino acid sequence, or variable region amino acid sequence disclosed in Table 10B. In some embodiments, binding of the NK cell engager, e.g., antigen binding domain that binds to NKG2D, to the NK cell activates the NK cell. An antigen binding domain that binds to NKG2D (e.g., NKG2D present, e.g., expressed or displayed, on the surface of an NK cell) may be said to target NKG2D, the NK cell, or both.

In some embodiments, the NK cell engager is an antigen binding domain that binds to CD16 (e.g., CD16 present, e.g., expressed or displayed, on the surface of an NK cell) and comprises any CDR amino acid sequence, framework region (FWR) amino acid sequence, or variable region amino acid sequence disclosed in Table 10B. In some embodiments, binding of the NK cell engager, e.g., antigen binding domain that binds to CD16, to the NK cell activates the NK cell. An antigen binding domain that binds to CD16 (e.g., CD16 present, e.g., expressed or displayed, on the surface of an NK cell) may be said to target CD16, the NK cell, or both.

TABLE 10B
Exemplary variable regions of NKp46, NKG2D, or CD16-targeting antigen binding
domains

| SEQ ID | | | |
|---|---|---|---|
| NO | Ab ID | Description | Sequence |
| SEQ ID | NKG2D_ | scFV that binds | QVHLQESGPGLVKPSETLSLTCTVSDDSISSYYWSWIR |
| NO: 6175 | 1scFV | NKG2D | QPPGKGLEWIGHISYSGSANYNPSLKSRVTISVDTSKN |
| | | | QFSLKLSSVTAADTAVYYCANWDDAFNIWGQGTMV |
| | | | TVSSGGGGSGGGGSGGGGSGGGGSEIVLTQSPGTLSL |
| | | | SPGERATLSCRASQSVSSSYLAWYQQKPGQAPRLLIY |
| | | | GASSRATGIPDRFSGSGSGTDFTLTISRLEPEDFAVYY |
| | | | CQQYGSSPWTFGQGTKVEIK |
| SEQ ID | NKG2D_ | VH that binds | QVHLQESGPGLVKPSETLSLTCTVSDDSISSYYWSWIR |
| NO: 6176 | 1VH | NKG2D | QPPGKGLEWIGHISYSGSANYNPSLKSRVTISVDTSKN |
| | | | QFSLKLSSVTAADTAVYYCANWDDAFNIWGQGTMV |
| | | | TVSS |
| SEQ ID | NKG2D_ | VL that binds | EIVLTQSPGTLSLSPGERATLSCRASQSVSSSYLAWYQ |
| NO: 6177 | 1VL | NKG2D | QKPGQAPRLLIYGASSRATGIPDRFSGSGSGTDFTLTIS |
| | | | RLEPEDFAVYYCQQYGSSPWTFGQGTKVEIK |
| SEQ ID | NKG2D_ | scFV that binds | EVQLVQSGAEVKEPGESLKISCKNSGYSFTNYWVGW |
| NO: 6178 | 2scFV | NKG2D | VRQMPGKGLEWMGIIYPGDSDTRYSPSFQGQVTISAD |
| | | | KSINTAYLQWSSLKASDTAMYYCGRLTMFRGIIIGYF |
| | | | DYWGQGTLVTVSSGGGGSGGGGSGGGGSGGGGSEI |
| | | | VLTQSPATLSLSPGERATLSCRASQSVSSYLAWYQQK |
| | | | PGQAPRLLIYDASNRATGIPARFSGSGSGTDFTLTISSL |
| | | | EPEDFAVYYCQQRSNWPWTFGQGTKVEIK |
| SEQ ID | NKG2D_ | VH that binds | EVQLVQSGAEVKEPGESLKISCKNSGYSFTNYWVGW |
| NO: 6179 | 2VH | NKG2D | VRQMPGKGLEWMGIIYPGDSDTRYSPSFQGQVTISAD |
| | | | KSINTAYLQWSSLKASDTAMYYCGRLTMFRGIIIGYF |
| | | | DYWGQGTLVTVSS |
| SEQ ID | NKG2D_ | VL that binds | EIVLTQSPATLSLSPGERATLSCRASQSVSSYLAWYQQ |
| NO: 6180 | 2VL | NKG2D | KPGQAPRLLIYDASNRATGIPARFSGSGSGTDFTLTISS |
| | | | LEPEDFAVYYCQQRSNWPWTFGQGTKVEIK |
| SEQ ID | NKp46scFV | scFV that binds | QVQLQQSGPELVKPGASVKMSCKASGYTFTDYVINW |
| NO: 6181 | | NKp46 | GKQRSGQGLEWIGEIYPGSGTNYYNEKFKAKATLTA |
| | | | DKSSNIAYMQLSSLTSEDSAVYFCARRGRYGLYAMD |
| | | | YWGQGTSVTVSSGGGGSGGGGSGGGGSGGGGSDIQ |
| | | | MTQTTSSLSASLGDRVTISCRASQDISNYLNWYQQKP |
| | | | DGTVKLLIYYTSRLHSGVPSRFSGSGSGTDYSLTINNL |
| | | | EQEDIATYFCQQGNTRPWTFGGGTKLEIK |
| SEQ ID | NKp46VH | VH that binds | QVQLQQSGPELVKPGASVKMSCKASGYTFTDYVINW |
| NO: 6182 | | NKp46 | GKQRSGQGLEWIGEIYPGSGTNYYNEKFKAKATLTA |
| | | | DKSSNIAYMQLSSLTSEDSAVYFCARRGRYGLYAMD |
| | | | YWGQGTSVTVSS |
| SEQ ID | NKp46VL | VL that binds | DIQMTQTTSSLSASLGDRVTISCRASQDISNYLNWYQ |
| NO: 6183 | | NKp46 | QKPDGTVKLLIYYTSRLHSGVPSRFSGSGSGTDYSLTI |
| | | | NNLEQEDIATYFCQQGNTRPWTFGGGTKLEIK |
| SEQ ID | CD16scFV | scFV that binds | EVQLVESGG GVVRPGGSLR LSCAASGFTF |
| NO: 6184 | | CD16 | DDYGMSWVRQ APGKGLEWVS |
| | | | GINWNGGSTG YADSVKGRFT ISRDNAKNSL |
| | | | YLQMNSLRAE DTAVYYCARG |
| | | | RSLLFDYWGQ GTLVTVSRGG GGSGGGGSGG |
| | | | GGSSELTQDP AVSVALGQTV |
| | | | RITCQGDSLR SYYASWYQQK PGQAPVLVIY |
| | | | GKNNRPSGIP DRFSGSSSGN |
| | | | TASLTITGAQ AEDEADYYCN SRDSSGNHVV |
| | | | FGGGTKLTVL |
| SEQ ID | CD16VH | VH that binds | EVQLVESGG GVVRPGGSLR LSCAASGFTF |
| NO: 6185 | | CD16 | DDYGMSWVRQ APGKGLEWVS |
| | | | GINWNGGSTG YADSVKGRFT ISRDNAKNSL |
| | | | YLQMNSLRAE DTAVYYCARG |
| | | | RSLLFDYWGQ GTLVTVSR |
| SEQ ID | CD16VL | VL that binds | SSELTQDP AVSVALGQTVRITCQGDSLR |
| NO: 6186 | | CD16 | SYYASWYQQK PGQAPVLVIY GKNNRPSGIP |
| | | | DRFSGSSSGNTASLTITGAQ AEDEADYYCN |
| | | | SRDSSGNHVV FGGGTKLTVL |

In one embodiment, the NK cell engager is a ligand of NKp30, e.g., is a B7-6, e.g., comprises the amino acid sequence of: DLKVEMMAGGTQITPLNDNVTIFCNIFYSQPLNITSMGITWFWKSLTFDKEVKVFEFFGDHQEAF RPGAIVSPWRLKSGDASLRLPGIQLEEAGEYRCEVVVTPLKAQGTVQLEVVASPASRLLLDQVG MKENEDKYMCESSGFYPEAINITWEKQTQKFPHPIEISEDVITGPTIKNMDGTFNVTSCLKLNSSQ EDPGTVYQCVVRHASLHTPLRSNFTLTAARHSLSETEKTDNFS (SEQ ID NO: 7233), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7233.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:
(i) MICA comprises the amino acid sequence: EPHSLRYNLTVLSWDGSVQSGFLTEVHLDGQPFLRCDRQKCRAKPQGQWAEDVLGNKTWDRE TRDLTGNGKDLRMTLAHIKDQKEGLHSLQEIRVCEIHEDNSTRSSQHFYYDGELFLSQNLETKEW TMPQSSRAQTLAMNVRNFLKEDAMKTKTHYHAMHADCLQELRRYLKSGVVLRRTVPPMVNVT RSEASEGNITVTCRASGFYPWNITLSWRQDGVSLSHDTQQWGDVLPDGNGTYQTWVATRICQG EEQRFTCYMEHSGNHSTHPVPSGKVLVLQSHW (SEQ ID NO: 7234), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7234;
(ii) MICB comprises the amino acid sequence: AEPHSLRYNLMVLSQDESVQSGFLAEGHLDGQPFLRYDRQKRRAKPQGQWAEDVLGAKTWDT ETEDLTENGQDLRRTLTHIKDQKGGLHSLQEIRVCEIHEDSSTRGSRHFYYDGELFLSQNLETQES TVPQSSRAQTLAMNVTNFWKEDAMKTKTHYRAMQADCLQKLQRYLKSGVAIRRTVPPMVNVT CSEVSEGNITVTCRASSFYPRNITLTWRQDGVSLSHNTQQWGDVLPDGNGTYQTWVATRIRQGE EQRFTCYMEHSGNHGTHPVPSGKVLVLQSQRTD (SEQ ID NO: 7235), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7235; or
(iii) ULBP1 comprises the amino acid sequence: GWVDTHCLCYDFIITPKSRPEPQWCEVQGLVDERPFLHYDCVNHKAKAFASLGKKVNVTKTWE EQTETLRDVVDFLKGQLLDIQVENLIPIEPLTLQARMSCEHEAHGHGRGSWQFLFNGQKFLLFDS NNRKWTALHPGAKKMTEKWEKNRDVTMFFQKISLGDCKMWLEEFLMYWEQMLDPTKPPSLA PG (SEQ ID NO: 7236), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7236.

In other embodiments, the NK cell engager is a ligand of DNAM1 chosen from NECTIN2 or NECL5, e.g., wherein:
(i) NECTIN2 comprises the amino acid sequence: QDVRVQVLPEVRGQLGGTVELPCHLLPPVPGLYISLVTWQRPDAPANHQNVAAFHPKMGPSFPS PKPGSERLSFVSAKQSTGQDTEAELQDATLALHGLTVEDEGNYTCEFATFPKGSVRGMTWLRVI AKPKNQAEAQKVTFSQDPTTVALCISKEGRPPARISWLSSLDWEAKETQVSGTLAGTVTVTSRFT LVPSGRADGVTVTCKVEHESFEEPALIPVTLSVRYPPEVSISGYDDNWYLGRTDATLSCDVRSNP EPTGYDWSTTSGTFPTSAVAQGSQLVIHAVDSLFNTTFVCTVTNAVGMGRAEQVIFVRETPNTA GAGATGG (SEQ ID NO: 7237), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7237; or
(ii) NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHSG ISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7238.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fe to trigger NK cells through CD16,the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSSELKVSL TNVSISDEGRYFCQLYTDPPQESYTTITVLVPPRNLMIDIQKDTAVEGEEIEVNCTAMASKPATTIR WFKGNTELKGKSEVEEWSDMYTVTSQLMLKVHKEDDGVPVICQVEHPAVTGNLQTQRYLEVQ YKPQVHIQMTYPLQGLTREGDALELTCEAIGKPQPVMVTWVRVDDEMPQHAVLSGPNLFINNL NKTDNGTYRCEASNIVGKAHSDYMLYVYDPPTTIPPPTTTTTTTTTTTTTILTIITDSRAGEEGSIR AVDH (SEQ ID NO: 7239), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7239.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQLRIHRD GIYMVHIQVTLAICSSTTASRHHPTTLAVGICSPASRSISLLRLSFHQGCTIASQRLTPLARGDTLCT NLTGTLLPSRNTDETFFGVQWVRP (SEQ ID NO: 7240), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7240.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGIWTWVG TNKSLTEEAENWGDGEPNNKKNKEDCVEIYIKRNKDAGKWNDDACHKLKAALCYTASCQPWS CSGHGECVEIINNYTCNCDVGYYGPQCQFVIQCEPLEAPELGTMDCTHPLGNFSFSSQCAFSCSEG TNLTGIEETTCGPFGNWSSPEPTCQVIQCEPLSAPDLGIMNCSHPLASFSFTSACTFICSEGTELIGK KKTICESSGIWSNPSPICQKLDKSFSMIKEGDYN (SEQ ID NO: 7241), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7241.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHSG ISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7239.

In other embodiments, the NK cell engager is a ligand of CD100 (SEMA4D), which is CD72, e.g., wherein CD72 comprises the amino acid sequence:

(SEQ ID NO: 7242)
RYLQVSQQLQQTNRVLEVINSSLRQQLRLKITQLGQSAEDLQGSRRELA
QSQEALQVEQRAHQAAEGQLQACQADRQKTKETLQSEEQQRRALEQKLS
NMENRLKPFFTCGSADTCCPSGWIMHQKSCFYISLTSKNWQESQKQCET
LSSKLATFSEIYPQSHSYYFLNSLLPNGGSGNSYWTGLSSNKDWKLTDD
TQRTRTYAQSSKCNKVHKTWSWWTLESESCRSSLPYICEMTAFRFPD,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7242.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRRYKCSSD HWIGLKMAKNRTGQWVDGATFTKSFGMRGSEGCAYLSDDGAATARCYTERKWICRKRIH (SEQ ID NO: 7243), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7243.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGRVRLDPQ SGALYISKVQKEDNSTYIMRVLKKTGNEQEWKIKLQVLDPVPKPVIKIEKIEDMDDNCYLKLSCV IPGESVNYTWYGDKRPFPKELQNSVLETTLMPHNYSRCYTCQVSNSVSSKNGTVCLSPPCTLARS (SEQ ID NO: 7244), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7244.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

### Death Receptor Signal Engagers

Death receptors, e.g., death receptors 4 and 5 (DR4 and DR5, also known as TRAIL-RI and TRAIL-R2 respectively), are trimeric type I transmembrane proteins widely expressed in normal human tissues. Activation of death receptors causes intracellular signaling that induces cell death. TNF-related apoptosis-inducing ligand (TRAIL) (also known as Apo2L) is a trimeric protein that binds to Death receptors, activating their cell death-inducing signaling (Amarante-Mendes and Griffith. Pharmacol Ther. 2015 November; 155:117-31).

The present disclosure provides, inter alia, TCR targeting molecules that are engineered to contain one or more death receptor signal engagers that mediate binding to death receptors and/or activation of death receptor signaling on a target cell (e.g., the T cell comprising a TCRAV or TCRBV antigen (e.g., a TCRAV or TCRBV antigen corresponding to a biased TCRBV clonotype). Accordingly, in some embodiments, the death receptor signal engager comprises one or more TRAIL polypeptides or a fragment thereof (TRAIL molecule), one or more death receptors or a fragment thereof (death receptor molecule), or one or more antigen binding domains that specifically binds to a death receptor (e.g., and activates death receptor signaling). Without wishing to be bound by theory, it is thought that a death receptor signal engager that can activate death receptor signaling on a target cell can induce the death of the target cell, e.g., a target disease cell (e.g., a T cell comprising a TCRAV or TCRBV antigen (e.g., a TCRAV or TCRBV antigen corresponding to a biased TCRAV or TCRBV clonotype).

Death receptor signal engagers may comprise TRAIL molecules and/or death receptor molecules from or derived from versions of TRAIL and death receptors known to those skilled in the art. In some embodiments, the death receptor signal engager comprises a human TRAIL molecule or death receptor molecule. In some embodiments, the death receptor signal engager comprises a mouse TRAIL molecule or death receptor molecule. In some embodiments, the death receptor signal engager comprises a mammalian TRAIL molecule or death receptor molecule. In some embodiments, the death receptor signal engager comprises a truncated TRAIL molecule or death receptor molecule (e.g., relative to a wild-type TRAIL molecule or death receptor molecule).

In some embodiments, the death receptor signal engager comprises a truncated TRAIL molecule comprising at least residues corresponding to amino acids 95-281 of human TRAIL, e.g., a truncated TRAIL molecule comprising residues corresponding to amino acids 95-281 of human TRAIL. In some embodiments, the death receptor signal engager comprises a truncated TRAIL molecule comprising residues of 95-281 of human TRAIL.

In some embodiments, the death receptor signal engager comprises a truncated TRAIL molecule comprising at least residues corresponding to amino acids 122-281 of human TRAIL, e.g., a truncated TRAIL molecule comprising residues corresponding to amino acids 122-281 of human TRAIL. In some embodiments, the death receptor signal engager comprises a truncated TRAIL molecule comprising residues of 122-281 of human TRAIL.

In some embodiments, the death receptor signal engager comprises one, two, or three TRAIL molecules (e.g., the death receptor signal engager is a monomeric, dimeric, or trimeric TRAIL molecule, respectively). In some embodiments, the death receptor signal engager comprises one, two, or three death receptor molecules (e.g., the death receptor signal engager is a monomeric, dimeric, or trimeric death receptor molecule, respectively). In some embodiments, the death receptor signal engager comprises one, two, or three antigen binding domains that specifically bind to a death receptor (e.g., to one or more death receptors, e.g., the same or different death receptors)

In some embodiments, the death receptor signal engager comprises an amino acid sequence selected from Table 11 A (or an amino acid sequence having at least about 77%, 80%, 85%, 90%, 95%, or 99% sequence identity to a sequence selected from Table 11A).

In some embodiments, the death receptor signal engager comprises an amino acid sequence of SEQ ID NO: 6157 (or an amino acid sequence having at least about 77%, 80%, 85%, 90%, 95%, or 99% sequence identity to SEQ ID NO: 6157).

In some embodiments, the death receptor signal engager comprises an amino acid sequence of SEQ ID NO: 6158 (or an amino acid sequence having at least about 77%, 80%, 85%, 90%, 95%, or 99% sequence identity to SEQ ID NO: 6158).

In some embodiments, the death receptor signal engager comprises an amino acid sequence of SEQ ID NO: 6159 (or an amino acid sequence having at least about 77%, 80%, 85%, 90%, 95%, or 99% sequence identity to SEQ ID NO: 6159).

In some embodiments, the death receptor signal engager comprises an amino acid sequence of SEQ ID NO: 6160 (or an amino acid sequence having at least about 77%, 80%, 85%, 90%, 95%, or 99% sequence identity to SEQ ID NO: 6160).

In some embodiments, the death receptor signal engager comprises an amino acid sequence of SEQ ID NO: 6161 (or an amino acid sequence having at least about 77%, 80%, 85%, 90%, 95%, or 99% sequence identity to SEQ ID NO: 6161).

In some embodiments, the death receptor signal engager comprises an amino acid sequence of SEQ ID NO: 6162 (or an amino acid sequence having at least about 77%, 80%, 85%, 90%, 95%, or 99% sequence identity to SEQ ID NO: 6162).

In some embodiments, the death receptor signal engager comprises an amino acid sequence of SEQ ID NO: 6163 (or an amino acid sequence having at least about 77%, 80%, 85%, 90%, 95%, or 99% sequence identity to SEQ ID NO: 6163).

In some embodiments, the death receptor signal engager comprises an amino acid sequence of SEQ ID NO: 6164 (or an amino acid sequence having at least about 77%, 80%, 85%, 90%, 95%, or 99% sequence identity to SEQ ID NO: 6164).

In some embodiments, the death receptor signal engager comprises an amino acid sequence of SEQ ID NO: 6165 (or an amino acid sequence having at least about 77%, 80%, 85%, 90%, 95%, or 99% sequence identity to SEQ ID NO: 6165).

In some embodiments, the death receptor signal engager is comprised on the same polypeptide chain as another component of a TCR targeting molecule of the present disclosure, e.g., the death receptor signal engager is comprised on the same polypeptide chain as a heavy and/or light chain of a first antigen binding domain that preferentially binds to a TCRBV antigen (e.g., a TCRBV antigen corresponding to a biased TCRBV clonotype) on a lymphocyte cell (e.g., T cell), an immune cell engager, a cytokine molecule, or a cytokine inhibitor molecule, e.g., as a fusion protein. In some embodiments, the TCR targeting molecule comprises a fusion protein comprising a death receptor signal engager and light chain of a first antigen binding domain that preferentially binds to a TCRBV antigen (e.g., a TCRBV antigen corresponding to a biased TCRBV clonotype). In some embodiments, the TCR targeting molecule comprises a fusion protein comprising a death receptor signal engager and a light chain of a first antigen binding domain that selectively targets lymphocytes expressing a TCRBV antigen (e.g., a TCRBV antigen corresponding to a biased TCRBV clonotype).

TABLE 11A
Exemplary death receptor signal engagers

| SEQ ID | | | |
|---|---|---|---|
| NO | ID Ref. | Description | Sequence |
| SEQ ID | monomeric_ | Monomeric | METDTLLLWVLLLWVPGSTGDYKDDDDKGGGGSGT |
| NO: 6157 | hTRAIL_ | human TRAIL | GGAAAHITGTRGRSNTLSSPNSKNEKALGRKINSWES |
| | aa122_281- | comprising | SRSGHSFLSNLHLRNGELVIHEKGFYYIYSQTYFRFQE |
| | hFc_Knob_ | residues 122- | EIKENTKNDKQMVQYIYKYTSYPDPILLMKSARNSC |
| | Cys- | 281 | WSKDAEYGLYSIYQGGIFELKENDRIFVSVTNEHLID |
| | Blank | | MDHEASFFGAFAVSGSGNGTSNGTSGSSGGDKTHTC |
| | | | PPCPAPELLGGPSVFLFPPKPKDTLMISRTPEVTCVVV |
| | | | DVSHEDPEVKFNWYVDGVEVHNAKTKPREEQYNST |
| | | | YRVVSVLTVLHQDWLNGKEYKCKVSNKALPAPIEKT |
| | | | ISKAKGQPREPQVYTLPPCREEMTKNQVSLWCLVKG |
| | | | FYPSDIAVEWESNGQPENNYKTTPPVLDSDGSFFLYS |
| | | | KLTVDKSRWQQGNVFSCSVMHEALHNHYTQKSLSLS |
| | | | PGK |
| SEQ ID | dimeric_ | Dimeric human | METDTLLLWVLLLWVPGSTGDYKDDDDKGGGGSGT |
| NO: 6158 | hTRAIL_ | TRAIL | GGAAAHITGTRGRSNTLSSPNSKNEKALGRKINSWES |
| | aa122_281- | comprising | SRSGHSFLSNLHLRNGELVIHEKGFYYIYSQTYFRFQE |
| | hFc_Knob_ | residues 122- | EIKENTKNDKQMVQYIYKYTSYPDPILLMKSARNSC |
| | Cys- | 281 | WSKDAEYGLYSIYQGGIFELKENDRIFVSVTNEHLID |
| | Blank | | MDHEASFFGAFAVSGAAAHITGTRGRSNTLSSPNSKN |
| | | | EKALGRKINSWESSRSGHSFLSNLHLRNGELVIHEKG |
| | | | FYYIYSQTYFRFQEEIKENTKNDKQMVQYIYKYTSYP |
| | | | DPILLMKSARNSCWSKDAEYGLYSIYQGGIFELKEND |
| | | | RIFVSVTNEHLIDMDHEASFFGAFAVSGSGNGTSNGT |
| | | | SGSSGGDKTHTCPPCPAPELLGGPSVFLFPPKPKDTLM |
| | | | ISRTPEVTCVVVDVSHEDPEVKFNWYVDGVEVHNAK |
| | | | TKPREEQYNSTYRVVSVLTVLHQDWLNGKEYKCKV |
| | | | SNKALPAPIEKTISKAKGQPREPQVYTLPPCREEMTKN |
| | | | QVSLWCLVKGFYPSDIAVEWESNGQPENNYKTTPPV |
| | | | LDSDGSFFLYSKLTVDKSRWQQGNVFSCSVMHEALH |
| | | | NHYTQKSLSLSPGK |
| SEQ ID | trimeric_ | Trimeric human | METDTLLLWVLLLWVPGSTGDYKDDDDKGGGGSGT |
| NO: 6159 | hTRAIL_ | TRAIL | GGAAAHITGTRGRSNTLSSPNSKNEKALGRKINSWES |
| | aa122_281- | comprising | SRSGHSFLSNLHLRNGELVIHEKGFYYIYSQTYFRFQE |
| | hFc_Knob_ | residues 122- | EIKENTKNDKQMVQYIYKYTSYPDPILLMKSARNSC |
| | Cys- | 281 | WSKDAEYGLYSIYQGGIFELKENDRIFVSVTNEHLID |
| | Blank | | MDHEASFFGAFAVSGAAAHITGTRGRSNTLSSPNSKN |
| | | | EKALGRKINSWESSRSGHSFLSNLHLRNGELVIHEKG |
| | | | FYYIYSQTYFRFQEEIKENTKNDKQMVQYIYKYTSYP |
| | | | DPILLMKSARNSCWSKDAEYGLYSIYQGGIFELKEND |
| | | | RIFVSVTNEHLIDMDHEASFFGAFAVSGAAAHITGTR |
| | | | GRSNTLSSPNSKNEKALGRKINSWESSRSGHSFLSNLH |
| | | | LRNGELVIHEKGFYYIYSQTYFRFQEEIKENTKNDKQ |
| | | | MVQYIYKYTSYPDPILLMKSARNSCWSKDAEYGLYSI |
| | | | YQGGIFELKENDRIFVSVTNEHLIDMDHEASFFGAFA |
| | | | VSGSGNGTSNGTSGSSGGDKTHTCPPCPAPELLGGPS |
| | | | VFLFPPKPKDTLMISRTPEVTCVVVDVSHEDPEVKFN |
| | | | WYVDGVEVHNAKTKPREEQYNSTYRVVSVLTVLHQ |
| | | | DWLNGKEYKCKVSNKALPAPIEKTISKAKGQPREPQ |
| | | | VYTLPPCREEMTKNQVSLWCLVKGFYPSDIAVEWES |
| | | | NGQPENNYKTTPPVLDSDGSFFLYSKLTVDKSRWQQ |
| | | | GNVFSCSVMHEALHNHYTQKSLSLSPGK |
| SEQ ID | monomeric_ | Monomeric | METDTLLLWVLLLWVPGSTGTSEETISTVQEKQQNIS |
| NO: 6160 | hTRAIL_ | human TRAIL | PLVRERGPQRVAAHITGTRGRSNTLSSPNSKNEKALG |
| | 95-281- | comprising | RKINSWESSRSGHSFLSNLHLRNGELVIHEKGFYYIYS |
| | hFc_Knob_ | residues 95-281 | QTYFRFQEEIKENTKNDKQMVQYIYKYTSYPDPILLM |
| | Cys- | | KSARNSCWSKDAEYGLYSIYQGGIFELKENDRIFVSV |
| | Blank | | TNEHLIDMDHEASFFGAFLVGGGGGSGGGGSDKTHT |
| | | | CPPCPAPELLGGPSVFLFPPKPKDTLMISRTPEVTCVV |
| | | | VDVSHEDPEVKFNWYVDGVEVHNAKTKPREEQYNS |
| | | | TYRVVSVLTVLHQDWLNGKEYKCKVSNKALPAPIEK |
| | | | TISKAKGQPREPQVYTLPPCREEMTKNQVSLWCLVK |
| | | | GFYPSDIAVEWESNGQPENNYKTTPPVLDSDGSFFLY |
| | | | SKLTVDKSRWQQGNVFSCSVMHEALHNHYTQKSLSL |
| | | | SPGK |
| SEQ ID | dimeric_ | Dimeric human | METDTLLLWVLLLWVPGSTGTSEETISTVQEKQQNIS |
| NO: 6161 | hTRAIL_ | TRAIL | PLVRERGPQRVAAHITGTRGRSNTLSSPNSKNEKALG |
| | 95-281- | comprising | RKINSWESSRSGHSFLSNLHLRNGELVIHEKGFYYIYS |
| | hFc_Knob_ | residues 95-281 | QTYFRFQEEIKENTKNDKQMVQYIYKYTSYPDPILLM |
| | Cys- | | KSARNSCWSKDAEYGLYSIYQGGIFELKENDRIFVSV |
| | Blank | | TNEHLIDMDHEASFFGAFLVGGGGGSGGGGSGTSEET |
| | | | ISTVQEKQQNISPLVRERGPQRVAAHITGTRGRSNTLS |
| | | | SPNSKNEKALGRKINSWESSRSGHSFLSNLHLRNGEL |
| | | | VIHEKGFYYIYSQTYFRFQEEIKENTKNDKQMVQYIY |
| | | | KYTSYPDPILLMKSARNSCWSKDAEYGLYSIYQGGIF |
| | | | ELKENDRIFVSVTNEHLIDMDHEASFFGAFLVGGGGG |
| | | | SGGGGSDKTHTCPPCPAPELLGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALPAPIEKTISKAKGQPREPQVYTLPPCREEMT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| SEQ ID | trimeric_ | Trimeric human | METDTLLLWVLLLWVPGSTGTSEETISTVQEKQQNIS |
| NO: 6162 | hTRAIL_ | TRAIL | PLVRERGPQRVAAHITGTRGRSNTLSSPNSKNEKALG |
| | 95-281- | comprising | RKINSWESSRSGHSFLSNLHLRNGELVIHEKGFYYIYS |
| | hFc_Knob_ | residues 95-281 | QTYFRFQEEIKENTKNDKQMVQYIYKYTSYPDPILLM |
| | Cys- | | KSARNSCWSKDAEYGLYSIYQGGIFELKENDRIFVSV |
| | Blank | | TNEHLIDMDHEASFFGAFLVGGGGGSGGGGSGTSEET |
| | | | ISTVQEKQQNISPLVRERGPQRVAAHITGTRGRSNTLS |
| | | | SPNSKNEKALGRKINSWESSRSGHSFLSNLHLRNGEL |
| | | | VIHEKGFYYIYSQTYFRFQEEIKENTKNDKQMVQYIY |
| | | | KYTSYPDPILLMKSARNSCWSKDAEYGLYSIYQGGIF |
| | | | ELKENDRIFVSVTNEHLIDMDHEASFFGAFLVGGGGG |
| | | | SGGGGSGTSEETISTVQEKQQNISPLVRERGPQRVAA |
| | | | HITGTRGRSNTLSSPNSKNEKALGRKINSWESSRSGHS |
| | | | FLSNLHLRNGELVIHEKGFYYIYSQTYFRFQEEIKENT |
| | | | KNDKQMVQYIYKYTSYPDPILLMKSARNSCWSKDAE |
| | | | YGLYSIYQGGIFELKENDRIFVSVTNEHLIDMDHEASF |
| | | | FGAFLVGGGGGSGGGGSDKTHTCPPCPAPELLGGPSV |
| | | | FLFPPKPKDTLMISRTPEVTCVVVDVSHEDPEVKFNW |
| | | | YVDGVEVHNAKTKPREEQYNSTYRVVSVLTVLHQD |
| | | | WLNGKEYKCKVSNKALPAPIEKTISKAKGQPREPQV |
| | | | YTLPPCREEMTKNQVSLWCLVKGFYPSDIAVEWESN |
| | | | GQPENNYKTTPPVLDSDGSFFLYSKLTVDKSRWQQG |
| | | | NVFSCSVMHEALHNHYTQKSLSLSPGK |
| SEQ ID | a_hDR5_ | Antigen binding | METDTLLLWVLLLWVPGSTGEVQLVESGGGLVQPG |
| NO: 6163 | Tigatuzumab_ | domain specific | GSLRLSCAASGFTFSSYVMSWVRQAPGKGLEWVATI |
| | scFv_ | to DR5, a.k.a. | SSGGSYTYYPDSVKGRFTISRDNAKNTLYLQMNSLRA |
| | VH_VL- | tigatuzumab | EDTAVYYCARRGDSMITTDYWGQGTLVTVSSGGGG |
| | hFc_Knob_ | | SGGGGSGGGGSGGGGSDIQMTQSPSSLSASVGDRVTI |
| | Cys- | | TCKASQDVGTAVAWYQQKPGKAPKLLIYWASTRHT |
| | Blank | | GVPSRFSGSGSGTDFTLTISSLQPEDFATYYCQQYSSY |
| | | | RTFGQGTKVEIKGGGGSGGGGSDKTHTCPPCPAPELL |
| | | | GGPSVFLFPPKPKDTLMISRTPEVTCVVVDVSHEDPE |
| | | | VKFNWYVDGVEVHNAKTKPREEQYNSTYRVVSVLT |
| | | | VLHQDWLNGKEYKCKVSNKALPAPIEKTISKAKGQP |
| | | | REPQVYTLPPCREEMTKNQVSLWCLVKGFYPSDIAVE |
| | | | WESNGQPENNYKTTPPVLDSDGSFFLYSKLTVDKSR |
| | | | WQQGNVFSCSVMHEALHNHYTQKSLSLSPGK |
| SEQ ID | a_hDR5_ | Antigen binding | METDTLLLWVLLLWVPGSTGEVQLVQSGGGVERPG |
| NO: 6164 | Drozitumab_ | domain specific | GSLRLSCAASGFTFDDYAMSWVRQAPGKGLEWVSGI |
| | scFv_ | to DR5, a.k.a. | NWQGGSTGYADSVKGRVTISRDNAKNSLYLQMNSL |
| | VH_VL- | drozitumab | RAEDTAVYYCAKILGAGRGWYFDYWGKGTTVTVSS |
| | hFc_Knob_ | | GGGGSGGGGSGGGGSGGGGSSELTQDPAVSVALGQT |
| | Cys | | VRITCSGDSLRSYYASWYQQKPGQAPVLVIYGANNR |
| | | | PSGIPDRFSGSSSGNTASLTITGAQAEDEADYYCNSAD |
| | | | SSGNHVVFGGGTKLTVLGGGGSGGGGSDKTHTCPPC |
| | | | PAPELLGGPSVFLFPPKPKDTLMISRTPEVTCVVVDVS |
| | | | HEDPEVKFNWYVDGVEVHNAKTKPREEQYNSTYRV |
| | | | VSVLTVLHQDWLNGKEYKCKVSNKALPAPIEKTISK |
| | | | AKGQPREPQVYTLPPCREEMTKNQVSLWCLVKGFYP |
| | | | SDIAVEWESNGQPENNYKTTPPVLDSDGSFFLYSKLT |
| | | | VDKSRWQQGNVFSCSVMHEALHNHYTQKSLSLSPG |
| | | | K |
| SEQ ID | a_hDR5_ | Antigen binding | METDTLLLWVLLLWVPGSTGQVQLQESGPGLVKPSQ |
| NO: 6165 | Conatumumab_ | domain specific | TLSLTCTVSGGSISSGDYFWSWIRQLPGKGLEWIGHIH |
| | scFv_VH_ | to DR5, a.k.a. | NSGTTYYNPSLKSRVTISVDTSKKQFSLRLSSVTAADT |
| | VL- | conatumumab | AVYYCARDRGGDYYYGMDVWGQGTTVTVSSGGGG |
| | hFc_Knob_ | | SGGGGSGGGGSGGGGSEIVLTQSPGTLSLSPGERATLS |
| | Cys | | CRASQGISRSYLAWYQQKPGQAPSLLIYGASSRATGIP |
| | | | DRFSGSGSGTDFTLTISRLEPEDFAVYYCQQFGSSPWT |
| | | | FGQGTKVEIKRGGGGSGGGGSDKTHTCPPCPAPELLG |
| | | | GPSVFLFPPKPKDTLMISRTPEVTCVVVDVSHEDPEV |
| | | | KFNWYVDGVEVHNAKTKPREEQYNSTYRVVSVLTV |
| | | | LHQDWLNGKEYKCKVSNKALPAPIEKTISKAKGQPR |
| | | | EPQVYTLPPCREEMTKNQVSLWCLVKGFYPSDIAVE |
| | | | WESNGQPENNYKTTPPVLDSDGSFFLYSKLTVDKSR |
| | | | WQQGNVFSCSVMHEALHNHYTQKSLSLSPGK |

### T Cell Engagers

The present disclosure provides, inter alia, TCR targeting molecules, that are engineered to further contain one or more T cell engager that mediate binding to and/or activation of a T cell. Accordingly, in some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226.

T cells remain an important arm of immunity against infection and disease. As used herein, the term “effector T cell” includes T helper (e.g., CD4+) cells and cytotoxic (e.g., CD8+) T cells. CD4+ effector T cells contribute to the development of several immunologic processes, including maturation of B cells into plasma cells and memory B cells, and activation of cytotoxic T cells and macrophages. CD8+ effector T cells destroy virus-infected cells, tumor cells, and other pathogenic cells. T cell markers or regions can be exploited to harness T cell functions to target, inhibit, and/or kill target cells in a subject in need thereof. These markers can include, but are not limited to, CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226. Without wishing to be bound by theory, molecules targeting markers for activating or stimulating T cell activity may be used to kill autoreactive T cells in autoimmune diseases or disorders. Described herein are molecules targeting the TCRαV and/or TCRβV chain of an autoreactive T cell and also containing a T cell engager that binds to one or more of a marker on a healthy, e.g. not autoreactive T cell, including, but not limited to, CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226. Without wishing to be bound by any particular theory, activation of the healthy, e.g. not autoreactive T cell, by binding of the agent, antibody molecule, multispecific molecule, multifunctional molecule, and/or TCR targeting molecule described herein may mediate inhibition or killing of the autoreactive T cell.

Disclosed herein are, inter alia, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRβ, e.g., a TCRβV region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226.

Disclosed herein are, inter alia, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRα, e.g., a TCRαV region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226.

Disclosed herein are methods of inhibiting or preventing the activation and/or expansion of autoreactive T cells in a subject comprising administering a multispecific molecule comprising an antigen binding domain that binds to TCRVα and a T cell engager. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226. In some embodiments, the T cell engager binds to (e.g. activates) a CD4+ and/or a CD8+ T cell. In some embodiments, the T cell engager binds to (e.g. activates) a CD4+ T cell. In some embodiments, the T cell engager binds to (e.g. activates) a CD8+ T cell. In some embodiments, the subject has an autoimmune disease.

Disclosed herein are methods of inhibiting or preventing the activation and/or expansion of autoreactive T cells in a subject comprising administering a multispecific molecule comprising an antigen binding domain that binds to TCRVβ and a T cell engager. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, CD154, CD134, KIR, or CD226. In some embodiments, the T cell engager binds to (e.g. activates) a CD4+ and/or a CD8+ T cell. In some embodiments, the T cell engager binds to (e.g. activates) a CD4+ T cell. In some embodiments, the T cell engager binds to (e.g. activates) a CD8+ T cell. In some embodiments, the subject has an autoimmune disease.

Disclosed herein are methods of reducing or depleting autoreactive T cells in a subject comprising administering a multispecific molecule comprising an antigen binding domain that binds to TCRVα and a T cell engager. In some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager binds to (e.g. activates) a CD4+ and/or a CD8+ T cell. In some embodiments, the T cell engager binds to (e.g. activates) a CD4+ T cell. In some embodiments, the T cell engager binds to (e.g. activates) a CD8+ T cell. In some embodiments, the subject has an autoimmune disease.

Disclosed herein are methods of reducing or depleting autoreactive T cells in a subject comprising administering a multispecific molecule comprising an antigen binding domain that binds to TCRVβ and a T cell engager. In some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager binds to (e.g. activates) a CD4+ and/or a CD8+ T cell. In some embodiments, the T cell engager binds to (e.g. activates) a CD4+ T cell. In some embodiments, the T cell engager binds to (e.g. activates) a CD8+ T cell. In some embodiments, the subject has an autoimmune disease.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, TCR targeting molecules that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

Disclosed herein are methods of inhibiting or preventing the activation and/or expansion of autoreactive T cells in a subject comprising administering a multispecific molecule comprising an antigen binding domain that binds to TCRVβ and a B cell engager, a macrophage engager, or a dendritic cell engager. In some embodiments, the subject has an autoimmune disease.

Disclosed herein are methods of reducing or depleting autoreactive T cells in a subject comprising administering a multispecific molecule comprising an antigen binding domain that binds to TCRVα and a B cell engager, a macrophage engager, or a dendritic cell engager. In some embodiments, the subject has an autoimmune disease.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence: QVSHRYPRIQSIKVQFTEYKKEKGFILTSQKEDEIMKVQNNSVIINCDGFYLISLKGYFSQEVNISL HYQKDEEPLFQLKKVRSVNSLMVASLTYKDKVYLNVTTDNTSLDDFHVNGGELILIHQNPGEFC VL (SEQ ID NO: 7245), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7245.

In another embodiment, the CD40L comprises the amino acid sequence: MQKGDQNPQIAAHVISEASSKTTSVLQWAEKGYYTMSNNLVTLENGKQLTVKRQGLYYIYAQV TFCSNREASSQAPFIASLCLKSPGRFERILLRAANTHSSAKPCGQQSIHLGGVFELQPGASVFVNVT DPSQVSHGTGFTSFGLLKL (SEQ ID NO: 7246), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7246.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In one embodiment, the immune cell engager includes 41BB ligand, e.g., comprising the amino acid sequence: ACPWAVSGARASPGSAASPRLREGPELSPDDPAGLLDLRQGMFAQLVAQNVLLIDGPLSWYSDP GLAGVSLTGGLSYKEDTKELVVAKAGVYYVFFQLELRRVVAGEGSGSVSLALHLQPLRSAAGA AALALTVDLPPASSEARNSAFGFQGRLLHLSAGQRLGVHLHTEARARHAWQLTQGATVLGLFR VTPEIPAGLPSPRSE (SEQ ID NO: 7247), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7247.

### Linkers

The TCR targeting molecule disclosed herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In embodiments, the linker is chosen from: a cleavable linker, a non-cleavable linker, a peptide linker, a flexible linker, a rigid linker, a helical linker, or a non-helical linker, or a combination thereof.

In one embodiment, the TCR targeting molecule can include one, two, three or four linkers, e.g., a peptide linker. In one embodiment, the peptide linker includes Gly and Ser. In some embodiments, the peptide linker is selected from GGGGS (SEQ ID NO: 7248); GGGGSGGGGS (SEQ ID NO: 7249); GGGGSGGGGSGGGGS (SEQ ID NO: 7250); and DVPSGPGGGGGSGGGGS (SEQ ID NO: 7251). In some embodiments, the peptide linker is a A(EAAAK)nA (SEQ ID NO: 7291) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 7252); AEAAAKEAAAKEAAAKAAA (SEQ ID NO: 7253); AEAAAKEAAAKEAAAKEAAAKAAA (SEQ ID NO: 77); and AEAAAKEAAAKEAAAKEAAAKEAAAKAAA(SEQ ID NO: 78).

### Nucleic Acids

Nucleic acids encoding the aforementioned TCR targeting molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding a TCR targeting molecule described herein. In one embodiment, the vectors comprise nucleotides encoding a TCR targeting molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Exemplary Multifunctional Polypeptide Molecule

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3523, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 3523, the sequence of SEQ ID NO: 2170, and the sequence of SEQ ID NO: 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3523 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 3523 operatively linked to the sequence of SEQ ID NO: 2170 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3523 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3524, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 3523 is operatively linked to the sequence of SEQ ID NO: 2170 via the sequence of SEQ ID NO: 3524, the sequence of SEQ ID NO: 2170 is operatively linked to the sequence of SEQ ID NO: 3648 via the sequence of SEQ ID NO: 3308, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3519; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising the sequence of SEQ ID NO: 3517; (ii) a second polypeptide comprising the sequence of SEQ ID NO: 3519; and (iii) a third polypeptide comprising to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 2170, and the sequence of SEQ ID NO: 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 2170 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2170 is operatively linked to the sequence of SEQ ID NO: 3648 via the sequence of SEQ ID NO: 3308, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3520; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising the sequence of SEQ ID NO: 3517; (ii) a second polypeptide comprising the sequence of SEQ ID NO: 3520; and (iii) a third polypeptide comprising to the sequence of SEQ ID NO: 3518.

In some embodiments, the IL-2 molecule or a functional fragment or a functional variant thereof or the IL-2 C125A mutant molecule or a functional fragment or a functional variant thereof is operatively linked to the immunoglobulin heavy chain constant region via a linker.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the IL-2 molecule or a functional fragment or a functional variant thereof or the IL-2 C125A mutant molecule or a functional fragment or a functional variant thereof is operatively linked to the immunoglobulin heavy chain constant region via a linker.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 2270, and the sequence of SEQ ID NO: 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 2270 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2270 is operatively linked to the sequence of SEQ ID NO: 3648 via the sequence of SEQ ID NO: 3308, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3521 or 7515; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising the sequence of SEQ ID NO: 3517; (ii) a second polypeptide comprising the sequence of SEQ ID NO: 3521 or 7515; and (iii) a third polypeptide comprising to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528.

In some embodiments, the multifunctional polypeptide molecule comprises: (i) a first polypeptide comprising the sequence of SEQ ID NO: 3530, and the sequence of SEQ ID NO: 3531; (ii) a second polypeptide comprising the sequence of SEQ ID NO: 2191, and the sequence of SEQ ID NO: 3533; and (iii) a third polypeptide comprising the sequence of SEQ ID NO: 3527, and the sequence of SEQ ID NO: 3528.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528.

In some embodiments, the multifunctional polypeptide molecule comprises: (i) a first polypeptide comprising, from the N-terminus to the C-terminus, the sequence of SEQ ID NO: 3530 operatively linked to the sequence of SEQ ID NO: 3531; (ii) a second polypeptide comprising, from the N-terminus to the C-terminus, the sequence of SEQ ID NO: 2191 operatively linked to the sequence of SEQ ID NO: 3533; and (iii) a third polypeptide comprising, from the N-terminus to the C-terminus, the sequence of SEQ ID NO: 3527 operatively linked to the sequence of SEQ ID NO: 3528.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 2191 is operatively linked to the sequence of SEQ ID NO: 3533 via the sequence of SEQ ID NO: 3308, or a combination thereof.

In some embodiments, the first polypeptide further comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3547 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3531, the second polypeptide further comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3534 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3533, or a combination thereof.

In some embodiments, the first polypeptide further comprises the sequence of SEQ ID NO: 3547 operatively linked to the sequence of SEQ ID NO: 3531, the second polypeptide further comprises the sequence of SEQ ID NO: 3534 operatively linked to the sequence of SEQ ID NO: 3533, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3529 or the sequence of SEQ ID NO: 3548; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3532 or the sequence of SEQ ID NO: 3549; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3526.

In some embodiments, the multifunctional polypeptide molecule comprises: (i) a first polypeptide comprising the sequence of SEQ ID NO: 3529 or the sequence of SEQ ID NO: 3548; (ii) a second polypeptide comprising the sequence of SEQ ID NO: 3532 or the sequence of SEQ ID NO: 3549; and (iii) a third polypeptide comprising to the sequence of SEQ ID NO: 3526.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 3540, and the sequence of SEQ ID NO: 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 3540 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 3540 is operatively linked to the sequence of SEQ ID NO: 3648 via the sequence of SEQ ID NO: 3308, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3539; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising the sequence of SEQ ID NO: 3517; (ii) a second polypeptide comprising the sequence of SEQ ID NO: 3539; and (iii) a third polypeptide comprising to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3542, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 3542, and the sequence of SEQ ID NO: 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3542 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 3542 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, the IL-12 molecule or a functional fragment or a functional variant thereof comprises a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3543 and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3545.

In some embodiments, the IL-12 molecule or a functional fragment or a functional variant thereof comprises the sequence of SEQ ID NO: 3543 and the sequence of SEQ ID NO: 3545.

In some embodiments, the IL-12 molecule or a functional fragment or a functional variant thereof comprises, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3543 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO:3545.

In some embodiments, the IL-12 molecule or a functional fragment or a functional variant thereof comprises, from the N-terminus to the C-terminus, the sequence of SEQ ID NO: 3543 operatively linked to the sequence of SEQ ID NO:3545.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3543 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3545 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3544, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3545 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, or a combination thereof.

In some embodiments, the sequence of SEQ ID NO: 3543 is operatively linked to the sequence of SEQ ID NO: 3545 via the sequence of SEQ ID NO: 3544, the sequence of SEQ ID NO: 3545 is operatively linked to the sequence of SEQ ID NO: 3648 via the sequence of SEQ ID NO: 3308, or a combination thereof.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3541; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising the sequence of SEQ ID NO: 3517; (ii) a second polypeptide comprising the sequence of SEQ ID NO: 3541; and (iii) a third polypeptide comprising to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3540, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising the sequence of SEQ ID NO: 1346, and the sequence of SEQ ID NO: 3649; (ii) a second polypeptide comprising the sequence of SEQ ID NO: 2193, and the sequence of SEQ ID NO: 3648; and (iii) a third polypeptide comprising the sequence of SEQ ID NO: 1349, and the sequence of SEQ ID NO: 3644.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649; (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2193 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648; and (iii) a third polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide comprising, from the N-terminus to the C-terminus, the sequence of SEQ ID NO: 1346 operatively linked to the sequence of SEQ ID NO: 3649; (ii) a second polypeptide comprising, from the N-terminus to the C-terminus, the sequence of SEQ ID NO: 2193 operatively linked to the sequence of SEQ ID NO: 3648; and (iii) a third polypeptide comprising, from the N-terminus to the C-terminus, the sequence of SEQ ID NO: 1349 operatively linked to the sequence of SEQ ID NO: 3644.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2193 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308.

In some embodiments, the sequence of SEQ ID NO: 2193 is operatively linked to the sequence of SEQ ID NO: 3648 via the sequence of SEQ ID NO: 3308.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3517; (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3546; and (iii) a third polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3518.

In some embodiments, the multifunctional polypeptide molecule comprises: (i) a first polypeptide comprising the sequence of SEQ ID NO: 3517; (ii) a second polypeptide comprising the sequence of SEQ ID NO: 3546; and (iii) a third polypeptide comprising to the sequence of SEQ ID NO: 3518.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3537; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528, and a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191.

In some embodiments, the multifunctional polypeptide molecule comprises: (i) a first polypeptide comprising the sequence of SEQ ID NO: 3530 and the sequence of SEQ ID NO: 3537; and (ii) a second polypeptide comprising the sequence of SEQ ID NO: 3527, the sequence of SEQ ID NO: 3528, and the sequence of SEQ ID NO: 2191.

In some embodiments, the multifunctional polypeptide molecule comprises: (i) a first polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3530 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3537; and (ii) a second polypeptide comprising, from the N-terminus to the C-terminus, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3527 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528 operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191.

In some embodiments, the multifunctional polypeptide molecule comprises: (i) a first polypeptide comprising, from the N-terminus to the C-terminus, the sequence of SEQ ID NO: 3530 operatively linked to the sequence of SEQ ID NO: 3537; and (ii) a second polypeptide comprising, from the N-terminus to the C-terminus, the sequence of SEQ ID NO: 3527 operatively linked to the sequence of SEQ ID NO: 3528 operatively linked to the sequence of SEQ ID NO: 2191.

In some embodiments, a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3528 is operatively linked to a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2191 via a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3309.

In some embodiments, the sequence of SEQ ID NO: 3528 is operatively linked to the sequence of SEQ ID NO: 2191 via the sequence of SEQ ID NO: 3309.

In some embodiments, the multifunctional polypeptide molecule comprises two first polypeptides and two second polypeptides.

In some embodiments, the multifunctional polypeptide molecule comprises: (i) a first polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3536; and (ii) a second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3535.

In some embodiments, the multifunctional polypeptide molecule comprises: (i) a first polypeptide comprising the sequence of SEQ ID NO: 3536; and (ii) a second polypeptide comprising the sequence of SEQ ID NO: 3535.

In some embodiments, the multifunctional polypeptide molecule comprises: (i) a first polypeptide comprising one or more components as listed in Table 15; and (ii) a second polypeptide comprising one or more components as listed in Table 15. In some embodiments, the multifunctional polypeptide molecule further comprises: (i) a third polypeptide comprising one or more components as listed in Table 15; and (ii) a fourth polypeptide comprising one or more components as listed in Table 15.

In some embodiments, the multifunctional polypeptide molecule comprises: (i) a first polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 15; and (ii) a second polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 15. In some embodiments, the multifunctional polypeptide molecule further comprises: (i) a third polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 15; and (ii) a fourth polypeptide comprising one or more components comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the component sequences as listed in Table 15.

In some embodiments, the multifunctional polypeptide molecule comprises: (i) a first polypeptide comprising one or more component sequences as listed in Table 15; and (ii) a second polypeptide comprising one or more component sequences as listed in Table 15. In some embodiments, the multifunctional polypeptide molecule further comprises: (i) a third polypeptide comprising one or more component sequences as listed in Table 15; and (ii) a fourth polypeptide comprising one or more component sequences as listed in Table 15.

In some embodiments, the multifunctional polypeptide molecule comprises: (i) a first polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 15; and (ii) a second polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 15. In some embodiments, the multifunctional polypeptide molecule further comprises: (i) a third polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 15; and (ii) a fourth polypeptide comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to any one of the polypeptide sequences as listed in Table 15.

In some embodiments, the multifunctional polypeptide molecule comprises: (i) a first polypeptide comprising any one of the polypeptide sequences as listed in Table 15; and (ii) a second polypeptide comprising any one of the polypeptide sequences as listed in Table 15. In some embodiments, the multifunctional polypeptide molecule further comprises: (i) a third polypeptide comprising any one of the polypeptide sequences as listed in Table 15; and (ii) a fourth polypeptide comprising one or more comprising any one of the polypeptide sequences as listed in Table 15.

In another aspect, described herein is an antibody comprising an anti-T cell receptor beta variable chain (TCRβV) binding domain comprising: (i) a heavy chain variable region (VH) comprising a heavy chain complementarity determining region 1 (HC CDR1), a heavy chain complementarity determining region 2 (HC CDR2), and a heavy chain complementarity determining region 3 (HC CDR3) comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 3650, SEQ ID NO: 3651, and SEQ ID NO: 5, respectively; (ii) a light chain variable region (VL) comprising a light chain complementarity determining region 1 (LC CDR1), a light chain complementarity determining region 2 (LC CDR2), and a light chain complementarity determining region 3 (LC CDR3) comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 3655, SEQ ID NO: 3653, and SEQ ID NO: 8, respectively; or (iii) a combination thereof.

In some embodiments, the TCRβV binding domain comprising: (i) a VH comprising a HC CDR1, a HC CDR2, and a HC CDR3 comprising the amino acid sequence of SEQ ID NO: 3650, SEQ ID NO: 3651, and SEQ ID NO: 5, respectively; (ii) a VL comprising a LC CDR1, a LC CDR2, and a LC CDR3 comprising the amino acid sequence of SEQ ID NO: 3655, SEQ ID NO: 3653, and SEQ ID NO: 8, respectively; or (iii) a combination thereof.

In some embodiments, the TCRβV binding domain comprising: (i) a VH comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 1346; (ii) a VL comprising an amino acid sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to SEQ ID NO: 1349; or (iii) a combination thereof.

In some embodiments, the TCRβV binding domain comprising: (i) a VH comprising the amino acid sequence of SEQ ID NO: 1346; (ii) a VL comprising the amino acid sequence of SEQ ID NO: 1349; or (iii) a combination thereof.

In some embodiments, the antibody comprising an anti-T cell receptor beta variable chain (TCRβV) binding domain further comprises a cytokine polypeptide or a functional fragment or a functional variant thereof. In some embodiments, the cytokine polypeptide may be IL-2, IL2-C125A. IL-15, IL-7, IL-12, or IL-21. In embodiments, the cytokine polypeptide may further comprise a cytokine receptor. In some embodiments, the cytokine polypeptide may comprise IL-15 linked to a IL-15Ra. In some embodiments, the cytokine polypeptide may comprise IL-15 linked to a IL-15Ra sushi domain. In some embodiments, the cytokine polypeptide may comprise a cytokine dimer. In some embodiments, the cytokine polypeptide may comprise an IL-12 beta subunit linked to an IL-12 alpha subunit.

TABLE 1
Amino acid and nucleotide sequences for murine, chimeric and humanized antibody
molecules which bind to TCRVB 6, e.g., TCRVB 6-5. The antibody molecules include murine
mAb Antibody A, and humanized mAb Antibody A-H Clones A-H.1 to A-H.85. The amino acid
the heavy and light chain CDRs, and the amino acid and nucleotide sequences of the heavy
and light chain variable regions, and the heavy and light chains are shown.
Antibody A (murine), also referred to as H131, TCRVB 6-5 binder

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: | HC CDR1 (Kabat) | TYYIH |
| 45 | | |
| SEQ ID NO: | HC CDR2 (Kabat) | WFFPGSGNIKYNEKFKG |
| 46 | | |
| SEQ ID NO: | HC CDR3 (Kabat) | SYYSYDVLDY |
| 47 | | |
| SEQ ID | HC CDR1 (Chothia) | GYSFTTY |
| NO: 48 | | |
| SEQ ID NO: | HC CDR2 (Chothia) | FPGSGN |
| 49 | | |
| SEQ ID NO: | HC CDR3 (Chothia) | SYYSYDVLDY |
| 50 | | |
| SEQ ID NO: 1 | VH | QVQLQQSGPELVKPGTSVKISCKASGYSFTTYYIHWVK |
| | | QRPGQGLEWIGWFFPGSGNIKYNEKFKGKATLTADTSS |
| | | STAYMQLSSLTSEESAVYFCAGSYYSYDVLDYWGHGT |
| | | TLTVSS |
| SEQ ID NO: 6 | LC CDR1 | KASQNVGINVV |
| | (Combined) | |
| SEQ ID NO: 7 | LC CDR2 | SSSHRYS |
| | (Combined) | |
| SEQ ID NO: 8 | LC CDR3 | QQFKSYPLT |
| | (Combined) | |
| SEQ ID NO: | LC CDR1 (Kabat) | KASQNVGINVV |
| 51 | | |
| SEQ ID NO: | LC CDR2 (Kabat) | SSSHRYS |
| 52 | | |
| SEQ ID NO: | LC CDR3 (Kabat) | QQFKSYPLT |
| 53 | | |
| SEQ ID NO: | LC CDR1 (Chothia) | KASQNVGINVV |
| 54 | | |
| SEQ ID NO: | LC CDR2 (Chothia) | SSSHRYS |
| 55 | | |
| SEQ ID NO: | LC CDR3 (Chothia) | QQFKSYPLT |
| 56 | | |
| SEQ ID NO: 2 | VL | DILMTQSQKFMSTSLGDRVSVSCKASQNVGINVVWHQ |
| | | QKPGQSPKALIYSSSHRYSGVPDRFTGSGSGTDFTLTIN |
| | | NVQSEDLAEYFCQQFKSYPLTFGAGTKLELK |

Antibody A humanized (A-H antibody), TCRVB 6-5 binder
A-H.1 antibody (also referred to as BHM1709)

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: 9 | VH | QVQLVQSGAEVKKPGSSVKVSCKASGYSFTTYYIHWV |
| | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |
| SEQ ID NO: | DNA VH | CAGGTGCAGCTGGTTCAGTCTGGCGCCGAAGTGAAG |
| 12 | | AAACCTGGCTCCTCCGTGAAGGTGTCCTGCAAGGCTT |
| | | CCGGCTACTCCTTCACCACCTACTACATCCACTGGGT |
| | | CCGACAGGCCCCTGGACAAGGATTGGAATGGATGGG |
| | | CTGGTTCTTCCCCGGCTCCGGCAACATCAAGTACAAC |
| | | GAGAAGTTCAAGGGCCGCGTGACCATCACCGCCGAC |
| | | ACCTCTACCTCTACCGCCTACATGGAACTGTCCAGCC |
| | | TGAGATCTGAGGACACCGCCGTGTACTACTGCGCCG |
| | | GCTCCTACTACTCTTACGACGTGCTGGATTACTGGGG |
| | | CCAGGGCACCACAGTGACAGTGTCCTCT |
| SEQ ID NO: | VH-IgM constant | METDTLLLWVLLLWVPGSTGQVQLVQSGAEVKKPGSS |
| 69 | delta CDC | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSGSASAPTLFPL |
| | | VSCENSPSDTSSVAVGCLAQDFLPDSITFSWKYKNNSDI |
| | | SSTRGFPSVLRGGKYAATSQVLLPSKDVMQGTDEHVV |
| | | CKVQHPNGNKEKNVPLPVIAELPPKVSVFVPPRDGFFG |
| | | NPRKSKLICQATGFSPRQIQVSWLREGKQVGSGVTTDQ |
| | | VQAEAKESGPTTYKVTSTLTIKESDWLGQSMFTCRVDH |
| | | RGLTFQQNASSMCVPDQDTAIRVFAIPPSFASIFLTKSTK |
| | | LTCLVTDLTTYDSVTISWTRQNGEAVKTHTNISESHPNA |
| | | TFSAVGEASICEDDWNSGERFTCTVTHTDLASSLKQTIS |
| | | RPKGVALHRPDVYLLPPAREQLNLRESATITCLVTGFSP |
| | | ADVFVQWMQRGQPLSPEKYVTSAPMPEPQAPGRYFAH |
| | | SILTVSEEEWNTGETYTCVVAHEALPNRVTERTVDKST |
| | | GKPTLYNVSLVMSDTAGTCY |
| SEQ ID NO: | VH-IgGA1 | METDTLLLWVLLLWVPGSTGQVQLVQSGAEVKKPGSS |
| 70 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASPTSPKVFPLS |
| | | LCSTQPDGNVVIACLVQGFFPQEPLSVTWSESGQGVTA |
| | | RNFPPSQDASGDLYTTSSQLTLPATQCLAGKSVTCHVK |
| | | HYTNPSQDVTVPCPVPSTPPTPSPSTPPTPSPSCCHPRLSL |
| | | HRPALEDLLLGSEANLTCTLTGLRDASGVTFTWTPSSG |
| | | KSAVQGPPERDLCGCYSVSSVLPGCAEPWNHGKTFTCT |
| | | AAYPESKTPLTATLSKSGNTFRPEVHLLPPPSEELALNEL |
| | | VTLTCLARGFSPKDVLVRWLQGSQELPREKYLTWASR |
| | | QEPSQGTTTFAVTSILRVAAEDWKKGDTFSCMVGHEAL |
| | | PLAFTQKTIDRLAGKPTHVNVSVVMAEVDGTCY |
| SEQ ID NO: | VH-IgGA2 | METDTLLLWVLLLWVPGSTGQVQLVQSGAEVKKPGSS |
| 71 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASPTSPKVFPLS |
| | | LDSTPQDGNVVVACLVQGFFPQEPLSVTWSESGQNVTA |
| | | RNFPPSQDASGDLYTTSSQLTLPATQCPDGKSVTCHVK |
| | | HYTNSSQDVTVPCRVPPPPPCCHPRLSLHRPALEDLLLG |
| | | SEANLTCTLTGLRDASGATFTWTPSSGKSAVQGPPERD |
| | | LCGCYSVSSVLPGCAQPWNHGETFTCTAAHPELKTPLT |
| | | ANITKSGNTFRPEVHLLPPPSEELALNELVTLTCLARGFS |
| | | PKDVLVRWLQGSQELPREKYLTWASRQEPSQGTTTYA |
| | | VTSILRVAAEDWKKGETFSCMVGHEALPLAFTQKTIDR |
| | | MAGKPTHINVSVVMAEADGTCY |
| SEQ ID NO: | Heavy chain | METDTLLLWVLLLWVPGSTGQVQLVQSGAEVKKPGSS |
| 3278 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASTKGPSVFPL |
| | | APSSKSTSGGTAALGCLVKDYFPEPVTVSWNSGALTSG |
| | | VHTFPAVLQSSGLYSLSSVVTVPSSSLGTQTYICNVNHK |
| | | PSNTKVDKRVEPKSCDKTHTCPPCPAPELLGGPSVFLFP |
| | | PKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVYTLPPSREE |
| | | MTKNQVSLTCLVKGFYPSDIAVEWESNGQPENNYKTTP |
| | | PVLDSDGSFFLYSKLTVDKSRWQQGNVFSCSVMHEAL |
| | | HNHYTQKSLSLSPGK |
| SEQ ID NO: 6 | LC CDR1 | KASQNVGINVV |
| | (Combined) | |
| SEQ ID NO: 7 | LC CDR2 | SSSHRYS |
| | (Combined) | |
| SEQ ID NO: 8 | LC CDR3 | QQFKSYPLT |
| | (Combined) | |
| SEQ ID NO: | VL | DIQMTQSPSFLSASVGDRVTITCKASQNVGINVVWHQQ |
| 10 | | KPGKAPKALIYSSSHRYSGVPSRFSGSGSGTEFTLTISSL |
| | | QPEDFATYFCQQFKSYPLTFGQGTKLEIK |
| SEQ ID NO: | DNA VL | GACATCCAGATGACCCAGTCTCCATCCTTCCTGTCCG |
| 13 | | CCTCTGTGGGCGACAGAGTGACCATCACATGCAAGG |
| | | CCTCTCAGAACGTGGGCATCAACGTCGTGTGGCACCA |
| | | GCAGAAGCCTGGCAAGGCTCCTAAGGCTCTGATCTA |
| | | CTCCTCCAGCCACCGGTACTCTGGCGTGCCCTCTAGA |
| | | TTTTCCGGCTCTGGCTCTGGCACCGAGTTTACCCTGA |
| | | CAATCTCCAGCCTGCAGCCTGAGGACTTCGCCACCTA |
| | | CTTTTGCCAGCAGTTCAAGAGCTACCCTCTGACCTTT |
| | | GGCCAGGGCACCAAGCTGGAAATCAAG |
| SEQ ID NO: | VL and kappa | METDTLLLWVLLLWVPGSTGDIQMTQSPSFLSASVGDR |
| 72 | constant region/light | VTITCKASQNVGINVVWHQQKPGKAPKALIYSSSHRYS |
| | chain | GVPSRFSGSGSGTEFTLTISSLQPEDFATYFCQQFKSYPL |
| | | TFGQGTKLEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFNR |
| | | GEC |

A-H.2 antibody (also referred to as BHM1710)

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: 9 | VH | QVQLVQSGAEVKKPGSSVKVSCKASGYSFTTYYIHWV |
| | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |
| SEQ ID NO: | DNA VH | CAGGTGCAGCTGGTTCAGTCTGGCGCCGAAGTGAAG |
| 12 | | AAACCTGGCTCCTCCGTGAAGGTGTCCTGCAAGGCTT |
| | | CCGGCTACTCCTTCACCACCTACTACATCCACTGGGT |
| | | CCGACAGGCCCCTGGACAAGGATTGGAATGGATGGG |
| | | CTGGTTCTTCCCCGGCTCCGGCAACATCAAGTACAAC |
| | | GAGAAGTTCAAGGGCCGCGTGACCATCACCGCCGAC |
| | | ACCTCTACCTCTACCGCCTACATGGAACTGTCCAGCC |
| | | TGAGATCTGAGGACACCGCCGTGTACTACTGCGCCG |
| | | GCTCCTACTACTCTTACGACGTGCTGGATTACTGGGG |
| | | CCAGGGCACCACAGTGACAGTGTCCTCT |
| SEQ ID NO: | Heavy chain | METDTLLLWVLLLWVPGSTGQVQLVQSGAEVKKPGSS |
| 3278 | | VKVSCKASGYSFTTYYIHWVRQAPGQGLEWMGWFFPG |
| | | SGNIKYNEKFKGRVTITADTSTSTAYMELSSLRSEDTAV |
| | | YYCAGSYYSYDVLDYWGQGTTVTVSSASTKGPSVFPL |
| | | APSSKSTSGGTAALGCLVKDYFPEPVTVSWNSGALTSG |
| | | VHTFPAVLQSSGLYSLSSVVTVPSSSLGTQTYICNVNHK |
| | | PSNTKVDKRVEPKSCDKTHTCPPCPAPELLGGPSVFLFP |
| | | PKPKDTLMISRTPEVTCVVVDVSHEDPEVKFNWYVDG |
| | | VEVHNAKTKPREEQYNSTYRVVSVLTVLHQDWLNGKE |
| | | YKCKVSNKALPAPIEKTISKAKGQPREPQVYTLPPSREE |
| | | MTKNQVSLTCLVKGFYPSDIAVEWESNGQPENNYKTTP |
| | | PVLDSDGSFFLYSKLTVDKSRWQQGNVFSCSVMHEAL |
| | | HNHYTQKSLSLSPGK |
| SEQ ID NO: 6 | LC CDR1 | KASQNVGINVV |
| | (Combined) | |
| SEQ ID NO: 7 | LC CDR2 | SSSHRYS |
| | (Combined) | |
| SEQ ID NO: 8 | LC CDR3 | QQFKSYPLT |
| | (Combined) | |
| SEQ ID NO: | VL | DIQMTQSPSSLSASVGDRVTITCKASQNVGINVVWHQQ |
| 11 | | KPGKVPKALIYSSSHRYSGVPSRFSGSGSGTDFTLTISSL |
| | | QPEDVATYFCQQFKSYPLTFGQGTKLEIK |
| SEQ ID NO: | DNA VL | GACATCCAGATGACCCAGTCTCCATCCTCTCTGTCCG |
| 14 | | CCTCTGTGGGCGACAGAGTGACCATCACATGCAAGG |
| | | CCTCTCAGAACGTGGGCATCAACGTCGTGTGGCACCA |
| | | GCAGAAACCTGGCAAGGTGCCCAAGGCTCTGATCTA |
| | | CTCCTCCAGCCACAGATACTCCGGCGTGCCCTCTAGA |
| | | TTCTCCGGCTCTGGCTCTGGCACCGACTTTACCCTGA |
| | | CAATCTCCAGCCTGCAGCCTGAGGACGTGGCCACCTA |
| | | CTTTTGCCAGCAGTTCAAGAGCTACCCTCTGACCTTT |
| | | GGCCAGGGCACCAAGCTGGAAATCAAG |
| SEQ ID NO: | Light chain | METDTLLLWVLLLWVPGSTGDIQMTQSPSSLSASVGDR |
| 3279 | | VTITCKASQNVGINVVWHQQKPGKVPKALIYSSSHRYS |
| | | GVPSRFSGSGSGTDFTLTISSLQPEDVATYFCQQFKSYPL |
| | | TFGQGTKLEIKRTVAAPSVFIFPPSDEQLKSGTASVVCLL |
| | | NNFYPREAKVQWKVDNALQSGNSQESVTEQDSKDSTY |
| | | SLSSTLTLSKADYEKHKVYACEVTHQGLSSPVTKSFNR |
| | | GEC |

A-H.3 antibody

| | | |
|---|---|---|
| SEQ ID NO: | VH + VL | QVQLVQSGAEVKKPGSSVKVSCKASGTDFKLTYIHWV |
| 80 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 81 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 82 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSS |

A-H.4

| | | |
|---|---|---|
| SEQ ID NO: | VH + VL | QVQLVQSGAEVKKPGSSVKVSCKASGTDFDKIYIHWVR |
| 83 | | QAPGQGLEWMGRISAGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVEDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 84 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 85 | | QAPGQGLEWMGRISAGSGNVKYNEKFKGRVTITADTS |
| | | TSTAYMELSSLRSEDTAVYYCAGSYYSYDVLDYWGQG |
| | | TTVTVSS |

A-H.5

| | | |
|---|---|---|
| SEQ ID NO: | VH + VL | QVQLVQSGAEVKKPGSSVKVSCKASGHDFRDFYIHWV |
| 86 | | RQAPGQGLEWMGRVYPGSGSYRYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 87 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFRDFYIHWV |
| 88 | | RQAPGQGLEWMGRVYPGSGSYRYNEKFKGRVTITADT |
| | | STSTAYMELSSLRSEDTAVYYCAGSYYSYDVLDYWGQ |
| | | GTTVTVSS |

A-H.6

| | | |
|---|---|---|
| SEQ ID NO: | VH + VL | QVQLVQSGAEVKKPGSSVKVSCKASGHDFKLTYIHWV |
| 89 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWYQQ |
| 90 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFKLTYIHWV |
| 91 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.7

| | | |
|---|---|---|
| SEQ ID NO: | VH + VL | QVQLVQSGAEVKKPGSSVKVSCKASGTDFKLTYIHWV |
| 92 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVENKVAWHQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVENKVAWHQQ |
| 93 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 94 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |

A-H.8

| | | |
|---|---|---|
| SEQ ID NO: | VH + VL | QVQLVQSGAEVKKPGSSVKVSCKASGTDFDKIYIHWVR |
| 95 | | QAPGQGLEWMGRIFAGSGNTKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVDDRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVDDRVAWYQQ |
| 96 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 97 | | QAPGQGLEWMGRIFAGSGNTKYNEKFKGRVTITADTST |
| | | STAYMELSSLRSEDTAVYYCAGSYYSYDVLDYWGQGT |
| | | TVTVSS |

A-H.9

| | | |
|---|---|---|
| SEQ ID NO: | VH + VL | QVQLVQSGAEVKKPGSSVKVSCKASGHDFDKFYIHWV |
| 98 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGNRVAWYQQKPGKAPKAL |
| | | IYSSSHRYSGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 99 | | KPGKAPKALIYSSSHRYSGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 100 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.10

| | | |
|---|---|---|
| SEQ ID NO: | VH + VL | QVQLVQSGAEVKKPGSSVKVSCKASGHDFDKFYIHWV |
| 101 | | RQAPGQGLEWMGRIFAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVAWYQQKPGKAPKAL |
| | | IYSSSHRYKGVPSRFSGSGSGTEFTLTISSLQPEDFATYFC |
| | | QQFKSYPLTFGQGTKLEIKs |
| SEQ ID NO: | VL | DIQMTQSPSFLSASVGDRVTITCKASQNVGDRVAWYQQ |
| 102 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 103 | | RQAPGQGLEWMGRIFAGSGNVKYNEKFKGRVTITADT |
| | | STSTAYMELSSLRSEDTAVYYCAGSYYSYDVLDYWGQ |
| | | GTTVTVSS |

A-H.11

| | | |
|---|---|---|
| SEQ ID NO: | VH + VL | QVQLVQSGAEVKKPGSSVKVSCKASGTDFKLTYIHWV |
| 104 | | RQAPGQGLEWMGRVSPGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 105 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 106 | | RQAPGQGLEWMGRVSPGSGNVKYNEKFKGRVTITADT |
| | | STSTAYMELSSLRSEDTAVYYCAGSYYSYDVLDYWGQ |
| | | GTTVTVSS |

A-H.12

| | | |
|---|---|---|
| SEQ ID NO: | VH + VL | QVQLVQSGAEVKKPGSSVKVSCKASGTDFDKIYIHWVR |
| 107 | | QAPGQGLEWMGRVSAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGNRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 108 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 109 | | QAPGQGLEWMGRVSAGSGNTKYNEKFKGRVTITADTS |
| | | TSTAYMELSSLRSEDTAVYYCAGSYYSYDVLDYWGQG |
| | | TTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWV |
| 110 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDNRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWYQQ |
| 111 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 112 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |

A-H.14

| | | |
|---|---|---|
| SEQ ID NO: | VH + VL | QVQLVQSGAEVKKPGSSVKVSCKASGTDFDKIYIHWVR |
| 113 | | QAPGQGLEWMGRISAGSGNTKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVDDRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVDDRVAWYQQ |
| 114 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 115 | | QAPGQGLEWMGRISAGSGNTKYNEKFKGRVTITADTST |
| | | STAYMELSSLRSEDTAVYYCAGSYYSYDVLDYWGQGT |
| | | TVTVSS |

A-H.15

| | | |
|---|---|---|
| SEQ ID NO: | VH + VL | QVQLVQSGAEVKKPGSSVKVSCKASGTDFRLTYIHWV |
| 116 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNKVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNKVAWHQ |
| 117 | | QKPGKAPKALIYSSSHRYKGVPSRFSGSGSGTEFTLTISS |
| | | LQPEDFATYFCQQFKSYPLTFGQGTKLEIK |
| SEQ ID NO: | VH | QVQLVQSGAEVKKPGSSVKVSCKASGTDFRLTYIHWV |
| 118 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSS |

A-H.16

| | | |
|---|---|---|
| SEQ ID NO: | VH + VL | QVQLVQSGAEVKKPGSSVKVSCKASGGTFRLTYIHWV |
| 119 | | RQAPGQGLEWMGRVYPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 120 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFRLTYIHWV |
| 121 | | RQAPGQGLEWMGRVYPGSGNTKYNEKFKGRVTITADT |
| | | STSTAYMELSSLRSEDTAVYYCAGSYYSYDVLDYWGQ |
| | | GTTVTVSS |

A-H.17

| | | |
|---|---|---|
| SEQ ID NO: | VH + VL | QVQLVQSGAEVKKPGSSVKVSCKASGTDFRLTYIHWV |
| 122 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 123 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFRLTYIHWV |
| 124 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSS |

A-H.18

| | | |
|---|---|---|
| SEQ ID NO: | VH + VL | QVQLVQSGAEVKKPGSSVKVSCKASGTDFKLTYIHWV |
| 125 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVEDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 126 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 127 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |

A-H.19

| | | |
|---|---|---|
| SEQ ID NO: | VH + VL | QVQLVQSGAEVKKPGSSVKVSCKASGGTFRLTYIHWV |
| 128 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 129 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFRLTYIHWV |
| 130 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.20

| | | |
|---|---|---|
| SEQ ID NO: | VH + VL | QVQLVQSGAEVKKPGSSVKVSCKASGGTFDKTYIHWV |
| 131 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 132 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFDKTYIHWV |
| 133 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSS |

A-H.21

| | | |
|---|---|---|
| SEQ ID NO: | VH + VL | QVQLVQSGAEVKKPGSSVKVSCKASGHDFDKFYIHWV |
| 134 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 135 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 136 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSS |

A-H.22

| | | |
|---|---|---|
| SEQ ID NO: | VH + VL | QVQLVQSGAEVKKPGSSVKVSCKASGTDFKLTYIHWV |
| 137 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNKVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNKVAWHQ |
| 138 | | QKPGKAPKALIYSSSHRYKGVPSRFSGSGSGTEFTLTISS |
| | | LQPEDFATYFCQQFKSYPLTFGQGTKLEIK |
| SEQ ID NO: | VH | QVQLVQSGAEVKKPGSSVKVSCKASGTDFKLTYIHWV |
| 139 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.23

| | | |
|---|---|---|
| SEQ ID NO: | VH + VL | QVQLVQSGAEVKKPGSSVKVSCKASGHDFRLTYIHWV |
| 140 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVADRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVADRVAWYQQ |
| 141 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFRLTYIHWV |
| 142 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.24

| | | |
|---|---|---|
| SEQ ID NO: | VH + VL | QVQLVQSGAEVKKPGSSVKVSCKASGHDFHLWYIHWV |
| 143 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNKVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNKVAWHQ |
| 144 | | QKPGKAPKALIYSSSHRYKGVPSRFSGSGSGTEFTLTISS |
| | | LQPEDFATYFCQQFKSYPLTFGQGTKLEIK |
| SEQ ID NO: | VH | QVQLVQSGAEVKKPGSSVKVSCKASGHDFHLWYIHWV |
| 145 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.25

| | | |
|---|---|---|
| SEQ ID NO: | VH + VL | QVQLVQSGAEVKKPGSSVKVSCKASGHDFHLWYIHWV |
| 146 | | RQAPGQGLEWMGRVFAGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDKVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDKVAWYQQ |
| 147 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFHLWYIHWV |
| 148 | | RQAPGQGLEWMGRVFAGSGNTKYNEKFKGRVTITADT |
| | | STSTAYMELSSLRSEDTAVYYCAGSYYSYDVLDYWGQ |
| | | GTTVTVSS |

A-H.26

| | | |
|---|---|---|
| SEQ ID NO: | VH + VL | QVQLVQSGAEVKKPGSSVKVSCKASGTDFKLTYIHWV |
| 149 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 150 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 151 | | RQAPGQGLEWMGRIFPGSGNTKYNEKFKGRVTITADTS |
| | | TSTAYMELSSLRSEDTAVYYCAGSYYSYDVLDYWGQG |
| | | TTVTVSS |

A-H.27

| | | |
|---|---|---|
| SEQ ID NO: | VH + VL | QVQLVQSGAEVKKPGSSVKVSCKASGTDFKLTYIHWV |
| 153 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGNRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 108 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 155 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.28

| | | |
|---|---|---|
| SEQ ID NO: | VH + VL | QVQLVQSGAEVKKPGSSVKVSCKASGTDFKLTYIHWV |
| 156 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 157 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 158 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSS |

A-H.29

| | | |
|---|---|---|
| SEQ ID NO: | VH + VL | QVQLVQSGAEVKKPGSSVKVSCKASGHDFHLWYIHWV |
| 159 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGDRVAWHQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWHQQ |
| 160 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFHLWYIHWV |
| 161 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSS |

A-H.31

| | | |
|---|---|---|
| SEQ ID NO: | VH + VL | QVQLVQSGAEVKKPGSSVKVSCKASGHDFKLTYIHWV |
| 162 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 163 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFKLTYIHWV |
| 164 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.31

| | | |
|---|---|---|
| SEQ ID NO: | VH + VL | QVQLVQSGAEVKKPGSSVKVSCKASGTDFHLWYIHWV |
| 165 | | RQAPGQGLEWMGRVFAGSGSYRYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 166 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFHLWYIHWV |
| 167 | | RQAPGQGLEWMGRVFAGSGSYRYNEKFKGRVTITADT |
| | | STSTAYMELSSLRSEDTAVYYCAGSYYSYDVLDYWGQ |
| | | GTTVTVSS |

A-H.32

| | | |
|---|---|---|
| SEQ ID NO: | VH + VL | QVQLVQSGAEVKKPGSSVKVSCKASGTDFDKIYIHWVR |
| 168 | | QAPGQGLEWMGRISAGSGNTKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVADRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVADRVAWYQQ |
| 169 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 170 | | QAPGQGLEWMGRISAGSGNTKYNEKFKGRVTITADTST |
| | | STAYMELSSLRSEDTAVYYCAGSYYSYDVLDYWGQGT |
| | | TVTVSS |

A-H.33

| | | |
|---|---|---|
| SEQ ID NO: | VH + VL | QVQLVQSGAEVKKPGSSVKVSCKASGTDFKLTYIHWV |
| 171 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVEDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 172 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 173 | | RQAPGQGLEWMGRISAGSGNTKYNEKFKGRVTITADTS |
| | | TSTAYMELSSLRSEDTAVYYCAGSYYSYDVLDYWGQG |
| | | TTVTVSS |

A-H.34

| | | |
|---|---|---|
| SEQ ID NO: | VH + VL | QVQLVQSGAEVKKPGSSVKVSCKASGTDFRLTYIHWV |
| 174 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGNRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGNRVAWYQQ |
| 108 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFRLTYIHWV |
| 176 | | RQAPGQGLEWMGRISPGSGNTKYNEKFKGRVTITADTS |
| | | TSTAYMELSSLRSEDTAVYYCAGSYYSYDVLDYWGQG |
| | | TTVTVSS |

A-H.35

| | | |
|---|---|---|
| SEQ ID NO: | VH + VL | QVQLVQSGAEVKKPGSSVKVSCKASGHDFDKTYIHWV |
| 177 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWYQQ |
| 178 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKTYIHWV |
| 179 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.36

| | | |
|---|---|---|
| SEQ ID NO: | VH + VL | QVQLVQSGAEVKKPGSSVKVSCKASGHDFKLTYIHWV |
| 180 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVEDRVAWHQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVEDRVAWHQQ |
| 181 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFKLTYIHWV |
| 182 | | RQAPGQGLEWMGRVSPGSGNTKYNEKFKGRVTITADT |
| | | STSTAYMELSSLRSEDTAVYYCAGSYYSYDVLDYWGQ |
| | | GTTVTVSS |

A-H.37

| | | |
|---|---|---|
| SEQ ID NO: | VH + VL | QVQLVQSGAEVKKPGSSVKVSCKASGHDFDKTYIHWV |
| 183 | | RQAPGQGLEWMGRIYPGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVADRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVADRVAWYQQ |
| 184 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKTYIHWV |
| 185 | | RQAPGQGLEWMGRIYPGSGNVKYNEKFKGRVTITADT |
| | | STSTAYMELSSLRSEDTAVYYCAGSYYSYDVLDYWGQ |
| | | GTTVTVSS |

A-H.38

| | | |
|---|---|---|
| SEQ ID NO: | VH + VL | QVQLVQSGAEVKKPGSSVKVSCKASGTDFDKTYIHWV |
| 186 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 7513 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKTYIHWV |
| 188 | | RQAPGQGLEWMGRISAGSGNVKYNEKFKGRVTITADT |
| | | STSTAYMELSSLRSEDTAVYYCAGSYYSYDVLDYWGQ |
| | | GTTVTVSS |

A-H.39

| | | |
|---|---|---|
| SEQ ID NO: | VH + VL | QVQLVQSGAEVKKPGSSVKVSCKASGTDFDKIYIHWVR |
| 189 | | QAPGQGLEWMGRISAGSGNIKYNEKFKGRVTITADTST |
| | | STAYMELSSLRSEDTAVYYCAGSYYSYDVLDYWGQGT |
| | | TVTVSSGGGGSGGGGSGGGGSGGGGSDIQMTQSPSFLS |
| | | ASVGDRVTITCKASQNVDDRVAWYQQKPGKAPKALIY |
| | | SSSHRYKGVPSRFSGSGSGTEFTLTISSLQPEDFATYFCQ |
| | | QFKSYPLTFGQGTKLEIK |
| SEQ ID NO: | VL | DIQMTQSPSFLSASVGDRVTITCKASQNVDDRVAWYQQ |
| 190 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 191 | | QAPGQGLEWMGRISAGSGNIKYNEKFKGRVTITADTST |
| | | STAYMELSSLRSEDTAVYYCAGSYYSYDVLDYWGQGT |
| | | TVTVSS |

A-H.40

| | | |
|---|---|---|
| SEQ ID NO: | VH + VL | QVQLVQSGAEVKKPGSSVKVSCKASGTDFDKIYIHWVR |
| 192 | | QAPGQGLEWMGRISAGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGDRVAWYQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVGDRVAWYQQ |
| 193 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFDKIYIHWVR |
| 194 | | QAPGQGLEWMGRISAGSGNVKYNEKFKGRVTITADTS |
| | | TSTAYMELSSLRSEDTAVYYCAGSYYSYDVLDYWGQG |
| | | TTVTVSS |

A-H.41

| | | |
|---|---|---|
| SEQ ID NO: | VH + VL | QVQLVQSGAEVKKPGSSVKVSCKASGGTFKLTYIHWV |
| 195 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDDRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDDRVAWYQQ |
| 196 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGGTFKLTYIHWV |
| 197 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.42

| | | |
|---|---|---|
| SEQ ID NO: | VH + VL | QVQLVQSGAEVKKPGSSVKVSCKASGTDFKLTYIHWV |
| 198 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDNRVAWHQQKPGKAPKALI |
| | | YSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWHQQ |
| 199 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGTDFKLTYIHWV |
| 200 | | RQAPGQGLEWMGRISPGSGNVKYNEKFKGRVTITADTS |
| | | TSTAYMELSSLRSEDTAVYYCAGSYYSYDVLDYWGQG |
| | | TTVTVSS |

A-H.43

| | | |
|---|---|---|
| SEQ ID NO: | VH + VL | QVQLVQSGAEVKKPGSSVKVSCKASGHDFDKFYIHWV |
| 201 | | RQAPGQGLEWMGRVSAGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVDNRVAWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VL | DIQMTQSPSFLSASVGDRVTITCKASQNVDNRVAWYQQ |
| 202 | | KPGKAPKALIYSSSHRYKGVPSRFSGSGSGTEFTLTISSL |
| | | QPEDFATYFCQQFKSYPLTFGQGTKLEIK |
| SEQ ID NO: | VH | QVQLVQSGAEVKKPGSSVKVSCKASGHDFDKFYIHWV |
| 203 | | RQAPGQGLEWMGRVSAGSGNTKYNEKFKGRVTITADT |
| | | STSTAYMELSSLRSEDTAVYYCAGSYYSYDVLDYWGQ |
| | | GTTVTVSS |

A-H.44

| | | |
|---|---|---|
| SEQ ID NO: | VH + VL | QVQLVQSGAEVKKPGSSVKVSCKASGTDFDKFYIHWV |
| 204 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGGGGGSGGGGSGGGGSDIQMTQSPSF |
| | | LSASVGDRVTITCKASQNVGDRVVWYQQKPGKAPKAL |
| | | IYSSSHRYKGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGTDFDKFYIHWV |
| 205 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSS |

A-H.45

| | | |
|---|---|---|
| SEQ ID NO: | VH + VL | QVQLVQSGAEVKKPGSSVKVSCKASGYSFTTYYIHWV |
| 206 | | RQAPGQGLEWMGWFSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 207 | | RQAPGQGLEWMGWFSAGSGNTKYNEKFKGRVTITADT |
| | | STSTAYMELSSLRSEDTAVYYCAVSYYSYDVLDYWGQ |
| | | GTTVTVSS |

A-H.46

| | | |
|---|---|---|
| SEQ ID NO: | VH + VL | QVQLVQSGAEVKKPGSSVKVSCKASGYSFTTYYIHWV |
| 208 | | RQAPGQGLEWMGWFSAGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 209 | | RQAPGQGLEWMGWFSAGSGNTKYNEKFKGRVTITADT |
| | | STSTAYMELSSLRSEDTAVYYCAGSYYSYDVLDYWGQ |
| | | GTTVTVSS |

A-H.47

| | | |
|---|---|---|
| SEQ ID NO: | VH + VL | QVQLVQSGAEVKKPGSSVKVSCKASGYSFTTYYIHWV |
| 210 | | RQAPGQGLEWMGWFFPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 211 | | RQAPGQGLEWMGWFFPGSGNTKYNEKFKGRVTITADT |
| | | STSTAYMELSSLRSEDTAVYYCAGSYYSYDVLDYWGQ |
| | | GTTVTVSS |

A-H.48

| | | |
|---|---|---|
| SEQ ID NO: | VH + VL | QVQLVQSGAEVKKPGSSVKVSCKASGYSFTTYYIHWV |
| 212 | | RQAPGQGLEWMGWFSPGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 213 | | RQAPGQGLEWMGWFSPGSGNTKYNEKFKGRVTITADT |
| | | STSTAYMELSSLRSEDTAVYYCAVSYYSYDVLDYWGQ |
| | | GTTVTVSS |

A-H.49

| | | |
|---|---|---|
| SEQ ID NO: | VH + VL | QVQLVQSGAEVKKPGSSVKVSCKASGYSFTTYYIHWV |
| 214 | | RQAPGQGLEWMGWFSPGSGNTKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTTYYIHWV |
| 215 | | RQAPGQGLEWMGWFSPGSGNTKYNEKFKGRVTITADT |
| | | STSTAYMELSSLRSEDTAVYYCAGSYYSYDVLDYWGQ |
| | | GTTVTVSS |

A-H.50

| | | |
|---|---|---|
| SEQ ID NO: | VH + VL | QVQLVQSGAEVKKPGSSVKVSCKASGYSFTTYYIHWV |
| 216 | | RQAPGQGLEWMGRIFPGSGNIKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVGINVVWHQQKPGKAPKALIY |
| | | SSSHRYSGVPSRFSGSGSGTEFTLTISSLQPEDFATYFCQ |
| | | QFKSYPLTFGQGTKLEIK |
| SEQ ID NO: | VH | QVQLVQSGAEVKKPGSSVKVSCKASGYSFTTYYIHWV |
| 217 | | RQAPGQGLEWMGRIFPGSGNIKYNEKFKGRVTITADTS |
| | | TSTAYMELSSLRSEDTAVYYCAGSYYSYDVLDYWGQG |
| | | TTVTVSS |

A-H.51

| | | |
|---|---|---|
| SEQ ID NO: | VH + VL | QVQLVQSGAEVKKPGSSVKVSCKASGYSFTTYYIHWV |
| 218 | | RQAPGQGLEWMGWFFPGSGNIKYNEKFKGRVTITADT |
| | | STSTAYMELSSLRSEDTAVYYCAGSIYSAGVLDYWGQG |
| | | TTVTVSSGGGGSGGGGSGGGGSGGGGSDIQMTQSPSFL |
| | | SASVGDRVTITCKASQNVGINVVWHQQKPGKAPKALIY |
| | | SSSHRYSGVPSRFSGSGSGTEFTLTISSLQPEDFATYFCQ |
| | | QFKSYPLTFGQGTKLEIK |
| SEQ ID NO: | VH | QVQLVQSGAEVKKPGSSVKVSCKASGYSFTTYYIHWV |
| 219 | | RQAPGQGLEWMGWFFPGSGNIKYNEKFKGRVTITADT |
| | | STSTAYMELSSLRSEDTAVYYCAGSIYSAGVLDYWGQG |
| | | TTVTVSS |

A-H.52

| | | |
|---|---|---|
| SEQ ID NO: | VH + VL | QVQLVQSGAEVKKPGSSVKVSCKASGYSFTLGYIHWV |
| 220 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFTLGYIHWV |
| 221 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |

A-H.53

| | | |
|---|---|---|
| SEQ ID NO: | VH + VL | QVQLVQSGAEVKKPGSSVKVSCKASGYSFRLTYIHWV |
| 222 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFRLTYIHWV |
| 223 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |

A-H.54

| | | |
|---|---|---|
| SEQ ID NO: | VH + VL | QVQLVQSGAEVKKPGSSVKVSCKASGYSFHNWYIHWV |
| 224 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGINVVWHQQKPGKAPKALI |
| | | YSSSHRYSGVPSRFSGSGSGTEFTLTISSLQPEDFATYFC |
| | | QQFKSYPLTFGQGTKLEIK |
| SEQ ID NO: | VH | QVQLVQSGAEVKKPGSSVKVSCKASGYSFHNWYIHWV |
| 225 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |

A-H.55 antibody

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: | HC CDR1 (Kabat) | TYYIH |
| 45 | | |
| SEQ ID NO: | HC CDR2 (Kabat) | WFFPGSGNIKYNEKFKG |
| 46 | | |
| SEQ ID NO: | HC CDR3 (Kabat) | SYYSYDVLDY |
| 47 | | |
| SEQ ID | HC CDR1 (Chothia) | GYSFTTY |
| NO: 48 | | |
| SEQ ID NO: | HC CDR2 (Chothia) | FPGSGN |
| 49 | | |
| SEQ ID NO: | HC CDR3 (Chothia) | SYYSYDVLDY |
| 50 | | |
| SEQ ID NO: | VH | QVQLVQSGAEVKKPGSSVKVSCKASGYSFTTYYIHWV |
| 1100 | | RQAPGQGLEWMGWFFPGSGNIKYNEKFKGRVTITADT |
| | | STSTAYMELSSLRSEDTAVYYCAGSYYSYDVLDYWGQ |
| | | GTTVTVSS |
| SEQ ID NO: 6 | LC CDR1 | KASQNVGINVV |
| | (Combined) | |
| SEQ ID NO: 7 | LC CDR2 | SSSHRYS |
| | (Combined) | |
| SEQ ID NO: 8 | LC CDR3 | QQFKSYPLT |
| | (Combined) | |
| SEQ ID NO: | LC CDR1 (Kabat) | KASQNVGINVV |
| 51 | | |
| SEQ ID NO: | LC CDR2 (Kabat) | SSSHRYS |
| 52 | | |
| SEQ ID NO: | LC CDR3 (Kabat) | QQFKSYPLT |
| 53 | | |
| SEQ ID NO: | LC CDR1 (Chothia) | KASQNVGINVV |
| 54 | | |
| SEQ ID NO: | LC CDR2 (chothia) | SSSHRYS |
| 55 | | |
| SEQ ID NO: | LC CDR3 (chothia) | QQFKSYPLT |
| 56 | | |
| SEQ ID NO: | VL | QSVLTQPPSVSEAPRQRVTISCKASQNVGINVVWHQQL |
| 1101 | | PGKAPKALIYSSSHRYSGVSDRFSGSGSGTSFSLAISGLQ |
| | | SEDEADYFCQQFKSYPLTFGTGTKVTVL |

A-H.56

| | | |
|---|---|---|
| SEQ ID NO: | VH+ VL (ScFv) | QVQLVQSGAEVKKPGSSVKVSCKASGHDFDKFYIHWV |
| 1309 | | RQAPGQGLEWMGRVSAGSGNVKYNEKFKGRVTITADT |
| | | STSTAYMELSSLRSEDTAVYYCAGSYYSYDVLDYWGQ |
| | | GTTVTVSSGGGGSGGGGSGGGGSGGGGSDIQMTQSPSF |
| | | LSASVGDRVTITCKASQNVGNRVAWYQQKPGKAPKAL |
| | | IYSSSHRYKGVPSRFSGSGSGTEFTLTISSLQPEDFATYFC |
| | | QQFKSYPLTFGQGTKLEIK |

A-H.57

| | | |
|---|---|---|
| SEQ ID NO: | VH+ VL (ScFv) | QVQLVQSGAEVKKPGSSVKVSCKASGHDFRLTYIHWV |
| 1326 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGDRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.58

| | | |
|---|---|---|
| SEQ ID NO: | VH+ VL (ScFv) | QVQLVQSGAEVKKPGSSVKVSCKASGHDFRLTYIHWV |
| 1327 | | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGNRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.59

| | | |
|---|---|---|
| SEQ ID NO: | VH+ VL (ScFv) | QVQLVQSGAEVKKPGSSVKVSCKASGHDFRLTYIHWV |
| 1328 | | RQAPGQGLEWMGRIYAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.60

| | | |
|---|---|---|
| SEQ ID NO: | VH+ VL (ScFv) | QVQLVQSGAEVKKPGSSVKVSCKASGHDFKLTYIHWV |
| 1329 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGDRVAWHQQKPGKAPKAL |
| | | IYSSSHRYKGVPSRFSGSGSGTEFTLTISSLQPEDFATYFC |
| | | QQFKSYPLTFGQGTKLEIK |

A-H.61

| | | |
|---|---|---|
| SEQ ID NO: | VH+ VL (ScFv) | QVQLVQSGAEVKKPGSSVKVSCKASGHDFKLTYIHWV |
| 1330 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVDNRVAWHQQKPGKAPKAL |
| | | IYSSSHRYKGVPSRFSGSGSGTEFTLTISSLQPEDFATYFC |
| | | QQFKSYPLTFGQGTKLEIK |

A-H.62

| | | |
|---|---|---|
| SEQ ID NO: | VH+ VL (ScFv) | QVQLVQSGAEVKKPGSSVKVSCKASGHDFRLTYIHWV |
| 1331 | | RQAPGQGLEWMGRVSAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.63

| | | |
|---|---|---|
| SEQ ID NO: | VH+ VL (ScFv) | QVQLVQSGAEVKKPGSSVKVSCKASGHDFRLTYIHWV |
| 1332 | | RQAPGQGLEWMGRVYAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVEDRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.64

| | | |
|---|---|---|
| SEQ ID NO: | VH+ VL (ScFv) | QVQLVQSGAEVKKPGSSVKVSCKASGHDFKLTYIHWV |
| 1333 | | RQAPGQGLEWMGRVSAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.65

| | | |
|---|---|---|
| SEQ ID NO: | VH+ VL (ScFv) | QVQLVQSGAEVKKPGSSVKVSCKASGHDFKLTYIHWV |
| 1334 | | RQAPGQGLEWMGRISAGSGNTKYNEKFKGRVTITADTS |
| | | TSTAYMELSSLRSEDTAVYYCAVSYYSYDVLDYWGQG |
| | | TTVTVSSGGGGSGGGGSGGGGSGGGGSDIQMTQSPSFL |
| | | SASVGDRVTITCKASQNVGDRVVWHQQKPGKAPKALI |
| | | YSSSHRYKGVPSRFSGSGSGTEFTLTISSLQPEDFATYFC |
| | | QQFKSYPLTFGQGTKLEIK |

A-H.66

| | | |
|---|---|---|
| SEQ ID NO: | VH+ VL (ScFv) | QVQLVQSGAEVKKPGSSVKVSCKASGHDFKLTYIHWV |
| 1335 | | RQAPGQGLEWMGRIYAGSGNTKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVGDRVVWHQQKPGKAPKAL |
| | | IYSSSHRYKGVPSRFSGSGSGTEFTLTISSLQPEDFATYFC |
| | | QQFKSYPLTFGQGTKLEIK |

A-H.67

| | | |
|---|---|---|
| SEQ ID NO: | VH+ VL (ScFv) | QVQLVQSGAEVKKPGSSVKVSCKASGTDFKLTYIHWV |
| 1336 | | RQAPGQGLEWMGRIFPGSGNVKYNEKFKGRVTITADTS |
| | | TSTAYMELSSLRSEDTAVYYCAVSYYSYDVLDYWGQG |
| | | TTVTVSSGGGGSGGGGSGGGGSGGGGSDIQMTQSPSFL |
| | | SASVGDRVTITCKASQNVDNRVAWYQQKPGKAPKALI |
| | | YSSSHRYKGVPSRFSGSGSGTEFTLTISSLQPEDFATYFC |
| | | QQFKSYPLTFGQGTKLEIK |

A-H.68

| | | |
|---|---|---|
| SEQ ID NO: | VH+ VL (ScFv) | QVQLVQSGAEVKKPGSSVKVSCKASGHDFRLTYIHWV |
| 1337 | | RQAPGQGLEWMGRISAGSGNVKYNEKFKGRVTITADT |
| | | STSTAYMELSSLRSEDTAVYYCAVSYYSYDVLDYWGQ |
| | | GTTVTVSSGGGGSGGGGSGGGGSGGGGSDIQMTQSPSF |
| | | LSASVGDRVTITCKASQNVADRVAWYQQKPGKAPKAL |
| | | IYSSSHRYKGVPSRFSGSGSGTEFTLTISSLQPEDFATYFC |
| | | QQFKSYPLTFGQGTKLEIK |

A-H.69 (also referred to as A-H.13)

| | | |
|---|---|---|
| SEQ ID NO: | VH+ VL (ScFv) | QVQLVQSGAEVKKPGSSVKVSCKASGTDFKLTYIHWV |
| 110 | | RQAPGQGLEWMGRIFPGSGNVKYNEKFKGRVTITADTS |
| | | TSTAYMELSSLRSEDTAVYYCAGSYYSYDVLDYWGQG |
| | | TTVTVSSGGGGSGGGGSGGGGSGGGGSDIQMTQSPSFL |
| | | SASVGDRVTITCKASQNVDNRVAWYQQKPGKAPKALI |
| | | YSSSHRYKGVPSRFSGSGSGTEFTLTISSLQPEDFATYFC |
| | | QQFKSYPLTFGQGTKLEIK |

A-H humanized-matured VH

| | | |
|---|---|---|
| SEQ ID NO: | VH-humanized | QVQLVQSGAEVKKPGSSVKVSCKASGTDFKLTYIHWV |
| 1310 | matured 1 | RQAPGQGLEWMGRIFPGSGNVKYNEKFKGRVTITADTS |
| | | TSTAYMELSSLRSEDTAVYYCAGSYYSYDVLDYWGQG |
| | | TTVTVSS |
| SEQ ID NO: | VH-humanized | QVQLVQSGAEVKKPGSSVKVSCKASGTDFKLTYIHWV |
| 1311 | matured 2 | RQAPGQGLEWMGRIFPGSGNVKYNEKFKGRVTITADTS |
| | | TSTAYMELSSLRSEDTAVYYCAVSYYSYDVLDYWGQG |
| | | TTVTVSS |
| SEQ ID NO: | VH-humanized | QVQLVQSGAEVKKPGSSVKVSCKASGHDFRLTYIHWV |
| 1312 | matured 3 | RQAPGQGLEWMGRISAGSGNVKYNEKFKGRVTITADT |
| | | STSTAYMELSSLRSEDTAVYYCAVSYYSYDVLDYWGQ |
| | | GTTVTVSS |

A-H humanized-matured VL

| | | |
|---|---|---|
| SEQ ID NO: | VL-humanized | DIQMTQSPSFLSASVGDRVTITCKASQNVDNRVAWYQQ |
| 1313 | matured 1 | KPGKAPKALIYSSSHRYKGVPSRFSGSGSGTEFTLTISSL |
| | | QPEDFATYFCQQFKSYPLTFGQGTKLEIK |
| SEQ ID NO: | VL-humanized | DIQMTQSPSFLSASVGDRVTITCKASQNVADRVAWYQQ |
| 1314 | matured 2 | KPGKAPKALIYSSSHRYKGVPSRFSGSGSGTEFTLTISSL |
| | | QPEDFATYFCQQFKSYPLTFGQGTKLEIK |

A-H.70

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVGNRVV |
| 3652 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWV |
| 1346 | (CDRs underlined) | RQAPGQGLEWMGRVSAGSGNVKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGNRVVWHQQ |
| 1347 | (CDRs underlined) | KPGKAPKALIYSSSHRYKGVPSRFSGSGSGTEFTLTISSL |
| | | QPEDFATYFCQQFKSYPLTFGQGTKLEIK |

A-H.71

| | | |
|---|---|---|
| SEQ ID NO: | HC CDR1 | GHDFRLTYIH |
| 3650 | | |
| SEQ ID NO: | HC CDR2 | RIYAGSGNVKYNEKFKG |
| 3654 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWV |
| 1348 | (CDRs underlined) | RQAPGQGLEWMGRIYAGSGNVKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVADRVVWHQQ |
| 1349 | (CDRs underlined) | KPGKAPKALIYSSSHRYKGVPSRFSGSGSGTEFTLTISSL |
| | | QPEDFATYFCQQFKSYPLTFGQGTKLEIK |

A-H.72

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVA |
| 3658 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1350 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGDRVAWHQQ |
| 1351 | (CDRs underlined) | KPGKAPKALIYSSSHRYKGVPSRFSGSGSGTEFTLTISSL |
| | | QPEDFATYFCQQFKSYPLTFGQGTKLEIK |

A-H.73

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1350 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVDNRVAWHQQ |
| 1353 | (CDRs underlined) | KPGKAPKALIYSSSHRYKGVPSRFSGSGSGTEFTLTISSL |
| | | QPEDFATYFCQQFKSYPLTFGQGTKLEIK |

A-H.74

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWV |
| 1346 | (CDRs underlined) | RQAPGQGLEWMGRVSAGSGNVKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVADRVVWHQQ |
| 1349 | (CDRs underlined) | KPGKAPKALIYSSSHRYKGVPSRFSGSGSGTEFTLTISSL |
| | | QPEDFATYFCQQFKSYPLTFGQGTKLEIK |

A-H.75

| | | |
|---|---|---|
| SEQ ID NO: | HC CDR1 | GHDFRLTYIH |
| 3650 | | |
| SEQ ID NO: | HC CDR2 | RVYAGSGNTKYNEKFKG |
| 3659 | | |
| SEQ ID NO: 5 | HC CDR3 | SYYSYDVLDY |
| SEQ ID NO: | LC CDR1 | KASQNVEDRVV |
| 3660 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWV |
| 1356 | (CDRs underlined) | RQAPGQGLEWMGRVYAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVEDRVVWHQQ |
| 1357 | (CDRs underlined) | KPGKAPKALIYSSSHRYKGVPSRFSGSGSGTEFTLTISSL |
| | | QPEDFATYFCQQFKSYPLTFGQGTKLEIK |

A-H.76

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1350 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVADRVVWHQQ |
| 1349 | (CDRs underlined) | KPGKAPKALIYSSSHRYKGVPSRFSGSGSGTEFTLTISSL |
| | | QPEDFATYFCQQFKSYPLTFGQGTKLEIK |

A-H.77

| | | |
|---|---|---|
| SEQ ID NO: | HC CDR1 | GHDFKLTYIH |
| 3656 | | |
| SEQ ID NO: | HC CDR2 | RISAGSGNTKYNEKFKG |
| 3661 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1360 | (CDRs underlined) | RQAPGQGLEWMGRISAGSGNTKYNEKFKGRVTITADTS |
| | | TSTAYMELSSLRSEDTAVYYCAVSYYSYDVLDYWGQG |
| | | TTVTVSS |
| SEQ ID NO: | VL | DIQMTQSPSFLSASVGDRVTITCKASQNVGDRVVWHQQ |
| 1361 | (CDRs underlined) | KPGKAPKALIYSSSHRYKGVPSRFSGSGSGTEFTLTISSL |
| | | QPEDFATYFCQQFKSYPLTFGQGTKLEIK |

A-H.78

| | | |
|---|---|---|
| SEQ ID NO: | HC CDR1 | GHDFKLTYIH |
| 3656 | | |
| SEQ ID NO: | HC CDR2 | RIYAGSGNTKYNEKFKG |
| 3663 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1362 | (CDRs underlined) | RQAPGQGLEWMGRIYAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGDRVVWHQQ |
| 1361 | (CDRs underlined) | KPGKAPKALIYSSSHRYKGVPSRFSGSGSGTEFTLTISSL |
| | | QPEDFATYFCQQFKSYPLTFGQGTKLEIK |

A-H.79

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1350 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCRASQNVDNRLGWHQQ |
| 1365 | (CDRs underlined) | KPGKAPKALIYSSSHRYKGVPSRFSGSGSGTEFTLTISSL |
| | | QPEDFATYFCQQFKSYPLTFGQGTKLEIK |

A-H.80

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1350 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVDNRVAWHQQ |
| 1367 | (CDRs underlined) | KPGKAPKALIYAASSLQKGVPSRFSGSGSGTEFTLTISSL |
| | | QPEDFATYFCQQFKSYPLTFGQGTKLEIK |

A-H.81

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWV |
| 1350 | (CDRs underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVDNRVAWHQQ |
| 1369 | (CDRs underlined) | KPGKAPKALIYSSSHRYKGVPSRFSGSGSGTEFTLTISSL |
| | | QPEDFATYFCLQHNSYPLTFGQGTKLEIK |

A-H.82

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1370 | (CDRs underlined) | RQAPGQGLEWMGRVSAGSGNVNYAQKFQGRVTITAD |
| | | TSTSTAYMELSSLRSEDTAVYYCAVSYYSYDVLDYWG |
| | | QGTTVTVSS |
| SEQ ID NO: | VL | DIQMTQSPSFLSASVGDRVTITCRASQNVDNRLGWHQQ |
| 1365 | (CDRs underlined) | KPGKAPKALIYSSSHRYKGVPSRFSGSGSGTEFTLTISSL |
| | | QPEDFATYFCQQFKSYPLTFGQGTKLEIK |

A-H.83

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWV |
| 1370 | (CDRs underlined) | RQAPGQGLEWMGRVSAGSGNVNYAQKFQGRVTITAD |
| | | TSTSTAYMELSSLRSEDTAVYYCAVSYYSYDVLDYWG |
| | | QGTTVTVSS |
| SEQ ID NO: | VL | DIQMTQSPSFLSASVGDRVTITCKASQNVDNRVAWHQQ |
| 1367 | (CDRs underlined) | KPGKAPKALIYAASSLQKGVPSRFSGSGSGTEFTLTISSL |
| | | QPEDFATYFCQQFKSYPLTFGQGTKLEIK |

A-H.84

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWV |
| 1370 | (CDRs underlined) | RQAPGQGLEWMGRVSAGSGNVNYAQKFQGRVTITAD |
| | | TSTSTAYMELSSLRSEDTAVYYCAVSYYSYDVLDYWG |
| | | QGTTVTVSS |
| SEQ ID NO: | VL | DIQMTQSPSFLSASVGDRVTITCKASQNVDNRVAWHQQ |
| 1369 | (CDRs underlined) | KPGKAPKALIYSSSHRYKGVPSRFSGSGSGTEFTLTISSL |
| | | QPEDFATYFCLQHNSYPLTFGQGTKLEIK |

A-H.85

| | | |
|---|---|---|
| SEQ ID NO: | HC CDR1 | GHDFRLTYIH |
| 3650 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH (CDRs | QVQLVQSGAEVKKPGSSVKVSCKASGHDFRLTYIHWV |
| 1344 | underlined) | RQAPGQGLEWMGRVSAGSGNTKYNEKFKGRVTITADT |
| | | STSTAYMELSSLRSEDTAVYYCAVSYYSYDVLDYWGQ |
| | | GTTVTVSS |
| SEQ ID NO: | VL | DIQMTQSPSFLSASVGDRVTITCKASQNVGDRVVWHQQ |
| 1361 | (CDRs underlined) | KPGKAPKALIYSSSHRYKGVPSRFSGSGSGTEFTLTISSL |
| | | QPEDFATYFCQQFKSYPLTFGQGTKLEIK |

TABLE 2
Amino acid and nucleotide sequences for murine and humanized antibody molecules
which bind to TCRVB 12, e.g., TCRVB 12-3 or TCRVB 12-4. The antibody molecules include
murine mAb Antibody B and humanized mAb Antibody B-H. 1 to B-H.6. The amino acid the
heavy and light chain CDRs, and the amino acid and nucleotide sequences of the heavy
and light chain variable regions, and the heavy and light chains are shown.
Antibody B (murine), also referred to as 16G8

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFG |
| | | MHWVRQAPDKGLEWVAYISSGSSTIYYADTLKG |
| | | RFTISRDNPKNTLFLQMTSLRSEDTAMYYCARRG |
| | | EGAMDYWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIYW |
| | | YQQKSDASPKLWIYYTSNLAPGVPTRFSGSGSGN |
| | | SYSLTISSMEGEDAATYYCQQFTSSPFTFGSGTKL |
| | | EIK |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 3438 | VH | EVQLVESGGGLVQPGGSLRLSCAASGFTFSNFGM |
| | | HWVRQAPGKGLEWVSYISSGSSTIYYADTLKGRF |
| | | TISRDNAKNSLYLQMNSLRAEDTAVYYCARRGE |
| | | GAMDYWGQGTTVTVSS |
| SEQ ID NO: 31 | DNA VH | GAGGTGCAGCTGGTTGAATCTGGCGGAGGATT |
| | | GGTTCAGCCTGGCGGCTCTCTGAGACTGTCTTG |
| | | TGCCGCTTCTGGCTTCACCTTCTCCAACTTCGG |
| | | CATGCACTGGGTCCGACAGGCCCCTGGAAAAG |
| | | GACTGGAATGGGTGTCCTACATCTCCTCCGGCT |
| | | CCTCCACCATCTACTACGCTGACACCCTGAAGG |
| | | GCAGATTCACCATCTCTCGGGACAACGCCAAG |
| | | AACTCCCTGTACCTGCAGATGAACAGCCTGAG |
| | | AGCCGAGGACACCGCCGTGTACTACTGTGCTA |
| | | GAAGAGGCGAGGGCGCCATGGATTATTGGGGC |
| | | CAGGGAACCACAGTGACCGTGTCTAGC |

Antibody B-H.1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | EVQLVESGGGLVQPGGSLRLSCAASGFTFSNFGM |
| | | HWVRQAPGKGLEWVSYISSGSSTIYYADTLKGRF |
| | | TISRDNSKNTLYLQMNSLRAEDTAVYYCARRGE |
| | | GAMDYWGQGTTVTVSS |
| SEQ ID NO: 32 | DNA VH | GAGGTGCAGCTGGTTGAATCTGGCGGAGGATT |
| | | GGTTCAGCCTGGCGGCTCTCTGAGACTGTCTTG |
| | | TGCCGCTTCTGGCTTCACCTTCTCCAACTTCGG |
| | | CATGCACTGGGTCCGACAGGCCCCTGGAAAAG |
| | | GACTGGAATGGGTGTCCTACATCTCCTCCGGCT |
| | | CCTCCACCATCTACTACGCTGACACCCTGAAGG |
| | | GCAGATTCACCATCAGCCGGGACAACTCCAAG |
| | | AACACCCTGTACCTGCAGATGAACTCCCTGAG |
| | | AGCCGAGGACACCGCCGTGTACTACTGTGCTA |
| | | GAAGAGGCGAGGGCGCCATGGATTATTGGGGC |
| | | CAGGGAACCACAGTGACCGTGTCTAGC |

Antibody B-H.1C HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | QVQLVESGGGVVQPGRSLRLSCAASGFTFSNFG |
| | | MHWVRQAPGKGLEWVAYISSGSSTIYYADTLKG |
| | | RFTISRDNSKNTLYLQMNSLRAEDTAVYYCARR |
| | | GEGAMDYWGQGTTVTVSS |
| SEQ ID NO: 33 | DNA VH | CAGGTGCAGCTGGTGGAATCTGGTGGCGGAGT |
| | | TGTGCAGCCTGGCAGATCCCTGAGACTGTCTTG |
| | | TGCCGCCTCTGGCTTCACCTTCTCCAACTTCGG |
| | | CATGCACTGGGTCCGACAGGCCCCTGGAAAAG |
| | | GATTGGAGTGGGTCGCCTACATCTCCTCCGGCT |
| | | CCTCCACCATCTACTACGCTGACACCCTGAAGG |
| | | GCAGATTCACCATCAGCCGGGACAACTCCAAG |
| | | AACACCCTGTACCTGCAGATGAACTCCCTGAG |
| | | AGCCGAGGACACCGCCGTGTACTACTGTGCTA |
| | | GAAGAGGCGAGGGCGCCATGGATTATTGGGGC |
| | | CAGGGAACCACAGTGACCGTGTCTAGC |

Antibody B-H.1D LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 26 | VL | DNQLTQSPSFLSASVGDRVTITCRASSSVNYIYW |
| | | YQQKPGKAPKLLIYYTSNLAPGVPSRFSGSGSGN |
| | | EYTLTISSLQPEDFATYYCQQFTSSPFTFGQGTKL |
| | | EIK |
| SEQ ID NO: 34 | DNA VL | GATAACCAGCTGACCCAGTCTCCTAGCTTCCTG |
| | | TCTGCCTCTGTGGGCGACAGAGTGACAATTAC |
| | | CTGCCGGGCCTCCTCCTCCGTGAACTACATCTA |
| | | CTGGTATCAGCAGAAGCCCGGCAAGGCCCCTA |
| | | AGCTGCTGATCTACTACACCTCCAATCTGGCCC |
| | | CTGGCGTGCCCTCTAGATTTTCCGGATCTGGCT |
| | | CCGGCAACGAGTATACCCTGACAATCTCCAGC |
| | | CTGCAGCCTGAGGACTTCGCCACCTACTACTGC |
| | | CAGCAGTTCACCTCCTCTCCATTCACCTTTGGC |
| | | CAGGGCACCAAGCTGGAAATCAAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 27 | VL | DNQLTQSPSSLSASVGDRVTITCRASSSVNYIYW |
| | | YQQKPGKAPKLLIYYTSNLAPGVPSRFSGSGSGN |
| | | DYTLTISSLQPEDFATYYCQQFTSSPFTFGQGTKL |
| | | EIK |
| SEQ ID NO: 35 | DNA VL | ATAACCAGCTGACCCAGTCTCCTTCCAGCCTGT |
| | | CTGCTTCTGTGGGCGACAGAGTGACAATTACCT |
| | | GCCGGGCCTCCTCCTCCGTGAACTACATCTACT |
| | | GGTATCAGCAGAAGCCCGGCAAGGCCCCTAAG |
| | | CTGCTGATCTACTACACCTCCAATCTGGCCCCT |
| | | GGCGTGCCCTCTAGATTTTCCGGATCTGGCTCC |
| | | GGCAACGACTATACCCTGACAATCTCCAGCCT |
| | | GCAGCCTGAGGACTTCGCCACCTACTACTGCC |
| | | AGCAGTTCACCTCCTCTCCATTCACCTTTGGCC |
| | | AGGGCACCAAGCTGGAAATCAAA |

Antibody B-H.1F LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 28 | VL | ENVLTQSPATLSVSPGERATLSCRASSSVNYIYW |
| | | YQQKPGQAPRLLIYYTSNLAPGIPARFSGSGSGNE |
| | | YTLTISSLQSEDFAVYYCQQFTSSPFTFGQGTKLEI |
| | | K |
| SEQ ID NO: 36 | DNA VL | GAGAATGTGCTGACCCAGTCTCCTGCCACACT |
| | | GTCTGTTAGCCCTGGCGAGAGAGCTACCCTGA |
| | | GCTGCAGAGCCTCTTCCTCCGTGAACTACATCT |
| | | ACTGGTATCAGCAGAAGCCCGGCCAGGCTCCT |
| | | AGACTGCTGATCTACTACACCTCCAATCTGGCC |
| | | CCTGGCATCCCTGCCAGATTTTCCGGATCTGGC |
| | | TCCGGCAACGAGTATACCCTGACCATCTCCAG |
| | | CCTGCAGTCCGAGGACTTTGCTGTGTACTATTG |
| | | CCAGCAGTTCACAAGCAGCCCTTTCACCTTTGG |
| | | CCAGGGCACCAAGCTGGAAATCAAA |

Antibody B-H.1G LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 29 | VL | QNVLTQPPSASGTPGQRVTISCRASSSVNYIYWY |
| | | QQLPGTAPKLLIYYTSNLAPGVPDRFSGSGSGNS |
| | | YSLAISGLRSEDEADYYCQQFTSSPFTFGTGTKVT |
| | | VL |
| SEQ ID NO: 37 | DNA VL | CAGAATGTGCTGACCCAACCTCCTTCCGCCTCT |
| | | GGCACACCTGGACAGAGAGTGACAATCTCCTG |
| | | CCGGGCCTCCTCCTCCGTGAACTACATCTACTG |
| | | GTATCAGCAGCTGCCCGGCACCGCTCCTAAAC |
| | | TGCTGATCTACTACACCTCCAATCTGGCCCCTG |
| | | GCGTGCCCGATAGATTTTCCGGATCTGGCTCCG |
| | | GCAACTCCTACAGCCTGGCTATCTCTGGCCTGA |
| | | GATCTGAGGACGAGGCCGACTACTACTGCCAG |
| | | CAGTTCACCTCCTCTCCATTCACCTTTGGCACC |
| | | GGCACCAAAGTGACAGTTCTT |

Antibody B-H.1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 30 | VL | SNELTQPPSVSVSPGQTARITCRASSSVNYIYWYQ |
| | | QKSGQAPVLVIYYTSNLAPGIPERFSGSGSGNMY |
| | | TLTISGAQVEDEADYYCQQFTSSPFTFGTGTKVT |
| | | VL |
| SEQ ID NO: 38 | DNA VL | TCTAATGAGCTGACCCAGCCTCCTTCCGTGTCC |
| | | GTGTCTCCTGGACAGACCGCCAGAATTACCTG |
| | | CCGGGCCTCCTCCTCCGTGAACTACATCTACTG |
| | | GTATCAGCAGAAGTCCGGCCAGGCTCCTGTGC |
| | | TCGTGATCTACTACACCTCCAATCTGGCCCCTG |
| | | GCATCCCTGAGAGATTCTCCGGATCTGGCTCCG |
| | | GCAACATGTACACCCTGACCATCTCTGGCGCCC |
| | | AGGTGGAAGATGAGGCCGACTACTACTGCCAG |
| | | CAGTTCACCTCCTCTCCATTCACCTTTGGCACC |
| | | GGCACCAAAGTGACAGTTCTT |

Antibody B-H.1

| | | |
|---|---|---|
| SEQ ID NO: 3280 | Chain 1: Fc only | METDTLLLWVLLLWVPGSTGDKTHTCPPCPAPE |
| | | LLGGPSVFLFPPKPKDTLMISRTPEVTCVVVDVSH |
| | | EDPEVKFNWYVDGVEVHNAKTKPREEQYNSTY |
| | | RVVSVLTVLHQDWLNGKEYKCKVSNKALPAPIE |
| | | KTISKAKGQPREPQVYTLPPCREEMTKNQVSLW |
| | | CLVKGFYPSDIAVEWESNGQPENNYKTTPPVLDS |
| | | DGSFFLYSKLTVDKSRWQQGNVFSCSVMHEALH |
| | | NRFTQKSLSLSPGK |
| SEQ ID NO: 3281 | Chain 2: humanized B- | METDTLLLWVLLLWVPGSTGEVQLVESGGGLVQ |
| | H scFv | PGGSLRLSCAASGFTFSNFGMHWVRQAPGKGLE |
| | | WVSYISSGSSTIYYADTLKGRFTISRDNSKNTLYL |
| | | QMNSLRAEDTAVYYCARRGEGAMDYWGQGTT |
| | | VTVSSGGGGSGGGGSGGGGSGGGGSDNQLTQSP |
| | | SFLSASVGDRVTITCRASSSVNYIYWYQQKPGKA |
| | | PKLLIYYTSNLAPGVPSRFSGSGSGNEYTLTISSLQ |
| | | PEDFATYYCQQFTSSPFTFGQGTKLEIKGGGGSD |
| | | KTHTCPPCPAPELLGGPSVFLFPPKPKDTLMISRT |
| | | PEVTCVVVDVSHEDPEVKFNWYVDGVEVHNAK |
| | | TKPREEQYNSTYRVVSVLTVLHQDWLNGKEYKC |
| | | KVSNKALPAPIEKTISKAKGQPREPQVCTLPPSRE |
| | | EMTKNQVSLSCAVKGFYPSDIAVEWESNGQPEN |
| | | NYKTTPPVLDSDGSFFLVSKLTVDKSRWQQGNV |
| | | FSCSVMHEALHNHYTQKSLSLSPGKGGGGSGGG |
| | | GSGLNDIFEAQKIEWHE |
| SEQ ID NO: 3642 | scFv | EVQLVESGGGLVQPGGSLRLSCAASGFTFSNFGM |
| | | HWVRQAPGKGLEWVSYISSGSSTIYYADTLKGRF |
| | | TISRDNSKNTLYLQMNSLRAEDTAVYYCARRGE |
| | | GAMDYWGQGTTVTVSSGGGGSGGGGSGGGGSG |
| | | GGGSDNQLTQSPSFLSASVGDRVTITCRASSSVN |
| | | YIYWYQQKPGKAPKLLIYYTSNLAPGVPSRFSGS |
| | | GSGNEYTLTISSLQPEDFATYYCQQFTSSPFTFGQ |
| | | GTKLEIK |

Antibody B-H.2

| | | |
|---|---|---|
| SEQ ID NO: 1338 | scFv | EVQLVESGGGLVQPGGSLRLSCAASGFTFSNFGM |
| | | HWVRQAPGKGLEWVSYISSGSSTIYYADTLKGRF |
| | | TISRDNSKNTLYLQMNSLRAEDTAVYYCARRGE |
| | | GAMDYWGQGTTVTVSSGGGGSGGGGSGGGGSG |
| | | GGGSDNQLTQSPSSLSASVGDRVTITCRASSSVN |
| | | YIYWYQQKPGKAPKLLIYYTSNLAPGVPSRFSGS |
| | | GSGNDYTLTISSLQPEDFATYYCQQFTSSPFTFGQ |
| | | GTKLEIK |

Antibody B-H.3

| | | |
|---|---|---|
| SEQ ID NO: 1339 | scFv | EVQLVESGGGLVQPGGSLRLSCAASGFTFSNFGM |
| | | HWVRQAPGKGLEWVSYISSGSSTIYYADTLKGRF |
| | | TISRDNSKNTLYLQMNSLRAEDTAVYYCARRGE |
| | | GAMDYWGQGTTVTVSSGGGGSGGGGSGGGGSG |
| | | GGGSSNELTQPPSVSVSPGQTARITCRASSSVNYI |
| | | YWYQQKSGQAPVLVIYYTSNLAPGIPERFSGSGS |
| | | GNMYTLTISGAQVEDEADYYCQQFTSSPFTFGTG |
| | | TKVTVL |

Antibody B-H.4

| | | |
|---|---|---|
| SEQ ID NO: 1340 | scFv | QVQLVESGGGVVQPGRSLRLSCAASGFTFSNFG |
| | | MHWVRQAPGKGLEWVAYISSGSSTIYYADTLKG |
| | | RFTISRDNSKNTLYLQMNSLRAEDTAVYYCARR |
| | | GEGAMDYWGQGTTVTVSSGGGGSGGGGSGGGG |
| | | SGGGGSDNQLTQSPSFLSASVGDRVTITCRASSSV |
| | | NYIYWYQQKPGKAPKLLIYYTSNLAPGVPSRFSG |
| | | SGSGNEYTLTISSLQPEDFATYYCQQFTSSPFTFG |
| | | QGTKLEIK |

Antibody B-H.5

| | | |
|---|---|---|
| SEQ ID NO: 1341 | scFv | QVQLVESGGGVVQPGRSLRLSCAASGFTFSNFG |
| | | MHWVRQAPGKGLEWVAYISSGSSTIYYADTLKG |
| | | RFTISRDNSKNTLYLQMNSLRAEDTAVYYCARR |
| | | GEGAMDYWGQGTTVTVSSGGGGSGGGGSGGGG |
| | | SGGGGSDNQLTQSPSSLSASVGDRVTITCRASSSV |
| | | NYIYWYQQKPGKAPKLLIYYTSNLAPGVPSRFSG |
| | | SGSGNDYTLTISSLQPEDFATYYCQQFTSSPFTFG |
| | | QGTKLEIK |

Antibody B-H.6

| | | |
|---|---|---|
| SEQ ID NO: 1342 | scFv | QVQLVESGGGVVQPGRSLRLSCAASGFTFSNFG |
| | | MHWVRQAPGKGLEWVAYISSGSSTIYYADTLKG |
| | | RFTISRDNSKNTLYLQMNSLRAEDTAVYYCARR |
| | | GEGAMDYWGQGTTVTVSSGGGGSGGGGSGGGG |
| | | SGGGGSSNELTQPPSVSVSPGQTARITCRASSSVN |
| | | YIYWYQQKSGQAPVLVIYYTSNLAPGIPERFSGS |
| | | GSGNMYTLTISGAQVEDEADYYCQQFTSSPFTFG |
| | | TGTKVTVL |

TABLE 3
Constant region amino acid sequences of human IgG heavy chains and human kappa
light chain

| | | |
|---|---|---|
| Human kappa | LC | RTVAAPSVFI FPPSDEQLKS GTASVVCLLN NFYPREAKVQ |
| constant region | | WKVDNALQSG NSQESVTEQD SKDSTYSLSS TLTLSKADYE |
| SEQ ID NO: 39 | | KHKVYACEVT HQGLSSPVTK SFNRGEC |
| Human | LC | RTVAAPSVFIFPPSDEQLKSGTASVVCLLNNFYPREAKVQWKVDNALQ |
| Immunoglobulin | | SGNSQESVTEQDSKDSTYSLSSTLTLSKADYEKHKVYACEVTHQGLSSP |
| kappa constant | | VTKSFNRGEC |
| C region | | |
| SEQ ID NO: | | |
| 3644 | | |
| IgG4 (S228P) | HC | ASTKGPSVFPLAPCSRSTSESTAALGCLVKDYFPEPVTVSWNSGALTSG |
| mutant constant | | VHTFPAVLQSSGLYSLSSVVTVPSSSLGTKTYTCNVDHKPSNTKVDKR |
| region (EU | | VESKYGPPCPPCPAPEFLGGPSVFLFPPKPKDTLMISRTPEVTCVVVDVS |
| Numbering) | | QEDPEVQFNWYVDGVEVHNAKTKPREEQFNSTYRVVSVLTVLHQDW |
| SEQ ID NO: 40 | | LNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLT |
| | | VDKSRWQEGNVFSCSVMHEALHNHYTQKSLSLSLG |
| IgG1 wild type | HC | ASTKGPSVFPLAPSSKSTSGGTAALGCLVKDYFPEPVTVSWNSGALTSG |
| SEQ ID NO: 41 | | VHTFPAVLQSSGLYSLSSVVTVPSSSLGTQTYICNVNHKPSNTKVDKRV |
| | | EPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| IgG1 (N297A) | HC | ASTKGPSVFPLAPSSKSTSGGTAALGCLVKDYFPEPVTVSWNSGALTSG |
| mutant constant | | VHTFPAVLQSSGLYSLSSVVTVPSSSLGTQTYICNVNHKPSNTKVDKRV |
| region (EU | | EPKSCDKTHTCPPCPAPELLGGPSVFLFPPKPKDTLMISRTPEVTCVVVD |
| Numbering) | | VSHEDPEVKFNWYVDGVEVHNAKTKPREEQYASTYRVVSVLTVLHQ |
| SEQ ID NO: 42 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| IgM constant | HC | GSASAPTLFPLVSCENSPSDTSSVAVGCLAQDFLPDSITFSWKYKNNSDI |
| delta CDC | | SSTRGFPSVLRGGKYAATSQVLLPSKDVMQGTDEHVVCKVQHPNGNK |
| (P311A, P313S) | | EKNVPLPVIAELPPKVSVFVPPRDGFFGNPRKSKLICQATGFSPRQIQVS |
| SEQ ID NO: 73 | | WLREGKQVGSGVTTDQVQAEAKESGPTTYKVTSTLTIKESDWLGQSM |
| | | FTCRVDHRGLTFQQNASSMCVPDQDTAIRVFAIPPSFASIFLTKSTKLTC |
| | | LVTDLTTYDSVTISWTRQNGEAVKTHTNISESHPNATFSAVGEASICED |
| | | DWNSGERFTCTVTHTDLASSLKQTISRPKGVALHRPDVYLLPPAREQL |
| | | NLRESATITCLVTGFSPADVFVQWMQRGQPLSPEKYVTSAPMPEPQAP |
| | | GRYFAHSILTVSEEEWNTGETYTCVVAHEALPNRVTERTVDKSTGKPT |
| | | LYNVSLVMSDTAGTCY |
| IgGA1 | HC | ASPTSPKVFPLSLCSTQPDGNVVIACLVQGFFPQEPLSVTWSESGQGVT |
| SEQ ID NO: 74 | | ARNFPPSQDASGDLYTTSSQLTLPATQCLAGKSVTCHVKHYTNPSQDV |
| | | TVPCPVPSTPPTPSPSTPPTPSPSCCHPRLSLHRPALEDLLLGSEANLTCT |
| | | LTGLRDASGVTFTWTPSSGKSAVQGPPERDLCGCYSVSSVLPGCAEPW |
| | | NHGKTFTCTAAYPESKTPLTATLSKSGNTFRPEVHLLPPPSEELALNELV |
| | | TLTCLARGFSPKDVLVRWLQGSQELPREKYLTWASRQEPSQGTTTFAV |
| | | TSILRVAAEDWKKGDTFSCMVGHEALPLAFTQKTIDRLAGKPTHVNVS |
| | | VVMAEVDGTCY |
| IgGA2 | HC | ASPTSPKVFPLSLDSTPQDGNVVVACLVQGFFPQEPLSVTWSESGQNVT |
| SEQ ID NO: 75 | | ARNFPPSQDASGDLYTTSSQLTLPATQCPDGKSVTCHVKHYTNSSQDV |
| | | TVPCRVPPPPPCCHPRLSLHRPALEDLLLGSEANLTCTLTGLRDASGATF |
| | | TWTPSSGKSAVQGPPERDLCGCYSVSSVLPGCAQPWNHGETFTCTAAH |
| | | PELKTPLTANITKSGNTFRPEVHLLPPPSEELALNELVTLTCLARGFSPK |
| | | DVLVRWLQGSQELPREKYLTWASRQEPSQGTTTYAVTSILRVAAEDW |
| | | KKGETFSCMVGHEALPLAFTQKTIDRMAGKPTHINVSVVMAEADGTC |
| | | Y |
| Human Ig_J | HC | MKNHLLFWGVLAVFIKAVHVKAQEDERIVLVDNKCKCARITSRIIRSSE |
| chain | | DPNEDIVERNIRIIVPLNNRENISDPTSPLRTRFVYHLSDLCKKCDPTEVE |
| SEQ ID NO: 76 | | LDNQIVTATQSNICDEDSATETCYTYDRNKCYTAVVPLVYGGETKMVE |
| | | TALTPDACYPD |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKKV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYASTYRVVSVLTVLHQ |
| 3645 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKKV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYASTYRVVSVLTVLHQ |
| 3646 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGKEPEA |
| Human | HC | ASTKGPSVFPLAPSSKSTSGGTAALGCLVKDYFPEPVTVSWNSGALTSG |
| Immunoglobulin | | VHTFPAVLQSSGLYSLSSVVTVPSSSLGTQTYICNVNHKPSNTKVDKRV |
| heavy chain | | EPKSCDKTHTCPPCPAPELLGGPSVFLFPPKPKDTLMISRTPEVTCVVVD |
| SEQ ID NO: | | VSHEDPEVKFNWYVDGVEVHNAKTKPREEQYNSTYRVVSVLTVLHQ |
| 3647 | | DWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| Human Fc Knob | HC | DKTHTCPPCPAPELLGGPSVFLEPPKPKDTLMISRTPEVTCVVVDVSHE |
| cys N297A | | DPEVKFNWYVDGVEVHNAKTKPREEQYASTYRVVSVLTVLHQDWLN |
| SEQ ID NO: | | GKEYKCKVSNKALPAPIEKTISKAKGQPREPQVYTLPPCREEMTKNQV |
| 3648 | | SLWCLVKGFYPSDIAVEWESNGQPENNYKTTPPVLDSDGSFFLYSKLT |
| | | VDKSRWQQGNVFSCSVMHEALHNHYTQKSLSLSPGK |
| Human IgG1 | HC | ASTKGPSVFPLAPSSKSTSGGTAALGCLVKDYFPEPVTVSWNSGALTSG |
| hole cys N297A | | VHTFPAVLQSSGLYSLSSVVTVPSSSLGTQTYICNVNHKPSNTKVDKRV |
| SEQ ID NO: | | EPKSCDKTHTCPPCPAPELLGGPSVFLFPPKPKDTLMISRTPEVTCVVVD |
| 3649 | | VSHEDPEVKFNWYVDGVEVHNAKTKPREEQYASTYRVVSVLTVLHQ |
| | | DWLNGKEYKCKVSNKALPAPIEKTISKAKGQPREPQVCTLPPSREEMT |
| | | KNQVSLSCAVKGFYPSDIAVEWESNGQPENNYKTTPPVLDSDGSFFLV |
| | | SKLTVDKSRWQQGNVFSCSVMHEALHNHYTQKSLSLSPGK |

TABLE 4
Exemplary Fc KiH mutations and optional Cysteine mutations

| | Knob | Hole |
|---|---|---|
| Position | Mutation | Mutation |
| T366 | T366W | T366S |
| L368 | — | L368A |
| Y407 | — | Y407V |

Additional Cysteine Mutations to form
a stabilizing disulfide bridge

| Position | Knob CH3 | Hole CH3 |
|---|---|---|
| S354 | S354C | — |
| Y349 | — | Y349C |

TABLE 5
TCRαV- and TCRβV-and related diseases or conditions

| Subfamily | Related conditions or diseases |
|---|---|
| TCRαV1 (including TCRαV1-1 and TCRα1-2) | multiple sclerosis |
| TCRαV2 | Crohn's disease |
| TCRαV4 | Celiac disease |
| TCRαV12 (TCRαV12-3) | Type I diabetes |
| TCRαV13 (including TCRαV13-1, TCRαV13-2) | Sjogren's syndrome |
| TCRαV20 | Celiac disease |
| TCRαV21 | ankylosing spondylitis |
| TCRαV22 | Crohn's disease |
| TCRαV26 (including TCRαV26-1, and | Celiac disease |
| TCRαV26-2) | |
| TCRαV40 | Crohn's disease |
| TCRαV41 | Joint implant failure |
| TCRβV4 | Celiac disease |
| TCRβV6 (including TCRβV6-1, TCRβV6-2/3, | Primary Sjogren's syndrome |
| and TCRβV6-5) | |
| TCRβV7 (including TCRβV7-1 and TCRβV7- | Amyotrophic lateral sclerosis; celiac disease; |
| 8/9) | multiple sclerosis |
| TCRβV9 | Ankylosing spondylitis |
| TCRβV10 (including TCRβV10-3) | Multiple sclerosis |
| TCRβV11 (including TCRβV11-2) | Covid-induced multisystem inflammatory |
| | syndrome in children |
| TCRβV12 (including TCRβV12-3/4) | Type I diabetes |
| TCRβV20 (including TCRβV20-1) | Multiple sclerosis |
| TCRβV23 | Amyotrophic lateral sclerosis |
| TCRβV29 (including TCRβV29-1) | Multiple sclerosis |

References:
He et al., (2012) Neoplasma. 59(6): 693-9
Howson et al. (2018) Nat. Commun. 9, 253
Tastan et al. (2018) Mucosal Immunol. 11, 1591-1605
Carnero et al., (2019) Front. Immunol. 10, 2690
Huang et al. (2019) Proc. Natl. Acad. Sci. U.S.A. 116, 8995-9001
Hong et al., (2022) Front Cell Infect Microbiol. 10; 12: 932373
Tan et al., (2010) Hematology, 15: 2, 81-87
Gedda et al., (2022) J Transl Med. 20(1): 587
Meermeier et al. (2016) Nat. Commun. 7, 12506
Yang et al., J Biol Chem. 2017 Nov. 10; 292(45): 18618-18627.
Surman et al., J Immunol. 2011 Jul. 15; 187(2): 835-41.
Motozono et al., 2014 Apr. 1; 192(7): 3428-34.
Brennan et al., (2007) J Virol. (13): 7269-73.
Huda et al., (2021) Leuk Lymphoma. 62(7): 1711-1720.
Petersen et al., 2016 Oct. 4; 24(10): 1643-1657.
Benati et al., J Clin Invest. 2016 Jun. 1; 126(6): 2093-108.
Frick et al., (2020) Eur J Immunol. 50(1): 142-145.
Valkenburg et al., (2016) Proc Natl Acad Sci USA. 113(16): 4440-5.
Lepore et al., (2017) Elife 6, e24476
Zhou et al., (2020) Cancer Cell Int. 20: 240.
Rowntree et al., (2020) J immunol; 205(6), 1524-1534
Crowther et al., (2020) Nat. Immunol. 21, 178-185
Xu et al., (2022) J Oncol. 3152114
Chen et al., (2021) Int J Mol Sci. 22(5): 2428
Bovay et al., (2018) Eur J Immunol. 48(2): 258-272.
Godfrey et al., (2015) Nat Immunol. 16(11): 1114-23.
Cole et al., (2009) J Biol Chem. 284(40): 27281-9.
Szeto et al., (2021) Cells. 2021 Oct. 3; 10(10): 2646.
Campisi et al., (2022) Nature. 606: 945-952.
Koirala et al., (2016) Mucosal Immunology; 9(3): 587-596.
Yohannes et al., (2017) Sci. Rep.; 7(17977).
Porritt et al., (2021) J Clin Invest.; 131(10): e146614.
Lu et al., (2022) eBioMedicine; 84: 104252.
Helliwell et al., (2005) Ann Rheum Dis; 64: 468-470.
Lossius et al., (2014) Eur. J. Immunol.; 44: 3439-3452.
Bulek et al., (2012) Nature Immunology; 13(3): 283-289.

TABLE 6
List of TCRβV subfamilies and subfamily members

| Subfamily | Subfamily members |
|---|---|
| TCRβ V6 | TCRβ V6-4*01, TCRβ V6-4*02, TCRβ V6-9*01, TCRβ V6-8*01, |
| Also referred to as: | TCRβ V6-5*01, TCRβ V6-6*02, TCRβ V6-6*01, TCRβ V6-2*01, |
| TCR VB 13.1 | TCRβ V6-3*01 or TCRβ V6-1*01. |
| TCRβ V10 | TCRβ V10-1*01, TCRβ V10-1*02, TCRβ V10-3*01 or TCRβ V10- |
| Also referred to as: | 2*01 |
| TCRβ V12 | |
| TCRβ V12 | TCRβ V12-4*01, TCRβ V12-3*01, or TCRβ V12-5*01 |
| Also referred to as: | |
| TCRβ V8.1 | |
| TCRβ V5 | TCRβ V5-5*01, TCRβ V5-6*01, TCRβ V5-4*01, TCRβ V5-8*01, |
| | TCRβ V5-1*01 |
| TCRβ V7 | TCRβ V7-7*01, TCRβ V7-6*01, TCRβ V7 -8*02, TCRβ V7 -4*01, |
| | TCRβ V7-2*02, TCRβ V7-2*03, TCRβ V7-2*01, TCRβ V7-3*01, |
| | TCRβ V7-9*03, or TCRβ V7-9*01 |
| TCRβ V11 | TCRβ V11-1*01, TCRβ V11-2*01 or TCRβ V11-3*01 |
| TCRβ V14 | TCRβ V14*01 |
| TCRβ V16 | TCRβ V16*01 |
| TCRβ V18 | TCRβ V18*01 |
| TCRβ V9 | TCRβ V9*01 or TCRβ V9*02 |
| TCRβ V13 | TCRβ V13*01 |
| TCRβ V4 | TCRβ V4-2*01, TCRβ V4-3*01, or TCRβ V4-1*01 |
| TCRβ V3 | TCRβ V3-1*01 |
| TCRβ V2 | TCRβ V2*01 |
| TCRβ V15 | TCRβ V15*01 |
| TCRβ V30 | TCRβ V30*01, or TCRβ V30*02 |
| TCRβ V19 | TCRβ V19*01, or TCRβ V19*02 |
| TCRβ V27 | TCRβ V27*01. |
| TCRβ V28 | TCRβ V28*01. |
| TCRβ V24 | TCRβ V24-1*01 |
| TCRβ V20 | TCRβ V20-1*01, or TCRβ V20-1*02 |
| TCRβ V25 | TCRβ V25-1*01 |
| TCRβ V29 | TCRβ V29-1*01 |

TABLE 7
Additional TCRβV subfamilies
Subfamily

TCRβ V1
TCRβ V17
TCRβ V21
TCRβ V23
TCRβ V26

TABLE 8
Alignment of TCRBV amino acid sequences (SEQ ID NOS 3457-3516, respectively, in
order of appearance)

| | FR1-IMGT CDR1-IMGT FR2-IMGT CDR2-IMGT FR3-IMGT CDR3-IMGT |
|---|---|
| | (1-26) (27-38) (39-55) (56-65) (66-104) (105-117) |
| Gene | A B BC C C′ C′C″ C″ D E F FG |
| | (1-15) (16-26) (27-38) (39-46) (47-55) (56-65) (66-74) (75-84) (85-96) |
| | (97-104) |
| | -------------------->------------------------->-------------->--- |
| | --------------->---------------->---------------------->----------- |
| | ----->------------------> |
| | 1 10 15 16 23 26 27 38 3941 46 47 55 56 65 66 74 75 84 85 89 96 97 |
| | 104 105 |
| | \|........\|....\|\|......\|..\|\|..........\|\|.\|....\|\|......\|\|........\|\|.......\| |
| | \|........\|\|...\|.....\|\|......\|\|..... |
| TRBV | DTGITQTPKYLVTAMGSKRTMKREHLGH........DSMYWYRQKAKKSLEFMFY |
| 1 | YNC....KEFIENKTVP.NHFTPECP.DSSRLYLHVVALQQEDSAAYLCTSSQ |
| TRBV | EPEVTQTPSHQVTQMGQEVILRCVPISNH.......LYFYWYRQILGQKVEFLVS |
| 2 | FYN....NEISEKSEIFDDQFSVERP.DGSNFTLKIRSTKLEDSAMYFCASSE |
| TRBV | DTAVSQTPKYLVTQMGNDKSIKCEQNLGH.......DTMYWYKQDSKKFLKIMFS |
| 3-1 | YNN....KELIINETVP.NRFSPKSP.DKAHLNLHINSLELGDSAVYFCASSQ |
| TRBV | DTAVSQTPKYLVTQMGKKESLK*EQNLGH.......NAMYWYKQDSKKFLKTMFI |
| 3-2 | YSN....KEPILNETVP.NRFSPDSP.DKAHLNLHINSLELGDSAVYFCASSQ |
| TRBV | DTEVTQTPKHLVMGMTNKKSLKCEQHMGH.......RAMYWYKQKAKKPPELMFV |
| 4-1 | YSY....EKLSINESVP.SRFSPECP.NSSLLNLHLHALQPEDSALYLCASSQ |
| TRBV | ETGVTQTPRHLVMGMTNKKSLKCEQHLGH.......NAMYWYKQSAKKPLELMFV |
| 4-2 | YNF....KEQTENNSVP.SRFSPECP.NSSHLFLHLHTLQPEDSALYLCASSQ |
| TRBV | ETGVTQTPRHLVMGMTNKKSLKCEQHLGH.......NAMYWYKQSAKKPLELMFV |
| 4-3 | YSL....EERVENNSVP.SRFSPECP.NSSHLFLHLHTLQPEDSALYLCASSQ |
| TRBV | KAGVTQTPRYLIKTRGQQVTLSCSPISGH.......RSVSWYQQTPGQGLQFLFE |
| 5-1 | YFS....ETQRNKGNFP.GRFSGRQF.SNSRSEMNVSTLELGDSALYLCASSL |
| TRBV | EAGVTQSPTHLIKTRGQQVTLRCSPISGH.......SSVSWYQQAPGQGPQFIFE |
| 5-3 | YAN....ELRRSEGNFP.NRFSGRQF.HDCCSEMNVSALELGDSALYLCARSL |
| TRBV | ETGVTQSPTHLIKTRGQQVTLRCSSQSGH.......NTVSWYQQALGQGPQFIFQ |
| 5-4 | YYR....EEENGRGNFP.PRFSGLQF.PNYSSELNVNALELDDSALYLCASSL |
| TRBV | DAGVTQSPTHLIKTRGQQVTLRCSPISGH.......KSVSWYQQVLGQGPQFIFQ |
| 5-5 | YYE....KEERGRGNFP.DRFSARQF.PNYSSELNVNALLLGDSALYLCASSL |
| TRBV | DAGVTQSPTHLIKTRGQQVTLRCSPKSGH.......DTVSWYQQALGQGPQFIFQ |
| 5-6 | YYE....EEERQRGNFP.DRFSGHQF.PNYSSELNVNALLLGDSALYLCASSL |
| TRBV | DAGVTQSPTHLIKTRGQHVTLRCSPISGH.......TSVSSYQQALGQGPQFIFQ |
| 5-7 | YYE....KEERGRGNFP.DQFSGHQF.PNYSSELNVNALLLGDSALYLCASSL |
| TRBV | EAGVTQSPTHLIKTRGQQATLRCSPISGH.......TSVYWYQQALGLGLQFLLW |
| 5-8 | YDE....GEERNRGNFP.PRFSGRQF.PNYSSELNVNALELEDSALYLCASSL |
| TRBV | NAGVTQTPKFQVLKTGQSMTLQCAQDMNH.......NSMYWYRQDPGMGLRLIYY |
| 6-1 | SAS....EGTTDKGEVP.NGYNVSRL.NKREFSLRLESAAPSQTSVYFCASSE |
| TRBV | NAGVTQTPKFRVLKTGQSMTLLCAQDMNH..EYMYWYRQDPGMGLRLIHY |
| 6-2 | SVG....EGTTAKGEVP.DGYNVSRL.KKQNFLLGLESAAPSQTSVYFCASSY |
| TRBV | NAGVTQTPKFRVLKTGQSMTLLCAQDMNH.......EYMYWYRQDPGMGLRLIHY |
| 6-3 | SVG....EGTTAKGEVP.DGYNVSRL.KKQNFLLGLESAAPSQTSVYFCASSY |
| TRBV | IAGITQAPTSQILAAGRRMTLRCTQDMRH.......NAMYWYRQDLGLGLRLIHY |
| 6-4 | SNT....AGTTGKGEVP.DGYSVSRA.NTDDFPLTLASAVPSQTSVYFCASSD |
| TRBV | NAGVTQTPKFQVLKTGQSMTLQCAQDMNH.......EYMSWYRQDPGMGLRLIHY |
| 6-5 | SVG....AGITDQGEVP.NGYNVSRS.TTEDFPLRLLSAAPSQTSVYFCASSY |
| TRBV | NAGVTQTPKFRILKIGQSMTLQCTQDMNH.......NYMYWYRQDPGMGLKLIYY |
| 6-6 | SVG....AGITDKGEVP.NGYNVSRS.TTEDFPLRLELAAPSQTSVYFCASSY |
| TRBV | NAGVTQTPKFHVLKTGQSMTLLCAQDMNH.......EYMYRYRQDPGKGLRLIYY |
| 6-7 | SVA....AALTDKGEVP.NGYNVSRS.NTEDFPLKLESAAPSQTSVYFCASSY |
| TRBV | NAGVTQTPKFHILKTGQSMTLQCAQDMNH.......GYMSWYRQDPGMGLRLIYY |
| 6-8 | SAA....AGTTDK.EVP.NGYNVSRL.NTEDFPLRLVSAAPSQTSVYLCASSY |
| TRBV | NAGVTQTPKFHILKTGQSMTLQCAQDMNH.......GYLSWYRQDPGMGLRRIHY |
| 6-9 | SVA....AGITDKGEVP.DGYNVSRS.NTEDFPLRLESAAPSQTSVYFCASSY |
| TRBV | GAGVSQSLRHKVAKKGKDVALRYDPISGH.......NALYWYRQSLGQGLEFPIY |
| 7-1 | FQG....KDAADKSGLPRDRFSAQRS.EGSISTLKFQRTQQGDLAVYLCASSS |
| TRBV | GAGVSQSPSNKVTEKGKDVELRCDPISGH.......TALYWYRQSLGQGLEFLIY |
| 7-2 | FQG....NSAPDKSGLPSDRFSAERT.GGSVSTLTIQRTQQEDSAVYLCASSL |
| TRBV | GAGVSQTPSNKVTEKGKYVELRCDPISGH.......TALYWYRQSLGQGPEFLIY |
| 7-3 | FQG....TGAADDSGLPNDRFFAVRP.EGSVSTLKIQRTERGDSAVYLCASSL |
| TRBV | GAGVSQSPRYKVAKRGRDVALRCDSISGH.......VTLYWYRQTLGQGSEVLTY |
| 7-4 | SQS....DAQRDKSGRPSGRFSAERP.ERSVSTLKIQRTEQGDSAVYLCASSL |
| TRBV | GAGVSQSPRYKVTKRGQDVALRCDPISGH......VSLYWYRQALGQGPEFLTY |
| 7-6 | FNY....EAQQDKSGLPNDRFSAERP.EGSISTLTIQRTEQRDSAMYRCASSL |
| TRBV | GAGVSQSPRYKVTKRGQDVTLRCDPISSH.......ATLYWYQQALGQGPEFLTY |
| 7-7 | FNY....EAQPDKSGLPSDRFSAERP.EGSISTLTIQRTEQRDSAMYRCASSL |
| TRBV | GAGVSQSPRYKVAKRGQDVALRCDPISGH....VSLFWYQQALGQGPEFLTY |
| 7-8 | FQN....EAQLDKSGLPSDRFFAERP.EGSVSTLKIQRTQQEDSAVYLCASSL |
| TRBV | DTGVSQNPRHKITKRGQNVTFRCDPISEH.......NRLYWYRQTLGQGPEFLTY |
| 7-9 | FQN....EAQLEKSRLLSDRFSAERP.KGSFSTLEIQRTEQGDSAMYLCASSL |
| TRBV | DSGVTQTPKHLITATGQRVTLRCSPRSGD.......LSVYWYQQSLDQGLQFLIQ |
| 9 | YYN....GEERAKGNIL.ERFSAQQF.PDLHSELNLSSLELGDSALYFCASSV |
| TRBV | DAEITQSPRHKITETGRQVTLACHQTWNH.......NNMFWYRQDLGHGLRLIHY |
| 10-1 | SYG....VQDTNKGEVS.DGYSVSRS.NTEDLPLTLESAASSQTSVYFCASSE |
| TRBV | DAGITQSPRYKITETGRQVTLMCHQTWSH.......SYMFWYRQDLGHGLRLIYY |
| 10-2 | SAA....ADITDKGEVP.DGYVVSRS.KTENFPLTLESATRSQTSVYFCASSE |
| TRBV | DAGITQSPRHKVTETGTPVTLRCHQTENH.......RYMYWYRQDPGHGLRLIHY |
| 10-3 | SYG....VKDTDKGEVS.DGYSVSRS.KTEDFLLTLESATSSQTSVYFCAISE |
| TRBV | EAEVAQSPRYKITEKSQAVAFWCDPISGH.......ATLYWYRQILGQGPELLVQ |
| 11-1 | FQD....ESVVDDSQLPKDRFSAERL.KGVDSTLKIQPAELGDSAMYLCASSL |
| TRBV | EAGVAQSPRYKIIEKRQSVAFWCNPISGH.......ATLYWYQQILGQGPKLLIQ |
| 11-2 | FQN....NGVVDDSQLPKDRFSAERL.KGVDSTLKIQPAKLEDSAVYLCASSL |
| TRBV | EAGVVQSPRYKIIEKKQPVAFWCNPISGH.......NTLYWYLQNLGQGPELLIR |
| 11-3 | YEN....EEAVDDSQLPKDRFSAERL.KGVDSTLKIQPAELGDSAVYLCASSL |
| TRBV | DAGVIQSPRHKVTEMGQSVTLRCEPISGH.......NDLLWYRQTFVQGLELLNY |
| 12-1 | FCS....WTLVDDSGVSKD*FSAQMP.DVSFSTLRIQPMEPRDLGLYFCASSF |
| TRBV | DAGIIQSPKHEVTEMGQTVTLRCEPIFGH.......NFLFWYRDTFVQGLELLSYFRS....*SI |
| 12-2 | IDNAGMPTERFSAERP.DGSFSTLKIQPAEQGDSAVYVCASRL |
| TRBV | DAGVIQSPRHEVTEMGQEVTLRCKPISGH.......NSLFWYRQTMMRGLELLIY |
| 12-3 | FNN....NVPIDDSGMPEDRFSAKMP.NASFSTLKIQPSEPRDSAVYFCASSL |
| TRBV | DAGVIQSPRHEVTEMGQEVTLRCKPISGH.......DYLFWYRQTMMRGLELLIY |
| 12-4 | FNN....NVPIDDSGMPEDRFSAKMP.NASFSTLKIQPSEPRDSAVYFCASSL |
| TRBV | DARVTQTPRHKVTEMGQEVTMRCQPILGH.......NTVFWYRQTMMQGLELLAY |
| 12-5 | FRN....RAPLDDSGMPKDRFSAEMP.DATLATLKIQPSEPRDSAVYFCASGL |
| TRBV | AAGVIQSPRHLIKEKRETATLKCYPIPRH.......DTVYWYQQGPGQDPQFLIS |
| 13 | FYE....KMQSDKGSIP.DRFSAQQF.SDYHSELNMSSLELGDSALYFCASSL |
| TRBV | EAGVTQFPSHSVIEKGQTVTLRCDPISGH.......DNLYWYRRVMGKEIKFLLH |
| 14 | FVK....ESKQDESGMPNNRFLAERT.GGTYSTLKVQPAELEDSGVYFCASSQ |
| TRBV | DAMVIQNPRYQVTQFGKPVTLSCSQTLNH.......NVMYWYQQKSSQAPKLLFH |
| 15 | YYD....KDFNNEADTP.DNFQSRRP.NTSFCFLDIRSPGLGDTAMYLCATSR |
| TRBV | GEEVAQTPKHLVRGEGQKAKLYCAPIKGH.......SYVFWYQQVLKNEFKFLIS |
| 16 | FQN....ENVFDETGMPKERFSAKCL.PNSPCSLEIQATKLEDSAVYFCASSQ |
| TRBV | EPGVSQTPRHKVTNMGQEVILRCDPSSGH.......MFVHWYRQNLRQEMKLLIS |
| 17 | FQY....QNIAVDSGMPKERFTAERP.NGTSSTLKIHPAEPRDSAVYLYSSG |
| TRBV | NAGVMQNPRHLVRRRGQEARLRCSPMKGH......SHVYWYRQLPEEGLKFMVY |
| 18 | LQK....ENIIDESGMPKERFSAEFP.KEGPSILRIQQVVRGDSAAYFCASSP |
| TRBV | DGGITQSPKYLFRKEGQNVTLSCEQNLNH.......DAMYWYRQDPGQGLRLIYY |
| 19 | SQI....VNDFQKGDIA.EGYSVSRE.KKESFPLTVTSAQKNPTAFYLCASSI |
| TRBV | GAVVSQHPSWVICKSGTSVKIECRSLDFQ......ATTMFWYRQFPKQSLMLMAT |
| 20-1 | SNEG...SKATYEQGVEKDKFLINHA.SLTLSTLTVTSAHPEDSSFYICSAR |
| TRBV | DTKVTQRPRLLVKASEQKAKMDCVPIKAH.......SYVYWYRKKLEEELKFLVY |
| 21-1 | FQN....EELIQKAEIINERFLAQCS.KNSSCTLEIQSTESGDTALYFCASSK |
| TRBV | HAKVTQTPGHLVKGKGQKTKMDCTPEKGH.......TFVYWYQQNQNKEFMLLIS |
| 23-1 | FQN....EQVLQETEMHKKRFSSQCP.KNAPCSLAILSSEPGDTALYLCASSQ |
| TRBV | DADVTQTPRNRITKTGKRIMLECSQTKGH.......DRMYWYRQDPGLGLRLIYY |
| 24-1 | SFD....VKDINKGEIS.DGYSVSRQ.AQAKFSLSLESAIPNQTALYFCATSDL |
| TRBV | EADIYQTPRYLVIGTGKKITLECSQTMGH.......DKMYWYQQDPGMELHLIHY |
| 25-1 | SYG....VNSTEKGDLS.SESTVSRI.RTEHFPLTLESARPSHTSQYLCASSE |
| TRBV | DAVVTQFPRHRIIGTGKEFILQCSQNMNH.......VTMYWYRQDPGLGLKLVYY |
| 26 | SPG....TGSTEKGDIS.EGYHVS*N.TIASFPLTLKSASTNQTSVYLYASSS |
| TRBV | EAQVTQNPRYLITVTGKKLTVTCSQNMNH.......EYMSWYRQDPGLGLRQIYY |
| 27 | SMN....VEVTDKGDVP.EGYKVSRK.EKRNFPLILESPSPNQTSLYFCASSL |
| TRBV | DVKVTQSSRYLVKRTGEKVFLECVQDMDH.......ENMFWYRQDPGLGLRLIYF |
| 28 | SYD....VKMKEKGDIP.EGYSVSRE.KKERFSLILESASTNQTSMYLCASSL |
| TRBV | SAVISQKPSRDICQRGTSLTIQCQVDSQV.......TMMFWYRQQPGQSLTLIAT |
| 29-1 | ANQG...SEATYESGFVIDKFPISRP.NLTFSTLTVSNMSPEDSSIYLCSVE |
| TRBV | SQTIHQWPATLVQPVGSPLSLECTVEGTS......NPNLYWYRQAAGRGLQLLFY |
| 30 | SVG.....IGQISSEVP.QNLSASRP.QDRQFILSSKKLLLSDSGFYLCAWS |

TABLE 9
Exemplary anti-TCRβV antibody molecules

| | | Reagents monoclonal antibodies | | |
|---|---|---|---|---|
| TRBV | TRBV | Clone name and | | |
| gene name | allele name | Specificity | Company product | Isotype |
| TRBV2 | TRBV2*01 | IsMMU 546 (TRBV2) | Serotec V BETA 22 | Mouse IgG1 |
| | TRBV2*02 | | Coulter Vbeta22 | |
| | TRBV2*03 | | | |
| TRBV3-1 | TRBV3-1*01 | FIN9 (TRBV3-1) | Serotec Vbeta9 | Mouse |
| | | | Coulter Vbeta9 | IgG2a |
| | TRBV3-1*02 | AMKB1-2 (TRBV3-1) | BD Biosciences Vbeta9 | Mouse IgG1 |
| TRBV4-1 | TRBV4-1*01 | ZOE (TRBV4-1, | Serotec V BETA 7 | Mouse |
| | | TRBV4-2, TRBV4-3) | Coulter Vbeta7 | IgG2a |
| | TRBV4-1*02 | 3G5 (TRBV4-1) | Pierce EndogenV beta 7.1 | Mouse |
| | | | | IgG2b |
| TRBV4-2 | TRBV4-2*01 | ZOE (TRBV4-1, | Serotec V BETA 7 | Mouse |
| | TRBV4-2*02 | TRBV4-2, TRBV4-3) | Coulter Vbeta7 | IgG2a |
| TRBV4-3 | TRBV4-3*01 | ZOE (TRBV4-1, | Serotec V BETA 7 | Mouse |
| | TRBV4-3*02 | TRBV4-2, TRBV4-3) | Coulter Vbeta7 | IgG2a |
| | TRBV4-3*03 | | | |
| | TRBV4-3*04 | ZIZOU4 (TRBV4-3) | Coulter Vbeta7.2 | Mouse |
| | | | | IgG2a |
| TRBV5-1 | TRBV5-1*01 | IMMU157 (TRBV5-1) | Serotec Vbeta5.1 | Mouse |
| | | | Coulter Vbeta5.1 | IgG2a |
| | TRBV5-1*02 | LC4 (TRBV5-1) | Pierce Endogen V beta 5(c) | Mouse IgG1 |
| | | | BD Biosciences Vbeta5(c) | |
| TRBV5-4 | TRBV5-4*01 | | | |
| | TRBV5-4*02 | | | |
| | TRBV5-4*03 | | | |
| | TRBV5-4*04 | | | |
| TRBV5-5 | TRBV5-5*01 | 3D11 (TRBV5-5) | Serotec VBETA5.3 | Mouse IgG1 |
| | | | Coulter Vbeta5.3 | |
| | TRBV5-5*02 | 1C1 (TRBV5-5, TRBV5-6) | Pierce Endogen V beta 5(a) | Mouse IgG1 |
| | | | BD Biosciences Vbeta5(a) | |
| | TRBV5-5*03 | W112 (TRBV5-5) | Pierce Endogen V beta 5(b) | Mouse IgG1 |
| | | | Serotec V beta 5.2/5.3 | |
| | | | BD Biosciences Vbeta5(b) | |
| | | MH3-2 (TRBV5-5, | BD Biosciences Vbeta5 | Mouse |
| | | TRBV5-6) | | IgG2a |
| | | 4H11 (TM27) as | | |
| | | disclosed in U.S. Pat. No. | | |
| | | 5,861,155 | | |
| TRBV5-6 | TRBV5-6*01 | 36213 (TRBV5-6) | Serotec Vbeta5.2 | Mouse IgG1 |
| | | 1C1 (TRBV5-5, TRBV5-6) | BD Biosciences Vbeta5(a) | Mouse IgG1 |
| | | MH3-2 (TRBV5-5, | BD Biosciences Vbeta5 | Mouse IgG2a |
| | | TRBV5-6) | | |
| TRBV5-8 | TRBV5-8*01 | | | |
| | TRBV5-8*02 | | | |
| TRBV6-1 | TRBV6-1*01 | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-2 | TRBV6-2*01 | H132 | Coulter Vbeta13.2 | Mouse IgG1 |
| TRBV6-3 | TRBV6-3*01 | | | |
| TRBV6-4 | TRBV6-4*01 | | | |
| | TRBV6-4*02 | | | |
| TRBV6-5 | TRBV6-5*01 | IMMU 222 (TRBV6-5, | Serotec V BETA 13.1 | Mouse |
| | | TRBV6-6 and TRBV6-9) | Coulter Vbeta13.1 | IgG2b |
| | | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-6 | TRBV6-6*01 | JU-74 (TRBV6-6) | Serotec Vbeta13.6 | Mouse IgG1 |
| | TRBV6-6*02 | JU74.3 (TRBV6-6) | Coulter Vbeta13.6 | |
| | TRBV6-6*03 | | | |
| | TRBV6-6*04 | IMMU 222 (TRBV6-5, | Serotec V BETA 13.1 | Mouse |
| | TRBV6-6*05 | TRBV6-6 and TRBV6-9) | Coulter Vbeta13.1 | IgG2b |
| TRBV6-8 | TRBV6-8*01 | | | |
| TRBV6-9 | TRBV6-9*01 | IMMU 222 (TRBV6-5, | Serotec V BETA 13.1 | Mouse |
| | | TRBV6-6 and TRBV6-9) | Coulter Vbeta13.1 | IgG2b |
| TRBV7-2 | TRBV7-2*01 | OT145 (TRBV7-2) | Pierce Endogen V beta 6.7 | Mouse IgG1 |
| | TRBV7-2*02 | | BD Biosciences Vbeta6.7 | |
| | TRBV7-2*03 | | | |
| | TRBV7-2*04 | | | |
| TRBV7-3 | TRBV7-3*01 | | | |
| | TRBV7-3*04 | | | |
| | TRBV7-3*05 | | | |
| TRBV7-4 | TRBV7-4*01 | | | |
| TRBV7-6 | TRBV7-6*01 | | | |
| | TRBV7-6*02 | | | |
| TRBV7-7 | TRBV7-7*01 | | | |
| | TRBV7-7*02 | | | |
| TRBV7-8 | TRBV7-8*01 | | | |
| | TRBV7-8*02 | | | |
| | TRBV7-8*03 | | | |
| TRBV7-9 | TRBV7-9*01 | | | |
| | TRBV7-9*02 | | | |
| | TRBV7-9*03 | | | |
| | TRVB7-9*04 | | | |
| | TRBV7-9*05 | | | |
| | TRBV7-9*06 | | | |
| | TRBV7-9*07 | | | |
| TRBV9 | TRBV9*01 | BL37.2 (TRBV9) | Serotec Vbeta1 | Rat IgG1 |
| | TRBV9*02 | | Coulter Vbeta1 | |
| | TRBV9*03 | | | |
| TRBV10-1 | TRBV10- | S511 (TRBV10-1, | Pierce Endogen V beta 12 | Mouse |
| | 1*01 | TRBV10-2, TRBV10-3) | BD Biosciences Vbeta12 | IgG2b |
| | TRBV10- | | | |
| | 1*02 | | | |
| TRBV10-2 | TRBV10- | | | |
| | 2*01 | | | |
| | TRBV10- | | | |
| | 2*02 | | | |
| TRBV10-3 | TRBV10- | VER2.32.1 (TRBV10-3) | Serotec Vbeta12 | Mouse |
| | 3*01 | S511 (TRBV10-1, | Coulter Vbeta12 | IgG2a |
| | TRBV10- | TRBV10-2, TRBV10-3) | | |
| | 3*02 | | | |
| | TRBV10- | | Pierce Endogen V beta 12 | Mouse |
| | 3*03 | | BD Biosciences Vbeta12 | IgG2b |
| | TRBV10- | | | |
| | 3*04 | | | |
| TRBV11-1 | TRBV11- | | | |
| | 1*01 | | | |
| TRBV11-2 | TRBV11- | IG125 (TRBV11-2) | Serotec Vbeta21.3 | Mouse |
| | 2*01 | | Coulter Vbeta21.3 | IgG2a |
| | TRBV11- | | | |
| | 2*02 | | | |
| | TRBV11- | | | |
| | 2*03 | | | |
| TRBV11-3 | TRBV11- | | | |
| | 3*01 | | | |
| | TRBV11- | | | |
| | 3*02 | | | |
| | TRBV11- | | | |
| | 3*03 | | | |
| | TRBV11- | | | |
| | 3*04 | | | |
| TRBV12-3 | TRBV12- | 56C5 (TRBV12-3, | Serotec Vbeta8.1/8.2 | Mouse |
| | 3*01 | TRBV12-4) | Coulter Vbeta8 | IgG2a |
| TRBV12-4 | TRBV12- | | Pierce Endogen V beta 8(a) | Mouse |
| | 4*01 | | BD Biosciences Vbeta8 | IgG2b |
| | TRBV12- | 56C5.2 (TRBV12-3, | Pierce Endogen V beta 8(b) | Mouse |
| | 4*02 | TRBV12-4) | | IgG2a |
| | | 16G8 (TRBV12-3, | BD Biosciences | Mouse |
| | | TRBV12-4) | Vbeta8 | IgG2b |
| | | MX-6 (TRBV12-3, | | |
| | | TRBV12-4) | | |
| | | JR2 (TRBV12-3, | | |
| | | TRBV12-4, TRBV12-5) | | |
| TRBV12-5 | TRBV12- | JR2 (TRBV12-3, | BD Biosciences Vbeta8 | Mouse |
| | 5*01 | TRBV12-4, TRBV12-5) | | IgG2b |
| TRBV13 | TRBV13*01 | AF-23 (TRBV13) | Serotec Vbeta23 | Mouse IgG1 |
| | TRBV13*02 | AF23 (TRBV13) | Coulter Vbeta23 | |
| | | AHUT7 (Vbeta23) | BD Biosciences Vbeta23 | |
| TRBV14 | TRBV14*01 | TAMAYA1.2 (TRBV14) | Serotec Vbeta16 | Mouse IgG1 |
| | TRBV14*02 | | Coulter Vbeta16 | |
| TRBV15 | TRBV15*01 | | | |
| | TRBV15*02 | | | |
| | TRBV15*03 | | | |
| TRBV16 | TRBV16*01 | | | |
| | TRBV16*03 | | | |
| TRBV18 | TRBV18*01 | BA62 (TRBV18) | Serotec V BETA 18 | Mouse IgG1 |
| | | BA62.6 (TRBV18) | Coulter Vbeta18 | |
| TRBV19 | TRBV19*01 | C1 (TRBV19) | Pierce Endogen V beta 17 | Mouse IgG1 |
| | | E17.5F3 (TRBV19) | BD Biosciences Vbeta17 | |
| | TRBV19*02 | | Serotec Vbeta17 | Mouse IgG1 |
| | TRBV19*03 | E17.5F3.15.13 | Coulter Vbeta17 | |
| | | (TRBV19) | | |
| TRBV20-1 | TRBV20- | MPB2D5 (TRBV20-1) | Serotec VBETA2 | Mouse IgG1 |
| | 1*01 | | Coulter Vbeta2 | |
| | TRBV20- | | | |
| | 1*02 | | | |
| | TRBV20- | | | |
| | 1*03 | | | |
| | TRBV20- | | | |
| | 1*04 | | | |
| | TRBV20- | | | |
| | 1*05 | | | |
| | TRBV20- | | | |
| | 1*06 | | | |
| | TRBV20- | | | |
| | 1*07 | | | |
| TRBV24-1 | TRBV24- | | | |
| | 1*01 | | | |
| TRBV25-1 | TRBV25- | C21 (TRBV25-1) | Serotec V BETA 11 | Mouse |
| | 1*01 | | Coulter Vbeta11 | IgG2a |
| TRBV27 | TRBV27*01 | CAS1.1.3 (TRBV27) | Serotec Vbeta14 | Mouse IgG1 |
| | | | Coulter Vbeta14 | |
| TRBV28 | TRBV28*01 | CH92 (TRBV28) | Serotec Vbeta3 | Mouse IgM |
| | | | Coulter Vbeta3 | |
| | | 8F10 (TRBV28) | Pierce Endogen V beta 3.1 | Mouse IgG1 |
| | | JOVI-3 (TRBV28) | BD Biosciences Vbeta3 | Mouse IgG2a |
| TRBV29-1 | TRBV29- | WJF24 | Coulter Vbeta4 | Rat IgM |
| | 1*01 | | | |
| | TRBV29- | | | |
| | 1*02 | | | |
| | TRBV29- | | | |
| | 1*03 | | | |
| TRBV30 | TRBV30*01 | ELL1.4 (TRBV30) | Serotec Vbeta20 | Mouse IgG1 |
| | TRBV30*02 | | Coulter Vbeta20 | |
| | TRBV30*04 | | | |
| | TRBV30*05 | | | |

TABLE 10
Amino acid sequences for anti TCRβ V5 antibodies. Amino acid and nucleotide
sequences for murine and humanized antibody molecules which bind to TCRVB 5 (e.g., TCRVB
5-5 or TCRVB 5-6). The amino acid the heavy and light chain CDRs, and the amino acid and
nucleotide sequences of the heavy and light chain variable regions, and the heavy and
light chains are shown.
Murine antibody C, also referred to as 4H11

| | | |
|---|---|---|
| SEQ ID NO: | HC CDR1 (Kabat) | AYGVN |
| 1315 | | |
| SEQ ID NO: | HC CDR2 (Kabat) | MIWGDGNTDYNSALKS |
| 1316 | | |
| SEQ ID NO: 1317 | HC CDR3 (Kabat) | DRVTATLYAMDY |
| SEQ ID NO: | HC CDR1 (Chothia) | GFSLTAY |
| 1318 | | |
| SEQ ID NO: | HC CDR2 (Chothia) | WGDGN |
| 1319 | | |
| SEQ ID NO: | HC CDR3 (Chothia) | DRVTATLYAMDY |
| 1317 | | |
| SEQ ID NO: | HC CDR1 | GFSLTAYGVN |
| 1320 | (Combined) | |
| SEQ ID NO: | HC CDR2 | MIWGDGNTDYNSALKS |
| 1316 | (Combined) | |
| SEQ ID NO: | HC CDR3(Combined) | DRVTATLYAMDY |
| 1317 | | |
| SEQ ID NO: | LC CDR1 (Kabat) | SASQGISNYLN |
| 1321 | | |
| SEQ ID NO: | LC CDR2 (Kabat) | YTSSLHS |
| 1322 | | |
| SEQ ID NO: | LC CDR3 (Kabat) | QQYSKLPRT |
| 1323 | | |
| SEQ ID NO: | LC CDR1 (Chothia) | SASQGISNYLN |
| 1321 | | |
| SEQ ID NO: | LC CDR2 (Chothia) | YTSSLHS |
| 1322 | | |
| SEQ ID NO: | LC CDR3 (Chothia) | QQYSKLPRT |
| 1323 | | |
| SEQ ID NO: | LC CDR1 (Combined) | SASQGISNYLN |
| 1321 | | |
| SEQ ID NO: | LC CDR2 (Combined) | YTSSLHS |
| 1322 | | |
| SEQ ID NO: | LC CDR3(Combined) | QQYSKLPRT |
| 1323 | | |
| SEQ ID NO: 232 | VH | DIQMTQTTSSLSASLGDRVTISCSASQGISNYLNWY |
| | | QQKPDGTVKLLIYYTSSLHSGVPSRFSGSGSGTDYS |
| | | LTISNLEPEDIATYYCQQYSKLPRTFGGGTKVEIK |
| SEQ ID NO: 233 | VL | QVQLKESGPGLVAPSQSLSITCTVSGFSLTAYGVN |
| | | WVRQPPGKGLEWLGMIWGDGNTDYNSALKSRLSI |
| | | SKDNSKSQVFLKMNSLQTDDTARYYCARDRVTAT |
| | | LYAMDYWGQGTSVTVSS |

Humanized antibody C
C-H-1 antibody

| | | |
|---|---|---|
| SEQ ID NO: | HC CDR1 (Kabat) | AYGVN |
| 1315 | | |
| SEQ ID NO: | HC CDR2 (Kabat) | MIWGDGNTDYNSALKS |
| 1316 | | |
| SEQ ID NO: 1317 | HC CDR3 (Kabat) | DRVTATLYAMDY |
| SEQ ID NO: | HC CDR1 (Chothia) | GFSLTAY |
| 1318 | | |
| SEQ ID NO: | HC CDR2 (Chothia) | WGDGN |
| 1319 | | |
| SEQ ID NO: | HC CDR3 (Chothia) | DRVTATLYAMDY |
| 1317 | | |
| SEQ ID NO: | HC CDR1 | GFSLTAYGVN |
| 1320 | (Combined) | |
| SEQ ID NO: | HC CDR2 | MIWGDGNTDYNSALKS |
| 1316 | (Combined) | |
| SEQ ID NO: | HC CDR3(Combined) | DRVTATLYAMDY |
| 1317 | | |
| SEQ ID NO: | LC CDR1 (Kabat) | SASQGISNYLN |
| 1321 | | |
| SEQ ID NO: | LC CDR2 (Kabat) | YTSSLHS |
| 1322 | | |
| SEQ ID NO: | LC CDR3 (Kabat) | QQYSKLPRT |
| 1323 | | |
| SEQ ID NO: | LC CDR1 (Chothia) | SASQGISNYLN |
| 1321 | | |
| SEQ ID NO: | LC CDR2 (Chothia) | YTSSLHS |
| 1322 | | |
| SEQ ID NO: | LC CDR3 (Chothia) | QQYSKLPRT |
| 1323 | | |
| SEQ ID NO: | LC CDR1 (Combined) | SASQGISNYLN |
| 1321 | | |
| SEQ ID NO: | LC CDR2 (Combined) | YTSSLHS |
| 1322 | | |
| SEQ ID NO: | LC CDR3(Combined) | QQYSKLPRT |
| 1323 | | |
| SEQ ID NO: | VL | DIQMTQSPSSLSASVGDRVTITCSASQGISNYLNWY |
| 1324 | | QQTPGKAPKLLIYYTSSLHSGVPSRFSGSGSGTDYTF |
| | | TISSLQPEDIATYYCQQYSKLPRTFGQGTKLQIT |
| SEQ ID NO: | VH | QVQLQESGPGLVRPSQTLSLTCTVSGFSLTAYGVN |
| 1325 | | WVRQPPGRGLEWLGMIWGDGNTDYNSALKSRVT |
| | | MLKDTSKNQFSLRLSSVTAADTAVYYCARDRVTA |
| | | TLYAMDYWGQGSLVTVSS |

Humanized antibody C Variable light chain (VL)

| | | | |
|---|---|---|---|
| SEQ ID NO: | VL | C-H-VL.1 | DIQMTQSPSFLSASVGDRVTITCSASQGISNYLNWY |
| 3000 | | | QQKPGKAVKLLIYYTSSLHSGVPSRFSGSGSGTEYT |
| | | | LTISSLQPEDFATYYCQQYSKLPRTFGGGTKVEIK |
| SEQ ID NO: | VL | C-H-VL.2 | DIQMTQSPSSLSASVGDRVTITCSASQGISNYLNWY |
| 3001 | | | QQKPGKAVKLLIYYTSSLHSGVPSRFSGSGSGTDY |
| | | | TLTISSLQPEDFATYYCQQYSKLPRTFGGGTKVEIK |
| SEQ ID NO: | VL | C-H-VL.3 | DIQMTQSPSSLSASVGDRVTITCSASQGISNYLNWY |
| 3002 | | | QQKPGKVVKLLIYYTSSLHSGVPSRFSGSGSGTDY |
| | | | TLTISSLQPEDVATYYCQQYSKLPRTFGGGTKVEIK |
| SEQ ID NO: | VL | C-H-VL.4 | DIQMTQSPSSLSASVGDRVTITCSASQGISNYLNWY |
| 3003 | | | QQKPGQAVKLLIYYTSSLHSGVPSRFSGSGSGTDY |
| | | | TLTISSLQPEDVATYYCQQYSKLPRTFGGGTKVEIK |
| SEQ ID NO: | VL | C-H-VL.5 | DIQMTQSPSSLSASVGDRVTITCSASQGISNYLNWY |
| 3004 | | | QQKPGKAVKLLIYYTSSLHSGVPSRFSGSGSGTDY |
| | | | TFTISSLQPEDIATYYCQQYSKLPRTFGGGTKVEIK |
| SEQ ID NO: | VL | C-H-VL.6 | DIQMTQSPSSLSASVGDRVTITCSASQGISNYLNWY |
| 3005 | | | QQKPGKTVKLLIYYTSSLHSGIPSRFSGSGSGTDYT |
| | | | LTIRSLQPEDFATYYCQQYSKLPRTFGGGTKVEIK |
| SEQ ID NO: | VL | C-H-VL.7 | AIQMTQSPSSLSASVGDRVTITCSASQGISNYLNWY |
| 3006 | | | QQKPGKAVKLLIYYTSSLHSGVPSRFSGSGSGTDY |
| | | | TLTISSLQPEDFATYYCQQYSKLPRTFGGGTKVEIK |
| SEQ ID NO: | VL | C-H-VL.8 | DIQMTQSPSSVSASVGDRVTITCSASQGISNYLNW |
| 3007 | | | YQQKPGKAVKLLIYYTSSLHSGVPSRFSGSGSGTD |
| | | | YTLTISSLQPEDFATYYCQQYSKLPRTFGGGTKVEI |
| | | | K |
| SEQ ID NO: | VL | C-H-VL.9 | DIQMTQSPSSLSASVGDRVTITCSASQGISNYLNWY |
| 3008 | | | QQKPGKAVKRLIYYTSSLHSGVPSRFSGSGSGTEY |
| | | | TLTISNLQPEDFATYYCQQYSKLPRTFGGGTKVEIK |
| SEQ ID NO: | VL | C-H-VL.10 | AIRMTQSPFSLSASVGDRVTITCSASQGISNYLNWY |
| 3009 | | | QQKPAKAVKLFIYYTSSLHSGVPSRFSGSGSGTDY |
| | | | TLTISSLQPEDFATYYCQQYSKLPRTFGGGTKVEIK |
| SEQ ID NO: | VL | C-H-VL.11 | DIQMTQSPSSLSASVGDRVTITCSASQGISNYLNWY |
| 3010 | | | QQKPGKAVKRLIYYTSSLHSGVPSRFSGSGSGTEY |
| | | | TLTISSLQPEDFATYYCQQYSKLPRTFGGGTKVEIK |
| SEQ ID NO: | VL | C-H-VL.12 | DIQMTQSPSTLSASVGDRVTITCSASQGISNYLNWY |
| 3011 | | | QQKPGKAVKLLIYYTSSLHSGVPSRFSGSGSGTEYT |
| | | | LTISSLQPDDFATYYCQQYSKLPRTFGGGTKVEIK |
| SEQ ID NO: 3012 | VL | C-H-VL.13 | DIQMTQSPSSLSASVGDRVTITCSASQGISNYLNWY |
| | | | QQKPGKAVKSLIYYTSSLHSGVPSRFSGSGSGTDY |
| | | | TLTISSLQPEDFATYYCQQYSKLPRTFGGGTKVEIK |
| SEQ ID NO: | VL | C-H-VL.14 | DIQMTQSPSSLSASVGDRVTITCSASQGISNYLNWY |
| 3013 | | | QQKPGKAVKSLIYYTSSLHSGVPSKFSGSGSGTDY |
| | | | TLTISSLQPEDFATYYCQQYSKLPRTFGGGTKVEIK |
| SEQ ID NO: | VL | C-H-VL.15 | DIQMTQSPSSLSASVGDRVTITCSASQGISNYLNWY |
| 3014 | | | QQKPEKAVKSLIYYTSSLHSGVPSRFSGSGSGTDYT |
| | | | LTISSLQPEDFATYYCQQYSKLPRTFGGGTKVEIK |
| SEQ ID NO: | VL | C-H-VL.16 | DIQMTQSPSAMSASVGDRVTITCSASQGISNYLNW |
| 3015 | | | YQQKPGKVVKRLIYYTSSLHSGVPSRFSGSGSGTE |
| | | | YTLTISSLQPEDFATYYCQQYSKLPRTFGGGTKVEI |
| | | | K |
| SEQ ID NO: | VL | C-H-VL.17 | DIVMTQSPDSLAVSLGERATINCSASQGISNYLNW |
| 3016 | | | YQQKPGQPVKLLIYYTSSLHSGVPDRFSGSGSGTD |
| | | | YTLTISSLQAEDVAVYYCQQYSKLPRTFGGGTKVE |
| | | | IK |
| SEQ ID NO: | VL | C-H-VL.18 | EIVMTQSPGTLSLSPGERATLSCSASQGISNYLNWY |
| 3017 | | | QQKPGQAVKLLIYYTSSLHSGIPDRFSGSGSGTDYT |
| | | | LTISRLEPEDFAVYYCQQYSKLPRTFGGGTKVEIK |
| SEQ ID NO: | VL | C-H-VL.19 | EIVMTQSPPTLSLSPGERVTLSCSASQGISNYLNWY |
| 3018 | | | QQKPGQAVKLLIYYTSSLHSGIPARFSGSGSGTDYT |
| | | | LTISSLQPEDFAVYYCQQYSKLPRTFGGGTKVEIK |
| SEQ ID NO: | VL | C-H-VL.20 | EIVMTQSPPTLSLSPGERVTLSCSASQGISNYLNWY |
| 3019 | | | QQKPGQAVKLLIYYTSSLHSSIPARFSGSGSGTDYT |
| | | | LTISSLQPEDFAVYYCQQYSKLPRTFGGGTKVEIK |
| SEQ ID NO: | VL | C-H-VL.21 | EIVMTQSPATLSLSPGERATLSCSASQGISNYLNWY |
| 3020 | | | QQKPGQAVKLLIYYTSSLHSGIPARFSGSGSGTDYT |
| | | | LTISSLEPEDFAVYYCQQYSKLPRTFGGGTKVEIK |
| SEQ ID NO: | VL | C-H-VL.22 | EIVMTQSPATLSLSPGERATLSCSASQGISNYLNWY |
| 3021 | | | QQKPGQAVKLLIYYTSSLHSGIPARFSGSGSGTDYT |
| | | | LTISRLEPEDFAVYYCQQYSKLPRTFGGGTKVEIK |
| SEQ ID NO: | VL | C-H-VL.23 | EIVMTQSPATLSLSPGERATLSCSASQGISNYLNWY |
| 3022 | | | QQKPGQAVKLLIYYTSSLHSGIPDRFSGSGSGTDYT |
| | | | LTISRLEPEDFAVYYCQQYSKLPRTFGGGTKVEIK |
| SEQ ID NO: | VL | C-H-VL.24 | EIVMTQSPATLSLSPGERATLSCSASQGISNYLNWY |
| 3023 | | | QQKPGLAVKLLIYYTSSLHSGIPDRFSGSGSGTDYT |
| | | | LTISRLEPEDFAVYYCQQYSKLPRTFGGGTKVEIK |
| SEQ ID NO: | VL | C-H-VL.25 | DIQMIQSPSFLSASVGDRVSIICSASQGISNYLNWYL |
| 3024 | | | QKPGKSVKLFIYYTSSLHSGVSSRFSGRGSGTDYTL |
| | | | TIISLKPEDFAAYYCQQYSKLPRTFGGGTKVEIK |
| SEQ ID NO: | VL | C-H-VL.26 | EIVMTQSPATLSLSPGERATLSCSASQGISNYLNWY |
| 3025 | | | QQKPGQAVKLLIYYTSSLHSGIPARFSGSGSGTDYT |
| | | | LTISSLQPEDFAVYYCQQYSKLPRTFGGGTKVEIK |
| SEQ ID NO: | VL | C-H-VL.27 | EIVMTQSPATLSLSPGERATLSCSASQGISNYLNWY |
| 3026 | | | QQKPGQAVKLLIYYTSSLHSGIPARFSGSGPGTDYT |
| | | | LTISSLEPEDFAVYYCQQYSKLPRTFGGGTKVEIK |
| SEQ ID NO: | VL | C-H-VL.28 | DIVMTQTPLSLSVTPGQPASISCSASQGISNYLNWY |
| 3027 | | | LQKPGQSVKLLIYYTSSLHSGVPDRFSGSGSGTDYT |
| | | | LKISRVEAEDVGVYYCQQYSKLPRTFGGGTKVEIK |
| SEQ ID NO: | VL | C-H-VL.29 | DIVMTQTPLSLSVTPGQPASISCSASQGISNYLNWY |
| 3028 | | | LQKPGQPVKLLIYYTSSLHSGVPDRFSGSGSGTDYT |
| | | | LKISRVEAEDVGVYYCQQYSKLPRTFGGGTKVEIK |
| SEQ ID NO: | VL | C-H-VL.30 | DIVMTQSPAFLSVTPGEKVTITCSASQGISNYLNWY |
| 3029 | | | QQKPDQAVKLLIYYTSSLHSGVPSRFSGSGSGTDY |
| | | | TFTISSLEAEDAATYYCQQYSKLPRTFGGGTKVEIK |
| SEQ ID NO: | VL | C-H-VL.31 | DIVMTQSPLSLPVTPGEPASISCSASQGISNYLNWY |
| 3030 | | | LQKPGQSVKLLIYYTSSLHSGVPDRFSGSGSGTDYT |
| | | | LKISRVEAEDVGVYYCQQYSKLPRTFGGGTKVEIK |
| SEQ ID NO: | VL | C-H-VL.32 | DIVMTQTPLSLPVTPGEPASISCSASQGISNYLNWY |
| 3031 | | | LQKPGQSVKLLIYYTSSLHSGVPDRFSGSGSGTDYT |
| | | | LKISRVEAEDVGVYYCQQYSKLPRTFGGGTKVEIK |
| SEQ ID NO: | VL | C-H-VL.33 | EIVMTQSPATLSVSPGERATLSCSASQGISNYLNWY |
| 3032 | | | QQKPGQAVKLLIYYTSSLHSGIPARFSGSGSGTEYT |
| | | | LTISILQSEDFAVYYCQQYSKLPRTFGGGTKVEIK |
| SEQ ID NO: | VL | C-H-VL.34 | EIVMTQSPATLSVSPGERATLSCSASQGISNYLNWY |
| 3033 | | | QQKPGQAVKLLIYYTSSLHSGIPARFSGSGSGTEYT |
| | | | LTISSLQSEDFAVYYCQQYSKLPRTFGGGTKVEIK |
| SEQ ID NO: | VL | C-H-VL.35 | DIVMTQSPLSLPVTLGQPASISCSASQGISNYLNWY |
| 3034 | | | QQRPGQSVKRLIYYTSSLHSGVPDRFSGSGSGTDY |
| | | | TLKISRVEAEDVGVYYCQQYSKLPRTFGGGTKVEI |
| | | | K |
| SEQ ID NO: | VL | C-H-VL.36 | EITMTQSPAFMSATPGDKVNISCSASQGISNYLNW |
| 3035 | | | YQQKPGEAVKFIIYYTSSLHSGIPPRFSGSGYGTDY |
| | | | TLTINNIESEDAAYYYCQQYSKLPRTFGGGTKVEIK |
| SEQ ID NO: | VL | C-H-VL.37 | DIVMTQTPLSSPVTLGQPASISCSASQGISNYLNWY |
| 3036 | | | QQRPGQPVKLLIYYTSSLHSGVPDRFSGSGAGTDY |
| | | | TLKISRVEAEDVGVYYCQQYSKLPRTFGGGTKVEI |
| | | | K |
| SEQ ID NO: | VL | C-H-VL.38 | EIVMTQSPDFQSVTPKEKVTITCSASQGISNYLNWY |
| 3037 | | | QQKPDQSVKLLIYYTSSLHSGVPSRFSGSGSGTDYT |
| | | | LTINSLEAEDAATYYCQQYSKLPRTFGGGTKVEIK |
| SEQ ID NO: | VL | C-H-VL.39 | EIVMTQTPLSLSITPGEQASISCSASQGISNYLNWYL |
| 3038 | | | QKARPVVKLLIYYTSSLHSGVPDRFSGSGSGTDYT |
| | | | LKISRVEAEDFGVYYCQQYSKLPRTFGGGTKVEIK |
| SEQ ID NO: | VL | C-H-VL.40 | EIVMTQTPLSLSITPGEQASMSCSASQGISNYLNWY |
| 3039 | | | LQKARPVVKLLIYYTSSLHSGVPDRFSGSGSGTDY |
| | | | TLKISRVEAEDFGVYYCQQYSKLPRTFGGGTKVEI |
| | | | K |

Humanized antibody C Variable HEAVY chain (VH)

| | | | |
|---|---|---|---|
| SEQ ID NO: | VH | C-H-VH.1 | QVTLKESGPVLVKPTETLTLTCTVSGFSLTAYGVN |
| 3040 | | | WVRQPPGKALEWLGMIWGDGNTDYNSALKSRLTI |
| | | | SKDNSKSQVVLTMTNMDPVDTATYYCARDRVTA |
| | | | TLYAMDYWGQGTLVTVSS |
| SEQ ID NO: | VH | C-H-VH.2 | QVTLKESGPALVKPTETLTLTCTVSGFSLTAYGVN |
| 3041 | | | WVRQPPGKALEWLGMIWGDGNTDYNSALKSRLII |
| | | | SKDNSKSQVVLTMTNMDPVDTATYYCARDRVTA |
| | | | TLYAMDYWGQGTLVTVSS |
| SEQ ID NO: | VH | C-H-VH.3 | QVTLKESGPALVKPTQTLTLTCTVSGFSLTAYGVN |
| 3042 | | | WVRQPPGKALEWLGMIWGDGNTDYNSALKSRLTI |
| | | | SKDNSKSQVVLTMTNMDPVDTATYYCARDRVTA |
| | | | TLYAMDYWGQGTLVTVSS |
| SEQ ID NO: | VH | C-H-VH.4 | QVQLQESGPGLVKPSGTLSLTCAVSGFSLTAYGVN |
| 3043 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.5 | QVTLKESGPTLVKPTQTLTLTCTVSGFSLTAYGVN |
| 3044 | | | WVRQPPGKALEWLGMIWGDGNTDYNSALKSRLTI |
| | | | TKDNSKSQVVLTMTNMDPVDTATYYCARDRVTA |
| | | | TLYAMDYWGQGTLVTVSS |
| SEQ ID NO: | VH | C-H-VH.6 | QVTLKESGPALVKPTQTLTLTCTVSGFSLTAYGVN |
| 3045 | | | WVRQPPGKALEWLGMIWGDGNTDYNSALKSRLTI |
| | | | TKDNSKSQVVLTMTNMDPVDTATYYCARDRVTA |
| | | | TLYAMDYWGQGTLVTVSS |
| SEQ ID NO: | VH | C-H-VH.7 | QVQLQESGPGLVKPSQTLSLTCTVSGFSLTAYGVN |
| 3046 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.8 | QVQLQESGPGLVKPSETLSLTCTVSGFSLTAYGVN |
| 3047 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.9 | QVQLQESGPGLVKPSQTLSLTCAVSGFSLTAYGVN |
| 3048 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.10 | QVQLQESGPGLVKPSDTLSLTCTVSGFSLTAYGVN |
| 3049 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.11 | QVQLQESGPGLVKPSQTLSLTCTVSGFSLTAYGVN |
| 3050 | | | WVRQHPGKGLEWLGMIWGDGNTDYNSALKSRLT |
| | | | ISKDNSKSQVSLKLSSVTAADTAVYYCARDRVTAT |
| | | | LYAMDYWGQGTLVTVSS |
| SEQ ID NO: | VH | C-H-VH.12 | QVQLQESGPGLVKPSQTLSLTCTVSGFSLTAYGVN |
| 3051 | | | WVRQPAGKGLEWLGMIWGDGNTDYNSALKSRLT |
| | | | ISKDNSKSQVSLKLSSVTAADTAVYYCARDRVTAT |
| | | | LYAMDYWGQGTLVTVSS |
| SEQ ID NO: | VH | C-H-VH.13 | QVQLQESGPGLVKPSQTLSLTCAVSGFSLTAYGVN |
| 3052 | | | WVRQPPGKGLEWLGMIWGDGNTDYNSALKSRLTI |
| | | | SKDNSKSQVSLKLSSVTAVDTAVYYCARDRVTAT |
| | | | LYAMDYWGQGTLVTVSS |
| SEQ ID NO: | VH | C-H-VH.14 | QVQLQESGPGLVKPSETLSLTCTVSGFSLTAYGVN |
| 3053 | | | WVRQPPGKGLEWLGMIWGDGNTDYNSALKSRLTI |
| | | | SKDNSKSHVSLKLSSVTAADTAVYYCARDRVTAT |
| | | | LYAMDYWGQGTLVTVSS |
| SEQ ID NO: | VH | C-H-VH.15 | QVQLQESGPGLVKPSETLSLTCAVSGFSLTAYGVN |
| 3054 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.16 | QVQLQESGPGLVKPSQTLSLTCAVYGFSLTAYGVN |
| 3055 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.17 | RVQLQESGPGLVKPSETLSLTCTVSGFSLTAYGVN |
| 3056 | | | WVRQPPGKGLEWLGMIWGDGNTDYNSALKSRLTI |
| | | | SKDNSKSQVPLKLSSVTAADTAVYYCARDRVTAT |
| | | | LYAMDYWGQGTLVTVSS |
| SEQ ID NO: | VH | C-H-VH.18 | QVQLQESGPGLVKPSQTLSLTCTVSGFSLTAYGVN |
| 3057 | | | WVRQHPGKGLEWLGMIWGDGNTDYNSALKSLLT |
| | | | ISKDNSKSQVSLKLSSVTAADTAVYYCARDRVTAT |
| | | | LYAMDYWGQGTLVTVSS |
| SEQ ID NO: | VH | C-H-VH.19 | QVQLQESGPGLVKPSDTLSLTCAVSGFSLTAYGVN |
| 3058 | | | WVRQPPGKGLEWLGMIWGDGNTDYNSALKSRLTI |
| | | | SKDNSKSQVSLKLSSVTALDTAVYYCARDRVTAT |
| | | | LYAMDYWGQGTLVTVSS |
| SEQ ID NO: | VH | C-H-VH.20 | QVQLQESGPGLVKPSDTLSLTCAVSGFSLTAYGVN |
| 3059 | | | WVRQPPGKGLEWLGMIWGDGNTDYNSALKSRLTI |
| | | | SKDNSKSQVSLKLSSVTAVDTAVYYCARDRVTAT |
| | | | LYAMDYWGQGTLVTVSS |
| SEQ ID NO: | VH | C-H-VH.21 | QVQLQESGSGLVKPSQTLSLTCAVSGFSLTAYGVN |
| 3060 | | | WVRQPPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.22 | EVQLVESGGGLVQPGRSLRLSCTVSGFSLTAYGVN |
| 3061 | | | WVRQAPGKGLEWLGMIWGDGNTDYNSALKSRLT |
| | | | ISKDNSKSIVYLQMNSLKTEDTAVYYCARDRVTAT |
| | | | LYAMDYWGQGTLVTVSS |
| SEQ ID NO: | VH | C-H-VH.23 | EVQLVESGGGLVQPGPSLRLSCTVSGFSLTAYGVN |
| 3062 | | | WVRQAPGKGLEWLGMIWGDGNTDYNSALKSRLT |
| | | | ISKDNSKSIVYLQMNSLKTEDTAVYYCARDRVTAT |
| | | | LYAMDYWGQGTLVTVSS |
| SEQ ID NO: | VH | C-H-VH.24 | QVQLQESGSGLVKPSQTLSLTCAVSGFSLTAYGVN |
| 3063 | | | WVRQSPGKGLEWLGMIWGDGNTDYNSALKSRLTI |
| | | | SKDNSKSQVSLKLSSVTAADTAVYYCARDRVTAT |
| | | | LYAMDYWGQGTLVTVSS |
| SEQ ID NO: | VH | C-H-VH.25 | QVQLQESGPGLVKPSETLSLTCTVSGFSLTAYGVN |
| 3064 | | | WVRQPAGKGLEWLGMIWGDGNTDYNSALKSRLT |
| | | | ISKDNSKSQVSLKLSSVTAADTAVYYCARDRVTAT |
| | | | LYAMDYWGQGTLVTVSS |
| SEQ ID NO: | VH | C-H-VH.26 | EVQLVESGGGLVKPGRSLRLSCTVSGFSLTAYGVN |
| 3065 | | | WVRQAPGKGLEWLGMIWGDGNTDYNSALKSRLT |
| | | | ISKDNSKSIVYLQMNSLKTEDTAVYYCARDRVTAT |
| | | | LYAMDYWGQGTLVTVSS |
| SEQ ID NO: | VH | C-H-VH.27 | QVQLQESGPGLVKPSETLSLTCAVYGFSLTAYGVN |
| 3066 | | | WVRQPPGKGLEWLGMIWGDGNTDYNSALKSRLTI |
| | | | SKDNSKSQVYLKLSSVTAADTAVYYCARDRVTAT |
| | | | LYAMDYWGQGTLVTVSS |
| SEQ ID NO: | VH | C-H-VH.28 | QVQLQESGPGLVKPSDTLSLTCAVSGFSLTAYGVN |
| 3067 | | | WVRQPPGKGLEWLGMIWGDGNTDYNSALKSRLTI |
| | | | SKDNSKSQVSLKLSSVTAVDTGVYYCARDRVTAT |
| | | | LYAMDYWGQGTLVTVSS |
| SEQ ID NO: | VH | C-H-VH.29 | EVQLVESGGGLVQPGGSLRLSCAVSGFSLTAYGVN |
| 3068 | | | WVRQAPGKGLEWLGMIWGDGNTDYNSALKSRLT |
| | | | ISKDNSKSSVYLQMNSLKTEDTAVYYCARDRVTA |
| | | | TLYAMDYWGQGTLVTVSS |
| SEQ ID NO: | VH | C-H-VH.30 | EVQLVESGGGLVKPGGSLRLSCAVSGFSLTAYGVN |
| 3069 | | | WVRQAPGKGLEWLGMIWGDGNTDYNSALKSRLT |
| | | | ISKDNSKSTVYLQMNSLKTEDTAVYYCARDRVTA |
| | | | TLYAMDYWGQGTLVTVSS |
| SEQ ID NO: | VH | C-H-VH.31 | QVQLQQSGPGLVKPSQTLSLTCAVSGFSLTAYGVN |
| 3070 | | | WVRQSPSRGLEWLGMIWGDGNTDYNSALKSRLTI |
| | | | NKDNSKSQVSLQLNSVTPEDTAVYYCARDRVTAT |
| | | | LYAMDYWGQGTLVTVSS |
| SEQ ID NO: | VH | C-H-VH.32 | QVQLVESGGGLVQPGGSLRLSCSVSGFSLTAYGVN |
| 3071 | | | WVRQAPGKGLEWLGMIWGDGNTDYNSALKSRLT |
| | | | ISKDNSKSTVYLQMNSLRAEDTAVYYCARDRVTA |
| | | | TLYAMDYWGQGTLVTVSS |
| SEQ ID NO: | VH | C-H-VH.33 | QVQLQQWGAGLLKPSETLSLTCAVYGFSLTAYGV |
| 3072 | | | NWVRQPPGKGLEWLGMIWGDGNTDYNSALKSRL |
| | | | TISKDNSKSQVSLKLSSVTAADTAVYYCARDRVTA |
| | | | TLYAMDYWGQGTLVTVSS |
| SEQ ID NO: | VH | C-H-VH.34 | QVQLVESGGGVVQPGRSLRLSCAVSGFSLTAYGV |
| 3073 | | | NWVRQAPGKGLEWLGMIWGDGNTDYNSALKSRL |
| | | | TISKDNSTSTVFLQMNSLRAEDTAVYYCARDRVTA |
| | | | TLYAMDYWGQGTLVTVSS |
| SEQ ID NO: | VH | C-H-VH.35 | EVQLVESGGGLVQPGGSLRLSCAVSGFSLTAYGVN |
| 3074 | | | WVRQAPGKGLEWLGMIWGDGNTDYNSALKSRLT |
| | | | ISKDNSKSTVYLQMNSLRAEDTAVYYCARDRVTA |
| | | | TLYAMDYWGQGTLVTVSS |
| SEQ ID NO: | VH | C-H-VH.36 | EVQLVESGGGLVQPGGSLRLSCAVSGFSLTAYGVN |
| 3075 | | | WVRQAPGKGLEWLGMIWGDGNTDYNSALKSRLT |
| | | | ISKDNAKSSVYLQMNSLRDEDTAVYYCARDRVTA |
| | | | TLYAMDYWGQGTLVTVSS |
| SEQ ID NO: | VH | C-H-VH.37 | EVQLLESGGGLVQPGGSLRLSCAVSGFSLTAYGVN |
| 3076 | | | WVRQAPGKGLEWLGMIWGDGNTDYNSALKSRLT |
| | | | ISKDNSKSTVYLQMNSLRAEDTAVYYCARDRVTA |
| | | | TLYAMDYWGQGTLVTVSS |
| SEQ ID NO: | VH | C-H-VH.38 | QVQLVESGGGLVKPGGSLRLSCAVSGFSLTAYGV |
| 3077 | | | NWVRQAPGKGLEWLGMIWGDGNTDYNSALKSRL |
| | | | TISKDNAKSSVYLQMNSLRAEDTAVYYCARDRVT |
| | | | ATLYAMDYWGQGTLVTVSS |
| SEQ ID NO: | VH | C-H-VH.39 | EVQLVESGGGLVQPGGSLKLSCAVSGFSLTAYGV |
| 3078 | | | NWVRQASGKGLEWLGMIWGDGNTDYNSALKSRL |
| | | | TISKDNSKSTVYLQMNSLKTEDTAVYYCARDRVT |
| | | | ATLYAMDYWGQGTLVTVSS |
| SEQ ID NO: | VH | C-H-VH.40 | QVQLLESGGGLVKPGGSLRLSCAVSGFSLTAYGVN |
| 3079 | | | WVRQAPGKGLEWLGMIWGDGNTDYNSALKSRLT |
| | | | ISKDNAKSSVYLQMNSLRAEDTAVYYCARDRVTA |
| | | | TLYAMDYWGQGTLVTVSS |
| SEQ ID NO: | VH | C-H-VH.41 | QVQLVESGGGVVQPGRSLRLSCAVSGFSLTAYGV |
| 3080 | | | NWVRQAPGKGLEWLGMIWGDGNTDYNSALKSRL |
| | | | TISKDNSKSTVYLQMNSLRAEDTAVYYCARDRVT |
| | | | ATLYAMDYWGQGTLVTVSS |
| SEQ ID NO: | VH | C-H-VH.42 | QVQLVESGGGVVQPGRSLRLSCAVSGFSLTAYGV |
| 3081 | | | NWVRQAPGKGLEWLGMIWGDGNTDYNSALKSRL |
| | | | TISKDNSKSRVYLQMNSLRAEDTAVYYCARDRVT |
| | | | ATLYAMDYWGQGTLVTVSS |
| SEQ ID NO: | VH | C-H-VH.43 | QVQLVESGGGVVQPGRSLRLSCAVSGFSLTAYGV |
| 3082 | | | NWVRQAPGKGLEWLGMIWGDGNTDYNSALKSRL |
| | | | AISKDNSKSTVYLQMNSLRAEDTAVYYCARDRVT |
| | | | ATLYAMDYWGQGTLVTVSS |
| SEQ ID NO: | VH | C-H-VH.44 | QVQLVESGGGVVQPGGSLRLSCAVSGFSLTAYGV |
| 3083 | | | NWVRQAPGKGLEWLGMIWGDGNTDYNSALKSRL |
| | | | TISKDNSKSTVYLQMNSLRAEDTAVYYCARDRVT |
| | | | ATLYAMDYWGQGTLVTVSS |
| SEQ ID NO: | VH | C-H-VH.45 | EVQLVESGGGLVQPGGSLRLSCAVSGFSLTAYGVN |
| 3084 | | | WVRQAPGKGLEWLGMIWGDGNTDYNSALKSRLT |
| | | | ISKDNAKSTVYLQMNSLRAEDTAVYYCARDRVTA |
| | | | TLYAMDYWGQGTLVTVSS |
| SEQ ID NO: | VH | C-H-VH.46 | EVQLVESGGGLVQPGGSLRLSCAVSGFSLTAYGVN |
| 3085 | | | WVRQAPGKGLEWLGMIWGDGNTDYNSALKSRLT |
| | | | ISKDNAKSSVYLQMNSLRAEDTAVYYCARDRVTA |
| | | | TLYAMDYWGQGTLVTVSS |
| SEQ ID NO: | VH | C-H-VH.47 | EVQLVESGGVVVQPGGSLRLSCAVSGFSLTAYGV |
| 3086 | | | NWVRQAPGKGLEWLGMIWGDGNTDYNSALKSRL |
| | | | TISKDNSKSSVYLQMNSLRTEDTALYYCARDRVTA |
| | | | TLYAMDYWGQGTLVTVSS |
| SEQ ID NO: | VH | C-H-VH.48 | EVQLVESGGGLVQPGGSLRLSCAVSGFSLTAYGVN |
| 3087 | | | WVRQAPGKGLEWLGMIWGDGNTDYNSALKSRLT |
| | | | ISKHNSKSTVYLQMNSLRAEDTAVYYCARDRVTA |
| | | | TLYAMDYWGQGTLVTVSS |
| SEQ ID NO: | VH | C-H-VH.49 | EVQLVESGGGLVKPGGSLRLSCAVSGFSLTAYGVN |
| 3088 | | | WVRQAPGKGLEWLGMIWGDGNTDYNSALKSRLT |
| | | | ISKDNAKSSVYLQMNSLRAEDTAVYYCARDRVTA |
| | | | TLYAMDYWGQGTLVTVSS |
| SEQ ID NO: | VH | C-H-VH.50 | EVQLVESGGGLIQPGGSLRLSCAVSGFSLTAYGVN |
| 3089 | | | WVRQPPGKGLEWLGMIWGDGNTDYNSALKSRLTI |
| | | | SKDNSKSTVYLQMNSLRAEDTAVYYCARDRVTAT |
| | | | LYAMDYWGQGTLVTVSS |

TABLE 11
Amino acid sequences for anti TCRβ V5 antibodies. Amino acid and nucleotide
sequences for murine and humanized antibody molecules which bind to TCRVB 5 (e.g., TCRVB
5-5 or TCRVB 5-6). The amino acid the heavy and light chain CDRs, and the amino acid and
nucleotide sequences of the heavy and light chain variable regions, and the heavy and
light chains are shown.
Murine antibody E, also referred to as MH3-2

| | | |
|---|---|---|
| SEQ ID NO: | HC CDR1 (Kabat) | SSWMN |
| 1298 | | |
| SEQ ID NO: | HC CDR2 (Kabat) | RIYPGDGDTKYNGKFKG |
| 1299 | | |
| SEQ ID NO: | HC CDR3 (Kabat) | RGTGGWYFDV |
| 1300 | | |
| SEQ ID NO: | HC CDR1 (Chothia) | GYAFSSS |
| 1302 | | |
| SEQ ID NO: | HC CDR2 (Chothia) | YPGDGD |
| 1303 | | |
| SEQ ID NO: | HC CDR3 (Chothia) | RGTGGWYFDV |
| 1301 | | |
| SEQ ID NO: | HC CDR1 | GYAFSSSWMN |
| 1304 | (Combined) | |
| SEQ ID NO: | HC CDR2 | RIYPGDGDTKYNGKFKG |
| 1299 | (Combined)) | |
| SEQ ID NO: | HC CDR3(Combined) | RGTGGWYFDV |
| 1301 | | |
| SEQ ID NO: | LC CDR1 (Kabat) | RASESVDSSGNSFMH |
| 1305 | | |
| SEQ ID NO: | LC CDR2 (Kabat) | RASNLES |
| 1306 | | |
| SEQ ID NO: | LC CDR3 (Kabat) | QQSFDDPFT |
| 1307 | | |
| SEQ ID NO: | LC CDR1 (Chothia) | SESVDSSGNSF |
| 1308 | | |
| SEQ ID NO: | LC CDR2 (Chothia) | RASNLES |
| 1306 | | |
| SEQ ID NO: | LC CDR3 (Chothia) | QQSFDDPFT |
| 1307 | | |
| SEQ ID NO: | LC CDR1 (Combined) | RASESVDSSGNSFMH |
| 1305 | | |
| SEQ ID NO: | LC CDR2 (Combined) | RASNLES |
| 1306 | | |
| SEQ ID NO: | LC CDR3(Combined) | QQSFDDPFT |
| 1307 | | |
| SEQ ID NO: | VH | QVQLQQSGPELVKPGASVKISCKASGYAFSSSWM |
| 3091 | | NWVKQRPGQGLEWIGRIYPGDGDTKYNGKFKGK |
| | | ATLTADKSSSTAYMHLSSLTSVDSAVYFCARRGTG |
| | | GWYFDVWGAGTTVTVSS |
| SEQ ID NO: | Heavy chain | METDTLLLWVLLLWVPGSTGQVQLQQSGPELVKP |
| 3284 | | GASVKISCKASGYAFSSSWMNWVKQRPGQGLEWI |
| | | GRIYPGDGDTKYNGKFKGKATLTADKSSSTAYMH |
| | | LSSLTSVDSAVYFCARRGTGGWYFDVWGAGTTVT |
| | | VSSAKTTAPSVYPLAPVCGDTTGSSVTLGCLVKGY |
| | | FPEPVTLTWNSGSLSSGVHTFPAVLQSDLYTLSSSV |
| | | TVTSSTWPSQSITCNVAHPASSTKVDKKIEPRGPTI |
| | | KPCPPCKCPAPNLLGGPSVFIFPPKIKDVLMISLSPIV |
| | | TCVVVDVSEDDPDVQISWFVNNVEVHTAQTQTHR |
| | | EDYNSTLRVVSALPIQHQDWMSGKEFKCKVNNKD |
| | | LPAPIERTISKPKGSVRAPQVYVLPPPEEEMTKKQV |
| | | TLTCMVTDFMPEDIYVEWTNNGKTELNYKNTEPV |
| | | LDSDGSYFMYSKLRVEKKNWVERNSYSCSVVHEG |
| | | LHNHHTTKSFSRTPGK |
| SEQ ID NO: | VL | DIVLTQSPASLAVSLGQRATISCRASESVDSSGNSF |
| 3092 | | MHWYQQKPGQPPQLLIYRASNLESGIPARFSGSGS |
| | | RTDFTLTINPVEADDVATFYCQQSFDDPFTFGSGTK |
| | | LEIK |
| SEQ ID NO: | Light chain | METDTLLLWVLLLWVPGSTGDIVLTQSPASLAVSL |
| 3285 | | GQRATISCRASESVDSSGNSFMHWYQQKPGQPPQL |
| | | LIYRASNLESGIPARFSGSGSRTDFTLTINPVEADDV |
| | | ATFYCQQSFDDPFTFGSGTKLEIKRADAAPTVSIFP |
| | | PSSEQLTSGGASVVCFLNNFYPKDINVKWKIDGSE |
| | | RQNGVLNSWTDQDSKDSTYSMSSTLTLTKDEYER |
| | | HNSYTCEATHKTSTSPIVKSFNRNEC |

Humanized antibody E (E-H antibody)
Variable light chain (VL)

| | | | |
|---|---|---|---|
| SEQ ID NO: | VL | E-H.1 | DIVLTQSPDSLAVSLGERATINCRASESVDSSGNSFMHW |
| 3093 | | | YQQKPGQPPQLLIYRASNLESGVPDRFSGSGSRTDFTLT |
| | | | ISSLQAEDVAVYYCQQSFDDPFTFGQGTKLEIK |
| SEQ ID NO: | VL | E-H.2 | EIVLTQSPATLSLSPGERATLSCRASESVDSSGNSFMHW |
| 3094 | | | YQQKPGQAPQLLIYRASNLESGIPARFSGSGSRTDFTLTI |
| | | | SSLEPEDFAVYYCQQSFDDPFTFGQGTKLEIK |
| SEQ ID NO: | VL | E-H.3 | EIVLTQSPATLSLSPGERATLSCRASESVDSSGNSFMHW |
| 3095 | | | YQQKPGQAPQLLIYRASNLESGIPARFSGSGSRTDFTLTI |
| | | | SRLEPEDFAVYYCQQSFDDPFTFGQGTKLEIK |
| SEQ ID NO: | VL | E-H.4 | EIVLTQSPATLSLSPGERATLSCRASESVDSSGNSFMHW |
| 3096 | | | YQQKPGQAPQLLIYRASNLESGIPARFSGSGSRTDFTLTI |
| | | | SSLQPEDFAVYYCQQSFDDPFTFGQGTKLEIK |
| SEQ ID NO: | VL | E-H.5 | DIQLTQSPSSLSASVGDRVTITCRASESVDSSGNSFMHW |
| 3097 | | | YQQKPGQAPQLLIYRASNLESGVPSRFSGSGSRTDFTLT |
| | | | ISSLQPEDVATYYCQQSFDDPFTFGQGTKLEIK |
| SEQ ID NO: | VL | E-H.6 | EIVLTQSPATLSLSPGERATLSCRASESVDSSGNSFMHW |
| 3098 | | | YQQKPGQAPQLLIYRASNLESGIPARFSGSGPRTDFTLTI |
| | | | SSLEPEDFAVYYCQQSFDDPFTFGQGTKLEIK |
| SEQ ID NO: | VL | E-H.7 | EIVLTQSPATLSLSPGERATLSCRASESVDSSGNSFMHW |
| 3099 | | | YQQKPGQAPQLLIYRASNLESGIPDRFSGSGSRTDFTLTI |
| | | | SRLEPEDFAVYYCQQSFDDPFTFGQGTKLEIK |
| SEQ ID NO: | VL | E-H.8 | DIQLTQSPSSLSASVGDRVTITCRASESVDSSGNSFMHW |
| 3100 | | | YQQKPGKVPQLLIYRASNLESGVPSRFSGSGSRTDFTLT |
| | | | ISSLQPEDVATYYCQQSFDDPFTFGQGTKLEIK |
| SEQ ID NO: | VL | E-H.9 | DIQLTQSPSSLSASVGDRVTITCRASESVDSSGNSFMHW |
| 3101 | | | YQQKPGKTPQLLIYRASNLESGIPSRFSGSGSRTDFTLTI |
| | | | RSLQPEDFATYYCQQSFDDPFTFGQGTKLEIK |
| SEQ ID NO: | VL | E-H.10 | EIVLTQSPGTLSLSPGERATLSCRASESVDSSGNSFMHW |
| 3102 | | | YQQKPGQAPQLLIYRASNLESGIPDRFSGSGSRTDFTLTI |
| | | | SRLEPEDFAVYYCQQSFDDPFTFGQGTKLEIK |
| SEQ ID NO: | VL | E-H.11 | EIVLTQSPATLSLSPGERATLSCRASESVDSSGNSFMHW |
| 3103 | | | YQQKPGLAPQLLIYRASNLESGIPDRFSGSGSRTDFTLTI |
| | | | SRLEPEDFAVYYCQQSFDDPFTFGQGTKLEIK |
| SEQ ID NO: | VL | E-H.12 | DIQLTQSPSSLSASVGDRVTITCRASESVDSSGNSFMHW |
| 3104 | | | YQQKPGKAPQLLIYRASNLESGVPSRFSGSGSRTDFTLT |
| | | | ISSLQPEDFATYYCQQSFDDPFTFGQGTKLEIK |
| SEQ ID NO: | VL | E-H.13 | DIQLTQSPSSVSASVGDRVTITCRASESVDSSGNSFMHW |
| 3105 | | | YQQKPGKAPQLLIYRASNLESGVPSRFSGSGSRTDFTLT |
| | | | ISSLQPEDFATYYCQQSFDDPFTFGQGTKLEIK |
| SEQ ID NO: | VL | E-H.14 | AIQLTQSPSSLSASVGDRVTITCRASESVDSSGNSFMHW |
| 3106 | | | YQQKPGKAPQLLIYRASNLESGVPSRFSGSGSRTDFTLT |
| | | | ISSLQPEDFATYYCQQSFDDPFTFGQGTKLEIK |
| SEQ ID NO: | VL | E-H.15 | DIQLTQSPSFLSASVGDRVTITCRASESVDSSGNSFMHW |
| 3107 | | | YQQKPGKAPQLLIYRASNLESGVPSRFSGSGSRTEFTLTI |
| | | | SSLQPEDFATYYCQQSFDDPFTFGQGTKLEIK |
| SEQ ID NO: | VL | E-H.16 | DIQLTQSPSSLSASVGDRVTITCRASESVDSSGNSFMHW |
| 3108 | | | YQQKPGKAPQLLIYRASNLESGVPSRFSGSGSRTDFTFTI |
| | | | SSLQPEDIATYYCQQSFDDPFTFGQGTKLEIK |
| SEQ ID NO: | VL | E-H.17 | EIVLTQSPATLSVSPGERATLSCRASESVDSSGNSFMHW |
| 3109 | | | YQQKPGQAPQLLIYRASNLESGIPARFSGSGSRTEFTLTI |
| | | | SILQSEDFAVYYCQQSFDDPFTFGQGTKLEIK |
| SEQ ID NO: | VL | E-H.18 | EIVLTQSPATLSVSPGERATLSCRASESVDSSGNSFMHW |
| 3110 | | | YQQKPGQAPQLLIYRASNLESGIPARFSGSGSRTEFTLTI |
| | | | SSLQSEDFAVYYCQQSFDDPFTFGQGTKLEIK |
| SEQ ID NO: | VL | E-H.19 | AIRLTQSPFSLSASVGDRVTITCRASESVDSSGNSFMHW |
| 3111 | | | YQQKPAKAPQLFIYRASNLESGVPSRFSGSGSRTDFTLTI |
| | | | SSLQPEDFATYYCQQSFDDPFTFGQGTKLEIK |
| SEQ ID NO: | VL | E-H.20 | DIQLTQSPSSLSASVGDRVTITCRASESVDSSGNSFMHW |
| 3112 | | | YQQKPGKAPQSLIYRASNLESGVPSRFSGSGSRTDFTLTI |
| | | | SSLQPEDFATYYCQQSFDDPFTFGQGTKLEIK |
| SEQ ID NO: | VL | E-H.21 | DIQLTQSPSSLSASVGDRVTITCRASESVDSSGNSFMHW |
| 3113 | | | YQQKPGKAPQRLIYRASNLESGVPSRFSGSGSRTEFTLTI |
| | | | SNLQPEDFATYYCQQSFDDPFTFGQGTKLEIK |
| SEQ ID NO: | VL | E-H.22 | DIQLTQSPSTLSASVGDRVTITCRASESVDSSGNSFMHW |
| 3114 | | | YQQKPGKAPQLLIYRASNLESGVPSRFSGSGSRTEFTLTI |
| | | | SSLQPDDFATYYCQQSFDDPFTFGQGTKLEIK |
| SEQ ID NO: | VL | E-H.23 | EIVLTQSPDFQSVTPKEKVTITCRASESVDSSGNSFMHW |
| 3115 | | | YQQKPDQSPQLLIYRASNLESGVPSRFSGSGSRTDFTLTI |
| | | | NSLEAEDAATYYCQQSFDDPFTFGQGTKLEIK |
| SEQ ID NO: | VL | E-H.24 | DIQLTQSPSSLSASVGDRVTITCRASESVDSSGNSFMHW |
| 3116 | | | YQQKPGKAPQSLIYRASNLESGVPSKFSGSGSRTDFTLT |
| | | | ISSLQPEDFATYYCQQSFDDPFTFGQGTKLEIK |
| SEQ ID NO: | VL | E-H.25 | DIQLTQSPSSLSASVGDRVTITCRASESVDSSGNSFMHW |
| 3117 | | | YQQKPGKAPQRLIYRASNLESGVPSRFSGSGSRTEFTLTI |
| | | | SSLQPEDFATYYCQQSFDDPFTFGQGTKLEIK |
| SEQ ID NO: | VL | E-H.26 | DIVLTQTPLSLSVTPGQPASISCRASESVDSSGNSFMHW |
| 3118 | | | YLQKPGQPPQLLIYRASNLESGVPDRFSGSGSRTDFTLK |
| | | | ISRVEAEDVGVYYCQQSFDDPFTFGQGTKLEIK |
| SEQ ID NO: | VL | E-H.27 | DIQLTQSPSSLSASVGDRVTITCRASESVDSSGNSFMHW |
| 3119 | | | YQQKPEKAPQSLIYRASNLESGVPSRFSGSGSRTDFTLTI |
| | | | SSLQPEDFATYYCQQSFDDPFTFGQGTKLEIK |
| SEQ ID NO: | VL | E-H.28 | EIVLTQSPPTLSLSPGERVTLSCRASESVDSSGNSFMHW |
| 3120 | | | YQQKPGQAPQLLIYRASNLESGIPARFSGSGSRTDFTLTI |
| | | | SSLQPEDFAVYYCQQSFDDPFTFGQGTKLEIK |
| SEQ ID NO: | VL | E-H.29 | DIQLTQSPSAMSASVGDRVTITCRASESVDSSGNSFMH |
| 3121 | | | WYQQKPGKVPQRLIYRASNLESGVPSRFSGSGSRTEFTL |
| | | | TISSLQPEDFATYYCQQSFDDPFTFGQGTKLEIK |
| SEQ ID NO: 3122 | VL | E-H.30 | DIVLTQSPLSLPVTPGEPASISCRASESVDSSGNSFMHW |
| | | | YLQKPGQSPQLLIYRASNLESGVPDRFSGSGSRTDFTLK |
| | | | ISRVEAEDVGVYYCQQSFDDPFTFGQGTKLEIK |
| SEQ ID NO: | VL | E-H.31 | DIVLTQTPLSLPVTPGEPASISCRASESVDSSGNSFMHW |
| 3123 | | | YLQKPGQSPQLLIYRASNLESGVPDRFSGSGSRTDFTLK |
| | | | ISRVEAEDVGVYYCQQSFDDPFTFGQGTKLEIK |
| SEQ ID NO: | VL | E-H.32 | DIVLTQTPLSLSVTPGQPASISCRASESVDSSGNSFMHW |
| 3124 | | | YLQKPGQSPQLLIYRASNLESGVPDRFSGSGSRTDFTLK |
| | | | ISRVEAEDVGVYYCQQSFDDPFTFGQGTKLEIK |
| SEQ ID NO: | VL | E-H.33 | EIVLTQSPPTLSLSPGERVTLSCRASESVDSSGNSFMHW |
| 3125 | | | YQQKPGQAPQLLIYRASNLESSIPARFSGSGSRTDFTLTI |
| | | | SSLQPEDFAVYYCQQSFDDPFTFGQGTKLEIK |
| SEQ ID NO: | VL | E-H.34 | DIVLTQSPLSLPVTLGQPASISCRASESVDSSGNSFMHW |
| 3126 | | | YQQRPGQSPQRLIYRASNLESGVPDRFSGSGSRTDFTLK |
| | | | ISRVEAEDVGVYYCQQSFDDPFTFGQGTKLEIK |
| SEQ ID NO: | VL | E-H.35 | DIVLTQTPLSSPVTLGQPASISCRASESVDSSGNSFMHW |
| 3127 | | | YQQRPGQPPQLLIYRASNLESGVPDRFSGSGARTDFTLK |
| | | | ISRVEAEDVGVYYCQQSFDDPFTFGQGTKLEIK |
| SEQ ID NO: | VL | E-H.36 | DIVLTQSPAFLSVTPGEKVTITCRASESVDSSGNSFMHW |
| 3128 | | | YQQKPDQAPQLLIYRASNLESGVPSRFSGSGSRTDFTFTI |
| | | | SSLEAEDAATYYCQQSFDDPFTFGQGTKLEIK |
| SEQ ID NO: | VL | E-H.37 | DIQLIQSPSFLSASVGDRVSIICRASESVDSSGNSFMHWY |
| 3129 | | | LQKPGKSPQLFIYRASNLESGVSSRFSGRGSRTDFTLTIIS |
| | | | LKPEDFAAYYCQQSFDDPFTFGQGTKLEIK |
| SEQ ID NO: | VL | E-H.38 | EIVLTQTPLSLSITPGEQASISCRASESVDSSGNSFMHWY |
| 3130 | | | LQKARPVPQLLIYRASNLESGVPDRFSGSGSRTDFTLKIS |
| | | | RVEAEDFGVYYCQQSFDDPFTFGQGTKLEIK |
| SEQ ID NO: | VL | E-H.39 | EIVLTQTPLSLSITPGEQASMSCRASESVDSSGNSFMHW |
| 3131 | | | YLQKARPVPQLLIYRASNLESGVPDRFSGSGSRTDFTLK |
| | | | ISRVEAEDFGVYYCQQSFDDPFTFGQGTKLEIK |
| SEQ ID NO: | VL | E-H.40 | EITLTQSPAFMSATPGDKVNISCRASESVDSSGNSFMHW |
| 3132 | | | YQQKPGEAPQFIIYRASNLESGIPPRFSGSGYRTDFTLTI |
| | | | NNIESEDAAYYYCQQSFDDPFTFGQGTKLEIK |

Variable HEAVY chain (VH)

| | | | |
|---|---|---|---|
| SEQ ID NO: | VH | E-H.1 | QVQLVQSGAEVKKPGASVKVSCKASGYAFSSSWMNW |
| 3133 | | | VRQAPGQGLEWIGRIYPGDGDTKYNGKFKGRATLTAD |
| | | | KSTSTAYMELSSLRSEDTAVYYCARRGTGGWYFDVW |
| | | | GQGTTVTVSS |
| SEQ ID NO: | VH | E-H.2 | QVQLVQSGAEVKKPGSSVKVSCKASGYAFSSSWMNW |
| 3134 | | | VRQAPGQGLEWIGRIYPGDGDTKYNGKFKGRATLTAD |
| | | | KSTSTAYMELSSLRSEDTAVYYCARRGTGGWYFDVW |
| | | | GQGTTVTVSS |
| SEQ ID NO: | VH | E-H.3 | QVQLVQSGAEVKKPGASVKVSCKASGYAFSSSWMNW |
| 3135 | | | VRQAPGKGLEWIGRIYPGDGDTKYNGKFKGRATLTAD |
| | | | KSTSTAYMELSSLRSEDTAVYYCARRGTGGWYFDVW |
| | | | GQGTTVTVSS |
| SEQ ID NO: | VH | E-H.4 | QVQLVQSGAEVKKPGASVKVSCKASGYAFSSSWMNW |
| 3136 | | | VRQAPGQELEWIGRIYPGDGDTKYNGKFKGRATLTAD |
| | | | KSISTAYMELSSLRSEDTATYYCARRGTGGWYFDVWG |
| | | | QGTTVTVSS |
| SEQ ID NO: | VH | E-H.5 | EVQLVQSGAEVKKPGATVKISCKASGYAFSSSWMNWV |
| 3137 | | | QQAPGKGLEWIGRIYPGDGDTKYNGKFKGRATLTADK |
| | | | STSTAYMELSSLRSEDTAVYYCARRGTGGWYFDVWG |
| | | | QGTTVTVSS |
| SEQ ID NO: | VH | E-H.6 | QVQLVQSGAEVKKTGSSVKVSCKASGYAFSSSWMNW |
| 3138 | | | VRQAPGQALEWIGRIYPGDGDTKYNGKFKGRATLTAD |
| | | | KSMSTAYMELSSLRSEDTAMYYCARRGTGGWYFDVW |
| | | | GQGTTVTVSS |
| SEQ ID NO: | VH | E-H.7 | QVQLVQSGAEVKKPGASVKVSCKASGYAFSSSWMNW |
| 3139 | | | VRQAPGQRLEWIGRIYPGDGDTKYNGKFKGRATLTAD |
| | | | KSASTAYMELSSLRSEDMAVYYCARRGTGGWYFDVW |
| | | | GQGTTVTVSS |
| SEQ ID NO: | VH | E-H.8 | QVQLVQSGAEVKKPGASVKVSCKASGYAFSSSWMNW |
| 3140 | | | VRQAPGQGLEWIGRIYPGDGDTKYNGKFKGRATLTAD |
| | | | KSTSTAYMELRSLRSDDMAVYYCARRGTGGWYFDVW |
| | | | GQGTTVTVSS |
| SEQ ID NO: | VH | E-H.9 | QVQLVQSGAEVKKPGASVKVSCKASGYAFSSSWMNW |
| 3141 | | | VRQAPGQRLEWIGRIYPGDGDTKYNGKFKGRATLTAD |
| | | | KSASTAYMELSSLRSEDTAVYYCARRGTGGWYFDVW |
| | | | GQGTTVTVSS |
| SEQ ID NO: | VH | E-H.10 | QVQLVQSGAEVKKPGASVKVSCKASGYAFSSSWMNW |
| 3142 | | | VRQAPGQGLEWIGRIYPGDGDTKYNGKFKGRATLTAD |
| | | | KSTSTAYMELRSLRSDDTAVYYCARRGTGGWYFDVW |
| | | | GQGTTVTVSS |
| SEQ ID NO: | VH | E-H.11 | QVQLVQSGAEVKKPGASVKVSCKASGYAFSSSWMNW |
| 3143 | | | VRQAPGQGLEWIGRIYPGDGDTKYNGKFKGRATLTAD |
| | | | KSISTAYMELSRLRSDDTAVYYCARRGTGGWYFDVWG |
| | | | QGTTVTVSS |
| SEQ ID NO: | VH | E-H.12 | QVQLVQSGAEVKKPGASVKVSCKASGYAFSSSWMNW |
| 3144 | | | VRQAPGQGLEWIGRIYPGDGDTKYNGKFKGRATLTAD |
| | | | KSISTAYMELSRLRSDDTVVYYCARRGTGGWYFDVWG |
| | | | QGTTVTVSS |
| SEQ ID NO: | VH | E-H.13 | QVQLVQSGAEVKKPGASVKVSCKASGYAFSSSWMNW |
| 3145 | | | VRQAPGQGLEWIGRIYPGDGDTKYNGKFKGWATLTAD |
| | | | KSISTAYMELSRLRSDDTAVYYCARRGTGGWYFDVWG |
| | | | QGTTVTVSS |
| SEQ ID NO: | VH | E-H.14 | QVQLVQSGAEVKKPGASVKVSCKASGYAFSSSWMNW |
| 3146 | | | VRQATGQGLEWIGRIYPGDGDTKYNGKFKGRATLTAN |
| | | | KSISTAYMELSSLRSEDTAVYYCARRGTGGWYFDVWG |
| | | | QGTTVTVSS |
| SEQ ID NO: | VH | E-H.15 | QVQLVQSGSELKKPGASVKVSCKASGYAFSSSWMNW |
| 3147 | | | VRQAPGQGLEWIGRIYPGDGDTKYNGKFKGRAVLSAD |
| | | | KSVSTAYLQISSLKAEDTAVYYCARRGTGGWYFDVWG |
| | | | QGTTVTVSS |
| SEQ ID NO: | VH | E-H.16 | QVQLVQSGPEVKKPGTSVKVSCKASGYAFSSSWMNW |
| 3148 | | | VRQARGQRLEWIGRIYPGDGDTKYNGKFKGRATLTAD |
| | | | KSTSTAYMELSSLRSEDTAVYYCARRGTGGWYFDVW |
| | | | GQGTTVTVSS |
| SEQ ID NO: | VH | E-H.17 | EVQLVQSGAEVKKPGESLKISCKASGYAFSSSWMNWV |
| 3149 | | | RQMPGKGLEWIGRIYPGDGDTKYNGKFKGQATLSADK |
| | | | SISTAYLQWSSLKASDTAMYYCARRGTGGWYFDVWG |
| | | | QGTTVTVSS |
| SEQ ID NO: | VH | E-H.18 | QVQLVQSGSELKKPGASVKVSCKASGYAFSSSWMNW |
| 3150 | | | VRQAPGQGLEWIGRIYPGDGDTKYNGKFKGRAVLSAD |
| | | | KSVSMAYLQISSLKAEDTAVYYCARRGTGGWYFDVW |
| | | | GQGTTVTVSS |
| SEQ ID NO: | VH | E-H.19 | QVQLVQSGHEVKQPGASVKVSCKASGYAFSSSWMNW |
| 3151 | | | VPQAPGQGLEWIGRIYPGDGDTKYNGKFKGRAVLSAD |
| | | | KSASTAYLQISSLKAEDMAMYYCARRGTGGWYFDVW |
| | | | GQGTTVTVSS |
| SEQ ID NO: | VH | E-H.20 | EVQLVQSGAEVKKPGESLKISCKASGYAFSSSWMNWV |
| 3152 | | | RQMPGKGLEWIGRIYPGDGDTKYNGKFKGQATLSADK |
| | | | PISTAYLQWSSLKASDTAMYYCARRGTGGWYFDVWG |
| | | | QGTTVTVSS |
| SEQ ID NO: | VH | E-H.21 | EVQLVQSGAEVKKPGESLRISCKASGYAFSSSWMNWV |
| 3153 | | | RQMPGKGLEWIGRIYPGDGDTKYNGKFKGQATLSADK |
| | | | SISTAYLQWSSLKASDTAMYYCARRGTGGWYFDVWG |
| | | | QGTTVTVSS |
| SEQ ID NO: | VH | E-H.22 | EVQLVQSGAEVKKPGESLRISCKASGYAFSSSWMNWV |
| 3154 | | | RQMPGKGLEWIGRIYPGDGDTKYNGKFKGHATLSADK |
| | | | SISTAYLQWSSLKASDTAMYYCARRGTGGWYFDVWG |
| | | | QGTTVTVSS |
| SEQ ID NO: | VH | E-H.23 | QVQLVQSGAEVKKTGSSVKVSCKASGYAFSSSWMNW |
| 3155 | | | VRQAPRQALEWIGRIYPGDGDTKYNGKFKGRATLTAD |
| | | | KSMSTAYMELSSLRSEDTAMYYCARRGTGGWYFDVW |
| | | | GQGTTVTVSS |
| SEQ ID NO: | VH | E-H.24 | EVQLVESGGGLVQPGRSLRLSCTASGYAFSSSWMNWV |
| 3156 | | | RQAPGKGLEWIGRIYPGDGDTKYNGKFKGRATLSADK |
| | | | SKSIAYLQMNSLKTEDTAVYYCARRGTGGWYFDVWG |
| | | | QGTTVTVSS |
| SEQ ID NO: | VH | E-H.25 | EVQLVESGGGLVQPGPSLRLSCTASGYAFSSSWMNWV |
| 3157 | | | RQAPGKGLEWIGRIYPGDGDTKYNGKFKGRATLSADK |
| | | | SKSIAYLQMNSLKTEDTAVYYCARRGTGGWYFDVWG |
| | | | QGTTVTVSS |
| SEQ ID NO: | VH | E-H.26 | QVQLQESGPGLVKPSQTLSLTCTASGYAFSSSWMNWV |
| 3158 | | | RQPPGKGLEWIGRIYPGDGDTKYNGKFKGRATLSADKS |
| | | | KSQASLKLSSVTAADTAVYYCARRGTGGWYFDVWGQ |
| | | | GTTVTVSS |
| SEQ ID NO: | VH | E-H.27 | QVQLQESGPGLVKPSGTLSLTCAASGYAFSSSWMNWV |
| 3159 | | | RQPPGKGLEWIGRIYPGDGDTKYNGKFKGRATLSADKS |
| | | | KSQASLKLSSVTAADTAVYYCARRGTGGWYFDVWGQ |
| | | | GTTVTVSS |
| SEQ ID NO: | VH | E-H.28 | EVQLVESGGGLVKPGRSLRLSCTASGYAFSSSWMNWV |
| 3160 | | | RQAPGKGLEWIGRIYPGDGDTKYNGKFKGRATLSADK |
| | | | SKSIAYLQMNSLKTEDTAVYYCARRGTGGWYFDVWG |
| | | | QGTTVTVSS |
| SEQ ID NO: | VH | E-H.29 | EVQLVESGGGLVQPGGSLKLSCAASGYAFSSSWMNWV |
| 3161 | | | RQASGKGLEWIGRIYPGDGDTKYNGKFKGRATLSADK |
| | | | SKSTAYLQMNSLKTEDTAVYYCARRGTGGWYFDVWG |
| | | | QGTTVTVSS |
| SEQ ID NO: | VH | E-H.30 | QVQLQESGPGLVKPSQTLSLTCAASGYAFSSSWMNWV |
| 3162 | | | RQPPGKGLEWIGRIYPGDGDTKYNGKFKGRATLSADKS |
| | | | KSQASLKLSSVTAADTAVYYCARRGTGGWYFDVWGQ |
| | | | GTTVTVSS |
| SEQ ID NO: | VH | E-H.31 | EVQLVESGGGLVKPGGSLRLSCAASGYAFSSSWMNWV |
| 3163 | | | RQAPGKGLEWIGRIYPGDGDTKYNGKFKGRATLSADK |
| | | | SKSTAYLQMNSLKTEDTAVYYCARRGTGGWYFDVWG |
| | | | QGTTVTVSS |
| SEQ ID NO: | VH | E-H.32 | EVQLVESGGALVKPGGSLRLSCAASGYAFSSSWMNWV |
| 3164 | | | RQAPGKGLEWIGRIYPGDGDTKYNGKFKGRATLSADK |
| | | | SKSTAYLQMNSLKTEDTAVYYCARRGTGGWYFDVWG |
| | | | QGTTVTVSS |
| SEQ ID NO: | VH | E-H.33 | QVQLQESGPGLVKPSQTLSLTCAAYGYAFSSSWMNWV |
| 3165 | | | RQPPGKGLEWIGRIYPGDGDTKYNGKFKGRATLSADKS |
| | | | KSQASLKLSSVTAADTAVYYCARRGTGGWYFDVWGQ |
| | | | GTTVTVSS |
| SEQ ID NO: | VH | E-H.34 | QVQLQESGSGLVKPSQTLSLTCAASGYAFSSSWMNWV |
| 3166 | | | RQPPGKGLEWIGRIYPGDGDTKYNGKFKGRATLSADKS |
| | | | KSQASLKLSSVTAADTAVYYCARRGTGGWYFDVWGQ |
| | | | GTTVTVSS |
| SEQ ID NO: | VH | E-H.35 | EVQLVESGGGLVQPGGSLRLSCAASGYAFSSSWMNWV |
| 3167 | | | RQAPGKGLEWIGRIYPGDGDTKYNGKFKGRATLSADK |
| | | | SKSSAYLQMNSLKTEDTAVYYCARRGTGGWYFDVWG |
| | | | QGTTVTVSS |
| SEQ ID NO: | VH | E-H.36 | QVQLQESGPGLVKPSDTLSLTCTASGYAFSSSWMNWV |
| 3168 | | | RQPPGKGLEWIGRIYPGDGDTKYNGKFKGRATLSADKS |
| | | | KSQASLKLSSVTAADTAVYYCARRGTGGWYFDVWGQ |
| | | | GTTVTVSS |
| SEQ ID NO: | VH | E-H.37 | QVQLQESGPGLVKPSQTLSLTCTASGYAFSSSWMNWV |
| 3169 | | | RQHPGKGLEWIGRIYPGDGDTKYNGKFKGRATLSADK |
| | | | SKSQASLKLSSVTAADTAVYYCARRGTGGWYFDVWG |
| | | | QGTTVTVSS |
| SEQ ID NO: | VH | E-H.38 | QVQLQESGPGLVKPSQTLSLTCTASGYAFSSSWMNWV |
| 3170 | | | RQHPGKGLEWIGRIYPGDGDTKYNGKFKGLATLSADK |
| | | | SKSQASLKLSSVTAADTAVYYCARRGTGGWYFDVWG |
| | | | QGTTVTVSS |
| SEQ ID NO: | VH | E-H.39 | QVQLVESGGGVVQPGRSLRLSCAASGYAFSSSWMNW |
| 3171 | | | VRQAPGKGLEWIGRIYPGDGDTKYNGKFKGRATLSAD |
| | | | KSKSTAYLQMSSLRAEDTAVYYCARRGTGGWYFDVW |
| | | | GQGTTVTVSS |
| SEQ ID NO: | VH | E-H.40 | QVQLVESGGGLVKPGGSLRLSCAASGYAFSSSWMNWV |
| 3172 | | | RQAPGKGLEWIGRIYPGDGDTKYNGKFKGRATLSADK |
| | | | AKSSAYLQMNSLRAEDTAVYYCARRGTGGWYFDVWG |
| | | | QGTTVTVSS |
| SEQ ID NO: | VH | E-H.41 | QVQLVESGGGLVQPGGSLRLSCSASGYAFSSSWMNWV |
| 3173 | | | RQAPGKGLEWIGRIYPGDGDTKYNGKFKGRATLSADK |
| | | | SKSTAYLQMNSLRAEDTAVYYCARRGTGGWYFDVWG |
| | | | QGTTVTVSS |
| SEQ ID NO: | VH | E-H.42 | QVQLLESGGGLVKPGGSLRLSCAASGYAFSSSWMNWV |
| 3174 | | | RQAPGKGLEWIGRIYPGDGDTKYNGKFKGRATLSADK |
| | | | AKSSAYLQMNSLRAEDTAVYYCARRGTGGWYFDVWG |
| | | | QGTTVTVSS |
| SEQ ID NO: | VH | E-H.43 | EVQLVESGGGLVQPGGSLRLSCSASGYAFSSSWMNWV |
| 3175 | | | RQAPGKGLEWIGRIYPGDGDTKYNGKFKGRATLSADK |
| | | | SKSTAYLQMSSLRAEDTAVYYCARRGTGGWYFDVWG |
| | | | QGTTVTVSS |
| SEQ ID NO: | VH | E-H.44 | QVQLQESGPGLVKPSDTLSLTCAASGYAFSSSWMNWV |
| 3176 | | | RQPPGKGLEWIGRIYPGDGDTKYNGKFKGRATLSADKS |
| | | | KSQASLKLSSVTAVDTAVYYCARRGTGGWYFDVWGQ |
| | | | GTTVTVSS |
| SEQ ID NO: | VH | E-H.45 | QVQLQESGPGLVKPSQTLSLTCAASGYAFSSSWMNWV |
| 3177 | | | RQPPGKGLEWIGRIYPGDGDTKYNGKFKGRATLSADKS |
| | | | KSQASLKLSSVTAVDTAVYYCARRGTGGWYFDVWGQ |
| | | | GTTVTVSS |
| SEQ ID NO: | VH | E-H.46 | EVQLVESGGGLVQPGGSLRLSCSASGYAFSSSWMNWV |
| 3178 | | | RQAPGKGLEWIGRIYPGDGDTKYNGKFKGRATLSADK |
| | | | SKSTAYVQMSSLRAEDTAVYYCARRGTGGWYFDVWG |
| | | | QGTTVTVSS |
| SEQ ID NO: | VH | E-H.47 | QVQLVDSGGGVVQPGRSLRLSCAASGYAFSSSWMNW |
| 3179 | | | VRQAPGKGLEWIGRIYPGDGDTKYNGKFKGRATLSAD |
| | | | KSKSTAYLQMNSLRAEDTAVYYCARRGTGGWYFDVW |
| | | | GQGTTVTVSS |
| SEQ ID NO: | VH | E-H.48 | QVQLVESGGGVVQPGRSLRLSCAASGYAFSSSWMNW |
| 3180 | | | VRQAPGKGLEWIGRIYPGDGDTKYNGKFKGRATLSAD |
| | | | KSKSTAYLQMNSLRAEGTAVYYCARRGTGGWYFDVW |
| | | | GQGTTVTVSS |
| SEQ ID NO: | VH | E-H.49 | QVQLVESGGGVVQPGRSLRLSCAASGYAFSSSWMNW |
| 3181 | | | VRQAPGKGLEWIGRIYPGDGDTKYNGKFKGRATLSAD |
| | | | KSKSTAYLQMNSLRAEDTAVYYCARRGTGGWYFDVW |
| | | | GQGTTVTVSS |
| SEQ ID NO: | VH | E-H.50 | EVQLVESGGGLVQPGGSLRLSCAASGYAFSSSWMNWV |
| 3182 | | | RQAPGKGLEWIGRIYPGDGDTKYNGKFKGRATLSADK |
| | | | SKSTAYLQMNSLRAEDTAVYYCARRGTGGWYFDVWG |
| | | | QGTTVTVSS |

TABLE 12
Amino acid sequences for anti TCRβ V10 antibodies. Amino acid and nucleotide
sequences for murine and humanized antibody molecules which bind to TCRBV 10 (e.g., TCRBV
10-1, TCRBV 10-2 or TCRBV 10-3). The amino acid the heavy and light chain CDRs, and the
amino acid and nucleotide sequences of the heavy and light chain variable regions, and the
heavy and light chains are shown.
Murine antibody D, also referred to as S511 antibody

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined)) | LISSGGSYTYYTDSVKG |
| SEQ ID NO: 1290 | HC CDR3(Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFR |
| | | SYGMSWVRQTPDKRLEWVALISSGGSYTY |
| | | YTDSVKGRFTISRDNAKNTLYLQMSSLKSE |
| | | DTAIYYCSRHGGNFFDYWGQGTTLTVSS |
| SEQ ID NO: 3184 | VL | QIVLTQSPSIMSASPGEKVTMTCSVSSSVSY |
| | | MHWYQQKSGTSPKRWIYDTSKLASGVPAR |
| | | FSGSGSGTSYSLTISSMEAEDAATYYCQQW |
| | | SSNPQYTFGGGTKLEIK |

Humanized antibody D (D-H antibody)
Variable light chain (VL)

| | | | |
|---|---|---|---|
| SEQ ID NO: 3185 | VL | D-H.1 | DIVLTQSPAFLSVTPGEKVTITCSVSSSVSYM |
| | | | HWYQQKPDQAPKLLIYDTSKLASGVPSRFS |
| | | | GSGSGTDYTFTISSLEAEDAATYYCQQWSSN |
| | | | PQYTFGQGTKLEIK |
| SEQ ID NO: 3186 | VL | D-H.2 | AIQLTQSPSSLSASVGDRVTITCSVSSSVSYM |
| | | | HWYQQKPGKAPKLLIYDTSKLASGVPSRFS |
| | | | GSGSGTDYTLTISSLQPEDFATYYCQQWSSN |
| | | | PQYTFGQGTKLEIK |
| SEQ ID NO: 3187 | VL | D-H.3 | DIQLTQSPSFLSASVGDRVTITCSVSSSVSYM |
| | | | HWYQQKPGKAPKLLIYDTSKLASGVPSRFS |
| | | | GSGSGTEYTLTISSLQPEDFATYYCQQWSSN |
| | | | PQYTFGQGTKLEIK |
| SEQ ID NO: 3188 | VL | D-H.4 | DIQLTQSPSSLSASVGDRVTITCSVSSSVSYM |
| | | | HWYQQKPGKAPKLLIYDTSKLASGVPSRFS |
| | | | GSGSGTDYTLTISSLQPEDFATYYCQQWSSN |
| | | | PQYTFGQGTKLEIK |
| SEQ ID NO: 3189 | VL | D-H.5 | DIQLTQSPSSVSASVGDRVTITCSVSSSVSYM |
| | | | HWYQQKPGKAPKLLIYDTSKLASGVPSRFS |
| | | | GSGSGTDYTLTISSLQPEDFATYYCQQWSSN |
| | | | PQYTFGQGTKLEIK |
| SEQ ID NO: 3190 | VL | D-H.6 | DIQLTQSPSSLSASVGDRVTITCSVSSSVSYM |
| | | | HWYQQKPGKVPKLLIYDTSKLASGVPSRFS |
| | | | GSGSGTDYTLTISSLQPEDVATYYCQQWSSN |
| | | | PQYTFGQGTKLEIK |
| SEQ ID NO: 3191 | VL | D-H.7 | DIQLTQSPSSLSASVGDRVTITCSVSSSVSYM |
| | | | HWYQQKPGQAPKLLIYDTSKLASGVPSRFS |
| | | | GSGSGTDYTLTISSLQPEDVATYYCQQWSSN |
| | | | PQYTFGQGTKLEIK |
| SEQ ID NO: 3192 | VL | D-H.8 | EIVLTQSPDFQSVTPKEKVTITCSVSSSVSYM |
| | | | HWYQQKPDQSPKLLIYDTSKLASGVPSRFSG |
| | | | SGSGTDYTLTINSLEAEDAATYYCQQWSSNP |
| | | | QYTFGQGTKLEIK |
| SEQ ID NO: 3193 | VL | D-H.9 | AIRLTQSPFSLSASVGDRVTITCSVSSSVSYM |
| | | | HWYQQKPAKAPKLFIYDTSKLASGVPSRFSG |
| | | | SGSGTDYTLTISSLQPEDFATYYCQQWSSNP |
| | | | QYTFGQGTKLEIK |
| SEQ ID NO: 3194 | VL | D-H.10 | DIQLTQSPSSLSASVGDRVTITCSVSSSVSYM |
| | | | HWYQQKPGKAPKLLIYDTSKLASGVPSRFS |
| | | | GSGSGTDYTFTISSLQPEDIATYYCQQWSSNP |
| | | | QYTFGQGTKLEIK |
| SEQ ID NO: 3195 | VL | D-H.11 | EIVLTQSPATLSLSPGERATLSCSVSSSVSYM |
| | | | HWYQQKPGQAPKLLIYDTSKLASGIPARFSG |
| | | | SGSGTDYTLTISSLEPEDFAVYYCQQWSSNP |
| | | | QYTFGQGTKLEIK |
| SEQ ID NO: 3196 | VL | D-H.12 | DIQLTQSPSTLSASVGDRVTITCSVSSSVSYM |
| | | | HWYQQKPGKAPKLLIYDTSKLASGVPSRFS |
| | | | GSGSGTEYTLTISSLQPDDFATYYCQQWSSN |
| | | | PQYTFGQGTKLEIK |
| SEQ ID NO: 3197 | VL | D-H.13 | DIQLTQSPSSLSASVGDRVTITCSVSSSVSYM |
| | | | HWYQQKPGKTPKLLIYDTSKLASGIPSRFSG |
| | | | SGSGTDYTLTIRSLQPEDFATYYCQQWSSNP |
| | | | QYTFGQGTKLEIK |
| SEQ ID NO: 3198 | VL | D-H.14 | EIVLTQSPPTLSLSPGERVTLSCSVSSSVSYM |
| | | | HWYQQKPGQAPKLLIYDTSKLASGIPARFSG |
| | | | SGSGTDYTLTISSLQPEDFAVYYCQQWSSNP |
| | | | QYTFGQGTKLEIK |
| SEQ ID NO: 3199 | VL | D-H.15 | DIQLTQSPSSLSASVGDRVTITCSVSSSVSYM |
| | | | HWYQQKPGKAPKRLIYDTSKLASGVPSRFS |
| | | | GSGSGTEYTLTISSLQPEDFATYYCQQWSSN |
| | | | PQYTFGQGTKLEIK |
| SEQ ID NO: 3200 | VL | D-H.16 | EIVLTQSPATLSLSPGERATLSCSVSSSVSYM |
| | | | HWYQQKPGQAPKLLIYDTSKLASGIPARFSG |
| | | | SGPGTDYTLTISSLEPEDFAVYYCQQWSSNP |
| | | | QYTFGQGTKLEIK |
| SEQ ID NO: 3201 | VL | D-H.17 | EIVLTQSPATLSLSPGERATLSCSVSSSVSYM |
| | | | HWYQQKPGQAPKLLIYDTSKLASGIPARFSG |
| | | | SGSGTDYTLTISRLEPEDFAVYYCQQWSSNP |
| | | | QYTFGQGTKLEIK |
| SEQ ID NO: 3202 | VL | D-H.18 | EIVLTQSPATLSLSPGERATLSCSVSSSVSYM |
| | | | HWYQQKPGQAPKLLIYDTSKLASGIPARFSG |
| | | | SGSGTDYTLTISSLQPEDFAVYYCQQWSSNP |
| | | | QYTFGQGTKLEIK |
| SEQ ID NO: 3203 | VL | D-H.19 | EIVLTQSPATLSVSPGERATLSCSVSSSVSYM |
| | | | HWYQQKPGQAPKLLIYDTSKLASGIPARFSG |
| | | | SGSGTEYTLTISSLQSEDFAVYYCQQWSSNP |
| | | | QYTFGQGTKLEIK |
| SEQ ID NO: 3204 | VL | D-H.20 | EIVLTQSPATLSVSPGERATLSCSVSSSVSYM |
| | | | HWYQQKPGQAPKLLIYDTSKLASGIPARFSG |
| | | | SGSGTEYTLTISILQSEDFAVYYCQQWSSNP |
| | | | QYTFGQGTKLEIK |
| SEQ ID NO: 3205 | VL | D-H.21 | EIVLTQSPPTLSLSPGERVTLSCSVSSSVSYM |
| | | | HWYQQKPGQAPKLLIYDTSKLASSIPARFSG |
| | | | SGSGTDYTLTISSLQPEDFAVYYCQQWSSNP |
| | | | QYTFGQGTKLEIK |
| SEQ ID NO: 3206 | VL | D-H.22 | DIQLTQSPSSLSASVGDRVTITCSVSSSVSYM |
| | | | HWYQQKPGKAPKSLIYDTSKLASGVPSRFSG |
| | | | SGSGTDYTLTISSLQPEDFATYYCQQWSSNP |
| | | | QYTFGQGTKLEIK |
| SEQ ID NO: 3207 | VL | D-H.23 | DIQLTQSPSSLSASVGDRVTITCSVSSSVSYM |
| | | | HWYQQKPGKAPKRLIYDTSKLASGVPSRFS |
| | | | GSGSGTEYTLTISNLQPEDFATYYCQQWSSN |
| | | | PQYTFGQGTKLEIK |
| SEQ ID NO: 3208 | VL | D-H.24 | DIQLTQSPSAMSASVGDRVTITCSVSSSVSY |
| | | | MHWYQQKPGKVPKRLIYDTSKLASGVPSRF |
| | | | SGSGSGTEYTLTISSLQPEDFATYYCQQWSS |
| | | | NPQYTFGQGTKLEIK |
| SEQ ID NO: 3209 | VL | D-H.25 | EIVLTQSPATLSLSPGERATLSCSVSSSVSYM |
| | | | HWYQQKPGQAPKLLIYDTSKLASGIPDRFSG |
| | | | SGSGTDYTLTISRLEPEDFAVYYCQQWSSNP |
| | | | QYTFGQGTKLEIK |
| SEQ ID NO: 3210 | VL | D-H.26 | EIVLTQSPATLSLSPGERATLSCSVSSSVSYM |
| | | | HWYQQKPGLAPKLLIYDTSKLASGIPDRFSG |
| | | | SGSGTDYTLTISRLEPEDFAVYYCQQWSSNP |
| | | | QYTFGQGTKLEIK |
| SEQ ID NO: 3211 | VL | D-H.27 | EIVLTQSPGTLSLSPGERATLSCSVSSSVSYM |
| | | | HWYQQKPGQAPKLLIYDTSKLASGIPDRFSG |
| | | | SGSGTDYTLTISRLEPEDFAVYYCQQWSSNP |
| | | | QYTFGQGTKLEIK |
| SEQ ID NO: 3212 | VL | D-H.28 | DIQLTQSPSSLSASVGDRVTITCSVSSSVSYM |
| | | | HWYQQKPGKAPKSLIYDTSKLASGVPSKFS |
| | | | GSGSGTDYTLTISSLQPEDFATYYCQQWSSN |
| | | | PQYTFGQGTKLEIK |
| SEQ ID NO: 3213 | VL | D-H.29 | DIQLTQSPSSLSASVGDRVTITCSVSSSVSYM |
| | | | HWYQQKPEKAPKSLIYDTSKLASGVPSRFSG |
| | | | SGSGTDYTLTISSLQPEDFATYYCQQWSSNP |
| | | | QYTFGQGTKLEIK |
| SEQ ID NO: 3214 | VL | D-H.30 | DIVLTQSPDSLAVSLGERATINCSVSSSVSYM |
| | | | HWYQQKPGQPPKLLIYDTSKLASGVPDRFS |
| | | | GSGSGTDYTLTISSLQAEDVAVYYCQQWSS |
| | | | NPQYTFGQGTKLEIK |
| SEQ ID NO: 3215 | VL | D-H.31 | EIVLTQTPLSLSITPGEQASMSCSVSSSVSYM |
| | | | HWYLQKARPVPKLLIYDTSKLASGVPDRFS |
| | | | GSGSGTDYTLKISRVEAEDFGVYYCQQWSS |
| | | | NPQYTFGQGTKLEIK |
| SEQ ID NO: 3216 | VL | D-H.32 | EIVLTQTPLSLSITPGEQASISCSVSSSVSYMH |
| | | | WYLQKARPVPKLLIYDTSKLASGVPDRFSGS |
| | | | GSGTDYTLKISRVEAEDFGVYYCQQWSSNP |
| | | | QYTFGQGTKLEIK |
| SEQ ID NO: 3217 | VL | D-H.33 | DIVLTQSPLSLPVTPGEPASISCSVSSSVSYMH |
| | | | WYLQKPGQSPKLLIYDTSKLASGVPDRFSGS |
| | | | GSGTDYTLKISRVEAEDVGVYYCQQWSSNP |
| | | | QYTFGQGTKLEIK |
| SEQ ID NO: 3218 | VL | D-H.34 | DIVLTQSPLSLPVTLGQPASISCSVSSSVSYM |
| | | | HWYQQRPGQSPKRLIYDTSKLASGVPDRFS |
| | | | GSGSGTDYTLKISRVEAEDVGVYYCQQWSS |
| | | | NPQYTFGQGTKLEIK |
| SEQ ID NO: 3219 | VL | D-H.35 | DIVLTQTPLSLPVTPGEPASISCSVSSSVSYM |
| | | | HWYLQKPGQSPKLLIYDTSKLASGVPDRFSG |
| | | | SGSGTDYTLKISRVEAEDVGVYYCQQWSSN |
| | | | PQYTFGQGTKLEIK |
| SEQ ID NO: 3220 | VL | D-H.36 | DIVLTQTPLSLSVTPGQPASISCSVSSSVSYM |
| | | | HWYLQKPGQSPKLLIYDTSKLASGVPDRFSG |
| | | | SGSGTDYTLKISRVEAEDVGVYYCQQWSSN |
| | | | PQYTFGQGTKLEIK |
| SEQ ID NO: 3221 | VL | D-H.37 | DIVLTQTPLSLSVTPGQPASISCSVSSSVSYM |
| | | | HWYLQKPGQPPKLLIYDTSKLASGVPDRFSG |
| | | | SGSGTDYTLKISRVEAEDVGVYYCQQWSSN |
| | | | PQYTFGQGTKLEIK |
| SEQ ID NO: 3222 | VL | D-H.38 | DIQLIQSPSFLSASVGDRVSIICSVSSSVSYMH |
| | | | WYLQKPGKSPKLFIYDTSKLASGVSSRFSGR |
| | | | GSGTDYTLTIISLKPEDFAAYYCQQWSSNPQ |
| | | | YTFGQGTKLEIK |
| SEQ ID NO: 3223 | VL | D-H.39 | DIVLTQTPLSSPVTLGQPASISCSVSSSVSYM |
| | | | HWYQQRPGQPPKLLIYDTSKLASGVPDRFSG |
| | | | SGAGTDYTLKISRVEAEDVGVYYCQQWSSN |
| | | | PQYTFGQGTKLEIK |
| SEQ ID NO: 3224 | VL | D-H.40 | EITLTQSPAFMSATPGDKVNISCSVSSSVSYM |
| | | | HWYQQKPGEAPKFIIYDTSKLASGIPPRFSGS |
| | | | GYGTDYTLTINNIESEDAAYYYCQQWSSNP |
| | | | QYTFGQGTKLEIK |

Variable HEAVY chain (VH)

| | | | |
|---|---|---|---|
| SEQ ID NO: 3225 | VH | D-H.1 | EVQLVESGGGLVKPGGSLRLSCAVSGFTFRS |
| | | | YGMSWVRQAPGKGLEWVALISSGGSYTYY |
| | | | TDSVKGRFTISRDNSKNTLYLQMNSLKTEDT |
| | | | AVYYCSRHGGNFFDYWGQGTTVTVSS |
| SEQ ID NO: 3226 | VH | D-H.2 | EVQLVESGGALVKPGGSLRLSCAVSGFTFRS |
| | | | YGMSWVRQAPGKGLEWVALISSGGSYTYY |
| | | | TDSVKGRFTISRDNSKNTLYLQMNSLKTEDT |
| | | | AVYYCSRHGGNFFDYWGQGTTVTVSS |
| SEQ ID NO: 3227 | VH | D-H.3 | EVQLVESGGGLVQPGGSLRLSCAVSGFTFRS |
| | | | YGMSWVRQAPGKGLEWVALISSGGSYTYY |
| | | | TDSVKGRFTISRDNAKNTLYLQMNSLRAED |
| | | | TAVYYCSRHGGNFFDYWGQGTTVTVSS |
| SEQ ID NO: 3228 | VH | D-H.4 | EVQLVESGGGLVQPGGSLRLSCAVSGFTFRS |
| | | | YGMSWVRQAPGKGLEWVALISSGGSYTYY |
| | | | TDSVKGRFTISRDNAKNSLYLQMNSLRAED |
| | | | TAVYYCSRHGGNFFDYWGQGTTVTVSS |
| SEQ ID NO: 3229 | VH | D-H.5 | EVQLVESGGGLVQPGGSLRLSCAVSGFTFRS |
| | | | YGMSWVRQAPGKGLEWVALISSGGSYTYY |
| | | | TDSVKGRFTISRDNSKNSLYLQMNSLKTEDT |
| | | | AVYYCSRHGGNFFDYWGQGTTVTVSS |
| SEQ ID NO: 3230 | VH | D-H.6 | EVQLVESGGGLVQPGGSLRLSCAVSGFTFRS |
| | | | YGMSWVRQAPGKGLEWVALISSGGSYTYY |
| | | | TDSVKGRFTISRDNAKNSLYLQMNSLRAED |
| | | | MAVYYCSRHGGNFFDYWGQGTTVTVSS |
| SEQ ID NO: 3231 | VH | D-H.7 | EVQLVESGGGLVQPGGSLRLSCAVSGFTFRS |
| | | | YGMSWVRQAPGKGLEWVALISSGGSYTYY |
| | | | TDSVKGQFTISRDNAKNTLYLQMNSLRAED |
| | | | MAVYYCSRHGGNFFDYWGQGTTVTVSS |
| SEQ ID NO: 3232 | VH | D-H.8 | EVQLVESGGGLVKPGRSLRLSCTVSGFTFRS |
| | | | YGMSWVRQAPGKGLEWVALISSGGSYTYY |
| | | | TDSVKGRFTISRDNSKNILYLQMNSLKTEDT |
| | | | AVYYCSRHGGNFFDYWGQGTTVTVSS |
| SEQ ID NO: 3233 | VH | D-H.9 | EVQLVESGGGLVKPGGSLRLSCAVSGFTFRS |
| | | | YGMSWVRQAPGKGLEWVALISSGGSYTYY |
| | | | TDSVKGRFTISRDNAKNSLYLQMNSLRAED |
| | | | TAVYYCSRHGGNFFDYWGQGTTVTVSS |
| SEQ ID NO: 3234 | VH | D-H.10 | EVQLVESGGGLVQPGGSLKLSCAVSGFTFRS |
| | | | YGMSWVRQASGKGLEWVALISSGGSYTYY |
| | | | TDSVKGRFTISRDNSKNTLYLQMNSLKTEDT |
| | | | AVYYCSRHGGNFFDYWGQGTTVTVSS |
| SEQ ID NO: 3235 | VH | D-H.11 | QVQLVESGGGVVQPGGSLRLSCAVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3236 | VH | D-H.12 | QVQLVESGGGVVQPGRSLRLSCAVSGFTFRS |
| | | | YGMSWVRQAPGKGLEWVALISSGGSYTYY |
| | | | TDSVKGRFTISRDNSKNTLYLQMSSLRAEDT |
| | | | AVYYCSRHGGNFFDYWGQGTTVTVSS |
| SEQ ID NO: 3237 | VH | D-H.13 | EVQLVESGGGLVQPGGSLRLSCPVSGFTFRS |
| | | | YGMSWVRQAPGKGLEWVALISSGGSYTYY |
| | | | TDSVKGRFTISRDNANNSLYLQMNSLRAED |
| | | | TAVYYCSRHGGNFFDYWGQGTTVTVSS |
| SEQ ID NO: 3238 | VH | D-H.14 | EVQLVESGGGLVQPGRSLRLSCTVSGFTFRS |
| | | | YGMSWVRQAPGKGLEWVALISSGGSYTYY |
| | | | TDSVKGRFTISRDNSKNILYLQMNSLKTEDT |
| | | | AVYYCSRHGGNFFDYWGQGTTVTVSS |
| SEQ ID NO: 3239 | VH | D-H.15 | EVQLVESGGGLVQPGPSLRLSCTVSGFTFRS |
| | | | YGMSWVRQAPGKGLEWVALISSGGSYTYY |
| | | | TDSVKGRFTISRDNSKNILYLQMNSLKTEDT |
| | | | AVYYCSRHGGNFFDYWGQGTTVTVSS |
| SEQ ID NO: 3240 | VH | D-H.16 | EVQLVESGGGLVQPGGSLRLSCAVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3241 | VH | D-H.17 | EVQLVESGGGLVQPGGSLRLSCAVSGFTFRS |
| | | | YGMSWVRQAPGKGLEWVALISSGGSYTYY |
| | | | TDSVKGRFTISRDNAKNSLYLQMNSLRDED |
| | | | TAVYYCSRHGGNFFDYWGQGTTVTVSS |
| SEQ ID NO: 3242 | VH | D-H.18 | QVQLVESGGGLVKPGGSLRLSCAVSGFTFRS |
| | | | YGMSWVRQAPGKGLEWVALISSGGSYTYY |
| | | | TDSVKGRFTISRDNAKNSLYLQMNSLRAED |
| | | | TAVYYCSRHGGNFFDYWGQGTTVTVSS |
| SEQ ID NO: 3243 | VH | D-H.19 | QVQLVESGGGVVQPGRSLRLSCAVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3244 | VH | D-H.20 | EVQLLESGGGLVQPGGSLRLSCAVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3245 | VH | D-H.21 | EVQLVESGGGLVQPGGSLRLSCAVSGFTFRS |
| | | | YGMSWVRQAPGKGLEWVALISSGGSYTYY |
| | | | TDSVKGRFTISRHNSKNTLYLQMNSLRAEDT |
| | | | AVYYCSRHGGNFFDYWGQGTTVTVSS |
| SEQ ID NO: 3246 | VH | D-H.22 | EVQLVESGGGLIQPGGSLRLSCAVSGFTFRS |
| | | | YGMSWVRQPPGKGLEWVALISSGGSYTYYT |
| | | | DSVKGRFTISRDNSKNTLYLQMNSLRAEDT |
| | | | AVYYCSRHGGNFFDYWGQGTTVTVSS |
| SEQ ID NO: 3247 | VH | D-H.23 | EVQLVESGGGLIQPGGSLRLSCAVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3248 | VH | D-H.24 | EVQLVESGGGLVQPGRSLRLSCAVSGFTFRS |
| | | | YGMSWVRQAPGKGLEWVALISSGGSYTYY |
| | | | TDSVKGRFTISRDNAKNSLYLQMNSLRAED |
| | | | TALYYCSRHGGNFFDYWGQGTTVTVSS |
| SEQ ID NO: 3249 | VH | D-H.25 | QVQLVESGGGVVQPGRSLRLSCAVSGFTFRS |
| | | | YGMSWVRQAPGKGLEWVALISSGGSYTYY |
| | | | TDSVKGRFTISRDNSKNRLYLQMNSLRAEDT |
| | | | AVYYCSRHGGNFFDYWGQGTTVTVSS |
| SEQ ID NO: 3250 | VH | D-H.26 | QVQLVESGGGVVQPGRSLRLSCAVSGFTFRS |
| | | | YGMSWVRQAPGKGLEWVALISSGGSYTYY |
| | | | TDSVKGRFTISRDNSKNTLYLQMNSLRAEGT |
| | | | AVYYCSRHGGNFFDYWGQGTTVTVSS |
| SEQ ID NO: 3251 | VH | D-H.27 | QVQLVESGGGVVQPGRSLRLSCAVSGFTFRS |
| | | | YGMSWVRQAPGKGLEWVALISSGGSYTYY |
| | | | TDSVKGRFAISRDNSKNTLYLQMNSLRAED |
| | | | TAVYYCSRHGGNFFDYWGQGTTVTVSS |
| SEQ ID NO: 3252 | VH | D-H.28 | QVQLVDSGGGVVQPGRSLRLSCAVSGFTFR |
| | | | SYGMSWVRQAPGKGLEWVALISSGGSYTY |
| | | | YTDSVKGRFTISRDNSKNTLYLQMNSLRAE |
| | | | DTAVYYCSRHGGNFFDYWGQGTTVTVSS |
| SEQ ID NO: 3253 | VH | D-H.29 | EVQLVESGGGVVRPGGSLRLSCAVSGFTFRS |
| | | | YGMSWVRQAPGKGLEWVALISSGGSYTYY |
| | | | TDSVKGRFTISRDNAKNSLYLQMNSLRAED |
| | | | TALYHCSRHGGNFFDYWGQGTTVTVSS |
| SEQ ID NO: 3254 | VH | D-H.30 | EVQLVESGGVVVQPGGSLRLSCAVSGFTFRS |
| | | | YGMSWVRQAPGKGLEWVALISSGGSYTYY |
| | | | TDSVKGRFTISRDNSKNSLYLQMNSLRAEDT |
| | | | ALYYCSRHGGNFFDYWGQGTTVTVSS |
| SEQ ID NO: 3255 | VH | D-H.31 | EVQLVESGGGVVQPGGSLRLSCAVSGFTFRS |
| | | | YGMSWVRQAPGKGLEWVALISSGGSYTYY |
| | | | TDSVKGRFTISRDNSKNSLYLQMNSLRTEDT |
| | | | ALYYCSRHGGNFFDYWGQGTTVTVSS |
| SEQ ID NO: 3256 | VH | D-H.32 | EVQLVESGGVVVQPGGSLRLSCAVSGFTFRS |
| | | | YGMSWVRQAPGKGLEWVALISSGGSYTYY |
| | | | TDSVKGRFTISRDNSKNSLYLQMNSLRTEDT |
| | | | ALYYCSRHGGNFFDYWGQGTTVTVSS |
| SEQ ID NO: 3257 | VH | D-H.33 | EVQLVETGGGLIQPGGSLRLSCAVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3258 | VH | D-H.34 | EVQLVESGGGLVQPGGSLRLSCAVSGFTFRS |
| | | | YGMSWVRQATGKGLEWVALISSGGSYTYY |
| | | | TDSVKGRFTISRENAKNSLYLQMNSLRAGD |
| | | | TAVYYCSRHGGNFFDYWGQGTTVTVSS |
| SEQ ID NO: 3259 | VH | D-H.35 | EVQLVESRGVLVQPGGSLRLSCAVSGFTFRS |
| | | | YGMSWVRQAPGKGLEWVALISSGGSYTYY |
| | | | TDSVKGRFTISRDNSKNTLHLQMNSLRAEDT |
| | | | AVYYCSRHGGNFFDYWGQGTTVTVSS |
| SEQ ID NO: 3260 | VH | D-H.36 | EVQLVESGGGLVQPGRSLRLSCAVSGFTFRS |
| | | | YGMSWVRQAPGKGLEWVALISSGGSYTYY |
| | | | TDSVKGRFTISRDNAKNSLYLQMNSLRAED |
| | | | MALYYCSRHGGNFFDYWGQGTTVTVSS |
| SEQ ID NO: 3261 | VH | D-H.37 | QVQLVESGGGLVQPGGSLRLSCSVSGFTFRS |
| | | | YGMSWVRQAPGKGLEWVALISSGGSYTYY |
| | | | TDSVKGRFTISRDNSKNTLYLQMNSLRAEDT |
| | | | AVYYCSRHGGNFFDYWGQGTTVTVSS |
| SEQ ID NO: 3262 | VH | D-H.38 | EVQLVESGGGLVQPGGSLRLSCSVSGFTFRS |
| | | | YGMSWVRQAPGKGLEWVALISSGGSYTYY |
| | | | TDSVKGRFTISRDNSKNTLYLQMSSLRAEDT |
| | | | AVYYCSRHGGNFFDYWGQGTTVTVSS |
| SEQ ID NO: 3263 | VH | D-H.39 | QVQLVESGGGVVQPGRSLRLSCAVSGFTFRS |
| | | | YGMSWVRQAPGKGLEWVALISSGGSYTYY |
| | | | TDSVKGRFTISRDNSTNTLFLQMNSLRAEDT |
| | | | AVYYCSRHGGNFFDYWGQGTTVTVSS |
| SEQ ID NO: 3264 | VH | D-H.40 | QVQLLESGGGLVKPGGSLRLSCAVSGFTFRS |
| | | | YGMSWVRQAPGKGLEWVALISSGGSYTYY |
| | | | TDSVKGRFTISRDNAKNSLYLQMNSLRAED |
| | | | TAVYYCSRHGGNFFDYWGQGTTVTVSS |
| SEQ ID NO: 3265 | VH | D-H.41 | EVQLVESGEGLVQPGGSLRLSCAVSGFTFRS |
| | | | YGMSWVRQAPGKGLEWVALISSGGSYTYY |
| | | | TDSVKGRFTISRDNSKNTLYLQMGSLRAED |
| | | | MAVYYCSRHGGNFFDYWGQGTTVTVSS |
| SEQ ID NO: 3266 | VH | D-H.42 | EVQLVESGGGLVQPGGSLRLSCAVSGFTFRS |
| | | | YGMSWVRQAPGKGLEWVALISSGGSYTYY |
| | | | TDSVKGRFTISRDNSKNTLYLQMGSLRAED |
| | | | MAVYYCSRHGGNFFDYWGQGTTVTVSS |
| SEQ ID NO: 3267 | VH | D-H.43 | EVQLVESGGGLVQPGGSLRLSCSVSGFTFRS |
| | | | YGMSWVRQAPGKGLEWVALISSGGSYTYY |
| | | | TDSVKGRFTISRDNSKNTLYVQMSSLRAEDT |
| | | | AVYYCSRHGGNFFDYWGQGTTVTVSS |
| SEQ ID NO: 3268 | VH | D-H.44 | EVQLVESGGGLVQPGGSLRLSCAVSGFTFRS |
| | | | YGMSWVRQAPGKGLEWVALISSGGSYTYY |
| | | | TDSVKGRFIISRDNSRNSLYLQKNRRRAEDM |
| | | | AVYYCSRHGGNFFDYWGQGTTVTVSS |
| SEQ ID NO: 3269 | VH | D-H.45 | EVQLVESGGGLVQPGGSLRLSCAVSGFTFRS |
| | | | YGMSWVHQAPGKGLEWVALISSGGSYTYY |
| | | | TDSVKGRFIISRDNSRNTLYLQTNSLRAEDT |
| | | | AVYYCSRHGGNFFDYWGQGTTVTVSS |
| SEQ ID NO: 3270 | VH | D-H.46 | EVHLVESGGGLVQPGGALRLSCAVSGFTFRS |
| | | | YGMSWVRQATGKGLEWVALISSGGSYTYY |
| | | | TDSVKGRFTISRENAKNSLYLQMNSLRAGD |
| | | | TAVYYCSRHGGNFFDYWGQGTTVTVSS |
| SEQ ID NO: 3271 | VH | D-H.47 | EVQLVESGGGLVQPRGSLRLSCAVSGFTFRS |
| | | | YGMSWVRQAPGKGLEWVALISSGGSYTYY |
| | | | TDSVKGRFTISRDNSKNTLYLQMNNLRAEG |
| | | | TAVYYCSRHGGNFFDYWGQGTTVTVSS |
| SEQ ID NO: 3272 | VH | D-H.48 | EVQLVESGGGLVQPRGSLRLSCAVSGFTFRS |
| | | | YGMSWVRQAPGKGLEWVALISSGGSYTYY |
| | | | TDSVKGRFTISRDNSKNTLYLQMNNLRAEG |
| | | | TAAYYCSRHGGNFFDYWGQGTTVTVSS |
| SEQ ID NO: 3273 | VH | D-H.49 | QVQLVQSGAEVKKPGASVKVSCKVSGFTFR |
| | | | SYGMSWVRQAPGKGLEWVALISSGGSYTY |
| | | | YTDSVKGRFTITRDNSTNTLYMELSSLRSED |
| | | | TAVYYCSRHGGNFFDYWGQGTTVTVSS |
| SEQ ID NO: 3274 | VH | D-H.50 | QVQLVQSGSELKKPGASVKVSCKVSGFTFRS |
| | | | YGMSWVRQAPGQGLEWVALISSGGSYTYY |
| | | | TDSVKGRFVISRDNSVNTLYLQISSLKAEDT |
| | | | AVYYCSRHGGNFFDYWGQGTTVTVSS |

TABLE 13
Amino acid sequences for additional anti-TCRβ V antibodies. Amino acid and
nucleotide sequences for murine and humanized antibody molecules which bind to various
TCRVB families are disclosed. The amino acid the heavy and light chain CDRs, and the amino
acid and nucleotide sequences of the heavy and light chain variable regions, and the heavy and
light chains are shown. Antibodies disclosed in the table include, MPB2D5, CAS1.1.3,
IMMU222, REA1062, and JOVI-3. MPB2D5 binds human TCRβV 20-1 (TCRβV2 per old
nomenclature). CAS1.1.3 binds human TCRβV 27 (TCRβV14 per old nomenclature). IMMU 222
binds human TCRβV 6-5, TCRβV 6-6, or TCRβV 6-9 (TCRβV13.1 per old nomenclature).
REA1062 binds human TCRβV 5-1). JOVI-3 binds human TCRβV 28 (TCRβV3. 1 per old
nomenclature). IMMU546 binds human TCRβV 2.
MPB2D5 (murine), also referred to here as BJ1188, BJ1190 and REA654; or Antibody G
Binds to human TCRVβ 20-1

| | | |
|---|---|---|
| SEQ ID NO: 1102 | HC CDR1 (Kabat) | SAYMH |
| SEQ ID NO: 1103 | HC CDR2 (Kabat) | RIDPATGKTKYAPKFQA |
| SEQ ID NO: 1104 | HC CDR3 (Kabat) | SLNWDYGLDY |
| SEQ ID NO: 1105 | HC CDR1 (Chothia) | GFNIKSA |
| SEQ ID NO: 1106 | HC CDR2 (Chothia) | DPATGK |
| SEQ ID NO: 1104 | HC CDR3 (Chothia) | SLNWDYGLDY |
| SEQ ID NO: 3640 | HC CDR1 (Combined) | GFNIKSAYMH |
| SEQ ID NO: 1103 | HC CDR2 (Combined) | RIDPATGKTKYAPKFQA |
| SEQ ID NO: 1104 | HC CDR3 (Combined) | SLNWDYGLDY |
| SEQ ID NO: 1107 | LC CDR1 (Kabat) | RASKSVSILGTHLIH |
| SEQ ID NO: 1108 | LC CDR2 (Kabat) | AASNLES |
| SEQ ID NO: 1109 | LC CDR3 (Kabat) | QQSIEDPWT |
| SEQ ID NO: 1110 | LC CDR1 (Chothia) | SKSVSILGTHL |
| SEQ ID NO: 1108 | LC CDR2 (Chothia) | AASNLES |
| SEQ ID NO: 1109 | LC CDR3 (Chothia) | QQSIEDPWT |
| SEQ ID NO: 1107 | LC CDR1 (Combined) | RASKSVSILGTHLIH |
| SEQ ID NO: 1108 | LC CDR2 (Combined) | AASNLES |
| SEQ ID NO: 1109 | LC CDR3(Combined) | QQSIEDPWT |
| SEQ ID NO: 1111 | VL | DIVLTQSPASLAVSLGQRATISCRASKSVSILGTH |
| | | LIHWYQQKPGQPPKLLIYAASNLESGVPARFSGS |
| | | GSETVFTLNIHPVEEEDAATYFCQQSIEDPWTFGG |
| | | GTKLGIK |
| SEQ ID NO: 1112 | VH | EVQLQQSVADLVRPGASLKLSCTASGFNIKSAYM |
| | | HWVIQRPDQGPECLGRIDPATGKTKYAPKFQAK |
| | | ATITADTSSNTAYLQLSSLTSEDTAIYYCTRSLNW |
| | | DYGLDYWGQGTSVTVSS |

VH for MPB2D5 (humanized) also referred to as Antibody G-H (humanized)
Binds to human TCRVβ 20-1

| | | |
|---|---|---|
| SEQ ID NO: 1113 | VH - 1 | QVQLVQSGAEVKKPGASVKVSCKASGFNIKSAY |
| | | MHWVRQAPGQGLEWMGRIDPATGKTKYAPKFQ |
| | | ARVTMTADTSTNTAYMELSSLRSEDTAVYYCAR |
| | | SLNWDYGLDYWGQGTLVTVSS |
| SEQ ID NO: 1114 | VH - 2 | QVQLVQSGAEVKKPGASVKVSCKASGFNIKSAY |
| | | MHWVRQAPGQEPGCMGRIDPATGKTKYAPKFQ |
| | | ARVTMTADTSINTAYTELSSLRSEDTATYYCARS |
| | | LNWDYGLDYWGQGTLVTVSS |
| SEQ ID NO: 1115 | VH - 3 | QVQLVQSGAEVKKPGSSVKVSCKASGFNIKSAY |
| | | MHWVRQAPGQGLEWMGRIDPATGKTKYAPKFQ |
| | | ARVTITADTSTNTAYMELSSLRSEDTAVYYCARS |
| | | LNWDYGLDYWGQGTLVTVSS |
| SEQ ID NO: 1116 | VH - 4 | QVQLVQSGAEVKKPGASVKVSCKASGFNIKSAY |
| | | MHWVRQAPGQRLEWMGRIDPATGKTKYAPKFQ |
| | | ARVTITADTSANTAYMELSSLRSEDTAVYYCARS |
| | | LNWDYGLDYWGQGTLVTVSS |

VL for MPB2D5 (humanized) also referred to as Antibody G-H (humanized)
Binds to human TCRβV 20-1

| | | |
|---|---|---|
| SEQ ID NO: 1117 | VL - 1 | EIVLTQSPATLSLSPGERATLSCRASKSVSILGTHL |
| | | IHWYQQKPGQAPRLLIYAASNLESGIPARFSGSGS |
| | | ETDFTLTISSLEPEDFAVYFCQQSIEDPFGGGTKV |
| | | EIK |
| SEQ ID NO: 1118 | VL - 2 | EIVLTQSPATLSLSPGERATLSCRASKSVSILGTHL |
| | | IHWYQQKPGLAPRLLIYAASNLESGIPDRFSGSGS |
| | | ETDFTLTISRLEPEDFAVYFCQQSIEDPFGGGTKV |
| | | EIK |
| SEQ ID NO: 1119 | VL - 3 | EIVLTQSPGTLSLSPGERATLSCRASKSVSILGTHL |
| | | IHWYQQKPGQAPRLLIYAASNLESGIPDRFSGSGS |
| | | ETDFTLTISRLEPEDFAVYFCQQSIEDPFGGGTKV |
| | | EIK |

CAS1.1.3 (murine) also referred to herein as BJ1460; or Antibody H
Binds to human TCRVβ 27

| | | |
|---|---|---|
| SEQ ID NO: 1120 | HC CDR1 (Kabat) | DTYMY |
| SEQ ID NO: 1121 | HC CDR2 (Kabat) | RIDPANGNTKYDPKFQD |
| SEQ ID NO: 1122 | HC CDR3 (Kabat) | GSYYYAMDY |
| SEQ ID NO: 1123 | HC CDR1 (Chothia) | GFKTEDT |
| SEQ ID NO: 1124 | HC CDR2 (Chothia) | DPANGN |
| SEQ ID NO: 1122 | HC CDR3 (Chothia) | GSYYYAMDY |
| SEQ ID NO: 1125 | HC CDR1 (Combined) | GFKTEDTYMY |
| SEQ ID NO: 1121 | HC CDR2 (Combined) | RIDPANGNTKYDPKFQD |
| SEQ ID NO: 1122 | HC CDR3(Combined) | GSYYYAMDY |
| SEQ ID NO: 1126 | LC CDR1 (Kabat) | RASESVDSYGNSFMH |
| SEQ ID NO: 1127 | LC CDR2 (Kabat) | RASNLES |
| SEQ ID NO: 1128 | LC CDR3 (Kabat) | QQSNEDPYT |
| SEQ ID NO: 3641 | LC CDR1 (Chothia) | SESVDSYGNSF |
| SEQ ID NO: 1127 | LC CDR2 (Chothia) | RASNLES |
| SEQ ID NO: 1128 | LC CDR3 (Chothia) | QQSNEDPYT |
| SEQ ID NO: 1126 | LC CDR1 (Combined) | RASESVDSYGNSFMH |
| SEQ ID NO: 1127 | LC CDR2 (Combined) | RASNLES |
| SEQ ID NO: 1128 | LC CDR3(Combined) | QQSNEDPYT |
| SEQ ID NO: 1129 | VL | DIVLTQSPASLAVSLGQRATISCRASESVDSYGNS |
| | | FMHWYQQKPGQPPKLLIYRASNLESGIPARFSGS |
| | | GSRTDFTLTINPVEADDVATYYCQQSNEDPYTFG |
| | | GGTKLEIK |
| SEQ ID NO: 1130 | VH | EVQLQQSGAELVKPGASVKLSCTASGFKTEDTY |
| | | MYWVKQRPEQGLEWIGRIDPANGNTKYDPKFQ |
| | | DKATITADSSSNTAYLQLSSLPSEDTAVYYCARG |
| | | SYYYAMDYWGQGTSVTVSS |

VH for CAS1.1.3 (humanized) also referred to as Antibody H-H (humanized)
Binds to human TCRVβ 27

| | | |
|---|---|---|
| SEQ ID NO: 1131 | VH - 1 | QVQLVQSGAEVKKPGSSVKVSCKASGFKTEDTY |
| | | MYWVRQAPGQGLEWIGRIDPANGNTKYDPKFQ |
| | | DRATITADSSTNTAYMELSSLRSEDTAVYYCARG |
| | | SYYYAMDYWGQGTLVTVSS |
| SEQ ID NO: 1132 | VH - 2 | QVQLVQSGAEVKKPGASVKVSCKASGFKTEDTY |
| | | MYWVRQAPGQRLEWIGRIDPANGNTKYDPKFQ |
| | | DRATITADSSANTAYMELSSLRSEDTAVYYCARG |
| | | SYYYAMDYWGQGTLVTVSS |
| SEQ ID NO: 1133 | VH - 3 | EVQLVESGGGLVQPGGSLKLSCAASGFKTEDTY |
| | | MYWVRQASGKGLEWIGRIDPANGNTKYDPKFQ |
| | | DRATISADSSKNTAYLQMNSLKTEDTAVYYCAR |
| | | GSYYYAMDYWGQGTLVTVSS |
| SEQ ID NO: 1134 | VH - 4 | EVQLVQSGAEVKKPGESLRISCKASGFKTEDTYM |
| | | YWVRQMPGKGLEWIGRIDPANGNTKYDPKFQD |
| | | QATISADSSINTAYLQWSSLKASDTAMYYCARGS |
| | | YYYAMDYWGQGTLVTVSS |
| SEQ ID NO: 1135 | VH - 5 | QVQLVQSGSELKKPGASVKVSCKASGFKTEDTY |
| | | MYWVRQAPGQGLEWIGRIDPANGNTKYDPKFQ |
| | | DRAVISADSSVNTAYLQISSLKAEDTAVYYCARG |
| | | SYYYAMDYWGQGTLVTVSS |

VL for CAS1.1.3 (humanized) also referred to as Antibody H-H (humanized)
Binds to human TCRVβ 27

| | | |
|---|---|---|
| SEQ ID NO: 1136 | VL - 1 | DIVLTQSPDSLAVSLGERATINCRASESVDSYGNS |
| | | FMHWYQQKPGQPPKLLIYRASNLESGVPDRFSGS |
| | | GSRTDFTLTISSLQAEDVAVYYCQQSNEDPYTFG |
| | | QGTKLEIK |
| SEQ ID NO: 1137 | VL - 2 | EIVLTQSPATLSLSPGERATLSCRASESVDSYGNS |
| | | FMHWYQQKPGQAPKLLIYRASNLESGIPARFSGS |
| | | GSRTDFTLTISRLEPEDFAVYYCQQSNEDPYTFGQ |
| | | GTKLEIK |
| SEQ ID NO: 1138 | VL - 3 | DIQLTQSPSSLSASVGDRVTITCRASESVDSYGNS |
| | | FMHWYQQKPGQAPKLLIYRASNLESGVPSRFSGS |
| | | GSRTDFTLTISSLQPEDVATYYCQQSNEDPYTFG |
| | | QGTKLEIK |
| SEQ ID NO: 1139 | VL - 4 | AIQLTQSPSSLSASVGDRVTITCRASESVDSYGNS |
| | | FMHWYQQKPGKAPKLLIYRASNLESGVPSRFSGS |
| | | GSRTDFTLTISSLQPEDFATYYCQQSNEDPYTFGQ |
| | | GTKLEIK |
| SEQ ID NO: 1140 | VL - 5 | EIVLTQSPDFQSVTPKEKVTITCRASESVDSYGNS |
| | | FMHWYQQKPDQSPKLLIYRASNLESGVPSRFSGS |
| | | GSRTDFTLTINSLEAEDAATYYCQQSNEDPYTFG |
| | | QGTKLEIK |

IMMU222 (murine) also referred to as BJ1461; or Antibody I
Binds to human TCRVβ 6-5, 6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3(Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3(Combined) | LQGSGFPLT |
| SEQ ID NO: 1151 | VL | ENVLTQSPAIMSASPGEKVTMTCSAGSSVSFMH |
| | | WYQQKSSTSPKLWIYDTSKLASGVPGRFSGSGSG |
| | | NSFSLTISSMEAEDVAIYYCLQGSGFPLTFGSGTK |
| | | LEIK |
| SEQ ID NO: 1152 | VH | DVKLVESGEGLVKPGGSLKLSCAASGFTFRSYA |
| | | MSWVRQTPEKRLEWVAHISNGGDYIYYADTVK |
| | | GRFTISRDNARNTLYLQMSSLKSEDTAMYYCTRP |
| | | SYYSDPWFFDVWGTGTTVTVSS |

VH for IMMU222 (humanized) also referred to as Antibody I-H
Binds to human TCRVβ 6-5, 6-6, 6-9

| | | |
|---|---|---|
| SEQ ID NO: 1153 | VH - 1 | EVQLVESGGGLVQPGGSLRLSCAASGFTFRSYA |
| | | MSWVRQAPGKGLEWVAHISNGGDYIYYADTVK |
| | | GRFTISRDNAKNSLYLQMNSLRAEDTAVYYCTR |
| | | PSYYSDPWFFDVWGQGTTVTVSS |
| SEQ ID NO: 1154 | VH - 2 | QVQLVESGGGVVQPGRSLRLSCAASGFTFRSYA |
| | | MSWVRQAPGKGLEWVAHISNGGDYIYYADTVK |
| | | GRFTISRDNSKNTLYLQMSSLRAEDTAVYYCTRP |
| | | SYYSDPWFFDVWGQGTTVTVSS |
| SEQ ID NO: 1155 | VH - 3 | EVQLVESGGGLVQPGGSLRLSCAASGFTFRSYA |
| | | MSWVRQAPGKGLEWVAHISNGGDYIYYADTVK |
| | | GRFTISRDNSKNTLYLQMNSLRAEDTAVYYCTRP |
| | | SYYSDPWFFDVWGQGTTVTVSS |
| SEQ ID NO: 1156 | VH - 4 | QVQLVQSGSELKKPGASVKVSCKASGFTFRSYA |
| | | MSWVRQAPGQGLEWVAHISNGGDYIYYADTVK |
| | | GRFVISRDNSVNTLYLQISSLKAEDTAVYYCTRPS |
| | | YYSDPWFFDVWGQGTTVTVSS |
| SEQ ID NO: 1157 | VH - 5 | QVQLVQSGAEVKKPGASVKVSCKASGFTFRSYA |
| | | MSWVRQAPGQRLEWVAHISNGGDYIYYADTVK |
| | | GRFTITRDNSANTLYMELSSLRSEDTAVYYCTRP |
| | | SYYSDPWFFDVWGQGTTVTVSS |

VL for IMMU222 (humanized) ) also referred to as Antibody I-H
Binds to human TCRVβ 6-5, 6-6, 6-9

| | | |
|---|---|---|
| SEQ ID NO: 1158 | VL - 1 | ENVLTQSPATLSLSPGERATLSCSAGSSVSFMHW |
| | | YQQKPGQAPKLLIYDTSKLASGIPARFSGSGSGN |
| | | DFTLTISSLEPEDFAVYYCLQGSGFPLTFGQGTKL |
| | | EIK |
| SEQ ID NO: 1159 | VL - 2 | ENVLTQSPDFQSVTPKEKVTITCSAGSSVSFMHW |
| | | YQQKPDQSPKLLIYDTSKLASGVPSRFSGSGSGN |
| | | DFTLTINSLEAEDAATYYCLQGSGFPLTFGQGTK |
| | | LEIK |
| SEQ ID NO: 1160 | VL - 3 | DNQLTQSPSSLSASVGDRVTITCSAGSSVSFMHW |
| | | YQQKPGKVPKLLIYDTSKLASGVPSRFSGSGSGN |
| | | DFTLTISSLQPEDVATYYCLQGSGFPLTFGQGTKL |
| | | EIK |
| SEQ ID NO: 1161 | VL - 4 | ANQLTQSPSSLSASVGDRVTITCSAGSSVSFMHW |
| | | YQQKPGKAPKLLIYDTSKLASGVPSRFSGSGSGN |
| | | DFTLTISSLQPEDFATYYCLQGSGFPLTFGQGTKL |
| | | EIK |
| SEQ ID NO: 1162 | VL - 5 | DNVLTQSPDSLAVSLGERATINCSAGSSVSFMHW |
| | | YQQKPGQPPKLLIYDTSKLASGVPDRFSGSGSGN |
| | | DFTLTISSLQAEDVAVYYCLQGSGFPLTFGQGTK |
| | | LEIK |

REA1062 (murine), also referred to as BJ1189 or as Antibody J
Binds to human TCRVβ 5-1

| | | |
|---|---|---|
| SEQ ID NO: 1163 | HC CDR1 (Kabat) | DYNIH |
| SEQ ID NO: 1164 | HC CDR2 (Kabat) | YINPYNGRTGYNQKFKA |
| SEQ ID NO: 1165 | HC CDR3 (Kabat) | WDGSSYFDY |
| SEQ ID NO: 1166 | HC CDR1 (Chothia) | GYTFTDYNIH |
| SEQ ID NO: 1167 | HC CDR2 (Chothia) | NPYNGR |
| SEQ ID NO: 1165 | HC CDR3 (Chothia) | WDGSSYFDY |
| SEQ ID NO: 1166 | HC CDR1 (Combined) | GYTFTDYNIH |
| SEQ ID NO: 1164 | HC CDR2 (Combined) | YINPYNGRTGYNQKFKA |
| SEQ ID NO: 1165 | HC CDR3(Combined) | WDGSSYFDY |
| SEQ ID NO: 1168 | LC CDR1 (Kabat) | SASSSVSYMH |
| SEQ ID NO: 1169 | LC CDR2 (Kabat) | EISKLAS |
| SEQ ID NO: 1170 | LC CDR3 (Kabat) | QQWNYPLLT |
| SEQ ID NO: 1297 | LC CDR1 (Chothia) | SSSVSY |
| SEQ ID NO: 1169 | LC CDR2 (Chothia) | EISKLAS |
| SEQ ID NO: 1170 | LC CDR3 (Chothia) | QQWNYPLLT |
| SEQ ID NO: 1168 | LC CDR1 (Combined) | SASSSVSYMH |
| SEQ ID NO: 1169 | LC CDR2 (Combined) | EISKLAS |
| SEQ ID NO: 1170 | LC CDR3(Combined) | QQWNYPLLT |
| SEQ ID NO: 1171 | VL | EIVLTQSPAITAASLGQKVTITCSASSSVSYMHWY |
| | | QQKSGTSPKPWIYEISKLASGVPARFSGSGSGTSY |
| | | SLTISSMEAEDAAIYYCQQWNYPLLTFGAGTKLE |
| | | LK |
| SEQ ID NO: 1172 | VH | EVQLQQSGPVLVKPGASVRMSCKASGYTFTDYN |
| | | IHWVKQSHGRSLEWVGYINPYNGRTGYNQKFKA |
| | | KATLTVDKSSSTAYMDLRSLTSEDSAVYYCARW |
| | | DGSSYFDYWGQGTTLTVSS |

VH for REA1062 (humanized) also referred to as Antibody J-H
Binds to human TCRVβ 5-1

| | | |
|---|---|---|
| SEQ ID NO: 1173 | VH - 1 | QVQLVQSGAEVKKPGSSVKVSCKASGYTFTDYN |
| | | IHWVRQAPGQGLEWVGYINPYNGRTGYNQKFK |
| | | ARATLTVDKSTSTAYMELSSLRSEDTAVYYCAR |
| | | WDGSSYFDYWGQGTTVTVSS |
| SEQ ID NO: 1174 | VH - 2 | QVQLVQSGAEVKKPGASVKVSCKASGYTFTDYN |
| | | IHWVRQAPGQGLEWVGYINPYNGRTGYNQKFK |
| | | ARATLTVDKSTSTAYMELRSLRSDDMAVYYCAR |
| | | WDGSSYFDYWGQGTTVTVSS |
| SEQ ID NO: 1175 | VH - 3 | QVQLVQSGAEVKKPGASVKVSCKASGYTFTDYN |
| | | IHWVRQATGQGLEWVGYINPYNGRTGYNQKFK |
| | | ARATLTVNKSISTAYMELSSLRSEDTAVYYCAR |
| | | WDGSSYFDYWGQGTTVTVSS |
| SEQ ID NO: 1176 | VH - 4 | EVQLVESGGGLVQPGRSLRLSCTASGYTFTDYNI |
| | | HWVRQAPGKGLEWVGYINPYNGRTGYNQKFKA |
| | | RATLSVDKSKSIAYLQMNSLKTEDTAVYYCARW |
| | | DGSSYFDYWGQGTTVTVSS |
| SEQ ID NO: 1177 | VH - 5 | QVQLVQSGSELKKPGASVKVSCKASGYTFTDYNI |
| | | HWVRQAPGQGLEWVGYINPYNGRTGYNQKFKA |
| | | RAVLSVDKSVSTAYLQISSLKAEDTAVYYCARW |
| | | DGSSYFDYWGQGTTVTVSS |

VL for REA1062 (humanized) also referred to as Antibody J-H
Binds to human TCRVβ 5-1

| | | |
|---|---|---|
| SEQ ID NO: 1178 | VL - 1 | EIVLTQSPATLSLSPGERATLSCSASSSVSYMHWY |
| | | QQKPGQAPKLLIYEISKLASGIPARFSGSGSGTDY |
| | | TLTISSLEPEDFAVYYCQQWNYPLLTFGQGTKLEI |
| | | K |
| SEQ ID NO: 1179 | VL - 2 | EIVLTQSPATLSLSPGERATLSCSASSSVSYMHWY |
| | | QQKPGQAPKLLIYEISKLASGIPARFSGSGSGTDY |
| | | TLTISRLEPEDFAVYYCQQWNYPLLTFGQGTKLE |
| | | IK |
| SEQ ID NO: 1180 | VL - 3 | EIVLTQSPDFQSVTPKEKVTITCSASSSVSYMHWY |
| | | QQKPDQSPKLLIYEISKLASGVPSRFSGSGSGTDY |
| | | TLTINSLEAEDAATYYCQQWNYPLLTFGQGTKLE |
| | | IK |
| SEQ ID NO: 1181 | VL - 4 | DIQLTQSPSFLSASVGDRVTITCSASSSVSYMHWY |
| | | QQKPGKAPKLLIYEISKLASGVPSRFSGSGSGTEY |
| | | TLTISSLQPEDFATYYCQQWNYPLLTFGQGTKLEI |
| | | K |
| SEQ ID NO: 1182 | VL - 5 | AIQLTQSPSSLSASVGDRVTITCSASSSVSYMHWY |
| | | QQKPGKAPKLLIYEISKLASGVPSRFSGSGSGTDY |
| | | TLTISSLQPEDFATYYCQQWNYPLLTFGQGTKLEI |
| | | K |
| SEQ ID NO: 1183 | VL - 6 | AIRLTQSPFSLSASVGDRVTITCSASSSVSYMHWY |
| | | QQKPAKAPKLFIYEISKLASGVPSRFSGSGSGTDY |
| | | TLTISSLQPEDFATYYCQQWNYPLLTFGQGTKLEI |
| | | K |
| SEQ ID NO: 1184 | VL - 7 | DIVLTQSPDSLAVSLGERATINCSASSSVSYMHW |
| | | YQQKPGQPPKLLIYEISKLASGVPDRFSGSGSGTD |
| | | YTLTISSLQAEDVAVYYCQQWNYPLLTFGQGTK |
| | | LEIK |

JOVI-3 (murine), also referred to as BJ1187 or Antibody K
Binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3(Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | SANSTVGYIH |
| SEQ ID NO: 1192 | LC CDR2 (Combined) | TTSNLAS |
| SEQ ID NO: 1193 | LC CDR3(Combined) | HQWSFYPT |
| SEQ ID NO: 1195 | VL | QIVLTQSPAIMSASLGEEIALTCSANSTVGYIHWY |
| | | QQKSGTSPKLLIYTTSNLASGVPSRFSGSGSGTFY |
| | | SLTISSVEAEDAADYFCHQWSFYPTFGGGTKLEI |
| | | K |
| SEQ ID NO: 1196 | VH | QIQLQQSGPEVVKPGASVQISCKASGYTFSGSWM |
| | | NWVKQRPGKGLEWIGRIYPGDGDTDYSGKFKGR |
| | | ATLTADKSSSTAYMRLSSLTSEDSAVYFCARSGY |
| | | FNYVPVFDYWGQGTTLSVSS |

VH for JOVI-3 (humanized) also referred to as Antibody K-H
Binds to human TCRVβ 28

| | | |
|---|---|---|
| SEQ ID NO: 1197 | VH - 1 | QIQLVQSGAEVKKPGASVKVSCKASGYTFSGSW |
| | | MNWVRQAPGQGLEWIGRIYPGDGDTDYSGKFK |
| | | GRATLTADKSTSTAYMELSSLRSEDTAVYYCARS |
| | | GYFNYVPVFDYWGQGTTVTVSS |
| SEQ ID NO: 1198 | VH - 2 | QIQLVQSGAEVKKPGSSVKVSCKASGYTFSGSW |
| | | MNWVRQAPGQGLEWIGRIYPGDGDTDYSGKFK |
| | | GRATLTADKSTSTAYMELSSLRSEDTAVYYCARS |
| | | GYFNYVPVFDYWGQGTTVTVSS |
| SEQ ID NO: 1199 | VH - 3 | EIQLVQSGAEVKKPGESLKISCKASGYTFSGSWM |
| | | NWVRQMPGKGLEWIGRIYPGDGDTDYSGKFKG |
| | | QATLSADKSISTAYLQWSSLKASDTAMYYCARS |
| | | GYFNYVPVFDYWGQGTTVTVSS |
| SEQ ID NO: 1200 | VH - 4 | QIQLVQSGSELKKPGASVKVSCKASGYTFSGSW |
| | | MNWVRQAPGQGLEWIGRIYPGDGDTDYSGKFK |
| | | GRAVLSADKSVSTAYLQISSLKAEDTAVYYCARS |
| | | GYFNYVPVFDYWGQGTTVTVSS |
| SEQ ID NO: 1201 | VH - 5 | QIQLVQSGSELKKPGASVKVSCKASGYTFSGSW |
| | | MNWVRQAPGQGLEWIGRIYPGDGDTDYSGKFK |
| | | GRAVLSADKSVSMAYLQISSLKAEDTAVYYCAR |
| | | SGYFNYVPVFDYWGQGTTVTVSS |
| SEQ ID NO: 1202 | VH - 6 | EIQLVESGGGLVQPGRSLRLSCTASGYTFSGSWM |
| | | NWVRQAPGKGLEWIGRIYPGDGDTDYSGKFKGR |
| | | ATLSADKSKSIAYLQMNSLKTEDTAVYYCARSG |
| | | YFNYVPVFDYWGQGTTVTVSS |

VL for JOVI-3 (humanized) also referred to as Antibody K-H
Binds to human TCRVβ 28

| | | |
|---|---|---|
| SEQ ID NO: 1203 | VL - 1 | EIVLTQSPATLSLSPGERATLSCSANSTVGYIHWY |
| | | QQKPGQAPKLLIYTTSNLASGIPARFSGSGSGTDY |
| | | TLTISSLEPEDFAVYFCHQWSFYPTFGQGTKLEIK |
| SEQ ID NO: 1204 | VL - 2 | DIQLTQSPSFLSASVGDRVTITCSANSTVGYIHWY |
| | | QQKPGKAPKLLIYTTSNLASGVPSRFSGSGSGTEY |
| | | TLTISSLQPEDFATYFCHQWSFYPTFGQGTKLEIK |
| SEQ ID NO: 1205 | VL - 3 | EIVLTQSPATLSLSPGERATLSCSANSTVGYIHWY |
| | | QQKPGQAPKLLIYTTSNLASGIPARFSGSGPGTDY |
| | | TLTISSLEPEDFAVYFCHOWSFYPTFGQGTKLEIK |
| SEQ ID NO: 1206 | VL - 4 | DIVLTQSPDSLAVSLGERATINCSANSTVGYIHW |
| | | YQQKPGQPPKLLIYTTSNLASGVPDRFSGSGSGT |
| | | DYTLTISSLQAEDVAVYFCHQWSFYPTFGQGTKL |
| | | EIK |
| SEQ ID NO: 1207 | VL - 5 | EIVLTQSPDFQSVTPKEKVTITCSANSTVGYIHWY |
| | | QQKPDQSPKLLIYTTSNLASGVPSRFSGSGSGTDY |
| | | TLTINSLEAEDAATYFCHQWSFYPTFGQGTKLEI |
| | | K |

ZOE (murine), also referred to as BJ1538 or as Antibody L
Binds to human TCRVβ 4-1, 4-2, 4-3

| | | |
|---|---|---|
| SEQ ID NO: 1208 | HC CDR1 (Kabat) | DYYMY |
| SEQ ID NO: 1209 | HC CDR2 (Kabat) | TISGGGSYTYSPDSVKG |
| SEQ ID NO: 1210 | HC CDR3 (Kabat) | ERDIYYGNFNAMVY |
| SEQ ID NO: 1211 | HC CDR1 (Chothia) | GFTFSDY |
| SEQ ID NO: 1212 | HC CDR2 (Chothia) | SGGGSY |
| SEQ ID NO: 1210 | HC CDR3 (Chothia) | ERDIYYGNFNAMVY |
| SEQ ID NO: 1213 | HC CDR1 (Combined) | GFTFSDYYMY |
| SEQ ID NO: 1209 | HC CDR2 (Combined) | TISGGGSYTYSPDSVKG |
| SEQ ID NO: 1210 | HC CDR3(Combined) | ERDIYYGNFNAMVY |
| SEQ ID NO: 1214 | LC CDR1 (Kabat) | RASKSVSTSGYSYMH |
| SEQ ID NO: 1215 | LC CDR2 (Kabat) | LASNLES |
| SEQ ID NO: 1216 | LC CDR3 (Kabat) | QHSRDLPWT |
| SEQ ID NO: 1217 | LC CDR1 (Chothia) | SKSVSTSGYSY |
| SEQ ID NO: 1215 | LC CDR2 (Chothia) | LASNLES |
| SEQ ID NO: 1216 | LC CDR3 (Chothia) | QHSRDLPWT |
| SEQ ID NO: 1214 | LC CDR1 (Combined) | RASKSVSTSGYSYMH |
| SEQ ID NO: 1215 | LC CDR2 (Combined) | LASNLES |
| SEQ ID NO: 1216 | LC CDR3(Combined) | QHSRDLPWT |
| SEQ ID NO: 1218 | VL | DIVLTQSPVSLTVSLGQRATISCRASKSVSTSGYS |
| | | YMHWYQQKPGQPPKLLIYLASNLESGVPARFSG |
| | | SGSGTDFTLNIHPVEEEDAATYYCQHSRDLPWTF |
| | | GGGTKLEIK |
| SEQ ID NO: 1219 | VH | EVQLVESGGGLVKPGGSLKLSCAASGFTFSDYY |
| | | MYWVRQTPEKRLEWVATISGGGSYTYSPDSVKG |
| | | RFTISRDNAKNNLYLQMSSLRSEDTAMYFCARER |
| | | DIYYGNFNAMVYWGRGTSVTVSS |

VH for ZOE (humanized) also referred to as Antibody L-H
Binds to human TCRVβ 4-1, 4-2, 4-3

| | | |
|---|---|---|
| SEQ ID NO: 1220 | VH - 1 | EVQLLESGGGLVQPGGSLRLSCAASGFTFSDYYM |
| | | YWVRQAPGKGLEWVATISGGGSYTYSPDSVKGR |
| | | FTISRDNSKNTLYLQMNSLRAEDTAVYYCARER |
| | | DIYYGNFNAMVYWGRGTLVTVSS |
| SEQ ID NO: 1221 | VH - 2 | EVQLVESGGGLVQPGGSLRLSCAASGFTFSDYY |
| | | MYWVRQAPGKGLEWVATISGGGSYTYSPDSVK |
| | | GRFTISRDNAKNSLYLQMNSLRAEDTAVYYCAR |
| | | ERDIYYGNFNAMVYWGRGTLVTVSS |
| SEQ ID NO: 1222 | VH - 3 | QVQLVESGGGVVQPGRSLRLSCAASGFTFSDYY |
| | | MYWVRQAPGKGLEWVATISGGGSYTYSPDSVK |
| | | GRFTISRDNSKNTLYLQMNSLRAEDTAVYYCAR |
| | | ERDIYYGNFNAMVYWGRGTLVTVSS |
| SEQ ID NO: 1223 | VH - 4 | QVQLVESGGGLVKPGGSLRLSCAASGFTFSDYY |
| | | MYWIRQAPGKGLEWVATISGGGSYTYSPDSVKG |
| | | RFTISRDNAKNSLYLQMNSLRAEDTAVYYCARE |
| | | RDIYYGNFNAMVYWGRGTLVTVSS |

VL for ZOE (humanized) also referred to as Antibody L-H
Binds to human TCRVβ 4-1, 4-2, 4-3

| | | |
|---|---|---|
| SEQ ID NO: 1224 | VL - 1 | EIVLTQSPGTLSLSPGERATLSCRASKSVSTSGYS |
| | | YMHWYQQKPGQAPRLLIYLASNLESGIPDRFSGS |
| | | GSGTDFTLTISRLEPEDFAVYYCQHSRDLPWTFG |
| | | GGTKVEIK |
| SEQ ID NO: 1225 | VL - 2 | EIVLTQSPATLSLSPGERATLSCRASKSVSTSGYS |
| | | YMHWYQQKPGQAPRLLIYLASNLESGIPARFSGS |
| | | GSGTDFTLTISSLEPEDFAVYYCQHSRDLPWTFG |
| | | GGTKVEIK |
| SEQ ID NO: 1226 | VL - 3 | DIQLTQSPSTLSASVGDRVTITCRASKSVSTSGYS |
| | | YMHWYQQKPGKAPKLLIYLASNLESGVPSRFSG |
| | | SGSGTEFTLTISSLQPDDFATYYCQHSRDLPWTFG |
| | | GGTKVEIK |
| SEQ ID NO: 1227 | VL - 4 | AIQLTQSPSSLSASVGDRVTITCRASKSVSTSGYS |
| | | YMHWYQQKPGKAPKLLIYLASNLESGVPSRFSG |
| | | SGSGTDFTLTISSLQPEDFATYYCQHSRDLPWTFG |
| | | GGTKVEIK |

Anti-TCRvb19 (murine), also referred to as BJ1465; or Antibody M
Binds to human TCRVβ 19

| | | |
|---|---|---|
| SEQ ID NO: 1229 | HC CDR1 (Kabat) | GYFWN |
| SEQ ID NO: 1230 | HC CDR2 (Kabat) | YISYDGSNNYNPSLKN |
| SEQ ID NO: 1231 | HC CDR3 (Kabat) | PSPGTGYAVDY |
| SEQ ID NO: 1232 | HC CDR1 (Chothia) | GYSITSGY |
| SEQ ID NO: 1233 | HC CDR2 (Chothia) | SYDGSN |
| SEQ ID NO: 1231 | HC CDR3 (Chothia) | PSPGTGYAVDY |
| SEQ ID NO: 1234 | HC CDR1 (Combined) | GYSITSGYFWN |
| SEQ ID NO: 1230 | HC CDR2 (Combined) | YISYDGSNNYNPSLKN |
| SEQ ID NO: 1231 | HC CDR3(Combined) | PSPGTGYAVDY |
| SEQ ID NO: 1235 | LC CDR1 (Kabat) | RSSQSLVHSNGNTYLH |
| SEQ ID NO: 1236 | LC CDR2 (Kabat) | KVSNRFS |
| SEQ ID NO: 1237 | LC CDR3 (Kabat) | SQSTHVPFT |
| SEQ ID NO: 1238 | LC CDR1 (Chothia) | SQSLVHSNGNTY |
| SEQ ID NO: 1236 | LC CDR2 (Chothia) | KVSNRFS |
| SEQ ID NO: 1237 | LC CDR3 (Chothia) | SQSTHVPFT |
| SEQ ID NO: 1235 | LC CDR1 (Combined) | RSSQSLVHSNGNTYLH |
| SEQ ID NO: 1236 | LC CDR2 (Combined) | KVSNRFS |
| SEQ ID NO: 1237 | LC CDR3(Combined) | SQSTHVPFT |
| SEQ ID NO: 1239 | VL | NVVMTQTPLSLPVSLGDQASISCRSSQSLVHSNG |
| | | NTYLHWYLQKPGQSPKFLIYKVSNRFSGVPDRFS |
| | | GGGSGTEFTLKISRVEAEDLGVYFCSQSTHVPFTF |
| | | GSGTKLEIK |
| SEQ ID NO: 1240 | VH | NVQLQESGPGLVKPSQSLSLTCSVAGYSITSGYF |
| | | WNWIRQFPGNKLEWMGYISYDGSNNYNPSLKN |
| | | RISITRDTSKNQFFLKLNSVTTEDTATYYCASPSP |
| | | GTGYAVDYWGQGTSVTVSS |

VH for Anti-TCRvb19 (humanized) also referred to as Antibody M-H
Binds to human TCRVβ 19

| | | |
|---|---|---|
| SEQ ID NO: 1241 | VH - 1 | QVQLQESGPGLVKPSETLSLTCTVSGYSITSGYF |
| | | WNWIRQPPGKGLEWIGYISYDGSNNYNPSLKNR |
| | | VTISRDTSKNQFSLKLSSVTAADTAVYYCASPSP |
| | | GTGYAVDYWGQGTLVTVSS |
| SEQ ID NO: 1242 | VH - 2 | QVQLQESGPGLVKPSETLSLTCTVSGYSITSGYF |
| | | WNWIRQPPGKGLEWIGYISYDGSNNYNPSLKNR |
| | | VTISRDTSKNQFSLKLSSVTAADTAVYYCASPSP |
| | | GTGYAVDYWGQGTLVTVSS |
| SEQ ID NO: 1243 | VH - 3 | QVQLVESGGGLVQPGGSLRLSCSVSGYSITSGYF |
| | | WNWVRQAPGKGLEWVGYISYDGSNNYNPSLKN |
| | | RFTISRDTSKNTFYLQMNSLRAEDTAVYYCASPS |
| | | PGTGYAVDYWGQGTLVTVSS |

VL for Anti-TCRvb19 (humanized) also referred to as Antibody M-H
Binds to human TCRVβ 19

| | | |
|---|---|---|
| SEQ ID NO: 1244 | VL - 1 | VVMTQSPGTLSLSPGERATLSCRSSQSLVHSNGN |
| | | TYLHWYQQKPGQAPRFLIYKVSNRFSGIPDRFSG |
| | | SGSGTDFTLTISRLEPEDFAVYFCSQSTHVPFTFG |
| | | QGTKLEIK |
| SEQ ID NO: 1245 | VL - 2 | EVVMTQSPATLSLSPGERATLSCRSSQSLVHSNG |
| | | NTYLHWYQQKPGQAPRFLIYKVSNRFSGIPARFS |
| | | GSGSGTDFTLTISSLEPEDFAVYFCSQSTHVPFTFG |
| | | QGTKLEIK |
| SEQ ID NO: 1246 | VL - 3 | EVVMTQSPATLSVSPGERATLSCRSSQSLVHSNG |
| | | NTYLHWYQQKPGQAPRFLIYKVSNRFSGIPARFS |
| | | GSGSGTEFTLTISSLQSEDFAVYFCSQSTHVPFTFG |
| | | QGTKLEIK |
| SEQ ID NO: 1247 | VL - 4 | DVQMTQSPSSLSASVGDRVTITCRSSQSLVHSNG |
| | | NTYLHWYQQKPGKAPKFLIYKVSNRFSGVPSRFS |
| | | GSGSGTDFTFTISSLQPEDIATYFCSQSTHVPFTFG |
| | | QGTKLEIK |

BL37.2 (murine), also referred to as BJ1539 or Antibody N
Binds to human TCRVβ 9

| | | |
|---|---|---|
| SEQ ID NO: 1248 | HC CDR1 (Kabat) | DYIVH |
| SEQ ID NO: 1249 | HC CDR2 (Kabat) | WINTYTGTPTYADDFEG |
| SEQ ID NO: 1250 | HC CDR3 (Kabat) | SWRRGIRGIGFDY |
| SEQ ID NO: 1251 | HC CDR1 (Chothia) | GYTFTDY |
| SEQ ID NO: 1252 | HC CDR2 (Chothia) | NTYTGT |
| SEQ ID NO: 1250 | HC CDR3 (Chothia) | SWRRGIRGIGFDY |
| SEQ ID NO: 1253 | HC CDR1 (Combined) | GYTFTDYIVH |
| SEQ ID NO: 1249 | HC CDR2 (Combined) | WINTYTGTPTYADDFEG |
| SEQ ID NO: 1250 | HC CDR3(Combined) | SWRRGIRGIGFDY |
| SEQ ID NO: 1254 | LC CDR1 (Kabat) | KASKSINKYLA |
| SEQ ID NO: 1255 | LC CDR2 (Kabat) | DGSTLQS |
| SEQ ID NO: 1256 | LC CDR3 (Kabat) | QQHNEYPPT |
| SEQ ID NO: 1257 | LC CDR1 (Chothia) | SKSINKY |
| SEQ ID NO: 1255 | LC CDR2 (Chothia) | DGSTLQS |
| SEQ ID NO: 1256 | LC CDR3 (Chothia) | QQHNEYPPT |
| SEQ ID NO: 1254 | LC CDR1 (Combined) | KASKSINKYLA |
| SEQ ID NO: 1255 | LC CDR2 (Combined) | DGSTLQS |
| SEQ ID NO: 1256 | LC CDR3(Combined) | QQHNEYPPT |
| SEQ ID NO: 1258 | VL | DVQMTQSPYNLAASPGESVSINCKASKSINKYLA |
| | | WYQQKPGKPNKLLIYDGSTLQSGIPSRFSGSGSG |
| | | TDFTLTIRGLEPEDFGLYYCQQHNEYPPTFGAGT |
| | | KLELK |
| SEQ ID NO: 1259 | VH | QLQLVQSGPELREPGESVKISCKASGYTFTDYIVH |
| | | WVKQAPGKGLKWMGWINTYTGTPTYADDFEGR |
| | | FVFSLEASASTANLQISNLKNEDTATYFCARSWR |
| | | RGIRGIGFDYWGQGVMVTVSS |

VH for BL37.2 (humanized) also referred to as Antibody N-H
Binds to human TCRVβ 9

| | | |
|---|---|---|
| SEQ ID NO: 1260 | VH - 1 | QLQLVQSGAEVKKPGASVKVSCKASGYTFTDYI |
| | | VHWVRQAPGQGLEWMGWINTYTGTPTYADDFE |
| | | GWVTMTLDASISTAYMELSRLRSDDTAVYYCAR |
| | | SWRRGIRGIGFDYWGQGTMVTVSS |
| SEQ ID NO: 1261 | VH - 2 | QLQLVQSGAEVKKPGASVKVSCKASGYTFTDYI |
| | | VHWVRQAPGQGLEWMGWINTYTGTPTYADDFE |
| | | GRVTMTLDASTSTAYMELSSLRSEDTAVYYCAR |
| | | SWRRGIRGIGFDYWGQGTMVTVSS |
| SEQ ID NO: 1262 | VH - 3 | QLQLVQSGAEVKKPGASVKVSCKASGYTFTDYI |
| | | VHWVRQAPGQRLEWMGWINTYTGTPTYADDFE |
| | | GRVTITLDASASTAYMELSSLRSEDMAVYYCARS |
| | | WRRGIRGIGFDYWGQGTMVTVSS |
| SEQ ID NO: 1263 | VH - 4 | QLQLVQSGAEVKKPGASVKVSCKASGYTFTDYI |
| | | VHWVRQATGQGLEWMGWINTYTGTPTYADDFE |
| | | GRVTMTLNASISTAYMELSSLRSEDTAVYYCARS |
| | | WRRGIRGIGFDYWGQGTMVTVSS |

VL for BL37.2 (humanized) also referred to as Antibody N-H
Binds to human TCRVβ 9

| | | |
|---|---|---|
| SEQ ID NO: 1264 | VL - 1 | EVVMTQSPGTLSLSPGERATLSCKASKSINKYLA |
| | | WYQQKPGQAPRLLIYDGSTLQSGIPDRFSGSGSG |
| | | TDFTLTISRLEPEDFAVYYCQQHNEYPPTFGQGT |
| | | KLEIK |
| SEQ ID NO: 1265 | VL - 2 | EVVMTQSPATLSLSPGERATLSCKASKSINKYLA |
| | | WYQQKPGQAPRLLIYDGSTLQSGIPARFSGSGSG |
| | | TDFTLTISSLEPEDFAVYYCQQHNEYPPTFGQGTK |
| | | LEIK |
| SEQ ID NO: 1266 | VL - 3 | DVQMTQSPSSLSASVGDRVTITCKASKSINKYLA |
| | | WYQQKPGKAPKLLIYDGSTLQSGVPSRFSGSGSG |
| | | TDFTLTISSLQPEDFATYYCQQHNEYPPTFGQGTK |
| | | LEIK |
| SEQ ID NO: 1267 | VL - 4 | AVRMTQSPSSFSASTGDRVTITCKASKSINKYLA |
| | | WYQQKPGKAPKLLIYDGSTLQSGVPSRFSGSGSG |
| | | TDFTLTISCLQSEDFATYYCQQHNEYPPTFGQGT |
| | | KLEIK |

IG125 (murine) binds to TRVβ 11-2; also referred to as Antibody O

| | | |
|---|---|---|
| SEQ ID NO: 1268 | HC CDR1 (Kabat) | NYGVH |
| SEQ ID NO: 1269 | HC CDR2 (Kabat) | VIWSDGSTDYDTAFIS |
| SEQ ID NO: 1270 | HC CDR3 (Kabat) | RAVVADFDY |
| SEQ ID NO: 1271 | HC CDR1 (Chothia) | GFSLTN |
| SEQ ID NO: 1272 | HC CDR2 (Chothia) | VIWSDGSTD |
| SEQ ID NO: 1270 | HC CDR3 (Chothia) | RAVVADFDY |
| SEQ ID NO: 1273 | HC CDR1 (combined) | GFSLTNYGVH |
| SEQ ID NO: 1269 | HC CDR2 (combined) | VIWSDGSTDYDTAFIS |
| SEQ ID NO: 1270 | HC CDR3 (combined) | RAVVADFDY |
| SEQ ID NO: 1274 | VH | QVQLKQSGPGLLQPSQSLSITCTVSGFSLTNYGV |
| | | HWVRQSPGKGLEWLGVIWSDGSTDYDTAFISRL |
| | | SISKDNSKSQVFFKLNSLQADDTAIYYCARRAVV |
| | | ADFDYWGQGTTLTVSS |
| SEQ ID NO:1275 | LC CDR1 (Kabat) | KASKEVTIFGSISALH |
| SEQ ID NO:1276 | LC CDR2 (Kabat) | NGAKLES |
| SEQ ID NO: 1277 | LC CDR3 (Kabat) | LQNKEVPFT |
| SEQ ID NO:1275 | LC CDR1 (Chothia) | KASKEVTIFGSISALH |
| SEQ ID NO:1276 | LC CDR2 (Chothia) | NGAKLES |
| SEQ ID NO: 1277 | LC CDR3 (Chothia) | LQNKEVPFT |
| SEQ ID NO: 1275 | LC CDR1 (combined) | KASKEVTIFGSISALH |
| SEQ ID NO: 1276 | LC CDR2 (combined) | NGAKLES |
| SEQ ID NO: 1277 | LC CDR3 (combined) | LQNKEVPFT |
| SEQ ID NO: 1278 | VL | DIVLTQSPASLAVSLGQKATISCKASKEVTIFGSI |
| | | SALHWYQQKPGQPPKLIYNGAKLESGVSARFSD |
| | | SGSQNRSPFGNQLSFTLTIAPVEADDAATYYCLQ |
| | | NKEVPFTFGSGTKLEIK |

VL for IG125 (humanized) also referred to as Antibody O-H
binds to TRVβ 11-2

| | | |
|---|---|---|
| SEQ ID NO: 1279 | VL-1 | DIVLTQSPDSLAVSLGERATINCKASKEVTIFGSI |
| | | SALHWYQQKPGQPPKLLYNGAKLESGVSARFG |
| | | VPDRFSRSGSGLDFTLTISSLQAEDVAVYYCLQN |
| | | KEVPFTFGQGTKLEIK |
| SEQ ID NO: 1280 | VL-2 | EIVLTQSPDFQSVTPKEKVTITCKASKEVTIFGSIS |
| | | ALHWYQQKPDQSPKLLYNGAKLESGVSARFGV |
| | | PSRFSRSGSGLDFTLTINSLEAEDAATYYCLQNK |
| | | EVPFTFGQGTKLEIK |
| SEQ ID NO: 1281 | VL-3 | AIQLTQSPSSLSASVGDRVTITCKASKEVTIFGSIS |
| | | ALHWYQQKPGKAPKLLYNGAKLESGVSARFGV |
| | | PSRFSRSGSGLDFTLTISSLQPEDFATYYCLQNKE |
| | | VPFTFGQGTKLEIK |
| SEQ ID NO: 1282 | VL-4 | DIVLTQTPLSLSVTPGQPASISCKASKEVTIFGSIS |
| | | ALHWYLQKPGQPPKLLYNGAKLESGVSARFGV |
| | | PDRFSRSGSGLDFTLKISRVEAEDVGVYYCLQN |
| | | KEVPFTFGQGTKLEIK |

VH for IG125 (humanized) also referred to as Antibody O-H
binds to TRVβ 11-2

| | | |
|---|---|---|
| SEQ ID NO: 1283 | VH-1 | QVTLKESGPVLVKPTETLTLTCTVSGFSLTNYGV |
| | | HWVRQPPGKALEWLGVIWSDGSTDYDTAFISRL |
| | | TISKDNSKSQVVLTMTNMDPVDTATYYCARRA |
| | | VVADFDYWGQGTTVTVSS |
| SEQ ID NO: 1284 | VH-2 | QVQLQESGPGLVKPSGTLSLTCAVSGFSLTNYG |
| | | VHWVRQPPGKGLEWLGVIWSDGSTDYDTAFIS |
| | | RLTISKDNSKSQVSLKLSSVTAADTAVYYCARR |
| | | AVVADFDYWGQGTTVTVSS |
| SEQ ID NO: 1285 | VH-3 | QVQLQQSGPGLVKPSQTLSLTCAVSGFSLTNYG |
| | | VHWVRQSPSRGLEWLGVIWSDGSTDYDTAFISR |
| | | LTINKDNSKSQVSLQLNSVTPEDTAVYYCARRA |
| | | VVADFDYWGQGTTVTVSS |
| SEQ ID NO: 1286 | VH-4 | EVQLVESGGGLVQPGPSLRLSCTVSGFSLTNYG |
| | | VHWVRQAPGKGLEWLGVIWSDGSTDYDTAFIS |
| | | RLTISKDNSKSIVYLQMNSLKTEDTAVYYCARR |
| | | AVVADFDYWGQGTTVTVSS |
| SEQ ID NO: 1287 | VH-5 | EVQLVQSGAEVKKPGESLRISCKVSGFSLTNYG |
| | | VHWVRQMPGKGLEWLGVIWSDGSTDYDTAFIS |
| | | QLTISKDNSISTVYLQWSSLKASDTAMYYCARR |
| | | AVVADFDYWGQGTTVTVSS |

MR5-2 (murine), Binds to mouse TCRVβ 13-2 and 13-3

| | | |
|---|---|---|
| SEQ ID NO: 1376 | SCFV (VH + VL) | QVQLQQSGTELMKPGASVKISCKASGYTFSNY |
| | | WIEWIKQRPGHGLEWVGEILPGAGPTNYNEKFK |
| | | GKATFTADSSSNTAYMQLSSLTSEDSAVYYCAR |
| | | TDYDYDWFAYWGQGTLVTVSAGGGGSGGGGS |
| | | GGGGSGGGGSDIVMSQSPSSLAVSVGEKVTMSC |
| | | KSSQSLLYSGNQKNYLAWYQQKPGQSPKLLIY |
| | | WASTRESGVPDRFTGSGSGTDFTLTINSVKAEDL |
| | | TVYYCQQYYGYPRTFGGGTKVEIK |
| SEQ ID NO: 7500 | HC CDR1 (Kabat) | NYWIE |
| SEQ ID NO: 7501 | HC CDR2 (Kabat) | EILPGAGPTNYNEKFKG |
| SEQ ID NO: 7502 | HC CDR3 (Kabat) | TDYDYDWFAY |
| SEQ ID NO: 7503 | HC CDR1 (Chothia) | GYTFSNY |
| SEQ ID NO: 7504 | HC CDR2 (Chothia) | LPGAGP |
| SEQ ID NO: 7502 | HC CDR3 (Chothia) | TDYDYDWFAY |
| SEQ ID NO: 7505 | HC CDR1 (combined) | GYTFSNYWIE |
| SEQ ID NO: 7501 | HC CDR2 (combined) | EILPGAGPTNYNEKFKG |
| SEQ ID NO: 7502 | HC CDR3 (combined) | TDYDYDWFAY |
| SEQ ID NO: 7506 | LC CDR1 (Kabat) | KSSQSLLYSGNQKNYLA |
| SEQ ID NO: 7507 | LC CDR2 (Kabat) | WASTRES |
| SEQ ID NO: 7508 | LC CDR3 (Kabat) | QQYYGYPRT |
| SEQ ID NO: 7506 | LC CDR1 (Chothia) | KSSQSLLYSGNQKNYLA |
| SEQ ID NO: 7507 | LC CDR2 (Chothia) | WASTRES |
| SEQ ID NO: 7508 | LC CDR3 (Chothia) | QQYYGYPRT |
| SEQ ID NO: 7506 | LC CDR1 (combined) | KSSQSLLYSGNQKNYLA |
| SEQ ID NO: 7507 | LC CDR2 (combined) | WASTRES |
| SEQ ID NO: 7508 | LC CDR3 (combined) | QQYYGYPRT |
| SEQ ID NO: 7509 | VH | QVQLQQSGTELMKPGASVKISCKASGYTFSNY |
| | | WIEWIKQRPGHGLEWVGEILPGAGPTNYNEKFK |
| | | GKATFTADSSSNTAYMQLSSLTSEDSAVYYCAR |
| | | TDYDYDWFAYWGQGTLVTVSA |
| SEQ ID NO: 3527 | VL | DIVMSQSPSSLAVSVGEKVTMSCKSSQSLLYSG |
| | | NQKNYLAWYQQKPGQSPKLLIYWASTRESGVP |
| | | DRFTGSGSGTDFTLTINSVKAEDLTVYYCQQYY |
| | | GYPRTFGGGTKVEIK |
| SEQ ID NO: 7511 | Heavy Chain | QVQLQQSGTELMKPGASVKISCKASGYTFSNY |
| | | WIEWIKQRPGHGLEWVGEILPGAGPTNYNEKFK |
| | | GKATFTADSSSNTAYMQLSSLTSEDSAVYYCAR |
| | | TDYDYDWFAYWGQGTLVTVSAAKTTAPSVYPL |
| | | APVCGDTTGSSVTLGCLVKGYFPEPVTLTWNSG |
| | | SLSSGVHTFPAVLQSDLYTLSSSVTVTSSTWPSQ |
| | | SITCNVAHPASSTKVDKKIEPRGPTIKPCPPCKCP |
| | | APNLLGGPSVFIFPPKIKDVLMISLSPIVTCVVVD |
| | | VSEDDPDVQISWFVNNVEVHTAQTQTHREDYN |
| | | STLRVVSALPIQHQDWMSGKEFKCKVNNKDLP |
| | | APIERTISKPKGSVRAPQVYVLPPPEEEMTKKQV |
| | | TLTCMVTDFMPEDIYVEWTNNGKTELNYKNTE |
| | | PVLDSDGSYFMYSKLRVEKKNWVERNSYSCSV |
| | | VHEGLHNHHTTKSFSRTPGK |
| SEQ ID NO: 7512 | Light Chain | DIVMSQSPSSLAVSVGEKVTMSCKSSQSLLYSG |
| | | NQKNYLAWYQQKPGQSPKLLIYWASTRESGVP |
| | | DRFTGSGSGTDFTLTINSVKAEDLTVYYCQQYY |
| | | GYPRTFGGGTKVEIKRADAAPTVSIFPPSSEQLTS |
| | | GGASVVCFLNNFYPKDINVKWKIDGSERQNGVL |
| | | NSWTDQDSKDSTYSMSSTLTLTKDEYERHNSYT |
| | | CEATHKTSTSPIVKSFNRNECEPEA |

TABLE 14
Exemplary amino acid sequences of TGF-beta polypeptides or TGF-beta receptor
polypeptides

| SEQ ID NO | Description | Amino acid sequence |
|---|---|---|
| SEQ ID | Immature | MPPSGLRLLLLLLPLLWLLVLTPGRPAAGLSTCKTIDMELVKRK |
| NO: 7254 | human TGF- | RIEAIRGQILSKLRLASPPSQGEVPPGPLPEAVLALYNSTRDRVA |
| | beta 1 | GESAEPEPEPEADYYAKEVTRVLMVETHNEIYDKFKQSTHSIYM |
| | (P01137-1) | FFNTSELREAVPEPVLLSRAELRLLRLKLKVEQHVELYQKYSNN |
| | | SWRYLSNRLLAPSDSPEWLSFDVTGVVRQWLSRGGEIEGFRLSA |
| | | HCSCDSRDNTLQVDINGFTTGRRGDLATIHGMNRPFLLLMATPL |
| | | ERAQHLQSSRHRRALDTNYCFSSTEKNCCVRQLYIDFRKDLGW |
| | | KWIHEPKGYHANFCLGPCPYIWSLDTQYSKVLALYNQHNPGAS |
| | | AAPCCVPQALEPLPIVYYVGRKPKVEQLSNMIVRSCKCS |
| SEQ ID | Human TGF- | LSTCKTIDMELVKRKRIEAIRGQILSKLRLASPPSQGEVPPGPLPE |
| NO: 7271 | beta 1 | AVLALYNSTRDRVAGESAEPEPEPEADYYAKEVTRVLMVETHN |
| | (P01137-1) | EIYDKFKQSTHSIYMFFNTSELREAVPEPVLLSRAELRLLRLKLK |
| | | VEQHVELYQKYSNNSWRYLSNRLLAPSDSPEWLSFDVTGVVRQ |
| | | WLSRGGEIEGFRLSAHCSCDSRDNTLQVDINGFTTGRRGDLATI |
| | | HGMNRPFLLLMATPLERAQHLQSSRHRRALDTNYCFSSTEKNC |
| | | CVRQLYIDFRKDLGWKWIHEPKGYHANFCLGPCPYIWSLDTQY |
| | | SKVLALYNQHNPGASAAPCCVPQALEPLPIVYYVGRKPKVEQL |
| | | SNMIVRSCKCS |
| SEQ ID | Immature | MHYCVLSAFLILHLVTVALSLSTCSTLDMDQFMRKRIEAIRGQIL |
| NO: 7255 | human TGF- | SKLKLTSPPEDYPEPEEVPPEVISIYNSTRDLLQEKASRRAAACE |
| | beta 2 | RERSDEEYYAKEVYKIDMPPFFPSENAIPPTFYRPYFRIVRFDVS |
| | (P61812-1) | AMEKNASNLVKAEFRVFRLQNPKARVPEQRIELYQILKSKDLTS |
| | | PTQRYIDSKVVKTRAEGEWLSFDVTDAVHEWLHHKDRNLGFKI |
| | | SLHCPCCTFVPSNNYIIPNKSEELEARFAGIDGTSTYTSGDQKTIK |
| | | STRKKNSGKTPHLLLMLLPSYRLESQQTNRRKKRALDAAYCFR |
| | | NVQDNCCLRPLYIDFKRDLGWKWIHEPKGYNANFCAGACPYL |
| | | WSSDTQHSRVLSLYNTINPEASASPCCVSQDLEPLTILYYIGKTP |
| | | KIEQLSNMIVKSCKCS |
| SEQ ID | Human TGF- | LSTCSTLDMDQFMRKRIEAIRGQILSKLKLTSPPEDYPEPEEVPPE |
| NO: 7272 | beta 2 | VISIYNSTRDLLQEKASRRAAACERERSDEEYYAKEVYKIDMPP |
| | (P61812-1) | FFPSENAIPPTFYRPYFRIVRFDVSAMEKNASNLVKAEFRVFRLQ |
| | | NPKARVPEQRIELYQILKSKDLTSPTQRYIDSKVVKTRAEGEWL |
| | | SFDVTDAVHEWLHHKDRNLGFKISLHCPCCTFVPSNNYIIPNKS |
| | | EELEARFAGIDGTSTYTSGDQKTIKSTRKKNSGKTPHLLLMLLPS |
| | | YRLESQQTNRRKKRALDAAYCFRNVQDNCCLRPLYIDFKRDLG |
| | | WKWIHEPKGYNANFCAGACPYLWSSDTQHSRVLSLYNTINPEA |
| | | SASPCCVSQDLEPLTILYYIGKTPKIEQLSNMIVKSCKCS |
| SEQ ID | Immature | MKMHLQRALVVLALLNFATVSLSLSTCTTLDFGHIKKKRVEAIR |
| NO: 7256 | human TGF- | GQILSKLRLTSPPEPTVMTHVPYQVLALYNSTRELLEEMHGERE |
| | beta 3 | EGCTQENTESEYYAKEIHKFDMIQGLAEHNELAVCPKGITSKVF |
| | (P10600-1) | RFNVSSVEKNRTNLFRAEFRVLRVPNPSSKRNEQRIELFQILRPD |
| | | EHIAKQRYIGGKNLPTRGTAEWLSFDVTDTVREWLLRRESNLG |
| | | LEISIHCPCHTFQPNGDILENIHEVMEIKFKGVDNEDDHGRGDLG |
| | | RLKKQKDHHNPHLILMMIPPHRLDNPGQGGQRKKRALDTNYCF |
| | | RNLEENCCVRPLYIDFRQDLGWKWVHEPKGYYANFCSGPCPYL |
| | | RSADTTHSTVLGLYNTLNPEASASPCCVPQDLEPLTILYYVGRTP |
| | | KVEQLSNMVVKSCKCS |
| SEQ ID | Human TGF- | LSTCTTLDFGHIKKKRVEAIRGQILSKLRLTSPPEPTVMTHVPYQ |
| NO: 7273 | beta 3 | VLALYNSTRELLEEMHGEREEGCTQENTESEYYAKEIHKFDMIQ |
| | (P10600-1) | GLAEHNELAVCPKGITSKVFRFNVSSVEKNRTNLFRAEFRVLRV |
| | | PNPSSKRNEQRIELFQILRPDEHIAKQRYIGGKNLPTRGTAEWLS |
| | | FDVTDTVREWLLRRESNLGLEISIHCPCHTFQPNGDILENIHEVM |
| | | EIKFKGVDNEDDHGRGDLGRLKKQKDHHNPHLILMMIPPHRLD |
| | | NPGQGGQRKKRALDTNYCFRNLEENCCVRPLYIDFRQDLGWK |
| | | WVHEPKGYYANFCSGPCPYLRSADTTHSTVLGLYNTLNPEASA |
| | | SPCCVPQDLEPLTILYYVGRTPKVEQLSNMVVKSCKCS |
| SEQ ID | Immature | MEAAVAAPRPRLLLLVLAAAAAAAAALLPGATALQCFCHLCT |
| NO: 7257 | human | KDNFTCVTDGLCFVSVTETTDKVIHNSMCIAEIDLIPRDRPFVCA |
| | TGFBR1 | PSSKTGSVTTTYCCNQDHCNKIELPTTVKSSPGLGPVELAAVIAG |
| | isoform 1 | PVCFVCISLMLMVYICHNRTVIHHRVPNEEDPSLDRPFISEGTTL |
| | (P36897-1) | KDLIYDMTTSGSGSGLPLLVQRTIARTIVLQESIGKGRFGEVWR |
| | | GKWRGEEVAVKIFSSREERSWFREAEIYQTVMLRHENILGFIAA |
| | | DNKDNGTWTQLWLVSDYHEHGSLFDYLNRYTVTVEGMIKLAL |
| | | STASGLAHLHMEIVGTQGKPAIAHRDLKSKNILVKKNGTCCIAD |
| | | LGLAVRHDSATDTIDIAPNHRVGTKRYMAPEVLDDSINMKHFE |
| | | SFKRADIYAMGLVFWEIARRCSIGGIHEDYQLPYYDLVPSDPSV |
| | | EEMRKVVCEQKLRPNIPNRWQSCEALRVMAKIMRECWYANGA |
| | | ARLTALRIKKTLSQLSQQEGIKM |
| SEQ ID | Human | LQCFCHLCTKDNFTCVTDGLCFVSVTETTDKVIHNSMCIAEIDLI |
| NO: 7274 | TGFBR1 | PRDRPFVCAPSSKTGSVTTTYCCNQDHCNKIELPTTVKSSPGLGP |
| | isoform 1 | VELAAVIAGPVCFVCISLMLMVYICHNRTVIHHRVPNEEDPSLD |
| | (P36897-1) | RPFISEGTTLKDLIYDMTTSGSGSGLPLLVQRTIARTIVLQESIGK |
| | | GRFGEVWRGKWRGEEVAVKIFSSREERSWFREAEIYQTVMLRH |
| | | ENILGFIAADNKDNGTWTQLWLVSDYHEHGSLFDYLNRYTVTV |
| | | EGMIKLALSTASGLAHLHMEIVGTQGKPAIAHRDLKSKNILVKK |
| | | NGTCCIADLGLAVRHDSATDTIDIAPNHRVGTKRYMAPEVLDD |
| | | SINMKHFESFKRADIYAMGLVFWEIARRCSIGGIHEDYQLPYYD |
| | | LVPSDPSVEEMRKVVCEQKLRPNIPNRWQSCEALRVMAKIMRE |
| | | CWYANGAARLTALRIKKTLSQLSQQEGIKM |
| SEQ ID | Immature | MEAAVAAPRPRLLLLVLAAAAAAAAALLPGATALQCFCHLCT |
| NO: 7258 | human | KDNFTCVTDGLCFVSVTETTDKVIHNSMCIAEIDLIPRDRPFVCA |
| | TGFBR1 | PSSKTGSVTTTYCCNQDHCNKIELPTTGPFSVKSSPGLGPVELAA |
| | isoform 2 | VIAGPVCFVCISLMLMVYICHNRTVIHHRVPNEEDPSLDRPFISE |
| | (P36897-2) | GTTLKDLIYDMTTSGSGSGLPLLVQRTIARTIVLQESIGKGRFGE |
| | | VWRGKWRGEEVAVKIFSSREERSWFREAEIYQTVMLRHENILG |
| | | FIAADNKDNGTWTQLWLVSDYHEHGSLFDYLNRYTVTVEGMI |
| | | KLALSTASGLAHLHMEIVGTQGKPAIAHRDLKSKNILVKKNGT |
| | | CCIADLGLAVRHDSATDTIDIAPNHRVGTKRYMAPEVLDDSINM |
| | | KHFESFKRADIYAMGLVFWEIARRCSIGGIHEDYQLPYYDLVPS |
| | | DPSVEEMRKVVCEQKLRPNIPNRWQSCEALRVMAKIMRECWY |
| | | ANGAARLTALRIKKTLSQLSQQEGIKM |
| SEQ ID | Human | LQCFCHLCTKDNFTCVTDGLCFVSVTETTDKVIHNSMCIAEIDLI |
| NO: 7275 | TGFBR1 | PRDRPFVCAPSSKTGSVTTTYCCNQDHCNKIELPTTGPFSVKSSP |
| | isoform 2 | GLGPVELAAVIAGPVCFVCISLMLMVYICHNRTVIHHRVPNEED |
| | (P36897-2) | PSLDRPFISEGTTLKDLIYDMTTSGSGSGLPLLVQRTIARTIVLQE |
| | | SIGKGRFGEVWRGKWRGEEVAVKIFSSREERSWFREAEIYQTV |
| | | MLRHENILGFIAADNKDNGTWTQLWLVSDYHEHGSLFDYLNR |
| | | YTVTVEGMIKLALSTASGLAHLHMEIVGTQGKPAIAHRDLKSK |
| | | NILVKKNGTCCIADLGLAVRHDSATDTIDIAPNHRVGTKRYMAP |
| | | EVLDDSINMKHFESFKRADIYAMGLVFWEIARRCSIGGIHEDYQ |
| | | LPYYDLVPSDPSVEEMRKVVCEQKLRPNIPNRWQSCEALRVMA |
| | | KIMRECWYANGAARLTALRIKKTLSQLSQQEGIKM |
| SEQ ID | Immature | MEAAVAAPRPRLLLLVLAAAAAAAAALLPGATALQCFCHLCT |
| NO: 7259 | human | KDNFTCVTDGLCFVSVTETTDKVIHNSMCIAEIDLIPRDRPFVCA |
| | TGFBR1 | PSSKTGSVTTTYCCNQDHCNKIELPTTGLPLLVQRTIARTIVLQE |
| | isoform 3 | SIGKGRFGEVWRGKWRGEEVAVKIFSSREERSWFREAEIYQTV |
| | (P36897-3) | MLRHENILGFIAADNKDNGTWTQLWLVSDYHEHGSLFDYLNR |
| | | YTVTVEGMIKLALSTASGLAHLHMEIVGTQGKPAIAHRDLKSK |
| | | NILVKKNGTCCIADLGLAVRHDSATDTIDIAPNHRVGTKRYMAP |
| | | EVLDDSINMKHFESFKRADIYAMGLVFWEIARRCSIGGIHEDYQ |
| | | LPYYDLVPSDPSVEEMRKVVCEQKLRPNIPNRWQSCEALRVMA |
| | | KIMRECWYANGAARLTALRIKKTLSQLSQQEGIKM |
| SEQ ID | Human | LQCFCHLCTKDNFTCVTDGLCFVSVTETTDKVIHNSMCIAEIDLI |
| NO: 7276 | TGFBR1 | PRDRPFVCAPSSKTGSVTTTYCCNQDHCNKIELPTTGLPLLVQR |
| | isoform 3 | TIARTIVLQESIGKGRFGEVWRGKWRGEEVAVKIFSSREERSWF |
| | (P36897-3) | REAEIYQTVMLRHENILGFIAADNKDNGTWTQLWLVSDYHEHG |
| | | SLFDYLNRYTVTVEGMIKLALSTASGLAHLHMEIVGTQGKPAIA |
| | | HRDLKSKNILVKKNGTCCIADLGLAVRHDSATDTIDIAPNHRVG |
| | | TKRYMAPEVLDDSINMKHFESFKRADIYAMGLVFWEIARRCSIG |
| | | GIHEDYQLPYYDLVPSDPSVEEMRKVVCEQKLRPNIPNRWQSC |
| | | EALRVMAKIMRECWYANGAARLTALRIKKTLSQLSQQEGIKM |
| SEQ ID | Human | LQCFCHLCTKDNFTCVTDGLCFVSVTETTDKVIHNSMCIAEIDLI |
| NO: 7266 | TGFBR1 | PRDRPFVCAPSSKTGSVTTTYCCNQDHCNKIELPTTVKSSPGLGP |
| | fragment 1 | VEL |
| SEQ ID | Human | ALQCFCHLCTKDNFTCVTDGLCFVSVTETTDKVIHNSMCIAEID |
| NO: 7267 | TGFBR1 | LIPRDRPFVCAPSSKTGSVTTTYCCNQDHCNKIEL |
| | fragment 2 | |
| SEQ ID | Immature | MGRGLLRGLWPLHIVLWTRIASTIPPHVQKSVNNDMIVTDNNG |
| NO: 7260 | human | AVKFPQLCKFCDVRFSTCDNQKSCMSNCSITSICEKPQEVCVAV |
| | TGFBR2 | WRKNDENITLETVCHDPKLPYHDFILEDAASPKCIMKEKKKPGE |
| | isoform B | TFFMCSCSSDECNDNIIFSEEYNTSNPDLLLVIFQVTGISLLPPLG |
| | (short | VAISVIIIFYCYRVNRQQKLSSTWETGKTRKLMEFSEHCAIILED |
| | isoform) | DRSDISSTCANNINHNTELLPIELDTLVGKGRFAEVYKAKLKQN |
| | (P37173-1) | TSEQFETVAVKIFPYEEYASWKTEKDIFSDINLKHENILQFLTAE |
| | | ERKTELGKQYWLITAFHAKGNLQEYLTRHVISWEDLRKLGSSL |
| | | ARGIAHLHSDHTPCGRPKMPIVHRDLKSSNILVKNDLTCCLCDF |
| | | GLSLRLDPTLSVDDLANSGQVGTARYMAPEVLESRMNLENVES |
| | | FKQTDVYSMALVLWEMTSRCNAVGEVKDYEPPFGSKVREHPC |
| | | VESMKDNVLRDRGRPEIPSFWLNHQGIQMVCETLTECWDHDPE |
| | | ARLTAQCVAERFSELEHLDRLSGRSCSEEKIPEDGSLNTTK |
| SEQ ID | Human | TIPPHVQKSVNNDMIVTDNNGAVKFPQLCKFCDVRFSTCDNQK |
| NO: 7277 | TGFBR2 | SCMSNCSITSICEKPQEVCVAVWRKNDENITLETVCHDPKLPYH |
| | isoform B | DFILEDAASPKCIMKEKKKPGETFFMCSCSSDECNDNIIFSEEYN |
| | (short | TSNPDLLLVIFQVTGISLLPPLGVAISVIIIFYCYRVNRQQKLSST |
| | isoform) | WETGKTRKLMEFSEHCAIILEDDRSDISSTCANNINHNTELLPIEL |
| | (P37173-1) | DTLVGKGRFAEVYKAKLKQNTSEQFETVAVKIFPYEEYASWKT |
| | | EKDIFSDINLKHENILQFLTAEERKTELGKQYWLITAFHAKGNL |
| | | QEYLTRHVISWEDLRKLGSSLARGIAHLHSDHTPCGRPKMPIVH |
| | | RDLKSSNILVKNDLTCCLCDFGLSLRLDPTLSVDDLANSGQVGT |
| | | ARYMAPEVLESRMNLENVESFKQTDVYSMALVLWEMTSRCNA |
| | | VGEVKDYEPPFGSKVREHPCVESMKDNVLRDRGRPEIPSFWLN |
| | | HQGIQMVCETLTECWDHDPEARLTAQCVAERFSELEHLDRLSG |
| | | RSCSEEKIPEDGSLNTTK |
| SEQ ID | Immature | MGRGLLRGLWPLHIVLWTRIASTIPPHVQKSDVEMEAQKDEIIC |
| NO: 7261 | human | PSCNRTAHPLRHINNDMIVTDNNGAVKFPQLCKFCDVRFSTCD |
| | TGFBR2 | NQKSCMSNCSITSICEKPQEVCVAVWRKNDENITLETVCHDPKL |
| | isoform A | PYHDFILEDAASPKCIMKEKKKPGETFFMCSCSSDECNDNIIFSE |
| | (long | EYNTSNPDLLLVIFQVTGISLLPPLGVAISVIIIFYCYRVNRQQKLS |
| | isoform) | STWETGKTRKLMEFSEHCAIILEDDRSDISSTCANNINHNTELLPI |
| | (P37173-2) | ELDTLVGKGRFAEVYKAKLKQNTSEQFETVAVKIFPYEEYASW |
| | | KTEKDIFSDINLKHENILQFLTAEERKTELGKQYWLITAFHAKG |
| | | NLQEYLTRHVISWEDLRKLGSSLARGIAHLHSDHTPCGRPKMPI |
| | | VHRDLKSSNILVKNDLTCCLCDFGLSLRLDPTLSVDDLANSGQV |
| | | GTARYMAPEVLESRMNLENVESFKQTDVYSMALVLWEMTSRC |
| | | NAVGEVKDYEPPFGSKVREHPCVESMKDNVLRDRGRPEIPSFW |
| | | LNHQGIQMVCETLTECWDHDPEARLTAQCVAERFSELEHLDRL |
| | | SGRSCSEEKIPEDGSLNTTK |
| SEQ ID | Human | TIPPHVQKSDVEMEAQKDEIICPSCNRTAHPLRHINNDMIVTDN |
| NO: 7278 | TGFBR2 | NGAVKFPQLCKFCDVRFSTCDNQKSCMSNCSITSICEKPQEVCV |
| | isoform A | AVWRKNDENITLETVCHDPKLPYHDFILEDAASPKCIMKEKKKP |
| | (long | GETFFMCSCSSDECNDNIIFSEEYNTSNPDLLLVIFQVTGISLLPPL |
| | isoform) | GVAISVIIIFYCYRVNRQQKLSSTWETGKTRKLMEFSEHCAIILE |
| | (P37173-2) | DDRSDISSTCANNINHNTELLPIELDTLVGKGRFAEVYKAKLKQ |
| | | NTSEQFETVAVKIFPYEEYASWKTEKDIFSDINLKHENILQFLTA |
| | | EERKTELGKQYWLITAFHAKGNLQEYLTRHVISWEDLRKLGSS |
| | | LARGIAHLHSDHTPCGRPKMPIVHRDLKSSNILVKNDLTCCLCD |
| | | FGLSLRLDPTLSVDDLANSGQVGTARYMAPEVLESRMNLENVE |
| | | SFKQTDVYSMALVLWEMTSRCNAVGEVKDYEPPFGSKVREHP |
| | | CVESMKDNVLRDRGRPEIPSFWLNHQGIQMVCETLTECWDHDP |
| | | EARLTAQCVAERFSELEHLDRLSGRSCSEEKIPEDGSLNTTK |
| SEQ ID | Human | TIPPHVQKSVNNDMIVTDNNGAVKFPQLCKFCDVRFSTCDNQK |
| NO: 7262 | TGFBR2 | SCMSNCSITSICEKPQEVCVAVWRKNDENITLETVCHDPKLPYH |
| | fragment 1 | DFILEDAASPKCIMKEKKKPGETFFMCSCSSDECNDNIIFSEEYN |
| | (ECD of | TSNPD |
| | human | |
| | TGFBR2 | |
| | isoform B) | |
| SEQ ID | Human | IPPHVQKSVNNDMIVTDNNGAVKFPQLCKFCDVRFSTCDNQKS |
| NO: 7263 | TGFBR2 | CMSNCSITSICEKPQEVCVAVWRKNDENITLETVCHDPKLPYHD |
| | fragment 2 | FILEDAASPKCIMKEKKKPGETFFMCSCSSDECNDNIIFSEEYNTS |
| | | NPD |
| SEQ ID | Human | TIPPHVQKSDVEMEAQKDEIICPSCNRTAHPLRHINNDMIVTDN |
| NO: 7264 | TGFBR2 | NGAVKFPQLCKFCDVRFSTCDNQKSCMSNCSITSICEKPQEVCV |
| | fragment 3 | AVWRKNDENITLETVCHDPKLPYHDFILEDAASPKCIMKEKKKP |
| | (ECD of | GETFFMCSCSSDECNDNIIFSEEYNTSNPD |
| | human | |
| | TGFBR2 | |
| | isoform A) | |
| SEQ ID | Human | QLCKFCDVRFSTCDNQKSCMSNCSITSICEKPQEVCVAVWRKN |
| NO: 7265 | TGFBR2 | DENITLETVCHDPKLPYHDFILEDAASPKCIMKEKKKPGETFFM |
| | fragment 4 | CSCSSDECNDNIIF |
| SEQ ID | Immature | MTSHYVIAIFALMSSCLATAGPEPGALCELSPVSASHPVQALME |
| NO: 7268 | human | SFTVLSGCASRGTTGLPQEVHVLNLRTAGQGPGQLQREVTLHL |
| | TGFBR3 | NPISSVHIHHKSVVFLLNSPHPLVWHLKTERLATGVSRLFLVSEG |
| | isoform 1 | SVVQFSSANFSLTAETEERNFPHGNEHLLNWARKEYGAVTSFTE |
| | (Q03167-1) | LKIARNIYIKVGEDQVFPPKCNIGKNFLSLNYLAEYLQPKAAEG |
| | | CVMSSQPQNEEVHIIELITPNSNPYSAFQVDITIDIRPSQEDLEVV |
| | | KNLILILKCKKSVNWVIKSFDVKGSLKIIAPNSIGFGKESERSMT |
| | | MTKSIRDDIPSTQGNLVKWALDNGYSPITSYTMAPVANRFHLRL |
| | | ENNAEEMGDEEVHTIPPELRILLDPGALPALQNPPIRGGEGONG |
| | | GLPFPFPDISRRVWNEEGEDGLPRPKDPVIPSIQLFPGLREPEEVQ |
| | | GSVDIALSVKCDNEKMIVAVEKDSFQASGYSGMDVTLLDPTCK |
| | | AKMNGTHFVLESPLNGCGTRPRWSALDGVVYYNSIVIQVPALG |
| | | DSSGWPDGYEDLESGDNGFPGDMDEGDASLFTRPEIVVFNCSL |
| | | QQVRNPSSFQEQPHGNITFNMELYNTDLFLVPSQGVFSVPENGH |
| | | VYVEVSVTKAEQELGFAIQTCFISPYSNPDRMSHYTIIENICPKDE |
| | | SVKFYSPKRVHFPIPQADMDKKRFSFVFKPVFNTSLLFLQCELTL |
| | | CTKMEKHPQKLPKCVPPDEACTSLDASIIWAMMQNKKTFTKPL |
| | | AVIHHEAESKEKGPSMKEPNPISPPIFHGLDTLTVMGIAFAAFVI |
| | | GALLTGALWYIYSHTGETAGRQQVPTSPPASENSSAAHSIGSTQ |
| | | STPCSSSSTA |
| SEQ ID | Human | GPEPGALCELSPVSASHPVQALMESFTVLSGCASRGTTGLPQEV |
| NO: 7279 | TGFBR3 | HVLNLRTAGQGPGQLQREVTLHLNPISSVHIHHKSVVFLLNSPH |
| | isoform 1 | PLVWHLKTERLATGVSRLFLVSEGSVVQFSSANFSLTAETEERN |
| | (Q03167-1) | FPHGNEHLLNWARKEYGAVTSFTELKIARNIYIKVGEDQVFPPK |
| | | CNIGKNFLSLNYLAEYLQPKAAEGCVMSSQPQNEEVHIIELITPN |
| | | SNPYSAFQVDITIDIRPSQEDLEVVKNLILILKCKKSVNWVIKSFD |
| | | VKGSLKIIAPNSIGFGKESERSMTMTKSIRDDIPSTQGNLVKWAL |
| | | DNGYSPITSYTMAPVANRFHLRLENNAEEMGDEEVHTIPPELRI |
| | | LLDPGALPALQNPPIRGGEGQNGGLPFPFPDISRRVWNEEGEDG |
| | | LPRPKDPVIPSIQLFPGLREPEEVQGSVDIALSVKCDNEKMIVAV |
| | | EKDSFQASGYSGMDVTLLDPTCKAKMNGTHFVLESPLNGCGTR |
| | | PRWSALDGVVYYNSIVIQVPALGDSSGWPDGYEDLESGDNGFP |
| | | GDMDEGDASLFTRPEIVVFNCSLQQVRNPSSFQEQPHGNITFNM |
| | | ELYNTDLFLVPSQGVFSVPENGHVYVEVSVTKAEQELGFAIQTC |
| | | FISPYSNPDRMSHYTIIENICPKDESVKFYSPKRVHFPIPQADMDK |
| | | KRFSFVFKPVFNTSLLFLQCELTLCTKMEKHPQKLPKCVPPDEA |
| | | CTSLDASIIWAMMQNKKTFTKPLAVIHHEAESKEKGPSMKEPNP |
| | | ISPPIFHGLDTLTVMGIAFAAFVIGALLTGALWYIYSHTGETAGR |
| | | QQVPTSPPASENSSAAHSIGSTQSTPCSSSSTA |
| SEQ ID | Immature | MTSHYVIAIFALMSSCLATAGPEPGALCELSPVSASHPVQALME |
| NO: 7269 | human | SFTVLSGCASRGTTGLPQEVHVLNLRTAGQGPGQLQREVTLHL |
| | TGFBR3 | NPISSVHIHHKSVVFLLNSPHPLVWHLKTERLATGVSRLFLVSEG |
| | isoform 2 | SVVQFSSANFSLTAETEERNFPHGNEHLLNWARKEYGAVTSFTE |
| | (Q03167-2) | LKIARNIYIKVGEDQVFPPKCNIGKNFLSLNYLAEYLQPKAAEG |
| | | CVMSSQPQNEEVHIIELITPNSNPYSAFQVDITIDIRPSQEDLEVV |
| | | KNLILILKCKKSVNWVIKSFDVKGSLKIIAPNSIGFGKESERSMT |
| | | MTKSIRDDIPSTQGNLVKWALDNGYSPITSYTMAPVANRFHLRL |
| | | ENNEEMGDEEVHTIPPELRILLDPGALPALQNPPIRGGEGQNGGL |
| | | PFPFPDISRRVWNEEGEDGLPRPKDPVIPSIQLFPGLREPEEVQGS |
| | | VDIALSVKCDNEKMIVAVEKDSFQASGYSGMDVTLLDPTCKAK |
| | | MNGTHFVLESPLNGCGTRPRWSALDGVVYYNSIVIQVPALGDS |
| | | SGWPDGYEDLESGDNGFPGDMDEGDASLFTRPEIVVFNCSLQQ |
| | | VRNPSSFQEQPHGNITFNMELYNTDLFLVPSQGVFSVPENGHVY |
| | | VEVSVTKAEQELGFAIQTCFISPYSNPDRMSHYTIIENICPKDESV |
| | | KFYSPKRVHFPIPQADMDKKRFSFVFKPVFNTSLLFLQCELTLCT |
| | | KMEKHPQKLPKCVPPDEACTSLDASIIWAMMQNKKTFTKPLAV |
| | | IHHEAESKEKGPSMKEPNPISPPIFHGLDTLTVMGIAFAAFVIGAL |
| | | LTGALWYIYSHTGETAGRQQVPTSPPASENSSAAHSIGSTQSTPC |
| | | SSSSTA |
| SEQ ID | Human | GPEPGALCELSPVSASHPVQALMESFTVLSGCASRGTTGLPQEV |
| NO: 7280 | TGFBR3 | HVLNLRTAGQGPGQLQREVTLHLNPISSVHIHHKSVVFLLNSPH |
| | isoform 2 | PLVWHLKTERLATGVSRLFLVSEGSVVQFSSANFSLTAETEERN |
| | (Q03167-2) | FPHGNEHLLNWARKEYGAVTSFTELKIARNIYIKVGEDQVFPPK |
| | | CNIGKNFLSLNYLAEYLQPKAAEGCVMSSQPQNEEVHIIELITPN |
| | | SNPYSAFQVDITIDIRPSQEDLEVVKNLILILKCKKSVNWVIKSFD |
| | | VKGSLKIIAPNSIGFGKESERSMTMTKSIRDDIPSTQGNLVKWAL |
| | | DNGYSPITSYTMAPVANRFHLRLENNEEMGDEEVHTIPPELRILL |
| | | DPGALPALQNPPIRGGEGQNGGLPFPFPDISRRVWNEEGEDGLP |
| | | RPKDPVIPSIQLFPGLREPEEVQGSVDIALSVKCDNEKMIVAVEK |
| | | DSFQASGYSGMDVTLLDPTCKAKMNGTHFVLESPLNGCGTRPR |
| | | WSALDGVVYYNSIVIQVPALGDSSGWPDGYEDLESGDNGFPGD |
| | | MDEGDASLFTRPEIVVFNCSLQQVRNPSSFQEQPHGNITFNMEL |
| | | YNTDLFLVPSQGVFSVPENGHVYVEVSVTKAEQELGFAIQTCFI |
| | | SPYSNPDRMSHYTIIENICPKDESVKFYSPKRVHFPIPQADMDKK |
| | | RFSFVFKPVFNTSLLFLQCELTLCTKMEKHPQKLPKCVPPDEAC |
| | | TSLDASIIWAMMQNKKTFTKPLAVIHHEAESKEKGPSMKEPNPI |
| | | SPPIFHGLDTLTVMGIAFAAFVIGALLTGALWYIYSHTGETAGR |
| | | QQVPTSPPASENSSAAHSIGSTQSTPCSSSSTA |
| SEQ ID | Human | GPEPGALCELSPVSASHPVQALMESFTVLSGCASRGTTGLPQEV |
| NO: 7270 | TGFBR3 | HVLNLRTAGQGPGQLQREVTLHLNPISSVHIHHKSVVFLLNSPH |
| | fragment 1 | PLVWHLKTERLATGVSRLFLVSEGSVVQFSSANFSLTAETEERN |
| | | FPHGNEHLLNWARKEYGAVTSFTELKIARNIYIKVGEDQVFPPK |
| | | CNIGKNFLSLNYLAEYLQPKAAEGCVMSSQPQNEEVHIIELITPN |
| | | SNPYSAFQVDITIDIRPSQEDLEVVKNLILILKCKKSVNWVIKSFD |
| | | VKGSLKIIAPNSIGFGKESERSMTMTKSIRDDIPSTQGNLVKWAL |
| | | DNGYSPITSYTMAPVANRFHLRLENNAEEMGDEEVHTIPPELRI |
| | | LLDPGALPALQNPPIRGGEGQNGGLPFPFPDISRRVWNEEGEDG |
| | | LPRPKDPVIPSIQLFPGLREPEEVQGSVDIALSVKCDNEKMIVAV |
| | | EKDSFQASGYSGMDVTLLDPTCKAKMNGTHFVLESPLNGCGTR |
| | | PRWSALDGVVYYNSIVIQVPALGDSSGWPDGYEDLESGDNGFP |
| | | GDMDEGDASLFTRPEIVVFNCSLQQVRNPSSFQEQPHGNITFNM |
| | | ELYNTDLFLVPSQGVFSVPENGHVYVEVSVTKAEQELGFAIQTC |
| | | FISPYSNPDRMSHYTIIENICPKDESVKFYSPKRVHFPIPQADMDK |
| | | KRFSFVFKPVFNTSLLFLQCELTLCTKMEKHPQKLPKCVPPDEA |
| | | CTSLDASIIWAMMQNKKTFTKPLAVIHHEAESKEKGPSMKEPNP |
| | | ISPPIFHGLDTLTV |
| SEQ ID | hCH1- | ASTKGPSVFPLAPSSKSTSGGTAALGCLVKDYFPEPVTVSWNSG |
| NO: 7281 | hFc Hole- | ALTSGVHTFPAVLQSSGLYSLSSVVTVPSSSLGTQTYICNVNHKP |
| | 3x4GS- | SNTKVDKRVEPKSCDKTHTCPPCPAPELLGGPSVFLFPPKPKDTL |
| | TGFbR2 | MISRTPEVTCVVVDVSHEDPEVKFNWYVDGVEVHNAKTKPRE |
| | | EQYNSTYRVVSVLTVLHQDWLNGKEYKCKVSNKALPAPIEKTI |
| | | SKAKGQPREPQVCTLPPSREEMTKNQVSLSCAVKGFYPSDIAVE |
| | | WESNGQPENNYKTTPPVLDSDGSFFLVSKLTVDKSRWQQGNVF |
| | | SCSVMHEALHNHYTQKSLSLSPGXGGGGSGGGGSGGGGSIPPH |
| | | VQKSVNNDMIVTDNNGAVKFPQLCKFCDVRFSTCDNQKSCMS |
| | | NCSITSICEKPQEVCVAVWRKNDENITLETVCHDPKLPYHDFILE |
| | | DAASPKCIMKEKKKPGETFFMCSCSSDECNDNIIFSEEYNTSNPD, |
| | | wherein X is K or absent |
| SEQ ID | hCH1- | ASTKGPSVFPLAPSSKSTSGGTAALGCLVKDYFPEPVTVSWNSG |
| NO: 7282 | hFc Knob- | ALTSGVHTFPAVLQSSGLYSLSSVVTVPSSSLGTQTYICNVNHKP |
| | 3×4GS- | SNTKVDKRVEPKSCDKTHTCPPCPAPELLGGPSVFLFPPKPKDTL |
| | TGFbR2 | MISRTPEVTCVVVDVSHEDPEVKFNWYVDGVEVHNAKTKPRE |
| | | EQYNSTYRVVSVLTVLHQDWLNGKEYKCKVSNKALPAPIEKTI |
| | | SKAKGQPREPQVYTLPPCREEMTKNQVSLWCLVKGFYPSDIAV |
| | | EWESNGQPENNYKTTPPVLDSDGSFFLYSKLTVDKSRWQQGNV |
| | | FSCSVMHEALHNHYTQKSLSLSPGXGGGGSGGGGSGGGGSIPP |
| | | HVQKSVNNDMIVTDNNGAVKFPQLCKFCDVRFSTCDNQKSCM |
| | | SNCSITSICEKPQEVCVAVWRKNDENITLETVCHDPKLPYHDFIL |
| | | EDAASPKCIMKEKKKPGETFFMCSCSSDECNDNIIFSEEYNTSNP |
| | | D, wherein X is K or absent |
| SEQ ID | hFc Hole- | DKTHTCPPCPAPELLGGPSVFLFPPKPKDTLMISRTPEVTCVVVD |
| NO: 7283 | 3×4GS- | VSHEDPEVKFNWYVDGVEVHNAKTKPREEQYNSTYRVVSVLT |
| | TGFbR2 | VLHQDWLNGKEYKCKVSNKALPAPIEKTISKAKGQPREPQVCT |
| | | LPPSREEMTKNQVSLSCAVKGFYPSDIAVEWESNGQPENNYKT |
| | | TPPVLDSDGSFFLVSKLTVDKSRWQQGNVFSCSVMHEALHNHY |
| | | TQKSLSLSPGXGGGGSGGGGSGGGGSIPPHVQKSVNNDMIVTD |
| | | NNGAVKFPQLCKFCDVRFSTCDNQKSCMSNCSITSICEKPQEVC |
| | | VAVWRKNDENITLETVCHDPKLPYHDFILEDAASPKCIMKEKK |
| | | KPGETFFMCSCSSDECNDNIIFSEEYNTSNPD, wherein X is K or |
| | | absent |
| SEQ ID | hFc Knob- | DKTHTCPPCPAPELLGGPSVFLFPPKPKDTLMISRTPEVTCVVVD |
| NO: 7284 | 3×4GS- | VSHEDPEVKFNWYVDGVEVHNAKTKPREEQYNSTYRVVSVLT |
| | TGFbR2 | VLHQDWLNGKEYKCKVSNKALPAPIEKTISKAKGQPREPQVYT |
| | | LPPCREEMTKNQVSLWCLVKGFYPSDIAVEWESNGQPENNYKT |
| | | TPPVLDSDGSFFLYSKLTVDKSRWQQGNVFSCSVMHEALHNHY |
| | | TQKSLSLSPGXGGGGSGGGGSGGGGSIPPHVQKSVNNDMIVTD |
| | | NNGAVKFPQLCKFCDVRFSTCDNQKSCMSNCSITSICEKPQEVC |
| | | VAVWRKNDENITLETVCHDPKLPYHDFILEDAASPKCIMKEKK |
| | | KPGETFFMCSCSSDECNDNIIFSEEYNTSNPD, wherein X is K or |
| | | absent |
| SEQ ID | TGFbR2- | IPPHVQKSVNNDMIVTDNNGAVKFPQLCKFCDVRFSTCDNQKS |
| NO: 7285 | 3×4GS- | CMSNCSITSICEKPQEVCVAVWRKNDENITLETVCHDPKLPYHD |
| | hCH1- | FILEDAASPKCIMKEKKKPGETFFMCSCSSDECNDNIIFSEEYNTS |
| | hFc Hole | NPDGGGGSGGGGSGGGGSASTKGPSVFPLAPSSKSTSGGTAAL |
| | | GCLVKDYFPEPVTVSWNSGALTSGVHTFPAVLQSSGLYSLSSVV |
| | | TVPSSSLGTQTYICNVNHKPSNTKVDKRVEPKSCDKTHTCPPCP |
| | | APELLGGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVCTLPPSREEMTK |
| | | NQVSLSCAVKGFYPSDIAVEWESNGQPENNYKTTPPVLDSDGSF |
| | | FLVSKLTVDKSRWQQGNVFSCSVMHEALHNHYTQKSLSLSPGX, |
| | | wherein X is K or absent |
| SEQ ID | TGFbR2- | IPPHVQKSVNNDMIVTDNNGAVKFPQLCKFCDVRFSTCDNQKS |
| NO: 7286 | 3×4GS- | CMSNCSITSICEKPQEVCVAVWRKNDENITLETVCHDPKLPYHD |
| | hCH1- | FILEDAASPKCIMKEKKKPGETFFMCSCSSDECNDNIIFSEEYNTS |
| | hFc Knob | NPDGGGGSGGGGSGGGGSASTKGPSVFPLAPSSKSTSGGTAAL |
| | | GCLVKDYFPEPVTVSWNSGALTSGVHTFPAVLQSSGLYSLSSVV |
| | | TVPSSSLGTQTYICNVNHKPSNTKVDKRVEPKSCDKTHTCPPCP |
| | | APELLGGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPCREEMTK |
| | | NQVSLWCLVKGFYPSDIAVEWESNGQPENNYKTTPPVLDSDGS |
| | | FFLYSKLTVDKSRWQQGNVFSCSVMHEALHNHYTQKSLSLSPG |
| | | X, wherein X is K or absent |
| SEQ ID | TGFbR2- | IPPHVQKSVNNDMIVTDNNGAVKFPQLCKFCDVRFSTCDNQKS |
| NO: 7287 | 3x4GS- | CMSNCSITSICEKPQEVCVAVWRKNDENITLETVCHDPKLPYHD |
| | hCLIg_vl | FILEDAASPKCIMKEKKKPGETFFMCSCSSDECNDNIIFSEEYNTS |
| | | NPDGGGGSGGGGSGGGGSGQPKANPTVTLFPPSSEELQANKAT |
| | | LVCLISDFYPGAVTVAWKADGSPVKAGVETTKPSKQSNNKYAA |
| | | SSYLSLTPEQWKSHRSYSCQVTHEGSTVEKTVAPTECS |
| SEQ ID | TGFBR2- | IPPHVQKSVNNDMIVTDNNGAVKFPQLCKFCDVRFSTCDNQKS |
| NO: 7288 | 3x4GS- | CMSNCSITSICEKPQEVCVAVWRKNDENITLETVCHDPKLPYHD |
| | hCLIg_vk | FILEDAASPKCIMKEKKKPGETFFMCSCSSDECNDNIIFSEEYNTS |
| | | NPDGGGGSGGGGSGGGGSRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |

TABLE 15
Exemplary construct sequences
Construct 1

| | | | |
|---|---|---|---|
| Chain 1 | 3529 | Full Sequence | QVQLQQSGTELMKPGASVKISCKASGYTFSNYWIEWIKQRP |
| (heavy | | | GHGLEWVGEILPGAGPTNYNEKFKGKATFTADSSSNTAYM |
| chain) | | | QLSSLTSEDSAVYYCARTDYDYDWFAYWGQGTLVTVSAA |
| | | | KTTAPSVYPLAPVSGDTTGSSVTLGCLVKGYFPEPVTLTWN |
| | | | SGSLSSGVHTFPAVLQSDLYTLSSSVTVTSSTWPSQSITCNV |
| | | | AHPASSTKVDKKIEPKSCDKTHTCPPCPAPNAAGGPSVFIFP |
| | | | PKIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVCVLPPPEEEMTKKQVTLSCA |
| | | | VTDFMPEDIYVEWTNNGKTELNYKNTEPVLDSDGSYFMVS |
| | | | KLRVEKKNWVERNSYSCSVVHEGLHNHHTTKSFSRTPGKE |
| | | | PEA |
| | 3548 | Full Sequence | QVQLQQSGTELMKPGASVKISCKASGYTFSNYWIEWIKQRP |
| | | without tag | GHGLEWVGEILPGAGPTNYNEKFKGKATFTADSSSNTAYM |
| | | | QLSSLTSEDSAVYYCARTDYDYDWFAYWGQGTLVTVSAA |
| | | | KTTAPSVYPLAPVSGDTTGSSVTLGCLVKGYFPEPVTLTWN |
| | | | SGSLSSGVHTFPAVLQSDLYTLSSSVTVTSSTWPSQSITCNV |
| | | | AHPASSTKVDKKIEPKSCDKTHTCPPCPAPNAAGGPSVFIFP |
| | | | PKIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVCVLPPPEEEMTKKQVTLSCA |
| | | | VTDFMPEDIYVEWTNNGKTELNYKNTEPVLDSDGSYFMVS |
| | | | KLRVEKKNWVERNSYSCSVVHEGLHNHHTTKSFSRTPGK |
| | 3530 | anti-TCRvβ | QVQLQQSGTELMKPGASVKISCKASGYTFSNYWIEWIKQRP |
| | | VH | GHGLEWVGEILPGAGPTNYNEKFKGKATFTADSSSNTAYM |
| | | from murine | QLSSLTSEDSAVYYCARTDYDYDWFAYWGQGTLVTVSAA |
| | | MR5-2 | KTTAPSVYPLAPVSGDTTGSSVTLGCLVKGYFPEPVTLTWN |
| | | | SGSLSSGVHTFPAVLQSDLYTLSSSVTVTSS |
| | 3531 | mouse Fc | TWPSQSITCNVAHPASSTKVDKKIEPKSCDKTHTCPPCPAPN |
| | | heavy chain | AAGGPSVFIFPPKIKDVLMISLSPIVTCVVVDVSEDDPDVQIS |
| | | comprising | WFVNNVEVHTAQTQTHREDYNSTLRVVSALPIQHQDWMS |
| | | LALAPG | GKEFKCKVNNKDLGAPIERTISKPKGSVRAPQVCVLPPPEEE |
| | | mutations | MTKKQVTLSCAVTDFMPEDIYVEWTNNGKTELNYKNTEPV |
| | | (L234A L235A | LDSDGSYFMVSKLRVEKKNWVERNSYSCSVVHEGLHNHH |
| | | and P329G) | TTKSFSRTPGK |
| | 3547 | Tag | EPEA |
| Chain 2 | 3532 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLTGGGGSGGGGSDKTHTCPPCPAPNAAGGPSVFIFPP |
| | | | KIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVYVLPPCEEEMTKKQVTLWC |
| | | | MVTDFMPEDIYVEWTNNGKTELNYKNTEPVLDSDGSYFMY |
| | | | SKLRVEKKNWVERNSYSCSVVHEGLHNHHTTKSFSRTPGK |
| | | | GGGGSHHHHHHHH |
| | 3549 | Full Sequence | APTSSSTKKTQLQLEHLLLDLQMILNGINNYKNPKLTRMLT |
| | | without tag | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLTGGGGSGGGGSDKTHTCPPCPAPNAAGGPSVFIFPP |
| | | | KIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVYVLPPCEEEMTKKQVTLWC |
| | | | MVTDFMPEDIYVEWTNNGKTELNYKNTEPVLDSDGSYFMY |
| | | | SKLRVEKKNWVERNSYSCSVVHEGLHNHHTTKSFSRTPGK |
| | 2191 | IL-2 | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLT |
| | 3308 | linker | GGGGSGGGGS |
| | 3533 | mouse Fc | DKTHTCPPCPAPNAAGGPSVFIFPPKIKDVLMISLSPIVTCVV |
| | | heavy chain | VDVSEDDPDVQISWFVNNVEVHTAQTQTHREDYNSTLRVV |
| | | comprising | SALPIQHQDWMSGKEFKCKVNNKDLGAPIERTISKPKGSVR |
| | | LALAPG | APQVYVLPPCEEEMTKKQVTLWCMVTDFMPEDIYVEWTN |
| | | mutations | NGKTELNYKNTEPVLDSDGSYFMYSKLRVEKKNWVERNS |
| | | (L234A L235A | YSCSVVHEGLHNHHTTKSFSRTPGK |
| | | and P329G) | |
| | 3534 | Tag | GGGGSHHHHHHHH |
| Chain 3 | 3526 | Full Sequence | DIVMSQSPSSLAVSVGEKVTMSCKSSQSLLYSGNQKNYLA |
| (light chain) | | | WYQQKPGQSPKLLIYWASTRESGVPDRFTGSGSGTDFTLTI |
| | | | NSVKAEDLTVYYCQQYYGYPRTFGGGTKVEIKRADAAPTV |
| | | | SIFPPSSEQLTSGGASVVCFLNNFYPKDINVKWKIDGSERQN |
| | | | GVLNSWTDQDSKDSTYSMSSTLTLTKDEYERHNSYTCEAT |
| | | | HKTSTSPIVKSFNRNEC |
| | 3527 | anti-TCRvß | DIVMSQSPSSLAVSVGEKVTMSCKSSQSLLYSGNQKNYLA |
| | | VL | WYQQKPGQSPKLLIYWASTRESGVPDRFTGSGSGTDFTLTI |
| | | from murine | NSVKAEDLTVYYCQQYYGYPRTFGGGTKVEIK |
| | | MR5-2 | |
| | 3528 | Immunoglobulin | RADAAPTVSIFPPSSEQLTSGGASVVCFLNNFYPKDINVKWK |
| | | constant | IDGSERQNGVLNSWTDQDSKDSTYSMSSTLTLTKDEYERHN |
| | | region | SYTCEATHKTSTSPIVKSFNRNEC |

Construct 2

| | | | |
|---|---|---|---|
| Chain 1 | 3536 | Full Sequence | QVQLQQSGTELMKPGASVKISCKASGYTFSNYWIEWIKQRP |
| (heavy | | | GHGLEWVGEILPGAGPTNYNEKFKGKATFTADSSSNTAYM |
| chain) | | | QLSSLTSEDSAVYYCARTDYDYDWFAYWGQGTLVTVSAA |
| | | | KTTAPSVYPLAPVSGDTTGSSVTLGCLVKGYFPEPVTLTWN |
| | | | SGSLSSGVHTFPAVLQSDLYTLSSSVTVTSSTWPSQSITCNV |
| | | | AHPASSTKVDKKIEPKSCDKTHTCPPCPAPNAAGGPSVFIFP |
| | | | PKIKDVLMISLSPIVTCVVVDVSEDDPDVQISWFVNNVEVHT |
| | | | AQTQTHREDYNSTLRVVSALPIQHQDWMSGKEFKCKVNNK |
| | | | DLGAPIERTISKPKGSVRAPQVYVLPPPEEEMTKKQVTLTC |
| | | | MVTDFMPEDIYVEWTNNGKTELNYKNTEPVLDSDGSYFMY |
| | | | SKLRVEKKNWVERNSYSCSVVHEGLHNHHTTKSFSRTPGK |
| | | | EPEA |
| | 3530 | anti-TCRvβ | QVQLQQSGTELMKPGASVKISCKASGYTFSNYWIEWIKQRP |
| | | VH | GHGLEWVGEILPGAGPTNYNEKFKGKATFTADSSSNTAYM |
| | | from murine | QLSSLTSEDSAVYYCARTDYDYDWFAYWGQGTLVTVSAA |
| | | MR5-2 | KTTAPSVYPLAPVSGDTTGSSVTLGCLVKGYFPEPVTLTWN |
| | | | SGSLSSGVHTFPAVLQSDLYTLSSSVTVTSS |
| | 3537 | mouse Fc | TWPSQSITCNVAHPASSTKVDKKIEPKSCDKTHTCPPCPAPN |
| | | heavy chain | AAGGPSVFIFPPKIKDVLMISLSPIVTCVVVDVSEDDPDVQIS |
| | | comprising | WFVNNVEVHTAQTQTHREDYNSTLRVVSALPIQHQDWMS |
| | | LALAPG | GKEFKCKVNNKDLGAPIERTISKPKGSVRAPQVYVLPPPEEE |
| | | mutations | MTKKQVTLTCMVTDFMPEDIYVEWTNNGKTELNYKNTEP |
| | | (L234A L235A | VLDSDGSYFMYSKLRVEKKNWVERNSYSCSVVHEGLHNH |
| | | and P329G) | HTTKSFSRTPGKEPEA |
| Chain 2 | 3535 | Full Sequence | DIVMSQSPSSLAVSVGEKVTMSCKSSQSLLYSGNQKNYLA |
| (light chain- | | | WYQQKPGQSPKLLIYWASTRESGVPDRFTGSGSGTDFTLTI |
| IL2) | | | NSVKAEDLTVYYCQQYYGYPRTFGGGTKVEIKRADAAPTV |
| | | | SIFPPSSEQLTSGGASVVCFLNNFYPKDINVKWKIDGSERQN |
| | | | GVLNSWTDQDSKDSTYSMSSTLTLTKDEYERHNSYTCEAT |
| | | | HKTSTSPIVKSFNRNECGGGGSGGGGSGGGGSAPTSSSTKK |
| | | | TQLQLEHLLLDLQMILNGINNYKNPKLTRMLTFKFYMPKK |
| | | | ATELKHLQCLEEELKPLEEVLNLAQSKNFHLRPRDLISNINVI |
| | | | VLELKGSETTFMCEYADETATIVEFLNRWITFCQSIISTLT |
| | 3527 | anti-TCRvβ | DIVMSQSPSSLAVSVGEKVTMSCKSSQSLLYSGNQKNYLA |
| | | VL | WYQQKPGQSPKLLIYWASTRESGVPDRFTGSGSGTDFTLTI |
| | | from murine | NSVKAEDLTVYYCQQYYGYPRTFGGGTKVEIK |
| | | MR5-2 | |
| | 3528 | Immunoglobulin | RADAAPTVSIFPPSSEQLTSGGASVVCFLNNFYPKDINVKWK |
| | | constant | IDGSERQNGVLNSWTDQDSKDSTYSMSSTLTLTKDEYERHN |
| | | region | SYTCEATHKTSTSPIVKSFNRNEC |
| | 3309 | linker | GGGGSGGGGSGGGGS |
| | 2191 | IL-2 | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLT |

Humanized Molecule 1

| | | | |
|---|---|---|---|
| Chain 1 | 3517 | Full Sequence | QVQLVQSGAEVKKPGSSVKVSCKASGHDFRLTYIHWVRQA |
| (heavy | | | PGQGLEWMGRVSAGSGNVKYNEKFKGRVTITADTSTSTAY |
| chain) | | | MELSSLRSEDTAVYYCAVSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 1346 | anti-TCRvβ | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 3649 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2 | 3521 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSPG |
| | | | K |
| | 2270 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 3308 | linker | GGGGSGGGGS |
| | 3648 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3 | 3518 | Full Sequence | DIQMTQSPSFLSASVGDRVTITCKASQNVADRVVWHQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 1349 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 2

| | | | |
|---|---|---|---|
| Chain 1 | 4000 | Full Sequence | QVQLVQSGAEVKKPGSSVKVSCKASGHDFRLTYIHWVRQA |
| (heavy | | | PGQGLEWMGRVSAGSGNVKYNEKFKGRVTITADTSTSTAY |
| chain) | | | MELSSLRSEDTAVYYCAVSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | P |
| | 1346 | anti-TCRvβ | QVQLVQSGAEVKKPGSSVKVSCKASGHDFRLTYIHWVRQA |
| | | VH | PGQGLEWMGRVSAGSGNVKYNEKFKGRVTITADTSTSTAY |
| | | | MELSSLRSEDTAVYYCAVSYYSYDVLDYWGQGTTVTVSS |
| | 4001 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SP |
| Chain 2 | 4002 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSP |
| | 2270 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 3308 | linker | GGGGSGGGGS |
| | 4003 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSP |
| Chain 3 | 3518 | Full Sequence | DIQMTQSPSFLSASVGDRVTITCKASQNVADRVVWHQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 1349 | anti-TCRvß | DIQMTQSPSFLSASVGDRVTITCKASQNVADRVVWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 3

| | | | |
|---|---|---|---|
| Chain 1 | 4004 | Full Sequence | QVQLVQSGAEVKKPGSSVKVSCKASGYSFTTYYIHWVRQA |
| (heavy | | | PGQGLEWMGWFFPGSGNIKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 9 | anti-TCRvβ | QVQLVQSGAEVKKPGSSVKVSCKASGYSFTTYYIHWVRQA |
| | | VH | PGQGLEWMGWFFPGSGNIKYNEKFKGRVTITADTSTSTAY |
| | | | MELSSLRSEDTAVYYCAGSYYSYDVLDYWGQGTTVTVSS |
| | 3649 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2 | 7515 | Full Sequence | APASSSTKKTQLQLEHLLLDLQMILNGINNYKNPKLTRMLT |
| (IL2-Fc) | | | AKFAMPKKATELKHLQCLEEELKPLEEVLNGAQSKNFHLR |
| | | | PRDLISNINVIVLELKGSETTFMCEYADETATIVEFLNRWITF |
| | | | AQSIISTLTGGGGSGGGGSDKTHTCPPCPAPELLGGPSVFLFP |
| | | | PKPKDTLMISRTPEVTCVVVDVSHEDPEVKFNWYVDGVEV |
| | | | HNAKTKPREEQYASTYRVVSVLTVLHQDWLNGKEYKCKV |
| | | | SNKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSPG |
| | | | K |
| | 2280 | IL-2 | APASSSTKKTQLQLEHLLLDLQMILNGINNYKNPKLTRMLT |
| | | | AKFAMPKKATELKHLQCLEEELKPLEEVLNGAQSKNFHLR |
| | | | PRDLISNINVIVLELKGSETTFMCEYADETATIVEFLNRWITF |
| | | | AQSIISTLT |
| | 3308 | linker | GGGGSGGGGS |
| | 3648 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3 | 4005 | Full Sequence | DIQMTQSPSSLSASVGDRVTITCKASQNVGINVVWHQQKPG |
| (light chain) | | | KVPKALIYSSSHRYSGVPSRFSGSGSGTDFTLTISSLQPEDVA |
| | | | TYFCQQFKSYPLTFGQGTKLEIKRTVAAPSVFIFPPSDEQLKS |
| | | | GTASVVCLLNNFYPREAKVQWKVDNALQSGNSQESVTEQ |
| | | | DSKDSTYSLSSTLTLSKADYEKHKVYACEVTHQGLSSPVTK |
| | | | SFNRGEC |
| | 11 | anti-TCRvβ | DIQMTQSPSSLSASVGDRVTITCKASQNVGINVVWHQQKPG |
| | | VL | KVPKALIYSSSHRYSGVPSRFSGSGSGTDFTLTISSLQPEDVA |
| | | | TYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 4

| | | | |
|---|---|---|---|
| Chain 1 | 4006 | Full Sequence | QVQLVQSGAEVKKPGSSVKVSCKASGYSFTTYYIHWVRQA |
| (heavy | | | PGQGLEWMGWFFPGSGNIKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | P |
| | 9 | anti-TCRvβ | QVQLVQSGAEVKKPGSSVKVSCKASGYSFTTYYIHWVRQA |
| | | VH | PGQGLEWMGWFFPGSGNIKYNEKFKGRVTITADTSTSTAY |
| | | | MELSSLRSEDTAVYYCAGSYYSYDVLDYWGQGTTVTVSS |
| | 4001 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SP |
| Chain 2 | 4007 | Full Sequence | APASSSTKKTQLQLEHLLLDLQMILNGINNYKNPKLTRMLT |
| (IL2-Fc) | | | AKFAMPKKATELKHLQCLEEELKPLEEVLNGAQSKNFHLR |
| | | | PRDLISNINVIVLELKGSETTFMCEYADETATIVEFLNRWITF |
| | | | AQSIISTLTGGGGSGGGGSDKTHTCPPCPAPELLGGPSVFLFP |
| | | | PKPKDTLMISRTPEVTCVVVDVSHEDPEVKFNWYVDGVEV |
| | | | HNAKTKPREEQYASTYRVVSVLTVLHQDWLNGKEYKCKV |
| | | | SNKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSP |
| | 2280 | IL-2 | APASSSTKKTQLQLEHLLLDLQMILNGINNYKNPKLTRMLT |
| | | | AKFAMPKKATELKHLQCLEEELKPLEEVLNGAQSKNFHLR |
| | | | PRDLISNINVIVLELKGSETTFMCEYADETATIVEFLNRWITF |
| | | | AQSIISTLT |
| | 3308 | linker | GGGGSGGGGS |
| | 4003 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSP |
| Chain 3 | 4005 | Full Sequence | DIQMTQSPSSLSASVGDRVTITCKASQNVGINVVWHQQKPG |
| (light chain) | | | KVPKALIYSSSHRYSGVPSRFSGSGSGTDFTLTISSLQPEDVA |
| | | | TYFCQQFKSYPLTFGQGTKLEIKRTVAAPSVFIFPPSDEQLKS |
| | | | GTASVVCLLNNFYPREAKVQWKVDNALQSGNSQESVTEQ |
| | | | DSKDSTYSLSSTLTLSKADYEKHKVYACEVTHQGLSSPVTK |
| | | | SFNRGEC |
| | 11 | anti-TCRvβ | DIQMTQSPSSLSASVGDRVTITCKASQNVGINVVWHQQKPG |
| | | VL | KVPKALIYSSSHRYSGVPSRFSGSGSGTDFTLTISSLQPEDVA |
| | | | TYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 5

| | | | |
|---|---|---|---|
| Chain 1 | 4008 | Full Sequence | QVQLVQSGAEVKKPGSSVKVSCKASGTDFDKIYIHWVRQA |
| (heavy | | | PGQGLEWMGRVSAGSGNTKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 109 | anti-TCRvβ | QVQLVQSGAEVKKPGSSVKVSCKASGTDFDKIYIHWVRQA |
| | | VH | PGQGLEWMGRVSAGSGNTKYNEKFKGRVTITADTSTSTAY |
| | | | MELSSLRSEDTAVYYCAGSYYSYDVLDYWGQGTTVTVSS |
| | 3649 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2 | 3521 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSPG |
| | | | K |
| | 2270 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 3308 | linker | GGGGSGGGGS |
| | 3648 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3 | 4009 | Full Sequence | DIQMTQSPSFLSASVGDRVTITCKASQNVGNRVAWYQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 108 | anti-TCRvβ | DIQMTQSPSFLSASVGDRVTITCKASQNVGNRVAWYQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 6

| | | | |
|---|---|---|---|
| Chain 1 | 4010 | Full Sequence | QVQLVQSGAEVKKPGSSVKVSCKASGTDFDKIYIHWVRQA |
| (heavy | | | PGQGLEWMGRVSAGSGNTKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | P |
| | 109 | anti-TCRvβ | QVQLVQSGAEVKKPGSSVKVSCKASGTDFDKIYIHWVRQA |
| | | VH | PGQGLEWMGRVSAGSGNTKYNEKFKGRVTITADTSTSTAY |
| | | | MELSSLRSEDTAVYYCAGSYYSYDVLDYWGQGTTVTVSS |
| | 4001 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SP |
| Chain 2 | 7516 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSP |
| | 2270 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 3308 | linker | GGGGSGGGGS |
| | 3648 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| | 4003 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSP |
| Chain 3 | 4009 | Full Sequence | DIQMTQSPSFLSASVGDRVTITCKASQNVGNRVAWYQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 108 | anti-TCRvβ | DIQMTQSPSFLSASVGDRVTITCKASQNVGNRVAWYQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 7

| | | | |
|---|---|---|---|
| Chain 1 | 4011 | Full Sequence | QVQLVQSGAEVKKPGSSVKVSCKASGHDFKLTYIHWVRQA |
| (heavy | | | PGQGLEWMGRVSPGSGNTKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 182 | anti-TCRvβ | QVQLVQSGAEVKKPGSSVKVSCKASGHDFKLTYIHWVRQA |
| | | VH | PGQGLEWMGRVSPGSGNTKYNEKFKGRVTITADTSTSTAY |
| | | | MELSSLRSEDTAVYYCAGSYYSYDVLDYWGQGTTVTVSS |
| | 3649 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2 | 4013 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSPG |
| | | | K |
| | 2191 | IL-2 | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLT |
| | 3308 | linker | GGGGSGGGGS |
| | 3648 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3 | 4012 | Full Sequence | DIQMTQSPSFLSASVGDRVTITCKASQNVEDRVAWHQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 181 | anti-TCRvβ | DIQMTQSPSFLSASVGDRVTITCKASQNVEDRVAWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 8

| | | | |
|---|---|---|---|
| Chain 1 | 4014 | Full Sequence | QVQLVQSGAEVKKPGSSVKVSCKASGHDFKLTYIHWVRQA |
| (heavy | | | PGQGLEWMGRVSPGSGNTKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | P |
| | 182 | anti-TCRvβ | QVQLVQSGAEVKKPGSSVKVSCKASGHDFKLTYIHWVRQA |
| | | VH | PGQGLEWMGRVSPGSGNTKYNEKFKGRVTITADTSTSTAY |
| | | | MELSSLRSEDTAVYYCAGSYYSYDVLDYWGQGTTVTVSS |
| | 4001 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SP |
| Chain 2 | 4015 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSP |
| | 2191 | IL-2 | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFC |
| | | | QSIISTLT |
| | 3308 | linker | GGGGSGGGGS |
| | 4003 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSP |
| Chain 3 | 4012 | Full Sequence | DIQMTQSPSFLSASVGDRVTITCKASQNVEDRVAWHQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 181 | anti-TCRvβ | DIQMTQSPSFLSASVGDRVTITCKASQNVEDRVAWHQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 9

| | | | |
|---|---|---|---|
| Chain 1 | 4016 | Full Sequence | QVQLVQSGAEVKKPGSSVKVSCKASGTDFDKTYIHWVRQA |
| (heavy | | | PGQGLEWMGRISAGSGNVKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | PGK |
| | 187 | anti-TCRvβ | QVQLVQSGAEVKKPGSSVKVSCKASGTDFDKTYIHWVRQA |
| | | VH | PGQGLEWMGRISAGSGNVKYNEKFKGRVTITADTSTSTAY |
| | | | MELSSLRSEDTAVYYCAGSYYSYDVLDYWGQGTTVTVSS |
| | 3649 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SPGK |
| Chain 2 | 3521 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSPG |
| | | | K |
| | 2270 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 3308 | linker | GGGGSGGGGS |
| | 3648 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| Chain 3 | 4017 | Full Sequence | DIQMTQSPSFLSASVGDRVTITCKASQNVDDRVAWYQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 7513 | anti-TCRvβ | DIQMTQSPSFLSASVGDRVTITCKASQNVDDRVAWYQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

Humanized Molecule 10

| | | | |
|---|---|---|---|
| Chain 1 | 4018 | Full Sequence | QVQLVQSGAEVKKPGSSVKVSCKASGTDFDKTYIHWVRQA |
| (heavy | | | PGQGLEWMGRISAGSGNVKYNEKFKGRVTITADTSTSTAY |
| chain) | | | MELSSLRSEDTAVYYCAGSYYSYDVLDYWGQGTTVTVSSA |
| | | | STKGPSVFPLAPSSKSTSGGTAALGCLVKDYFPEPVTVSWN |
| | | | SGALTSGVHTFPAVLQSSGLYSLSSVVTVPSSSLGTQTYICN |
| | | | VNHKPSNTKVDKRVEPKSCDKTHTCPPCPAPELLGGPSVFL |
| | | | FPPKPKDTLMISRTPEVTCVVVDVSHEDPEVKFNWYVDGV |
| | | | EVHNAKTKPREEQYASTYRVVSVLTVLHQDWLNGKEYKC |
| | | | KVSNKALPAPIEKTISKAKGQPREPQVCTLPPSREEMTKNQV |
| | | | SLSCAVKGFYPSDIAVEWESNGQPENNYKTTPPVLDSDGSF |
| | | | FLVSKLTVDKSRWQQGNVFSCSVMHEALHNHYTQKSLSLS |
| | | | P |
| | 187 | anti-TCRvβ | QVQLVQSGAEVKKPGSSVKVSCKASGTDFDKTYIHWVRQA |
| | | VH | PGQGLEWMGRISAGSGNVKYNEKFKGRVTITADTSTSTAY |
| | | | MELSSLRSEDTAVYYCAGSYYSYDVLDYWGQGTTVTVSS |
| | 4001 | Human IgG1 | ASTKGPSVFPLAPSSKSTSGGTAALGCLVKDYFPEPVTVSW |
| | | hole cys | NSGALTSGVHTFPAVLQSSGLYSLSSVVTVPSSSLGTQTYIC |
| | | N297A | NVNHKPSNTKVDKRVEPKSCDKTHTCPPCPAPELLGGPSVF |
| | | | LFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVCTLPPSREEMTKNQ |
| | | | VSLSCAVKGFYPSDIAVEWESNGQPENNYKTTPPVLDSDGS |
| | | | FFLVSKLTVDKSRWQQGNVFSCSVMHEALHNHYTQKSLSL |
| | | | SP |
| Chain 2 | 4002 | Full Sequence | APTSSSTKKTQLQLEHLLLDLQMILNGINNYKNPKLTRMLT |
| (IL2-Fc) | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLTGGGGSGGGGSDKTHTCPPCPAPELLGGPSVFLFPP |
| | | | KPKDTLMISRTPEVTCVVVDVSHEDPEVKFNWYVDGVEVH |
| | | | NAKTKPREEQYASTYRVVSVLTVLHQDWLNGKEYKCKVS |
| | | | NKALPAPIEKTISKAKGQPREPQVYTLPPCREEMTKNQVSL |
| | | | WCLVKGFYPSDIAVEWESNGQPENNYKTTPPVLDSDGSFFL |
| | | | YSKLTVDKSRWQQGNVFSCSVMHEALHNHYTQKSLSLSP |
| | 2270 | IL-2 C125A | APTSSSTKKTQLQLEHLLLDLQMILNGINNYKNPKLTRMLT |
| | | | FKFYMPKKATELKHLQCLEEELKPLEEVLNLAQSKNFHLRP |
| | | | RDLISNINVIVLELKGSETTFMCEYADETATIVEFLNRWITFA |
| | | | QSIISTLT |
| | 3308 | linker | GGGGSGGGGS |
| | 4003 | Human Fc | DKTHTCPPCPAPELLGGPSVFLFPPKPKDTLMISRTPEVTCV |
| | | Knob cys | VVDVSHEDPEVKFNWYVDGVEVHNAKTKPREEQYASTYR |
| | | N297A | VVSVLTVLHQDWLNGKEYKCKVSNKALPAPIEKTISKAKG |
| | | | QPREPQVYTLPPCREEMTKNQVSLWCLVKGFYPSDIAVEW |
| | | | ESNGQPENNYKTTPPVLDSDGSFFLYSKLTVDKSRWQQGN |
| | | | VFSCSVMHEALHNHYTQKSLSLSP |
| Chain 3 | 4017 | Full Sequence | DIQMTQSPSFLSASVGDRVTITCKASQNVDDRVAWYQQKP |
| (light chain) | | | GKAPKALIYSSSHRYKGVPSRFSGSGSGTEFTLTISSLQPEDF |
| | | | ATYFCQQFKSYPLTFGQGTKLEIKRTVAAPSVFIFPPSDEQL |
| | | | KSGTASVVCLLNNFYPREAKVQWKVDNALQSGNSQESVTE |
| | | | QDSKDSTYSLSSTLTLSKADYEKHKVYACEVTHQGLSSPVT |
| | | | KSFNRGEC |
| | 7513 | anti-TCRvβ | DIQMTQSPSFLSASVGDRVTITCKASQNVDDRVAWYQQKP |
| | | VL | GKAPKALIYSSSHRYKGVPSRFSGSGSGTEFTLTISSLQPEDF |
| | | | ATYFCQQFKSYPLTFGQGTKLEIK |
| | 3644 | Immunoglobulin | RTVAAPSVFIFPPSDEQLKSGTASVVCLLNNFYPREAKVQW |
| | | kappa | KVDNALQSGNSQESVTEQDSKDSTYSLSSTLTLSKADYEKH |
| | | constant C | KVYACEVTHQGLSSPVTKSFNRGEC |
| | | region | |

### EXAMPLES

### Example 1: Selective Depletion of TRBV+ T Cells In Vivo

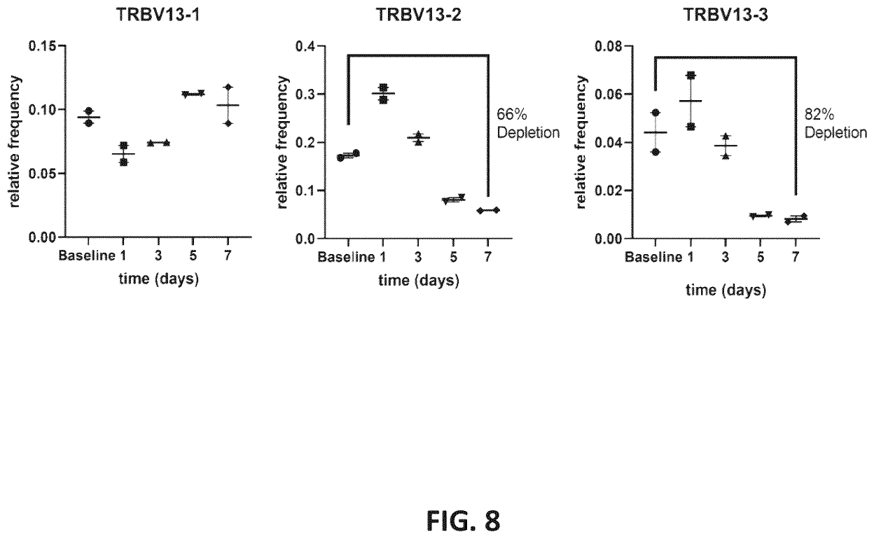

Naïve mice were treated with one injection of anti-Vβ13 (mouse) (Fc-wt) depleting antibody (heavy chain and light chain sequences as shown in SEQ ID NO: 7511 and 7512, respectively) and FACS analysis was performed on day 1, 3, 5 or 7 post-treatment. Leukocytes were isolated from spleens and processed for total RNA and TRBV repertoire was measured using a NanoString mouse TCRvβ profiling panel. The Vβ13 antibody had previously been shown to exhibit selectivity towards TRBV13-2 and 13-3 and 65-80 percent depletion of cells expressing these TRBV13 transcripts was observed (FIG. 8). The non-targeted TRBV13-1 was found to be unaffected by this depleting antibody. These results indicated an anti-TRBV antibody was effective to achieve selective depletion of the TRBV+ T cells (healthy and malignant).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1.-127. (canceled)

128. A method of treating a disease or condition in a subject in need thereof comprising: administering to the subject a therapeutically effective amount of an agent comprising a first moiety that binds to a first TCR variable region, wherein the disease or condition is ankylosing spondylitis and the first TCR variable region is TCRα V21.

129. The method of claim 128, wherein the agent comprises an antibody molecule or antigen binding domain thereof.

130. The method of claim 129, wherein the antibody molecule or antigen binding domain thereof comprises an Fc region.

131. The method of claim 130, wherein the Fc region comprises no binding mutation.

132. The method of claim 130, wherein the Fc region has enhanced effector function.

133. The method of claim 132, wherein the enhanced effector function comprises antibody dependent cell-mediated cytotoxicity (ADCC), antibody-dependent cellular phagocytosis (ADCP) or complement dependent cytotoxicity (CDC).

134. The method of claim 133, wherein the enhanced effector function mediates killing of one or more autoreactive T cells in the subject.

135. The method of claim 130, wherein the Fc region comprises a sequence having at least 90% sequence identity to the amino acid sequence as set forth in SEQ ID NO: 41.

136. The method of claim 128, wherein the agent further comprises a second moiety.

137. The method of claim 136, wherein the second moiety binds to a second TCR variable region.

138. The method of claim 137, wherein the second TCR variable region is TCRβ V9.

139. The method of claim 128, further comprising administering a second therapeutic agent to the subject.

140. The method of claim 139, wherein the second therapeutic agent comprises a second moiety that binds to a TCRβ V9 region.

141. The method of claim 130, wherein the agent further comprises an NK cell engager that binds to a cell surface protein of an NK cell, wherein the NK cell engager activates the NK cell.

142. The method of claim 141, wherein the cell surface protein is NKp30, NKp40, NKp44, NKp46, NKG2D, DNAM1, DAP10, CD16, CRTAM, CD27, PSGL1, CD96, CD100 (SEMA4D), NKp80, CD244, SLAMF6, SLAMF7, KIR2DS2, KIR2DS4, KIR3DS1, KIR2DS3, KIR2DS5, KIR2DS1, CD94, NKG2C, NKG2E, or CD160.

143. The method of claim 142, wherein the Fc region is a variant Fc region having an amino acid mutation.

144. The method of claim 143, wherein the amino acid mutation is configured to reduce or ablate affinity for at least one Fc receptor.

145. The method of claim 142, wherein the variant Fc region comprises a sequence having at least 90% sequence identity to the amino acid sequence as set forth in SEQ ID NO: 42.

146. A method of treating a disease or condition in a subject in need thereof comprising: administering to the subject a therapeutically effective amount of an agent comprising a first moiety that binds to a first TCR variable region, wherein the disease or condition is ankylosing spondylitis and the first TCR variable region is TCRβ V9, and wherein the first moiety comprises:
(a) a heavy chain variable (VH) domain comprising a heavy chain complementarity determining region 1 (HC CDR1) having a sequence of SEQ ID NO: 1248, a HC CDR2 having a sequence of SEQ ID NO: 1249, and a HC CDR3 having a sequence of SEQ ID NO: 1250, and a light chain variable (VL) domain comprising a light chain complementarity determining region 1 (LC CDR1) having a sequence of SEQ ID NO: 1254, a LC CDR2 having a sequence of SEQ ID NO: 1255, and a LC CDR3 having a sequence of SEQ ID NO: 1256;
(b) a VH domain comprising a HC CDR1 having a sequence of SEQ ID NO: 1251, a HC CDR2 having a sequence of SEQ ID NO: 1252, and a HC CDR3 having a sequence of SEQ ID NO: 1250, and a VL domain comprising a LC CDR1 having a sequence of SEQ ID NO: 1257, a LC CDR2 having a sequence of SEQ ID NO: 1255, and a LC CDR3 having a sequence of SEQ ID NO: 1256; or
(c) a VH domain comprising a HC CDR1 having a sequence of SEQ ID NO: 1253, a HC CDR2 having a sequence of SEQ ID NO: 1249, and a HC CDR3 having a sequence of SEQ ID NO: 1250, and a VL domain comprising a LC CDR1 having a sequence of SEQ ID NO: 1254, a LC CDR2 having a sequence of SEQ ID NO: 1255, and a LC CDR3 having a sequence of SEQ ID NO: 1256.

147. A method of treating a disease or condition in a subject in need thereof comprising: administering to the subject a therapeutically effective amount of an agent comprising a first moiety that binds to a first TCR variable region, wherein the disease or condition is Celiac disease and the first TCR variable region is TCRβ V4 or TCRβ V7.
